(19) United States
(12) Patent Application Publication
(10) Pub. No.: US 2022/0387608 A1
(43) Pub. Date: Dec. 8, 2022

(54) ADRENOMEDULLIN-ANALOGUES FOR LONG-TERM STABILIZATION AND THEIR USE

(71) Applicant: Bayer Aktiengesellschaft, Leverkusen (DE)

(72) Inventors: Johannes KÖBBERLING, Neuss (DE); Donald BIERER, Haan (DE); Ingo FLAMME, Reichshof (DE); Bernd RIEDL, Wuppertal (DE); Lucas Hudson HOFMEISTER, Berlin (DE); Annette BECK-SICKINGER, Leipzig (DE); Sylvia ELS-HEINDL, Leipzig (DE); Jan-Patrick FISCHER, Leipzig (DE); Eva-Maria JÜLKE, Leipzig (DE)

(73) Assignee: Bayer Aktiengesellschaft, Leverkusen (DE)

(21) Appl. No.: 17/596,655

(22) PCT Filed: Jun. 12, 2020
(86) PCT No.: PCT/EP2020/066253

§ 371 Date: Dec. 15, 2021

(30) Foreign Application Priority Data
Jun. 18, 2019 (EP) 19180955.7

(51) Int. Cl. A61K 47/64; A61K 47/54; A61K 31/616; A61K 45/06; A61P 9/12

(52) U.S. Cl. CPC A61K 47/64; A61K 47/542; A61K 47/545; A61K 31/616; A61K 45/06; A61P 9/12

(57) ABSTRACT

The invention relates to stabilized Adrenomedullin derivatives and use thereof. In particular, the invention relates to novel, biologically active, stabilized Adrenomedullin (ADM) compounds. The invention further relates to the compounds for use in a method for the treatment and/or prevention of diseases, especially of cardiovascular, edematous and/or inflammatory disorders, and to medicaments comprising the compounds for treatment and/or prevention of cardiovascular, edematous and/or inflammatory disorders.

The present invention relates to novel, biologically active, stabilized Adrenomedullin (ADM) peptide derivatives. The compounds of the invention are stabilized by substitution of the intramolecular disulfide bond and optionally one or more further modifications selected from replacement of amino acids by natural or unnatural amino acids, covalently linking the peptide derivative to a heterologous moiety selected from the group consisting of a polymer, a Fc, a FcRn binding ligand, albumin and an albumin-binding ligand, and N-methylation of at least one amide bond. The invention further relates to the compounds for use in a method for the treatment and/or prevention of diseases, especially of cardiovascular, edematous and/or inflammatory disorders, and to medicaments comprising the compounds for treatment and/or prevention of cardiovascular, edematous and/or inflammatory disorders.

The 52 amino acid peptide hormone adrenomedullin (ADM) is produced in adrenal gland, lung, kidney, heart muscle and other organs. The plasma levels of ADM are in the lower picomolar range. ADM is a member of the calcitonin gene-related peptide (CGRP) family of peptides and as such binds to a heterodimeric G-protein coupled receptor that consists of CRLR and RAMP 2 or 3 (Calcitonin-receptor-like receptor and receptor activity modifying protein 2 or 3). Activation of the ADM receptor leads to intracellular elevation of adenosine 3′, 5′-cyclic monophosphate (cAMP) in the receptor-bearing cells.

ADM receptors are present on different cell types in almost all organs including endothelial cells. ADM is thought to be metabolized by neutral endopeptidase and is predominantly cleared in the lung where ADM-receptors are highly expressed [for review see Gibbons C, Dackor R, Dunworth W, Fritz-Six K, Caron KM, Mol Endocrinol 21(4), 783-796 (2007)].

Experimental data from the literature suggests that ADM is involved in a variety of functional roles that include, among others, blood pressure regulation, bronchodilatation, renal function, hormone secretion, cell growth, differentiation, neurotransmission, and modulation of the immune response. Moreover ADM plays a crucial role as autocrine factor during proliferation and regeneration of endothelial cells [for review see Garcia M. A., Martin-Santamaria S., de Pascual-Teresa B., Ramos A., Juliin M., Martinez A., Expert Opin Ther Targets, 10(2), 303-317 (2006)].

There is an extensive body of evidence from the literature which shows that ADM is indispensable for an intact endothelial barrier function and that administration of ADM to supra-physiological levels exerts strong anti-edematous and anti-inflammatory functions in a variety of inflammatory conditions in animal experiments including sepsis, acute lung injury and inflammation of the intestine [for review see Temmesfeld-Wollbruck B, Hocke A., Suttorp N, Hippenstiel S, Thromb Haemost; 98, 944-951 (2007)].

Clinical testing of ADM was so far conducted in cardiovascular indications with a measurable hemodynamic end point such as pulmonary hypertension, hypertension, heart failure and acute myocardial infarction. ADM showed hemodynamic effects in several studies in patients suffering from the aforementioned conditions. However, effects were only short lasting and immediately ceasing after the end of administration. This findings correlated well with the known pharmacokinetic profile of ADM. Pharmacodynamic effects comprised among others lowering of systemic and pulmonary arterial blood pressure and increase of cardiac output [Troughton RW, Lewis LK, Yandle TG, Richards AM, Nicholls MG, Hypertension, 36(4), 588-93 (2000); Nagaya N, Kangawa K, Peptides., 25(11), 2013-8 (2004); Kataoka Y, Miyazaki S, Yasuda S, Nagaya N, Noguchi T, Yamada N, Morii I., Kawamura A, Doi K, Miyatake K, Tomoike H, Kangawa K, J Cardiovasc Pharmacol, 56(4), 413-9 (2010)].

In summary, based on evidence from a wealth of experimental data in animals and first clinical trials in man elevation of ADM to supraphysiological levels might be considered as a target mechanism for the treatment of a variety of disease conditions in man and animals. However, the major limitations of the use of ADM as therapeutic agent are the inconvenient applicability of continuous infusion therapy which precludes its use for most of the potential indications and the potentially limited safety margins with respect to hypotension which may result from bolus administrations of ADM.

The invention relates to novel biologically active, stabilized ADM peptide derivatives which can be employed for the treatment of diseases, in particular cardiovascular, edematous and inflammatory disorders.

Many therapeutically active peptides or proteins suffer from high clearance in vivo. Several approaches to increase the stability of therapeutically active peptides or proteins and reduce their clearance exist, including the alteration of disulfide bonds, N-methylation of amide bonds, and conjugation with heterologous moieties such as polymers and proteins.

Peptide therapeutics containing disulfide bonds may be problematic in their application in vivo. Disulfide bridges are unstable towards reducing agents and disulfide isomerases. Reduction of the disulfide bond results in a structural rearrangement and in a loss of activity. Protein-disulfide isomerase (PDI) is an enzyme of the endoplasmatic reticulum. Protein folding pathways contain intermediates with non-native disulfide bridges. The essential PDI function is to rearrange these intermediates to reach the final conformation [Laboissiere MC, Sturley SL, Raines RT, The essential function of protein-disulfide isomerase is to unscramble non-native disulfide bonds, J Biol Chem., 270(47), 28006-28009, 1995].

Glutathione (GSH) reacts with somatostatin to form mixed disulfides, further reaction with a second GSH molecule leads to the reduced dithiol form of somatostatin and GSSG. Thiol/disulfide exchange occurs readily; however, the formed mixed disulfides rapidly undergo reformation of the intramolecular disulfide bonds [Rabenstein DL, Weaver KH, Kinetics and equilibria of the thiol/disulfide exchange reactions of somatostatin with glutathione, J Org Chem., 61(21), 7391-7397, 1996]. The role of disulfide bonds in structural stability of peptides is described in Gehrmann J, Alewood PF, Craik DJ, Structure determination of the three disulfide bond isomers of α-conotoxin GI: a model for the role of disulfide bonds in structural stability, J Mol Biol., 278(2), 401-415, 1998.

Cystathiones are resistant towards thiol reduction. Therefore, substitutions of disulfides with thioethers are interesting in drug discovery, as they provide protection against reduction while the structure is only minimally perturbed. Thioether analogues of the complement inhibitor peptide compstatin were synthesized. The inhibitory potential was largely retained, whereas the stability to reduction was improved [Knerr PJ, Tzekou A, Ricklin D, Qu H, Chen H, van der Donk WA, Lambris JD, Synthesis and activity of thioether-containing analogues of the complement inhibitor compstatin, ACS Chem 10 Biol., 6(7), 753-760, 2011]. Peptide disulfide bond mimics based on diaminodiacids are described e.g. in Cui HK, Guo Y, He Y, Wang FL, Chang HN, Wang YJ, Wu FM, Tian CL, Liu L, Diaminodiacid-based solid-phase synthesis of peptide disulfide bond mimics, Angew Chem, 125, 9737-9741, 2013. Thioether and biscarba diaminodiacids were applied in the synthesis of peptide disulfide bond mimics of tachyplesin I analogues. The derivatives exhibited a decreased antimicrobial activity, but improved serum stability.

Kowalczyk R, Harris PW, Brimble MA, Callon KE, Watson M, Cornish J, Synthesis and evaluation of disulfide bond mimetics of amylin-(1-8) as agents to treat osteoporosis, Bioorg Med Chem., 20(8), 2661-2668, 2012, pertains to the octapeptide amylin. The native peptide (1-8) is stable for 6 month only at -80° C. under argon atmosphere. Analogues of the peptide were synthesized, wherein the disulfide bridge was modified either by the insertion of linkers or bridges of a different nature. All analogues were bench stable and therefore exhibited an improved stability. Muttenthaler M, Andersson A, de Araujo AD, Dekan Z, Lewis RJ, Alewood PF, Modulating oxytocin activity and plasma stability by disulfide bond engineering, J Med Chem., 53(24), 8585-8596, 2010, pertains to the synthesis of oxytocin analogues with disulfide bond replacements (thioether, selenosulfide, diselenide and ditelluride bridges) in order to improve the metabolic half-life of cysteine-containing peptides. Compared to oxytocin, some analogues retained affinity and functional potency and all mimetics exhibited an increase (1.5-3-fold) in plasma stability. Pakkala M, Weisell J, Hekim C, Vepsalainen J, Wallen EA, Stenman UH, Koistinen H, Närvänen A, Mimetics of the disulfide bridge between the N- and C-terminal cysteines of the KLK3-stimulating peptide B-2, Amino Acids., 39(1), 233-242, 2010, pertains to kallikrein-related peptidase 3 (KLK3). The proteolytic activity of kallikrein-related peptidase 3 (KLK3) is promoted by the synthetic cyclic, disulfide-bridged peptide B-2. Replacement of the disulfide with a lactam bridge between γ-butyric acid and aspartic acid was performed. The resulting peptide had an improved stability in plasma and against degradation by KLK3, as well as a higher activity than B-2 at high concentrations. Watkins HA, Rathbone DL, Barwell J, Hay DL, Poyner DR, Structure-activity relationships for α-calcitonin gene-related peptide, Br J Pharmacol., 170(7), 1308-1322, 2013, summarizes SAR studies performed with the α-calcitonin gene-related peptide (CGRP), the closest analogue of adrenomedullin. Referred is a disulfide mimic with a lactam as substitute (cyclo [Asp2, Lys7]-CGRP), which is originally described in: Dennis T, Fournier A, St Pierre S, Quirion R, Structure-activity profile of calcitonin gene-related peptide in peripheral and brain tissues. Evidence for receptor multiplicity. J Pharmacol Exp Ther., 251(2), 718-725, 1989. This peptide showed 50% decrease in affinity to the receptor in rat spleen membranes. Measurements of biological activity in guinea pig atria indicate a loss of agonist function.

Further, several approaches to form an injectable depot of such drugs exist that involve the use of macromolecules.

Polymer matrices that contain a drug molecule in a non covalently bound state are well known. These can also be injectable as gels, hydrogels, micro particles or micelles. The release kinetics of such drug products can be quite unreliable with high inter patient variability. Production of such polymers can harm the sensitive drug substance or it can undergo side reactions with the polymer during its degradation [D. H. Lee et al., J. Contr. Rel., 92, 291-299, 2003].

Permanent PEGylation of peptides or proteins to enhance their solubility, reduce immunogenicity and increase half live by reducing renal clearance is a well known concept since early 1980s [Caliceti P., Veronese F. M., Adv. Drug Deliv. Rev., 55, 1261-1277, 2003]. For several drugs this has been used with success, but with many examples the PEGylation reduces efficacy of drug substance to an extent that this concept is not suitable any more [T. Peleg-Shulman et al., J. Med. Chem., 47, 4897-4904, 2004]. Suitable alternative are polymer based prodrugs.

Several examples of PEG-based carrier prodrugs exist, most of them with the need for enzymatic activation of the linker between the active drug and the carrier, mostly initiated by enzymatic hydrolysis. Since esters are cleaved very readily and unpredictably in vivo, direct ester linkers for carrier pro drug have limitations to their usability [J. Rautio et al., Nature Reviews Drug discovery, 7, 255-270, 2008].

Commonly used alternative approaches are cascading linkers attached to an amine functionality in the peptide or protein. In cascading linkers a masking group has to be removed as the rate limiting step in the cascade. This activates the linker to decompose in a second position to release the peptide or protein.

Commonly the masking group can be removed by an enzymatic mechanism [R. B. Greenwald et al. in WO 2002/089789, Greenwald, et al., J. Med. Chem. 1999, 42, 3657-3667, F. M. H. DeGroot et al. in WO 2002/083180 and WO 2004/043493, and D. Shabat et al. in WO 2004/019993].

An alternative method not relying on enzymatic activation is the concept of U. Hersel et al. in WO 2005/099768. In their approach the masking group on a phenol is removed in a purely pH dependent manner by the attack of an internal nucleophile. This activates the linker for further decomposition.

As mentioned by U. Hersel et al. in WO 2005/099768, “The disadvantage in the abovementioned prodrug systems described by Greenwald, DeGroot and Shabat is the release of potentially toxic aromatic small molecule side products like quinone methides after cleavage of the temporary linkage. The potentially toxic entities are released in a 1:1 stoichiometry with the drug and can assume high in vivo concentrations.” The same problem holds true for the system by Hersel et al. as well.

For small organic molecules a plethora of different prodrug approaches exist [J. Rautio et al., Nature Reviews Drug discovery, 7, 255-270, 2008]. The approach used by U. Hersel et al. as release mechanism for their masking group has been used as a prodrug approach for phenolic groups of small molecules since the late 1980s. [W. S. Saari in EP 0 296 811 and W. S. Saari et al., J. Med. Chem., Vol 33, No 1, p 97-101, 1990].

Alternative amine based prodrug systems are based on the slow hydrolysis of bis-hydroxyethyl glycine as a cascading prodrug. The hydroxy groups of the bis-hydroxyethyl glycine are masked by esters that are prone to hydrolysis by esterases [R. Greenwald et al., J. Med. Chem., 47, 726-734, 2004, and D. Vetter et al. in WO 2006/136586].

Purely pH dependent cleavage of linkers is more reliable then enzymatic cleavage of linkers as it is not dependent on enzyme concentrations that may vary in living systems.

One concept for linkers that are cleaved pH dependently are prodrugs based on beta elimination with adjustable decomposition rates as described by Santi et al. in U.S. Pat. No. 8,680,315. The described linker technology to reversibly attach macromolecules to peptides and small molecules is applicable to several functional groups in the released drug. Amines, alcohols, carboxylic acids and thiols are attachable via an adaptor system to the beta eliminating moiety. Upon pH triggered decomposition the drug is released upon release of CO2 and an unsaturated fragment attached to the macromolecule.

Another approach optimized for phenols namely tyrosine in peptides is based on a carbamate that is pH dependently attacked by a nucleophilic amine under release of the phenol and generation of a cyclic urea attached to the macromolecule as described by Flamme I. et al in WO 2013/064455.

Further heterologous moieties established for the adjustment of the pharmacokinetic properties of peptides include polymers, including linear or branched C3-C100 carboxylic acids (lipidation), a polyethyleneglycol (PEG) moiety, a polypropylenglycol (PPG) moiety, a PAS moiety, which is an amino acid sequence comprising mainly alanine and serine residues or comprising mainly alanine, serine, and proline residues, the amino acid sequence forming random coil conformation under physiological conditions [US No. 2010/0292130 and WO 2008/155134], and a hydroxyethylstarch (HES) moiety [WO 02/080979], a Fc, a FcRn binding ligand, albumin and an albumin-binding ligand.

The adjustment of the pharmacokinetic properties of peptides by lipidation is a well-developed methodology. Lipidation can occur to the N-terminus or to the side chain functionalities of amino acids within the peptide sequence. Lipidation is described in a plethora of publications and patents as exemplified in the following reviews: Zhang L, Bulaj G, Converting peptides into drug leads by lipidation, Curr Med Chem.;19(11):1602-18, 2012, or M. Gerauer, S. Koch, H. Waldmann, L. Brunsveld, Lipidated peptide synthesis: Wiley Encyclopedia of Chemical Biology, Volume 2, 520-530, 2009, (Hrsg. Begley, T. P.). John Wiley & Sons, Hoboken, N.J. The lipidation of a truncated ADM fragment is described in WO 2012/138867.

Labeled Adrenomedullin derivatives for use as imaging and also therapeutic agent are known [J. Depuis et al. in CA 2567478 and WO 2008/138141]. In these ADM derivatives a complexating cage like molecular structure capable of binding radioactive isotopes was attached to the N terminus of ADM in a direct manner or via a spacer unit potentially also including short PEG spacers. The diagnostic or therapeutic value of these drugs arises from the targeted delivery of the radioactive molecule.

In contrast to the prodrug approaches listed above, which are all based on masking amine functionalities, another approach described in WO 2013/064508 is based on masking the phenolic group of a tyrosine in ADM. A carrier-linked prodrug is used, based on the internal nucleophile assisted cleavage of a carbamate on this phenolic group. The key advantage to other prodrug classes mentioned above is the toxicological harmlessness of the linker decomposition product, a cyclic urea permanently attached to the carrier. Furthermore, the decomposition of the prodrug is not dependent on enzymatic mechanisms that might cause a high inter patient variability of cleavage kinetics. The cleavage mechanism is solely pH dependent as an internal amine that is protonated at acidic pH gets activated at higher (neutral) pH to act as a nucleophile attacking the phenolic carbamate based on the tyrosine.

In the context of the present invention, stabilized, biologically active ADM peptide derivatives are now described wherein the disulfide bridging of the ADM peptide derivatives was replaced. Further modifications can be performed to peptide side chain by introducing further moieties as described below. Optionally, these modified ADM peptide derivatives were further modified by N-Methylation or by covalently linking the peptide derivative to a heterologous moiety selected from the group consisting of a polymer, an Fc, an FcRn binding ligand, albumin and an albumin-binding ligand. The polymer that is covalently linked to the peptide derivative is selected from the group consisting of optionally substituted, saturated, or mono- or di-unsaturated, linear or branched C3-C100 carboxylic acids, preferably C4-C30 carboxylic acids, a PEG moiety, a PPG moiety, a PAS moiety and a HES moiety. The analogues were investigated by means of activity and stability. It was shown that the activity of the ADM derivatives according to the invention is retained as compared to wt ADM. Further, the stabilized ADM peptide derivatives show an increased half-life in blood and liver, as can be shown by stability assays in serum and liver homogenates. The stabilized ADM derivatives according to the inventions show extended duration of pharmacological action as compared to ADM and on the basis of this specific action mechanism—after parenteral administration—exert in vivo sustained anti-inflammatory and hemodynamic effects such as stabilization of endothelial barrier function, and reduction of blood pressure, respectively.

The invention refers to a compound according to formula (I)

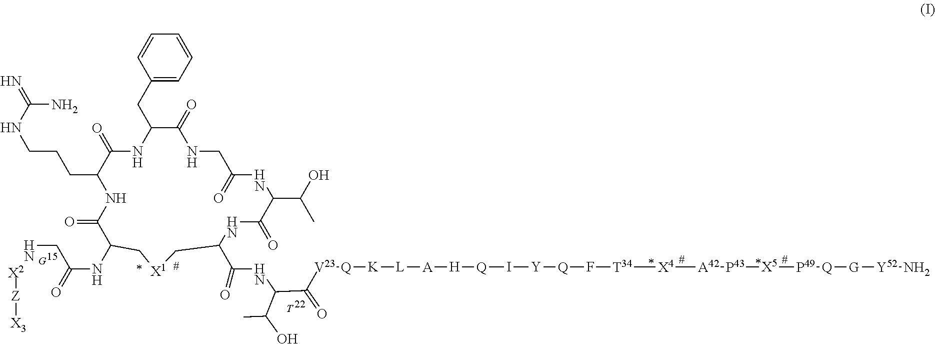

a physiologically acceptable salt, a solvate or a solvate of a salt thereof, wherein
X¹ is selected from the group consisting of
*—(CH₂)ₘ₁—S—S—(CH₂)ₙ₁—^{#}, wherein m1 is 0-6, n1 is 0-6, with the proviso that m1+n1=0-6;
*—(CH₂)ₘ₂—S—(CH₂)ₙ₂—^{#}, wherein m2 is 0-6, n2 is 0-6, with the proviso that m2+n2=0-6;
*—(CH₂)ₘ₃—#, wherein m3 is 1-8;
*—(CH₂)ₘ₄—(CH₂═CH₂)—(CH₂)ₙ₃—#, wherein m4 is 0-6, n3 is 0-6, with the proviso that m4+n3=0-6;
*—(CH₂)ₘ₅—(CH—CH)—(CH₂)ₙ₄—#, wherein m5 is 0-6, and n4 is 0-6, with the proviso that m5+n4=0-6;
*—(CH₂)ₘ₆—CO—NH—(CH₂)ₙ₅—^{#} wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
^{#}—(CH₂)ₘ₈—SO—(CH₂)ₙ₇—*, wherein m8 is 0-4, and n7 is 0-4, with the proviso that m8+n7=0-6;
^{#}—(CH₂)ₘ₉—SO₂—(CH₂)ₙ₈—*, wherein m9 is 0-4, and n8 is 0-4, with the proviso that m9+n8=0-6;
*-5-6 membered heteroaryl-^{#};
*—(CH₂)ₘ₁₀—O—(CH₂)ₙ₉ #, wherein m10 is 0-6, n9 is 0-6, with the proviso that m10+n9=0-6;
*—(CH₂)ₘ₁₈—NH—CO—CH₂—NH—CO—(CH₂)ₙ₈—#, wherein m18 is 0-3, and n5 is 0 or 1, with the proviso that m18+n5=0-3; ^{#}—(CH₂)ₘ₁₉—NH—CO—CH₂—NH—CO—(CH₂)ₙ₆—*, wherein m19 is 0-3, and n6 is 0 or 1, with the proviso that m19+n6=0-3;
*—(CH₂)ₘ₂₀—NH—CO—CH(CH₃)—NH—CO—(CH₂)ₙ₇ #, wherein m20 is 0-3, and n7 is 0 or 1, with the proviso that m20+n7=0-3; ^{#}—(CH₂)ₘ₂₁—NH—CO—CH(CH₃)—NH—CO—(CH₂)ₙ₈—*, wherein m21 is 0-3, and n8 is 0 or 1, with the proviso that m21+n8=0-3;
*—(CH₂)ₘ₂₂—NH—CO—CH(CH₂—C(CH₃)₂)—NH—CO—(CH₂)ₙ₉—^{#}, wherein m22 is 0-3, and n9 is 0 or 1, with the proviso that m22+n9=0-3; ^{#}—(CH₂)ₘ₂₃—NH—CO—CH(CH₂—C(CH₃)₂)—NH—CO—(CH₂)ₙ₁₀—*, wherein m23 is 0-3, and n10 is 0 or 1, with the proviso that m23+n10=0-3;
*—(CH₂)ₘ₂₄—NH—CO—CH(CH(CH₃)C2H₅)—NH—CO—(CH₂)ₙ₁₁—^{#}, wherein m24 is 0-3, and n11 is 0 or 1, with the proviso that m24+n11=0-3; ^{#}—(CH₂)ₘ₂₅—NH—CO—CH(CH(CH₃)C2H₅)—NH—CO—(CH₂)ₙ₁₂—*, wherein m25 is 0-3, and n12 is 0 or 1, with the proviso that m25+n12=0-3;
*—(CH₂)ₘ₂₆—NH—CO—CH(CH₂(C₆H₅))—NH—CO—(CH₂)ₙ₁₃—^{#} wherein m26 is 0-3, and n13 is 0 or 1, with the proviso that m26+n13=0-3; ^{#}—(CH₂)ₘ₂₇—NH—CO—CH(CH₂(C₆H₅))—NH—CO—(CH₂)ₙ₁₄—*, wherein m27 is 0-3, and n14 is 0 or 1, with the proviso that m27+n14=0-3;
*—(CH₂)ₘ₂8—NH—CO—(CH₂)₃—NH—CO—(CH₂)ₙ₁₅—^{#}, wherein m28 is 0 or 1, and n15 is 0 or 1, with the proviso that m28+n15=0-1; ^{#}—(CH₂)ₘ₂₉—NH—CO—(CH₂)₃—NH—CO—(CH₂)ₙ₁₆—*, wherein m29 is 0 or 1, and n16 is 0 or 1, with the proviso that m29+n16=0-1;
*—(CH₂)ₘ₃₀—NH—CO—NH—(CH₂)ₙ₁₇—^{#}, wherein m30 is 0-5, and n17 is 0-5, with the proviso that m30+n17=0-5; ^{#}—(CH₂)ₘ₃₁—NH—CO—NH—(CH₂)ₙ₁₅—*, wherein m31 is 0-5, and n18 is 0-5, with the proviso that m31+n18=0-5;
*—(CH₂)ₘ₃₂—O—CO—NH—(CH₂)ₙ₁₉—^{#}, wherein m32 is 0-5, and n19 is 0-5, with the proviso that m32+n19=0-5; ^{#}—(CH₂)ₘ₃₃—O—CO—NH—(CH₂)ₙ₂₀—*, wherein m33 is 0-5, and n20 is 0-5, with the proviso that m33+n20=0-5;
*—(CH₂)ₘ₃₄—O—CO—O—(CH₂)ₙ₂₁—^{#}, wherein m34 is 0-5, and n21 is 0-5, with the proviso that m34+n21=0-5;
*—(CH₂)ₘ₃₅—NH—CO—(CH₂)ₙ₂₂—NH—(CH₂)ₚ₁—^{#}, wherein m35 is 0-4, n22 is 0-4, and p1 is 0-4, with the proviso that m35+n22+p1=0-4; and
*—(CH₂)ₘ₃₆—NH—CO—(CH═CH)—CO—NH—(CH₂)ₙ₂₃—^{#}, wherein m36 is 0-2, and n23 is 0-2, with the proviso that m36+n23=0-2;
wherein * and ^{#} reflect where X¹ is bound within the ring structure; and X² is absent, is hydrogen, or is an amino acid or an amino acid sequence selected from the group consisting of
G¹⁴, K¹⁴, F¹⁴, SEQ ID NO:1 [Y¹RQSMNNFQGLRSF¹⁴], SEQ ID NO:2 [R²QSMNNFQGLRSF¹⁴], SEQ ID NO:3 [Q³SMNNFQGLRSF¹⁴], SEQ ID NO:4 [S⁴MNNFQGLRSF¹⁴], SEQ ID NO:5 [M⁵NNFQGLRSF¹⁴], SEQ ID NO:6 [N⁶NFQGLRSF¹⁴], SEQ ID NO:7 [N⁷FQGLRSF¹⁴], SEQ ID NO:8 [F₈QGLRSF¹⁴], SEQ ID NO:9 [Q⁹GLRSF¹⁴], SEQ ID NO:10 [G¹⁰LRSF¹⁴], SEQ ID NO:11 [L¹¹RSF¹⁴], SEQ ID NO: 12 [R¹²SF¹⁴], and SEQ ID NO: 13 [S¹³F¹⁴], wherein any one of the SEQ ID NO:1 to SEQ ID NO: 13 is covalently linked between F¹⁴ of said sequences by an amide bond to the N-terminal G¹⁵ of the amino acid sequence of formula (I), wherein any amino acid of X² can optionally be replaced by a natural or unnatural amino acid;
wherein A is L-Alanine; R is L-Arginine; N is L-Asparagine; D is L-Aspartic acid; Q is L-Glutamine; G is L-Glycine; H is L-Histidine; I is L-Isoleucine; L is L-Leucine; K is L-Lysine; M is L-Methionine; F is L-Phenylalanine; P is L-Proline; S is L-Serine; T is L-Threonine; Y is L-Tyrosine; V is L-Valine;
X³ is absent or is a heterologous moiety which is covalently linked to the N-terminus or to a functional group of the side chain of any amino acid of X², to the N-terminus of G¹⁵ or to Z;
Z is absent or is a cleavable linker covalently bound between the N terminus of any amino acid of X² or of G¹⁵ and X³ or between a functional group of the side chain of any amino acid of X² and X³;
wherein if X³ is absent, then Z is also absent and X² is hydrogen or is an amino acid or amino acid sequence as defined above for X²;
wherein if X³ is a heterologous moiety, then X² is absent or is an amino acid or amino acid sequence as defined above for X²;
X⁴ is the amino sequence * [D³⁵ K³⁶ D³⁷ K³⁸ D³⁹ N⁴⁰ V⁴¹]#, wherein at least one amino acid of said sequence can optionally be replaced by a natural or unnatural amino acid and wherein * indicates the binding site to T³⁴ and # indicates the binding site to A⁴², or X⁴ is a moiety according to formula (A), wherein * indicates the binding site to T³⁴ and # indicates the binding site to A⁴²

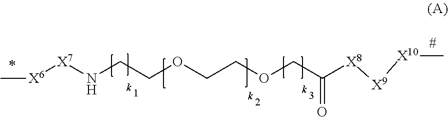

wherein X⁶, X⁷, X⁸, X⁹ and X¹⁰ are independently from another absent or an amino acid selected from L-Alanine; L-Arginine; L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or L-Valine,
wherein k1 is 1, 2, 3 or 4,
wherein k2 is 0, 1, 2, 3, 4, 5, 6, 7 or 8,
wherein k3 is 1, 2, 3 or 4,

X⁵ is the amino sequence * [R⁴⁴ S⁴⁵ K⁴⁶ I⁴⁷ S⁴⁸]#, wherein the sequence can optionally comprise at least one amino acid replaced by a natural or unnatural amino acid and wherein * indicates the binding site to P⁴³ and # indicates the binding site to P⁴⁹, or X⁵ is a moiety according to formula (B), wherein * and ^{#}reflect where X⁵ is bound within the amino acid chain and wherein * indicates the binding site of X⁵ to P⁴³ and # indicates the binding site to P⁴⁹,

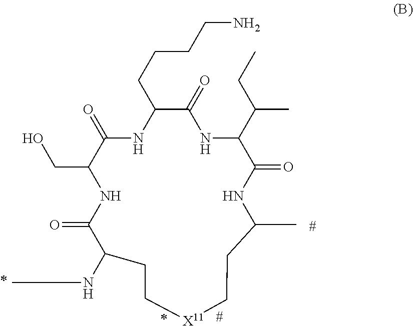

wherein X¹¹ is selected from the group consisting of
*—(CH₂)ₚ₁—S—(CH₂)ᵣ₁—^{#}, wherein p1 is 0-6; r1 is 0-6 with the proviso that p1+r1=0-6;
*—(CH₂)ₚ₂—O—(CH₂)ᵣ₂—^{#}, wherein p2 is 0-6; r2 is 0-6 with the proviso that p1+r2=0-6;
*—(CH₂)ₚ₃—^{#}, wherein p3 is 1-8;
*—(CH₂)ₚ₄—CO—NH—(CH₂)ᵣ₄—^{#}, wherein p4 is 0-4, and r4 is 0-4, with the proviso that p4+r4=0-6;
^{#}—(CH₂)ₚ₅—CO—NH—(CH₂)ᵣ₅*, wherein p5 is 0-4, and r5 is 0-4, with the proviso that p5+r5=0-6;
wherein * and ^{#} reflect where X¹¹ is bound within the ring structure;
wherein the numbering of amino acids in formula (I) refers to the corresponding human adrenomedullin (ADM) sequence;
wherein if X³ is not a di-carboxylic acid, then at least X⁴ is a moiety according to formula (A) as defined above and/or X⁵ is a moiety according to formula (B) as defined above.

The numbering of amino acids in formula (I) refers to the corresponding human adrenomedullin (ADM) sequence, which carries a disulfide bridge between C¹⁶-C²¹:

Y¹R²Q³S⁴M⁵N⁶N⁷F⁸Q⁹G¹⁰L¹¹R¹²S¹³F¹⁴C¹⁵C¹⁶R¹⁷F¹⁸G¹⁹T²⁰C²¹T²²V²³Q²⁴K²⁵L²⁶A²⁷H²⁸Q²⁹I30Y31Q³²F³³ T³⁴D³⁵K³⁶D³⁷K³⁸D³⁹N⁴⁰V⁴¹A⁴²P⁴³R⁴⁴S⁴⁵K⁴⁶I⁴⁷S⁴⁸P⁴⁹Q⁵⁰G¹⁵¹Y⁵²—NH₂

When comparing the human ADM sequence with formula (I), it can be seen that that that formula (I) is a modified and/or shorter ADM analogue. The stabilized ADM according to the invention, thus, show a lower molecular weight as compared to naturally occurring ADM.

When comparing formula (I) with the human sequence it can be seen that some parts of the formula (I) and the human ADM sequence match, whereas some parts of formula (I) are modified in comparison to the human ADM. For example the disulfide bridge between C¹⁶-C²¹ of the human ADM is modified to the bridge according to X¹.

There are reports that the substitution of disulfide bonds with lactam bridges, as well as the introduction of N-methylation and palmitoylation may increase the metabolic stability of the peptides while retaining the biological activity. However, as reported e.g. by Watkins HA, Rathbone DL, Barwell J, Hay DL, Poyner DR, Structure-activity relationships for α-calcitonin gene-related peptide, Br J Pharmacol. 2013, 170(7), 1308-1322 and Dennis T, Fournier A, St Pierre S, Quirion R, Structure-activity profile of calcitonin gene-related peptide in peripheral and brain tissues. Evidence for receptor multiplicity. 20 J. Pharmacol Exp Ther. 1989, 251(2), 718-725, the replacement of the disulfide bridge in members of the calcitonin superfamily of peptides was not correlated with retained activity. Also, while single changes of peptide structures are described, combinations of e.g. disulfide bond mimics, N-methylation and/or palmitoylation are not predictable with regard to structure-activity relationships.

ADM and other members of the calcitonin related peptides are known for fast inactivation by cleavage of the disulfide bridge. However, the activity retaining and at the same time half-life extending substitution of this disulfide bridge—even with alteration of the size of the intramolecular ring—is not known in the art and would not have been expected.

Compounds according to the invention are the compounds of the formula (I) and the salts thereof, solvates thereof and solvates of the salts thereof, the compounds which are embraced by formula (I) and are of the formulae specified below and the salts thereof, solvates thereof and solvates of the salts thereof, and the compounds which are embraced by formula (I) and are specified below as working examples and salts thereof, solvates thereof and solvates of the salts thereof, if the compounds which are embraced by formula (I) and are specified below are not already salts, solvates and solvates of the salts.

Depending on their structure, the compounds according to the invention may exist in stereoisomeric forms (enantiomers, diastereomers). The invention therefore embraces the enantiomers or diastereomers and the particular mixtures thereof. The stereoisomerically homogeneous constituents can be isolated in a known manner from such mixtures of enantiomers and/or diastereomers.

When the compounds according to the invention can occur in tautomeric forms, the present invention embraces all tautomeric forms.

Examples of stereoisomeric forms of the compounds of formula (I) according to the invention are compounds of the formulae (I) as defined above, wherein all amino acids have the L-configuration:

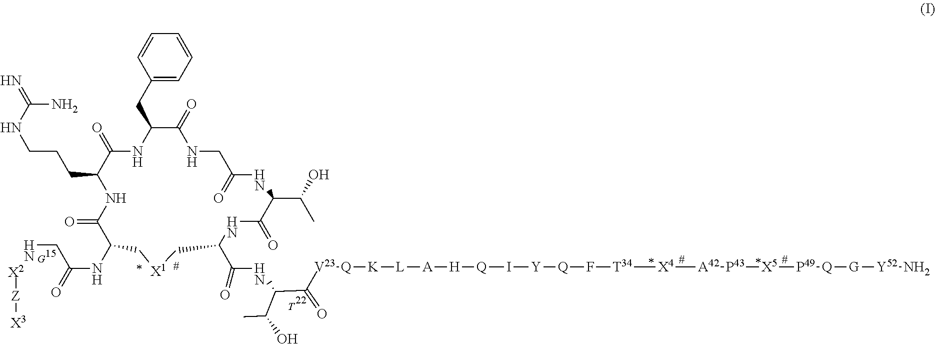

Examples of stereoisomeric forms of the compounds of formula (I), wherein X⁵ is a moiety according to formula (B) as defined above, wherein all amino acids have the L-configuration:

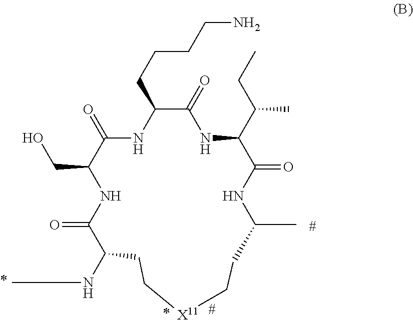

The present invention comprises all possible stereoisomeric forms, also in cases where no stereoisomerism is indicated.

The present invention also encompasses all suitable isotopic variants of the compounds of formula (I) according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of formula (I) according to the invention may therefore in some cases also constitute a preferred embodiment of the present invention. Isotopic variants of the compounds of formula (I) according to the invention can be prepared by processes known to those skilled in the art, for example by the methods described below and the methods described in the working examples, by using corresponding isotopic modifications of the particular reagents and/or starting compounds therein.

The current definitions for prodrugs by the IUPAC state the following terms [International Union of Pure and Applied Chemistry and International Union of Biochemistry: GLOSSARY OF TERMS USED IN MEDICINAL CHEMISTRY (Recommendations 1998); in Pure & Appl. Chem. Vol 70, No. 5, p. 1129-1143, 1998]:

The present invention moreover also includes prodrugs of the compounds of formula (I) according to the invention. The term “prodrugs” here designates compounds which themselves can be biologically active or inactive, but are converted (for example metabolically or hydrolytically) into compounds of formula (I) according to the invention during their dwell time in the body.

A carrier-linked prodrug or carrier prodrug is a prodrug that contains a temporary linkage of a given active substance with a transient carrier group that produces improved physicochemical or pharmacokinetic properties and that can be easily removed in vivo, usually by a hydrolytic cleavage.

A cascade prodrug is a prodrug for which the cleavage of the carrier group becomes effective only after unmasking an activating group.

In the context of the present invention, preferred salts are physiologically acceptable salts of the compounds according to the invention. Also included are salts which are not suitable themselves for pharmaceutical applications, but, for example, can be used for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluene-sulfonic acid, benzenesulfonic acid, naphthalenedisulfonic acid, acetic acid, trifluoroacetic acid, propionic acid, lactic acid, tartaric acid, maleic acid, citric acid, fumaric acid, maleic acid and benzoic acid.

Physiologically acceptable salts of the compounds according to the invention also include salts of customary bases, for example and with preference alkali metal salts (e.g. sodium and potassium salts), alkaline earth metal salts (e.g. calcium and magnesium salts) and ammonium salts derived from ammonia or organic amines having 1 to 16 carbon atoms, for example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methyl-morpholine, arginine, lysine, ethylenediamine and N-methylpiperidine.

In the context of the invention, solvates refer to those forms of the compounds according to the invention which, in the solid or liquid state, form a complex by coordination with solvent molecules. Hydrates are a specific form of the solvates, in which the coordination is with water. Preferred solvates in the context of the present invention are hydrates.

The specific radical definitions given in the particular combinations or preferred combinations of radicals are, irrespective of the particular combination of the radical specified, also replaced by any radical definitions of other combinations.

Very particular preference is given to combinations of two or more of the above or below mentioned preferred ranges.

Nomenclature of amino acids and peptide sequences is according to “International Union of Pure and Applied Chemistry and International Union of Biochemistry: Nomenclature and Symbolism for Amino Acids and Peptides (Recommendations 1983)”. In: Pure & Appl. Chem. 56, Vol. 5, 1984, p. 595-624

| Trivial | | One-letter |
|---|---|---|
| Name | Symbol | Symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Within the meaning of the present invention, natural amino acids are defined as peptidogenic amino acids. Within the meaning of the present invention, unnatural amino acids are defined as non-peptidogenic amino acids inserted in the peptides according to the invention, including: Diaminodiacids, which are within the meaning of this invention defined as amino acids having two amino and two carboxyl groups. Diaminodiacids can form amide bonds with two further amino acids.

Examples for diaminodiacids are cystathionine and 2,7-diaminosuberic acid; Diaminoacids, which are within the meaning of this invention defined as amino acids having a second amino group. Examples for Diaminoacids are 3-aminoalanine (Dpr), 2,4-diaminobutyric acid (Dab), alpha, gamma diamino butyric acid (Dbu), and 2,5 Diaminopentanoic acid (Orn); D-amino acids, heterocyclic substituted alanine being used as replacement for phenylalanine, and halogenated amino acids.

Within the meaning of the present invention, the term “heterologous moieties” includes a polymer, a Fc, a FcRn binding ligand, albumin and an albumin-binding ligand.

Within the meaning of the present invention, the term “Fc” is to be understood as immunoglobulin constant region or a portion thereof, such as an Fc region or a FcRn binding partner. In certain embodiments, the compound or conjugate is linked to one or more truncated Fc regions that are nonetheless sufficient to confer Fc receptor (FcR) binding properties to the Fc region. For example, the portion of an Fc region that binds to FcRn (i.e., the FcRn binding portion) comprises from about amino acids 282-438 of IgGI, EU numbering (with the primary contact sites being amino acids 248, 250-257, 272, 285, 288, 290-291, 308-311, and 314 of the CH₂ domain and amino acid residues 385-387, 428, and 433-436 of the CH₃ domain. Thus, an Fc region in a biologically active ADM peptide derivative of the invention may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used.

In certain embodiments, an Fc region comprises at least one of: a hinge (e.g., upper, middle, and/or lower hinge region) domain (about amino acids 216-230 of an antibody Fc region according to EU numbering), a CH2 domain (about amino acids 231-340 of an antibody Fc region according to EU numbering), a CH3 domain (about amino acids 341-438 of an antibody Fc region according to EU numbering), a CH4 domain, or a variant, portion, or fragment thereof. In other embodiments, an Fc region comprises a complete Fc domain (i.e., a hinge domain, a CH2 domain, and a CH3 domain). In some embodiments, an Fc region comprises, consists essentially of, or consists of a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), a CH2 domain (or a portion thereof) fused to both a hinge domain (or a portion thereof) and a CH3 domain (or a portion thereof). In still other embodiments, an Fc region lacks at least a portion of a CH2 domain (e.g., all or part of a CH2 domain). In a particular embodiment, an Fc region comprises or consists of amino acids corresponding to EU numbers 221 to 447.

An Fc in a biologically active ADM peptide derivative of the invention can include, for example, a change (e.g., a substitution) at one or more of the amino acid positions disclosed in Int'l. PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; U.S. Pat. Publ. Nos. US 2007/0231329, US2007/0231329, US2007/0237765, US2007/0237766, US2007/0237767, US2007/0243188, US2007/0248603, US2007/0286859, US2008/0057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

An Fc region used in the invention may also comprise an art recognized amino acid substitution which alters its glycosylation. For example, the Fc has a mutation leading to reduced glycosylation (e.g., N- or O-linked glycosylation) or may comprise an altered glycoform of the wild-type Fc moiety (e.g., a low fucose or fucose-free glycan).

According to a further embodiment of the invention, the heterologous moiety is a polyethyleneglycol (PEG) or polypropyleneglycol (PPG) moiety known in the art. The polymer can be of any molecular weight, and can be branched or unbranched.

For polyethylene glycol, in one embodiment, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a peptide or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa. In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al, Nucleosides Nucleotides 18:2745-2750 (1999); and Caliceti et al., Bioconjug. Chem. 10:638-646 (1999).

In other embodiments, the heterologous moiety is a PAS sequence. A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the procoagulant compound. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%>, i.e., about 9 of 100 amino acids, up to about 8%>, i.e., about 8 of 100 amino acids, about 6%>, i.e., about 6 of 100 amino acids, about 5%>, i.e., about 5 of 100 amino acids, about 4%>, i.e., about 4 of 100 amino acids, about 3%>, i.e., about 3 of 100 amino acids, about 2%>, i.e., about 2 of 100 amino acids, about 1%>, i.e., about 1 of 100 of the amino acids. The amino acids different from alanine, serine and proline may be selected from the group consisting of Arg, Asn, Asp, Cys, Gin, Glu, Gly, His, He, Leu, Lys, Met, Phe, Thr, Trp, Tyr, and Val. Under physiological conditions, the PAS sequence stretch forms a random coil conformation and thereby can mediate an increased in vivo and/or in vitro stability to procoagulant compound. Since the random coil domain does not adopt a stable structure or function by itself, the biological activity mediated by the Pep1 and/or Pep2 polypeptides in the procoagulant compound is essentially preserved. In other embodiments, the PAS sequences that form random coil domain are biologically inert, especially with respect to proteolysis in blood plasma, immunogenicity, isoelectric point/electrostatic behaviour, binding to cell surface receptors or internalisation, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA, AAPASPAPAAPSAPAPAAPS, APSSPSPSAPSSPSPASPSS, APSSPSPSAPSSPSPASPS, SSPSAPSPSSPASPSPSSPA, AASPAAPSAPPAAASPAAPSAPPA, and AS AAAP AAAS AAAS AP S AAA, or any combinations thereof. Additional examples of PAS sequences are known from, e.g., US Pat. Publ. No. 2010/0292130 A1 and PCT Appl. Publ. No. WO 2008/155134 A1.

In certain embodiments, the heterologous moiety is hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al, Arzneim.-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et ah, Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia.

The characteristics of VOLUVEN® are a mean molecular weight of 130,000+/−20,000 D, a molar substitution of 0.4 and a C2:C6 ratio of about 9: 1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be a mixture of hydroxyethyl starches having different mean molecular weights and/or different degrees of substitution and/or different ratios of C2: C6 substitution. Therefore, mixtures of hydroxyethyl starches may be employed having different mean molecular weights and different degrees of substitution and different ratios of C2: C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2: C6 substitution, or having about the same mean molecular weight and about the same degree of substitution and about the same ratio of C2:C6 substitution.

In certain embodiments, the heterologous moiety is a polysialic acids (PSAs) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp335-348. They can be produced in various degrees of polymerisation from n=about 80 or more sialic acid residues down to n=2 by limited acid hydrolysis or by digestion with neuraminidases, or by fractionation of the natural, bacterially derived forms of the polymer. The composition of different polysialic acids also vanes such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist-such as the alternating alpha-2,8 alpha-2,9 polysialic acid of E. coli strain K92 and group C polysaccharides of N. meningitidis. Sialic acid may also be found in alternating copolymers with monomers other than sialic acid such as group W 135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals. The alpha-2,8-linked polysialic acid of E. coli strain K¹ is also known as ‘colominic acid’ and is used (in various lengths) to exemplify the present invention. Various methods of attaching or conjugating polysialic acids to a peptide or polypeptide have been described (for example, see U.S. Pat. No. 5,846,951; WO-A-0187922, and US 2007/0191597 A1.

In certain embodiments, the heterologous moiety is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)n, (Gly4Ser)n or S(Gly4Ser)n, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200.

In certain aspects, a compound of the invention is covalently linked to at least one heterologous moiety that is or comprises an XTEN polypeptide or fragment, variant, or derivative thereof. As used here “XTEN polypeptide” refers to extended length polypeptides with non-naturally occurring, substantially non-repetitive sequences that are composed mainly of small hydrophilic amino acids, with the sequence having a low degree or no secondary or tertiary structure under physiologic conditions. As a heterologous moiety, XTENs can serve as a half-life extension moiety. In addition, XTEN can provide desirable properties including but are not limited to enhanced pharmacokinetic parameters and solubility characteristics.

The incorporation of a heterologous moiety comprising an XTEN sequence into a conjugate of the invention can confer one or more of the following advantageous properties to the resulting conjugate: conformational flexibility, enhanced aqueous solubility, high degree of protease resistance, low immunogenicity, low binding to mammalian receptors, or increased hydrodynamic (or Stokes) radii.

In certain aspects, an XTEN moiety can increase pharmacokinetic properties such as longer in vivo half-life or increased area under the curve (AUC), so that a compound or conjugate of the invention stays in vivo and has procoagulant activity for an increased period of time compared to a compound or conjugate with the same but without the XTEN heterologous moiety.

Examples of XTEN moieties that can be used as heterologous moieties in procoagulant conjugates of the invention are disclosed, e.g., in U.S. Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2.

In certain embodiments, the compound or conjugate of the invention is linked to a heterologous moiety comprising albumin or a functional fragment thereof. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ.

Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2.

In one embodiment, the heterologous moiety is albumin, a fragment, or a variant thereof which is further linked to a heterologous moiety selected from the group consisting of an immunoglobulin constant region or portion thereof (e.g., an Fc region), a PAS sequence, HES, and PEG.

In certain embodiments, the heterologous moiety is an albumin binding moiety, which comprises an albumin binding peptide, a bacterial albumin binding domain, an albumin-binding antibody fragment, or any combinations thereof.

For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa i -Xaa 2-Xaa 3-Xaa 4-Cys consensus sequence, wherein Xaa i is Asp, Asn, Ser, Thr, or Trp; Xaa 2 is Asn, Gin, H is, He, Leu, or Lys; Xaa 3 is Ala, Asp, Phe, Trp, or Tyr; and Xaa 4 is Asp, Gly, Leu, Phe, Ser, or Thr as described in US patent application 2003/0069395 or Dennis et al. (Dennis et al. (2002) J. Biol. Chem. 277, 35035-35043). Domain 3 from streptococcal protein G, as disclosed by Kraulis et al, FEBS Lett. 378: 190-194 (1996) and Linhult et al, Protein Sci. 11:206-213 (2002) is an example of a bacterial albumin-binding domain. Examples of albumin-binding peptides include a series of peptides having the core sequence DICLPRWGCLW (SEQ ID NO:45). See, e.g., Dennis et al, J. Biol. Chem. 2002, 277: 35035-35043 (2002). Examples of albumin-binding antibody fragments are disclosed in Muller and Kontermann, Curr. Opin. Mol. Ther. 9:319-326 (2007); Rooverset al, Cancer Immunol. Immunother. 56:303-317 (2007), and Holt et al, Prot. Eng. Design Sci., 21:283-288 (2008), which are incorporated herein by reference in their entireties. An example of such albumin binding moiety is 2-(3-maleimidopropanamido)-6-(4-(4-iodophenyl)butanamido) hexanoate (“Albu” tag) as disclosed by Trusselet al, Bioconjugate Chem. 20:2286-2292 (2009).

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or oxygen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, the term “at least one” means “1” or “one or more”.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein” or “disclosed herein”, it means that it may be mentioned or disclosed anywhere in the present text.

The term “halo”, “halogen” or “halogen atom” means a fluorine, chlorine, bromine or iodine atom, particularly a fluorine, chlorine or bromine atom.

Generally, terms such as “alkyl”, “hydroxyl”, “hydroxyl”, “amino”, “carboxy”, “carboxyl” are to understood as commonly used in the art unless otherwise defined in the present text.

A “di carboxylic acid” comprises two carboxyl functional groups (—COOH). The general molecular formula for dicarboxylic acids can be written as HO₂C—R—CO₂H, where R can be aliphatic or aromatic.

Other examples of dicarboxylic acids include aspartic acid and glutamic acid, two amino acids in the human body. The name can be abbreviated to diacid. Di carboxylic acid can be linear saturated dicarboxylic acids. Di carboxylic acid can be unsaturated dicarboxylic acids. Di carboxylic acid can be branched-chain dicarboxylic acids, substituted dicarboxylic acids, aromatic dicarboxylic acids.

For example, if a di carboxylic acid is
a C1-di carboxylic acid, then the acid is malonic acid,
a C2-di carboxylic acid, then the acid is succinic acid,
a C3-di carboxylic acid, then the acid is glutaric acid,
a C3-di carboxylic acid, then the acid is adipic acid,

and so on.

Di carboxylic acid used in the compounds according to invention can be includes e.g. as a moiety according to formula (C):

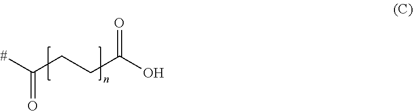

The moiety according to formula (C) can be modified to be a linear saturated dicarboxylic acid, unsaturated dicarboxylic acid, substituted dicarboxylic acid, aromatic dicarboxylic acid or branched-chain dicarboxylic acid. Examples of dicarboxylic acids are C16-di-carboxylic acid; C18-di-carboxyclic acid; C20-di-carboxyclic.

“C₁-C₆ alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2 dimethylbutyl or 1,3-dimethylbutyl group, or an isomer thereof. Particularly, said group has 1, 2, 3 or 4 carbon atoms (“C₁-C₄ alkyl”), e.g. a methyl, ethyl, propyl, isopropyl, butyl, sec-butyl isobutyl, or tert-butyl group, more particularly 1, 2 or 3 carbon atoms (“C₁-C₃ alkyl”), e.g. a methyl, ethyl, n-propyl or isopropyl group.

The term “C₁-C₆ hydroxyalkyl” means a linear or branched, saturated, monovalent hydrocarbon group in which the term “C₁-C₆ alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan- 2-yl, 3-hydroxy-2-methyl propyl, 2-hydroxy 2-methyl propyl, 1-hydroxy-2-methyl propyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, particularly 2, 3 or 4 carbon atoms (“C₂-C₄-alkenyl”), it being understood that in the case in which said alkenyl group contains more than one double bond, then it is possible for said double bonds to be isolated from, or conjugated with, each other, or to form an allene. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms. The analogous definition applies for other ranges mentioned herein, e.g. “C₁-C₃₀” (e.g. C3-C30 carboxylic acid or di carboxylic acid), “C₄-C₂₂” or “C₁₄-C₁₈” and so on.

When a range of values is given, said range encompasses each value and sub-range within said range. For example: “C₁-C₆” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;

All embodiments described herein can be independently from another combined.

According to an embodiment of the present invention, the compounds of formula (I) are further modified by N-methylation of at least one amide bond.

The influence of N-methylation on the metabolic stability of peptides has been described for various peptides. For example, cyclosporine is a naturally occurring, cyclic, multiply N-methylated peptide that exhibits an excellent pharmacokinetic profile. N-methylation in general blocks enzymatic degradation by proteases as they are unable to cleave N-methylated peptide bonds. Multiple N-methylation was shown to improve the metabolic stability and intestinal permeability of peptides [Chatterjee J, Gilon C, Hoffman A, Kessler H, N-methylation of peptides: a new perspective in medicinal chemistry, Acc Chem Res., 41(10), 1331-1342, 2008]. Cyclization combined with N-methylation was used to modulate physicochemical properties of peptides, including metabolic stability, membrane permeability and oral bioavailability [Chatterjee J, Laufer B, Kessler H, Synthesis of N-methylated cyclic peptides, Nat Protoc., 7(3), 432-444, 2012]. Dong QG, Zhang Y, Wang MS, Feng J, Zhang HH, Wu YG, Gu TJ, Yu XH, Jiang CL, Chen Y, Li W, Kong W, Improvement of enzymatic stability and intestinal permeability of deuterohemin-peptide conjugates by specific multi-site N-methylation, Amino Acids., 43(6), 2431-2441, 2012, describe that N-Methylation at selected sites showed high resistance against proteolytic degradation. In diluted serum and intestinal preparation 50- to 140-fold higher half-life values were observed. However, Linde Y, Ovadia 0, Safrai E, Xiang Z, Portillo FP, Shalev DE, Haskell-Luevano C, Hoffman A, Gilon C, Structure-activity relationship and metabolic stability studies of backbone cyclization and N-methylation of melanocortin peptides, Biopolymers., 90(5), 671-682, 2008, describe that cyclic N-methylated analogues of the α-melanocyte stimulating hormone were more stable, however less biologically active than the parent peptide.

In one embodiment the compound has compounds of formula (I) according to the invention are compounds of the formula (I) as defined above having the following stereoisomerism L-configuration

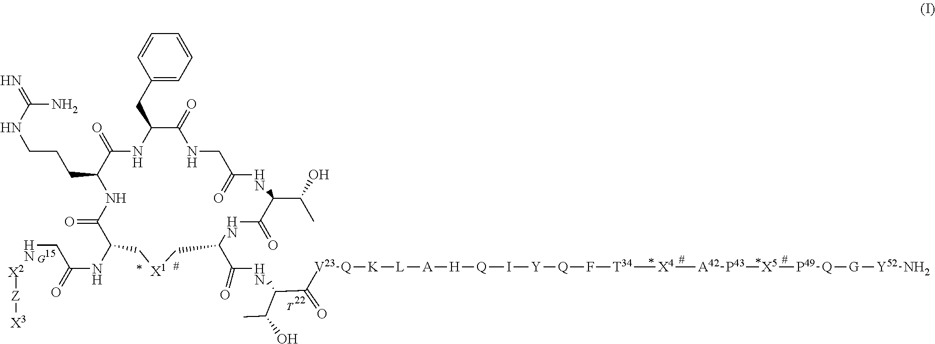

In one embodiment the compound has compounds of formula (I) according to the invention, wherein X⁵ is a moiety according to formula (B) as defined above having the following stereoisomerism L-configuration

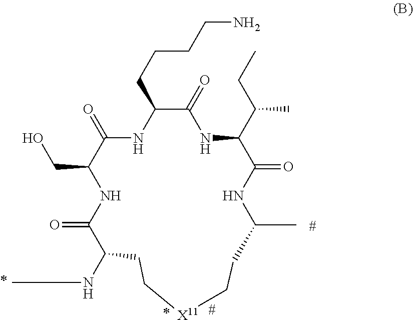

According to an embodiment of the invention, the compounds of formula (I), a physiologically acceptable salt, a solvate or a solvate of a salt thereof are defined as follows:

X¹ is selected from

*—(CH₂)ₘ₁—S—S—(CH₂)ₙ₁—^{#}, wherein m1 is 0-6, n1 is 0-6, with the proviso that m1+n1=0-6;

*—(CH₂)ₘ₂—S—(CH₂)ₙ₂—^{#}, wherein m2 is 0-6, n2 is 0-6, with the proviso that m2+n2=0-6;

*—(CH₂)ₘ₃—^{#}, wherein m3 is 1-8;

*—(CH₂)ₘ₆—CO—NH—(CH₂)ₙ₈—^{#}, wherein m6 is 0-4, and n5 is 0-4, with the proviso that m6+n5=0-6;

^{#}—(CH₂)ₘ₇—CO—NH—(CH₂)ₙ₆—*, wherein m7 is 0-4, and n6 is 0-4, with the proviso that m7+n6=0-6;

*—(CH₂)ₘ₁₀—O—(CH₂)ₙ₉—^{#}, wherein m10 is 0-6, n9 is 0-6, with the proviso that m10+n9=0-6,

wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is selected from

*—(CH₂)ₘ₁—S—S—(CH₂)ₙ₁—^{#}, wherein m1 is 0, 1 or 2, n1 is 0, 1 or 2, with the proviso that m1+n1=0-6;

*—(CH₂)ₘ₂—S—(CH₂)ₙ₂—^{#}, wherein m2 is 0, 1 or 2, n2 is 0, 1 or 2, with the proviso that m2+n2=0-6;

*—(CH₂)ₘ₃—^{#}, wherein m3 is 0, 1 or 2;

*—(CH₂)ₘ₆—CO—NH—(CH₂)ₙ₈—^{#}, wherein m6 is 0, 1 or 2, and n5 is 0, 1 or 2, with the proviso that m6+n5=0-6;

^{#}—(CH₂)ₘ₇—CO—NH—(CH₂)ₙ₆—*, wherein m7 is 0, 1 or 2, and n6 is 0, 1 or 2, with the proviso that m7+n6=0-6;

*—(CH₂)ₘ₁₀—O—(CH₂)ₙ₉—^{#}, wherein m10 is 0, 1 or 2, n9 is 0, 1 or 2, with the proviso that m10+n9=0-6,

wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is *—(CH₂)ₘ₁—S—S—(CH₂)ₙ₁—^{#}, wherein m1 is 0-6, n1 is 0-6 with the proviso that m1+n1=0-6, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, 1 or 2 and n1 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, 1 or 2 and n1 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₁—S—S—(CH₂)ₙ₁—*, wherein m1 is 0, 1 or 2 and n1 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, 1 or 2 and n1 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₁—S—S—(CH₂)ₙ₁—*, wherein m1 is 0, or 1 and n1 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, or 1 and n1 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, or 1 and n1 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0, or 1 and n1 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0 and n1 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0 and n1 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0 and n1 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 0 and n1 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1, or 2 and n1 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1, or 2 and n1 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1 and n1 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1 and n1 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1 and n1 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₁—S—S—(CH₂)ₙ₁—*, wherein m1 is 1 and n1 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is *—(CH₂)ₘ₆—CO—NH—(CH₂)ₙ₅—^{#}, wherein m6 is 0-6, n5 is 0-6 with the proviso that m6+n5=0-6, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

According to an embodiment of the invention, the compounds of formula (I), a physiologically acceptable salt, a solvate or a solvate of a salt thereof are defined as follows: X¹ is ^{#}—(CH₂)ₘ₆—CO—NH—(CH₂)ₙ₅*, wherein m6 is 0, 1 or 2 and n5 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅*, wherein m6 is 0, 1 or 2 and n5 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0, 1 or 2 and n5 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0, 1 or 2 and n5 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0, or 1 and n5 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅*, wherein m6 is 0, or 1 and n5 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0, or 1 and n5 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0, or 1 and n5 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0 and n5 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0 and n5 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0 and n5 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 0 and n5 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 1, or 2 and n5 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅*, wherein m6 is 1, or 2 and n5 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 1 and n5 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 1 and n5 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 1 and n5 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is ^{#}—(CH₂)ₘ₆—CO—NH—(CH₂)ₙ₅—*, wherein m6 is 1 and n5 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0-6, n6 is 0-6 with the proviso that m7+n6=0-6, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, 1 or 2 and n6 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, 1 or 2 and n6 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, 1 or 2 and n6 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, 1 or 2 and n6 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, or 1 and n6 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, or 1 and n6 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, or 1 and n6 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0, or 1 and n6 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0 and n6 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0 and n6 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0 and n6 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 0 and n6 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1, or 2 and n6 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1, or 2 and n6 is 0 or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1 and n6 is 0, 1 or 2, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1 and n6 is 0, or 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1 and n6 is 1, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X¹ is ^{#}—(CH₂)ₘ₇—CO—NH—(CH₂)ₙ₆—*, wherein m7 is 1 and n6 is 0, and wherein * and ^{#} reflect where X¹ is bound within the ring structure, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X² is X² is absent, is hydrogen, or is an amino acid or an amino acid sequence selected from the group consisting of G¹⁴ and K¹⁴, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X² is G¹⁴ or K¹⁴, which is covalently linked by an amide bond to the N-terminal G¹⁵ of the compound of formula (I).

According to an embodiment of the invention, the compounds of formula (I), a physiologically acceptable salt, a solvate or a solvate of a salt thereof are defined as follows: X³ is a heterologous moiety selected from the group consisting of a polymer, a Fc, a FcRn binding ligand, albumin and an albumin-binding ligand; or a physiologically acceptable salt, a solvate or a solvate of a salt thereof, and wherein X², X³, X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X³ is a moiety according to Formula (C)

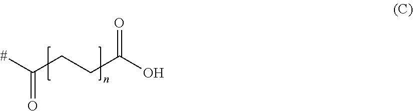

wherein n is 1 to 15, and wherein X¹, X², X⁴ and X⁵ and are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 2 to 11, and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 4 to 10, and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 6 to 9, preferably 7 to 9, and wherein X, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 7 to 8, and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 6 and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 7 and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 8 and wherein X¹, X², X⁴ and X⁵ are as defined according to any one of the proceeding embodiments disclosed above or below and wherein # indicates the binding site to Z. If Z is absent, # indicates the binding site to X².

According to an embodiment of the invention, the compounds of formula (I), a physiologically acceptable salt, a solvate or a solvate of a salt thereof are as defined as follows: X³ is a moiety according to Formula (C), wherein n is 9 and wherein X¹, X², X⁴ and X⁵ are as defined according to any one of the proceeding embodiments disclosed above or below and wherein # indicates the binding site to Z. If Z is absent, # indicates the binding site to X².

Within the meaning of the present invention, the term “heterologous moieties” includes a polymer, a Fc, a FcRn binding ligand, albumin and an albumin-binding ligand.

According to an embodiment of the invention, the compounds of formula (I), a physiologically acceptable salt, a solvate or a solvate of a salt thereof are defined as follows: X³ is a polymer and the polymer is selected from the group consisting of linear or branched C₁-C₁₀₀ carboxylic acids and carboxylic di-acids, preferably C₄-C₃₀ carboxylic acids and carboxylic di-acids, optionally substituted with halo, hydroxy, alkoxy, amino, alkylamino, dialky lamino, sulfate, or phosphate, and which may be saturated, or mono- or di-unsaturated, a PEG moiety, a PPG moiety, a PAS moiety and a HES moiety; or a physiologically acceptable salt, a solvate or a solvate of a salt thereof, and wherein X¹, X², X⁴, X⁵ and Z are as defined according to any one of the proceeding embodiments disclosed above or below.

In one embodiment according to the invention, X³ can be halo or halogen, di carboxylic acid, C₁-C₆ alkyl, C₁-C₆ hydroxyalkyl, C₁-C₆-haloalkyl, C₁-C₆-alkoxy, C₁-C₆-haloalkoxy, C₂-C₆-alkenyl. The definitions and specific embodiments are disclosed above.

According to an embodiment of the invention, the compounds of formula (I), a physiologically acceptable salt, a solvate or a solvate of a salt thereof are defined as follows: X³ is selected from the group consisting of linear or branched C3-C30 carboxylic acid or di carboxylic acid, preferably C4-C20 carboxylic acids or di carboxylic acid, more preferably C16-C18 carboxylic acid or di-carboxylic acid, most preferably C16-C18 di carboxylic acid, optionally substituted with halo, hydroxy, alkoxy, amino, alkylamino, dialkylamino, sulfate, or phosphate, and which may be saturated, or mono- or di-unsaturated, a PEG moiety, a PPG moiety, a PAS moiety and a HES moiety, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: the carboxylic acid is selected from the group consisting of arachidic acid, arachidonic acid, behenic acid, capric acid, caproic acid, caprylic acid, ceroplastic acid, cerotic acid, docosahexaenoic acid, eicosapentaenoic acid, elaidic acid, enanthic acid, erucic acid, geddic acid, hen atriacontylic acid, heneicosylic acid, heptacosylic acid, hexatriacontylic acid, lacceroic acid, lauric acid, lignoceric acid, linoelaidic acid, linoleic acid, margaric acid, melissic acid, montanic acid, myristic acid, myristoleic acid, nona cosylic acid, nonadecylic acid, oleic acid, palmitic acid, palmitoleic acid, pantothenic acid, pelargonic acid, penta cosylic acid, pentadecylic acid, psyllic acid, sapienic acid, stearic acid, tricosylic acid, tridecylic acid, undecylic acid, vaccenic acid, valeric acid, a—linolenic acid, C14-C22 carboxylic di-acids and derivatives thereof; or a physiologically acceptable salt, a solvate or a solvate of a salt thereof, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X³ is a carboxylic di-acid, preferably a C14-C22 carboxylic di-acid, more preferably a C14-C18 carboxylic di-acid or derivatives thereof, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X³ is a C-14 carboxylic di-acid or derivatives thereof, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X³ is a C-16 carboxylic di-acid or derivatives thereof, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X³ is a C-18 carboxylic di-acid or derivatives thereof, and wherein X¹, X², X⁴, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X² is G¹⁴ or K¹⁴, which is covalently linked by an amide bond to the N-terminal G¹⁵ of the compound of formula (I);

X³ is absent or is a heterologous moiety, which is covalently linked to the N-terminus of G¹⁴ or K¹⁴ or to a functional group of the side chain of K¹⁴, or to Z;

Z is absent or is a cleavable linker covalently bound between the N terminus of G¹⁴ or K¹⁴ and X³, or between a functional group of the side chain of K¹⁴ and X³;

wherein if X³ is absent, then Z is also absent;

wherein if X³ is a heterologous moiety, then Z is absent or is a cleavable linker covalently bound between the N terminus of G¹⁴ or K¹⁴ and X³, or between a functional group of the side chain of K¹⁴ and X³,

and wherein X¹ is as defined above.

According to an embodiment of the invention, the compounds of formula (I), a physiologically acceptable salt, a solvate or a solvate of a salt thereof are defined as follows: X³ is absent or X³ is a dicarboxylic acid.

According to an embodiment of the invention, the compounds of formula (I), a physiologically acceptable salt, a solvate or a solvate of a salt thereof are defined as follows:

X² is G¹⁴ or K¹⁴, which is covalently linked by an amide bond to the N-terminal G¹⁵ of the compound of formula (I);

X³ is absent;

Z is absent;

and wherein X¹ is as defined above.

According to an embodiment of the invention, the compounds of formula (I), a physiologically acceptable salt, a solvate or a solvate of a salt thereof are defined as follows:

X⁴ is the amino sequence *[D³⁵ K³⁶ D³⁷ K³⁸ D³⁹ N⁴⁰ V⁴¹]#, wherein * indicates the binding site to T³⁴ and # indicates the binding site to A⁴², and wherein X¹, X², X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is the following amino sequence * [D³⁵ K³⁶ D³⁷ K³⁸ D³⁹ N⁴⁰ V⁴¹]#, wherein * indicates the binding site to T³⁴ and # indicates the binding site to A⁴², wherein one or more of the amino acids of said sequence is substituted by a natural or unnatural amino acid, and wherein X¹, X², X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X⁴ is the following amino sequence * [D³⁵ K³⁶ D³⁷ K³⁸ D³⁹ N⁴⁰ V⁴¹]#, wherein * indicates the binding site to T³⁴ and # indicates the binding site to A⁴², wherein V⁴¹ is substituted by a natural or unnatural amino acids and/or A⁴² is substituted by natural or unnatural amino acids, and wherein X¹, X², X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X⁴ is a moiety according to formula (A)

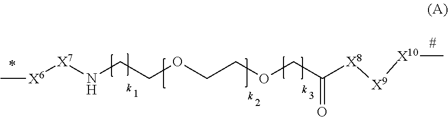

wherein X⁶, X⁷, X⁸, X⁹ and X¹⁰ are independently from another absent or an amino acid selected from L-Alanine; is L-Arginine; is L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or V is L-Valine,

^{#} reflect where X⁴ is bound within the amino acid chain, wherein * indicates the binding site to T³⁴ and # indicates the binding site to A⁴²

and wherein X¹, X², X³, X⁵, Z, k1, k2 and k3 are as defined according to any one of the proceeding embodiments disclosed above or below.

According to an embodiment of the invention, the compounds of formula (I), a physiologically acceptable salt, a solvate or a solvate of a salt thereof are defined as follows:

X⁴ is a moiety according to formula (A)

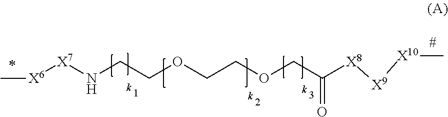

wherein X⁶, X⁷, X⁸, X⁹ and X¹⁰ are independently from another absent or an amino acid selected from L-Alanine; is L-Arginine; is L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or V is L-Valine,

wherein k1 is 1 or 2; wherein k2 is 0, 1, 2, 3, or 4; wherein k3 is 1 or 2, and ^{#} reflect where X⁴ is bound within the amino acid chain, wherein * indicates the binding site to T³⁴ and # indicates the binding site to A⁴², and wherein X¹, X², X³, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)×

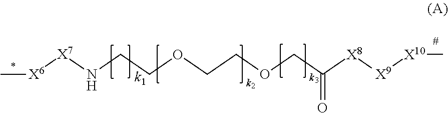

wherein X⁶ is absent or selected from the group consisting of D, N and V;

wherein X⁷ is absent or is selected from the group consisting of D, N and V;

wherein X⁸ is absent or is selected from the group consisting of D, N and V;

wherein X⁹ is absent or is selected from the group consisting of D, N and;

and wherein X¹⁰ is absent or is selected from the group consisting of D, N and V;

wherein k1 is 1 or 2; wherein k2 is 0, 1, 2, 3, or 4; wherein k3 is 1 or 2,

and ^{#} reflect where X⁴ is bound within the amino acid chain, wherein * indicates the binding site to T³⁴ and # indicates the binding site to A⁴², and wherein X¹, X², X³, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)

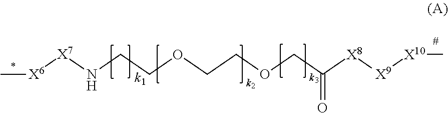

wherein k1 is 1; wherein k2 is 2, 3, or 4; wherein k3 is 1 or 2,

wherein * and ^{#} reflect where X⁴ is bound within the amino acid chain, wherein * indicates the binding site to T³⁴ and # indicates the binding site to A⁴², and wherein X¹, X², X³, X⁵ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)

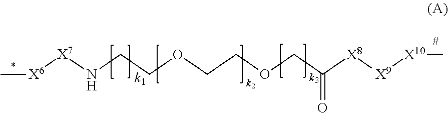

wherein k1 is 1; wherein k2 is 2; wherein k3 is 1,

wherein * and ^{#} reflect where X⁴ is bound within the amino acid chain, wherein * indicates the binding site to T³⁴ and # indicates the binding site to A⁴², and wherein X¹, X², X³, X⁵, Z, X⁶, X⁷, X⁸, X⁹ and X¹⁰ are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)

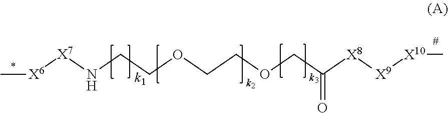

wherein k1 is 1; wherein k2 is 3; wherein k3 is 2,

wherein * and ^{#} reflect where X⁴ is bound within the amino acid chain, wherein * indicates the binding site to T³⁴ and # indicates the binding site to A⁴², and wherein X¹, X², X³, X⁵, Z, X⁶, X⁷, X⁸, X⁹ and X¹⁰ are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)

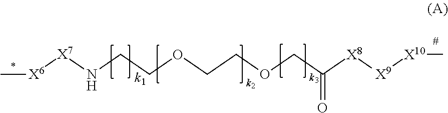

wherein k1 is 1; wherein k2 is 4; wherein k3 is 2,

wherein * and ^{#} reflect where X⁴ is bound within the amino acid chain, wherein * indicates the binding site to T³⁴ and # indicates the binding site to A⁴², and wherein X¹, X², X³, X⁵, Z, X⁶, X⁷, X⁸, X⁹ and X¹⁰ are as defined according to any one of the proceeding embodiments disclosed above or below.

According to an embodiment of the invention, the compounds of formula (I), a physiologically acceptable salt, a solvate or a solvate of a salt thereof are defined as follows:

X⁴ is a moiety according to formula (A)

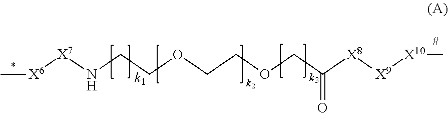

wherein X⁶ is absent or selected from the group consisting of D, N and V;

wherein X⁷ is absent or is selected from the group consisting of D, N and V;

wherein * and ^{#} reflect where X⁴ is bound within the amino acid chain, wherein * indicates the binding site to T³⁴ and # indicates the binding site to A⁴², and wherein X¹, X², X³, X⁵, Z, X⁸, X⁹, X¹⁰, k1, k2 and 15 k3 are as defined according to any one of the proceeding embodiments disclosed above or below.

According to an embodiment of the invention, the compounds of formula (I), a physiologically acceptable salt, a solvate or a solvate of a salt thereof are defined as follows: X⁴ is a moiety according to formula (A)

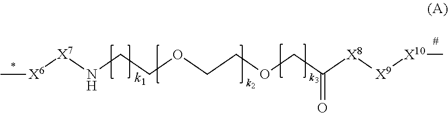

wherein X⁸ is absent or is selected from the group consisting of D, N and V;

wherein X⁹ is absent or is selected from the group consisting of D, N and;

and wherein X¹⁰ is absent or is selected from the group consisting of D, N and V;

and ^{#} reflect where X⁴ is bound within the amino acid chain, wherein * indicates the binding site to T³⁴ and # indicates the binding site to A⁴², and wherein X¹, X², X³, X⁵, Z, X⁶, X⁷, k1, k2 and k3 are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the amino sequence *[R⁴⁴ S⁴⁵ K⁴⁶ I⁴⁷ S⁴⁸]#, wherein * indicates the binding site to P⁴³ and # indicates the binding site to P⁴⁹ and wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the amino sequence *[R⁴⁴ S⁴⁵ K⁴⁶ I⁴⁷ S⁴⁸]#, wherein * indicates the binding site to P⁴³ and # indicates the binding site to P49, wherein at least one amino acid of said sequence is substituted by a natural or unnatural amino acid, and wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the amino sequence *[R⁴⁴ S⁴⁵ K⁴⁶ I⁴⁷ S⁴⁸]#, wherein * indicates the binding site to P⁴³ and # indicates the binding site to P⁴⁹, wherein S⁴⁵ and/or S^{4U} are independently substituted by a natural or unnatural amino acid, and wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according to formula (B), wherein * and ^{#} reflect where X⁵ is bound within the amino acid chain and wherein * indicates the binding site of X⁵ to P⁴³ and # indicates the binding site to P⁴⁹,

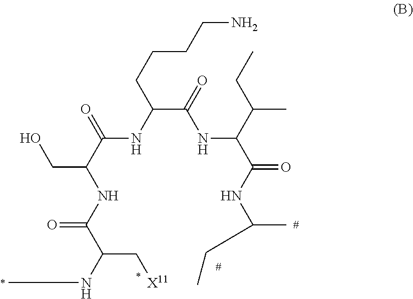

wherein X¹¹ is selected from the group consisting of

*—(CH₂)ₚ₁—S—(CH₂)ᵣ₁ #, wherein p1 is 0-4 and r1 is 0 or 1;

#—(CH₂)ₚ₂—S—(CH₂)ᵣ₂*, wherein p2 is 0-4 and r2 is 0 or 1;

*—(CH₂)ₚ₃—^{#}, wherein p3 is 1-4;

*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;

#—(CH₂)ₚ₅—CO—NH—(CH₂)ᵣ₅*, wherein p5 is 0, 1, 2 or 3, and r5 is 0, 1, 2 or 3, with the proviso that p5+r5=0-4;

wherein * and ^{#} reflect where X¹¹ is bound within the ring structure, and

wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according 15 to formula (B), wherein * and ^{#} reflect where X⁵ is bound within the amino acid chain and wherein * indicates the binding site of X⁵ to P⁴³ and # indicates the binding site to P⁴⁹

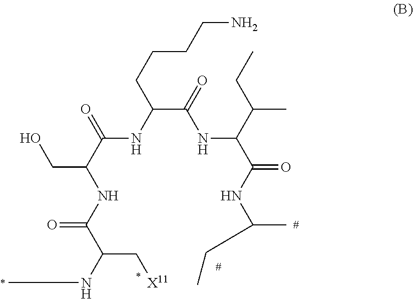

wherein X¹¹ is selected from the group consisting of

*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;

#—(CH₂)ₚ₅—CO—NH—(CH₂)ᵣ₅*, wherein p5 is 0, 1, 2 or 3, and r5 is 0, 1, 2 or 3, with the proviso that p5+r5=0-4;

wherein * indicates the binding site to P⁴³ and # indicates the binding site to P⁴⁹, and

wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according to formula (B), wherein * and ^{#} reflect where X⁵ is bound within the amino acid chain and wherein * indicates the binding site of X⁵ to P⁴³ and # indicates the binding site to P⁴⁹,

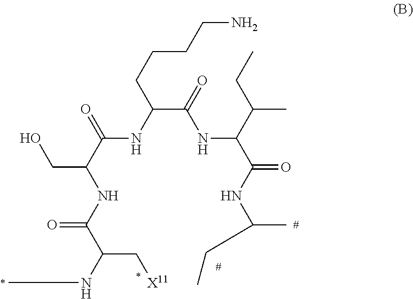

wherein X¹¹ is selected from the group consisting of

*—(CH₂)ₚ₄—CO—NH—(CH₂)ᵣ₄—^{#}, wherein p4 is 0 or 1 or, and r4 is 0, 1, 2 or 3, with the proviso that p4+r4=0-4;

^{#}—(CH₂)ₚ₅—CO—NH—(CH₂)ᵣ₅*, wherein p5 is 0 or 1, and r5 is 0, 1, 2 or 3, with the proviso that p5+r5=0-4;

wherein * and ^{#} reflect where X¹¹ is bound within the ring structure;

and wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according to formula (B), wherein * and ^{#} reflect where X⁵ is bound within the amino acid chain and wherein * indicates the binding site of X⁵ to P⁴³ and # indicates the binding site to P⁴⁹,

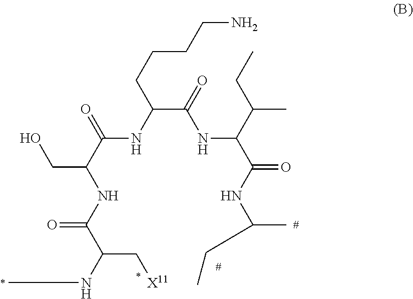

wherein X¹¹ is selected from the group consisting of

*—(CH₂)ₚ₄—CO—NH—(CH₂)ᵣ₄—^{#}, wherein p4 is 0 or 1 or, and r4 is 1, with the proviso that p4+r4=0-4;

^{#}—(CH₂)ₚ₅—CO—NH—(CH₂)ᵣ₅*, wherein p5 is 0 or 1, and r5 is 1, with the proviso that p5+r5=0-4;

wherein * and ^{#} reflect where X¹¹ is bound within the ring structure, and

wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according to formula (B), wherein * and ^{#} reflect where X⁵ is bound within the amino acid chain and wherein * indicates the binding site of X⁵ to P⁴³ and # indicates the binding site to P⁴⁹,

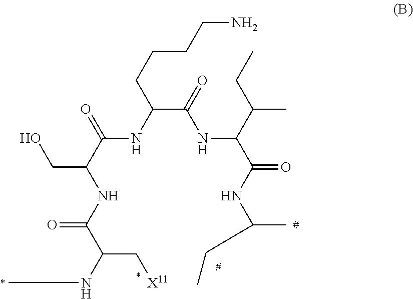

wherein X¹¹ is selected from the group consisting of

*—(CH₂)ₚ₄—CO—NH—(CH₂)ᵣ₄ #, wherein p4 is 0 or 1 or, and r4 is 2, with the proviso that p4+r4=0-4;

^{#}—(CH₂)ₚ₅—CO—NH—(CH₂)ᵣ₅—*, wherein p5 is 0 or 1, and r5 is 2, with the proviso that p5+r5=0-4;

wherein * and ^{#} reflect where X¹¹ is bound within the ring structure;

and wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows: X⁵ is the moiety according to formula (B), wherein * and ^{#} reflect where X⁵ is bound within the amino acid chain and wherein * indicates the binding site of X⁵ to P⁴³ and # indicates the binding site to P⁴⁹,

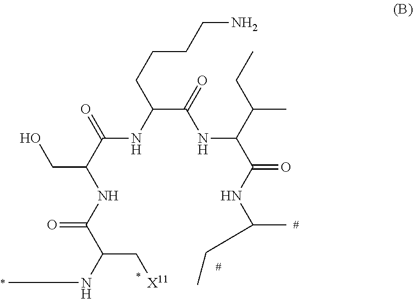

wherein X¹¹ is selected from the group consisting of

*—(CH₂)ₚ₄—CO—NH—(CH₂)ᵣ₄—^{#}, wherein p4 is 0 or 1 or, and r4 is 3, with the proviso that p4+r4=0-4;

^{#}—(CH₂)ₚ₅—CO—NH—(CH₂)ᵣ₅—*, wherein p5 is 0 or 1, and r5 is 3, with the proviso that p5+r5=0-4;

wherein * and ^{#} reflect where X¹¹ is bound within the ring structure, and

wherein X¹, X², X³, X⁴ and Z are as defined according to any one of the proceeding embodiments disclosed above or below.

According to an embodiment of the invention, the compounds of formula (I), a physiologically acceptable salt, a solvate or a solvate of a salt thereof are defined as follows:

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₈—^{#}, wherein m6 is 0, 1 or 2, and n5 is 0, 1 or 2;

^{#}—(CH₂)ₘ—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴, K¹⁴, which is covalently linked by an amide bond to the N-terminal G¹⁵ of the amino acid sequence of formula (I)

X³ is absent or is a C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X²;

wherein if X³ is a C14-C22 di-carboxylic acid, then X² is absent or is an amino acid or amino acid sequence as defined in any one of the proceeding claims above for X²;

X⁴ is the amino sequence *[D³⁵ K³⁶ D³⁷ K³⁸ D³⁹ N⁴⁰ V⁴¹]#, wherein * indicates the binding site to T³⁴ and # indicates the binding site to A⁴², or X⁴ is a moiety according to formula (A), wherein * indicates the binding site to T³⁴ and # indicates the binding site to A⁴²

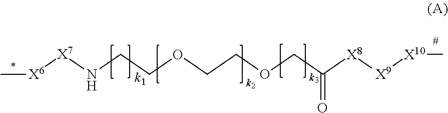

wherein

X⁶ is absent or selected from D, N, V; X⁷ is absent or selected from D, N, V; X⁸ is absent or selected from D, N, V; X⁹ is absent or selected from D, N, V; X¹⁰ is absent or selected from D, N, V;

wherein k1 is 1 or 2; k2 is 0, 1, 2, 3, or 4; k3 is 1 or 2.

X⁵ is the amino sequence * [R⁴⁴ S⁴⁵ K⁴⁶ I⁴⁷ S⁴⁸]#, wherein * indicates the binding site to P⁴³ and # indicates the binding site to P⁴⁹, or X⁵ is a moiety according to formula (B), wherein * and ^{#} reflect where X⁵ is bound within the amino acid chain and wherein * indicates the binding site of X⁵ to P⁴³ and # indicates the binding site to P⁴⁹,

wherein X¹¹ is selected from the group consisting of

*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;

^{#}—(CH₂)ₚ₅—CO—NH—(CH₂)ᵣ₅—*, wherein p5 is 0, 1, 2 or 3, and r5 is 0, 1, 2 or 3, with the proviso that p5+r5=0-4

wherein * and ^{#} reflect where X¹¹ is bound within the ring structure.

According to an embodiment of the invention, the compounds of formula (I), a physiologically acceptable salt, a solvate or a solvate of a salt thereof are defined as follows and can be selected from: the compound is a compound according to formula (Ia),

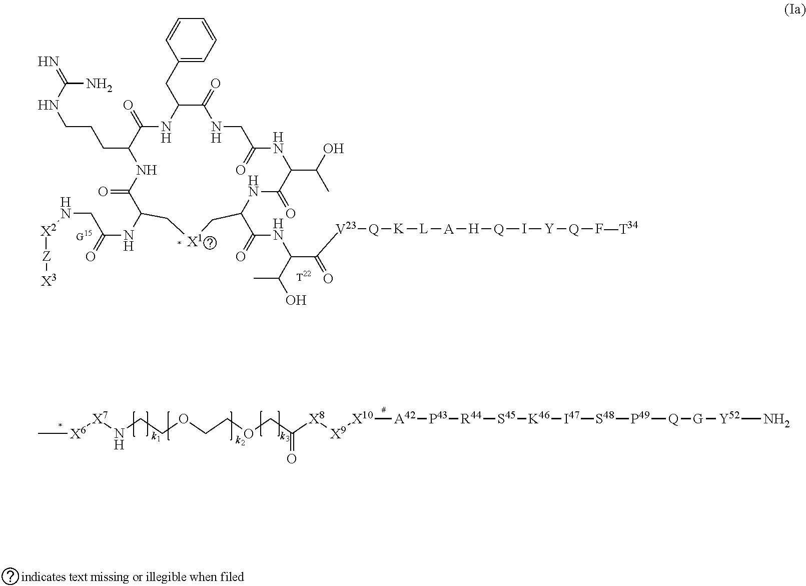

wherein X¹, X², X³, X⁶, X⁷, X⁸, X⁹, X¹⁰, k1, k2, and k3 are as defined according to any one of the proceeding embodiments disclosed above or below,

a compound according to formula (Ib)

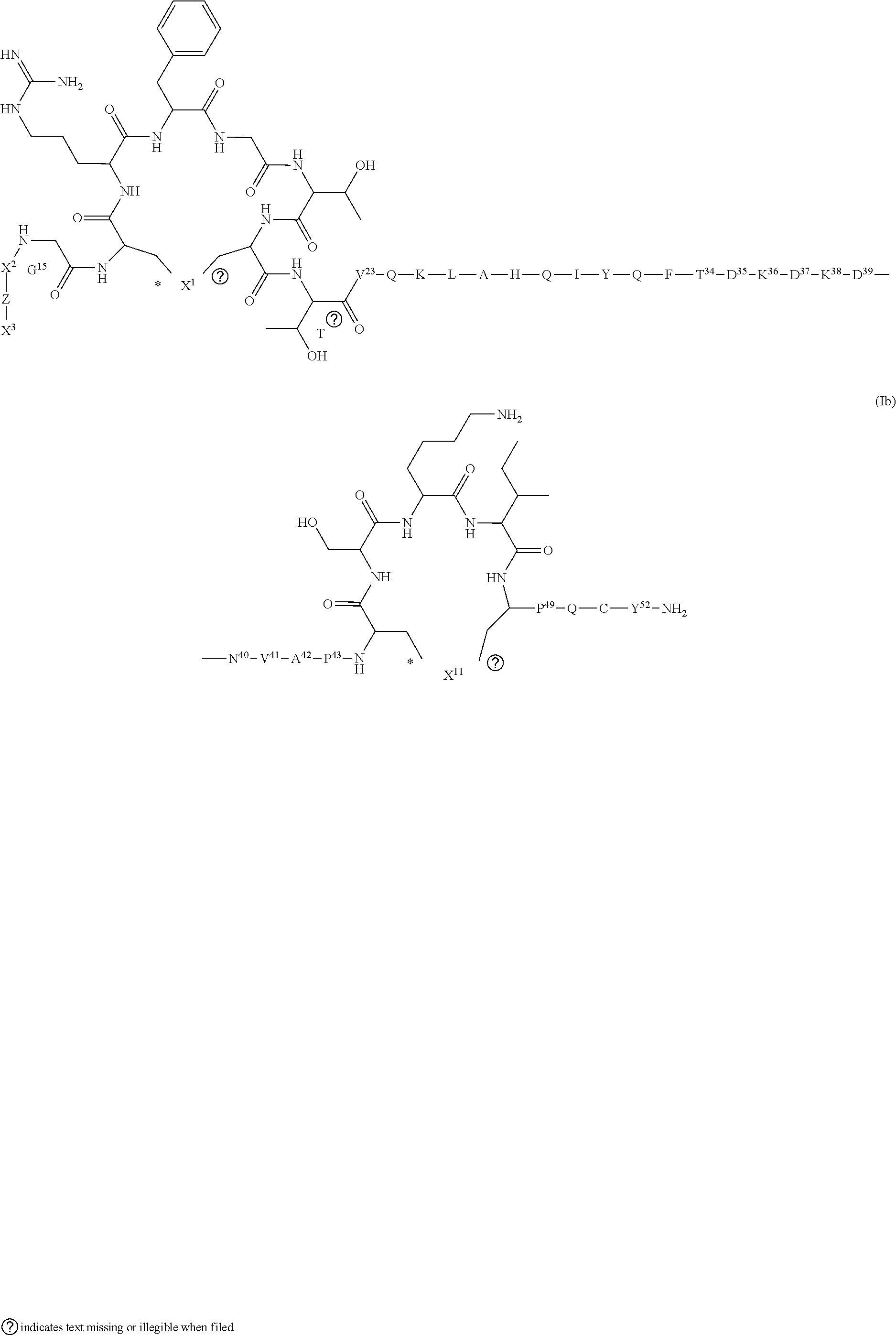

wherein X¹, X², X³ and X¹¹ are as defined according to any one of the proceeding embodiments disclosed above or below,

a compound according to formula (Ic)

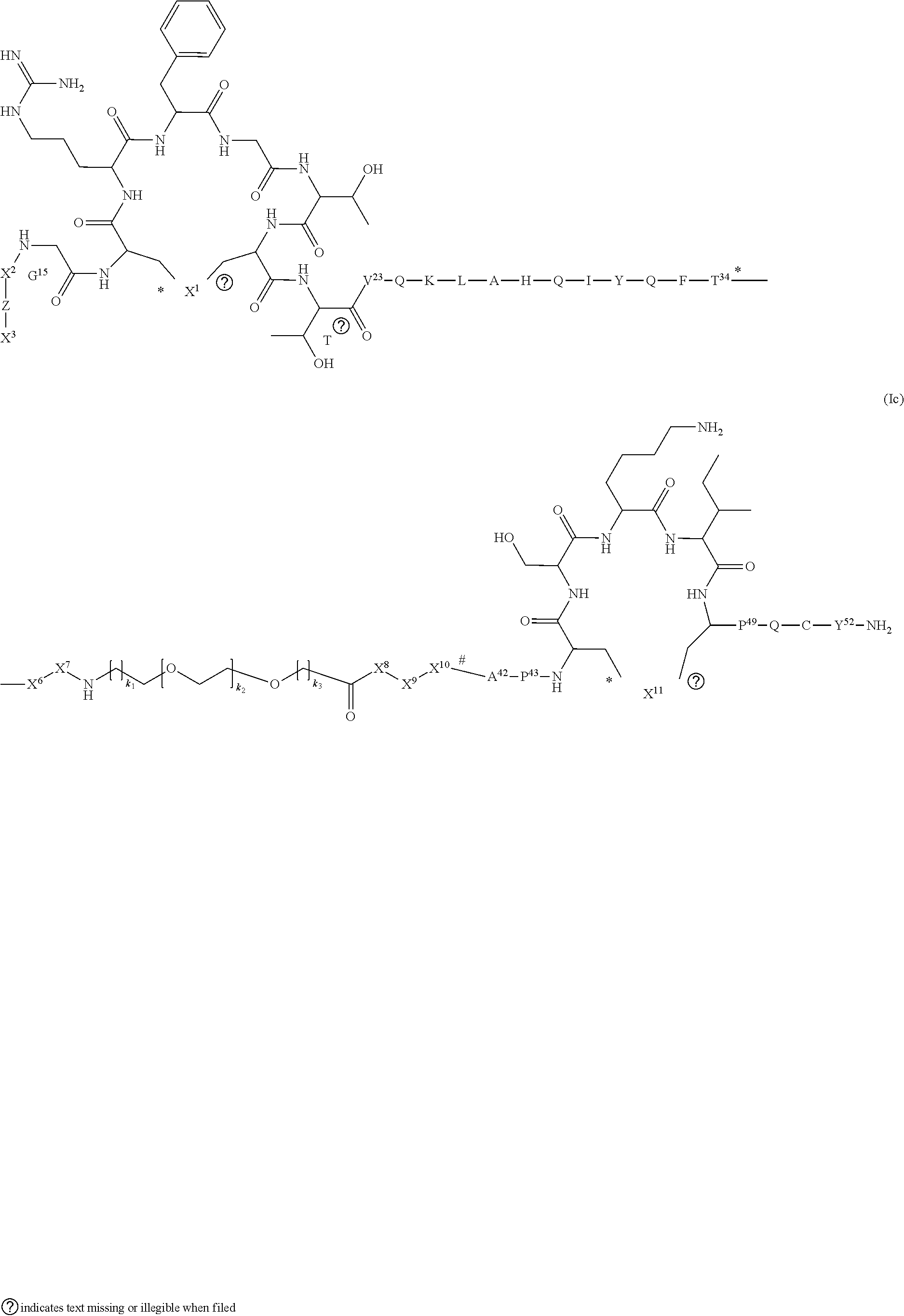

wherein X¹, X², X³, Z, X⁶, X⁷, X⁸, X⁹, X¹⁰, k1, k2, k3 and X¹¹ are as defined according to any one of the proceeding embodiments disclosed above or below,

a compound according to formula (Id)

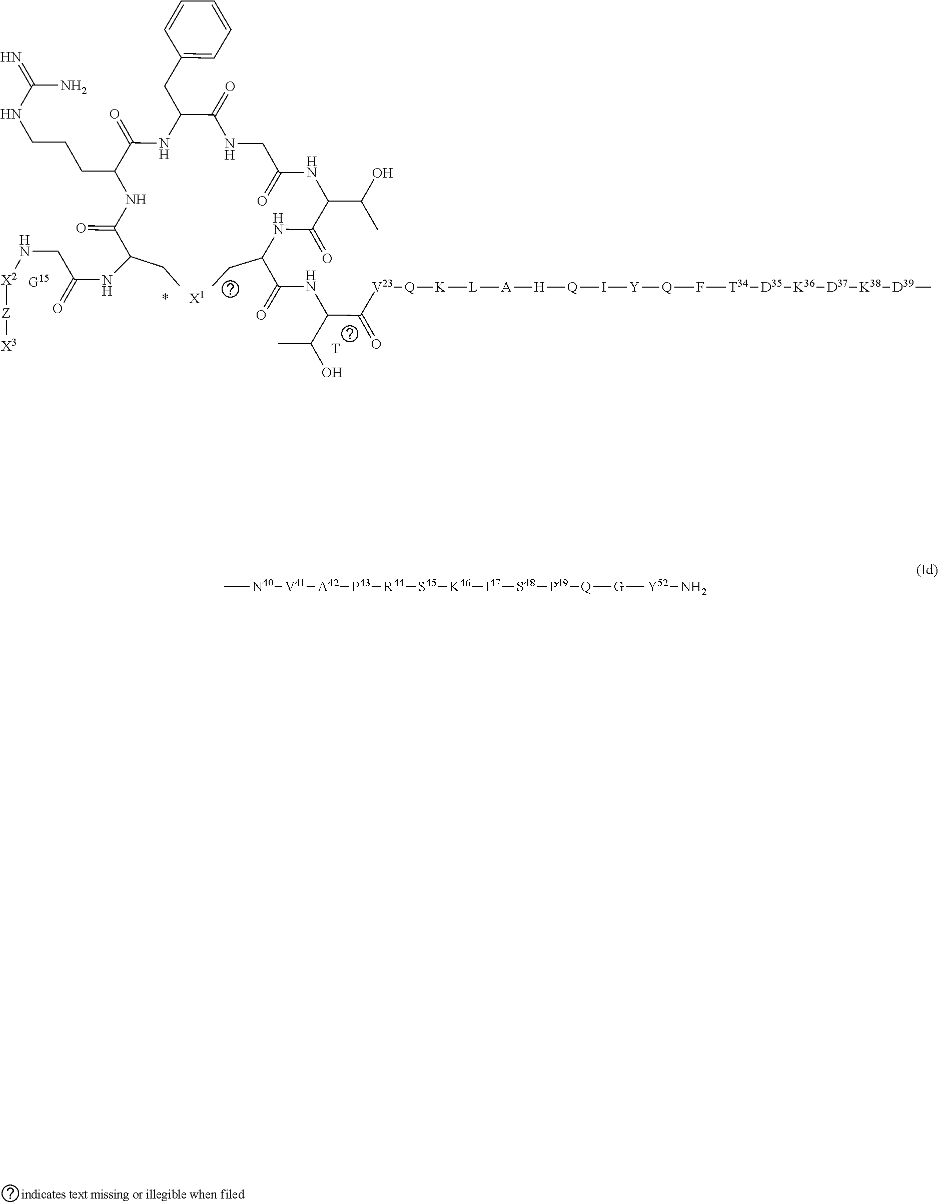

wherein X³ is a di-carboxylic acid, and X¹, X², Z are as defined according to any one of the proceeding embodiments disclosed above or below,

a compound according to formula (Ie)

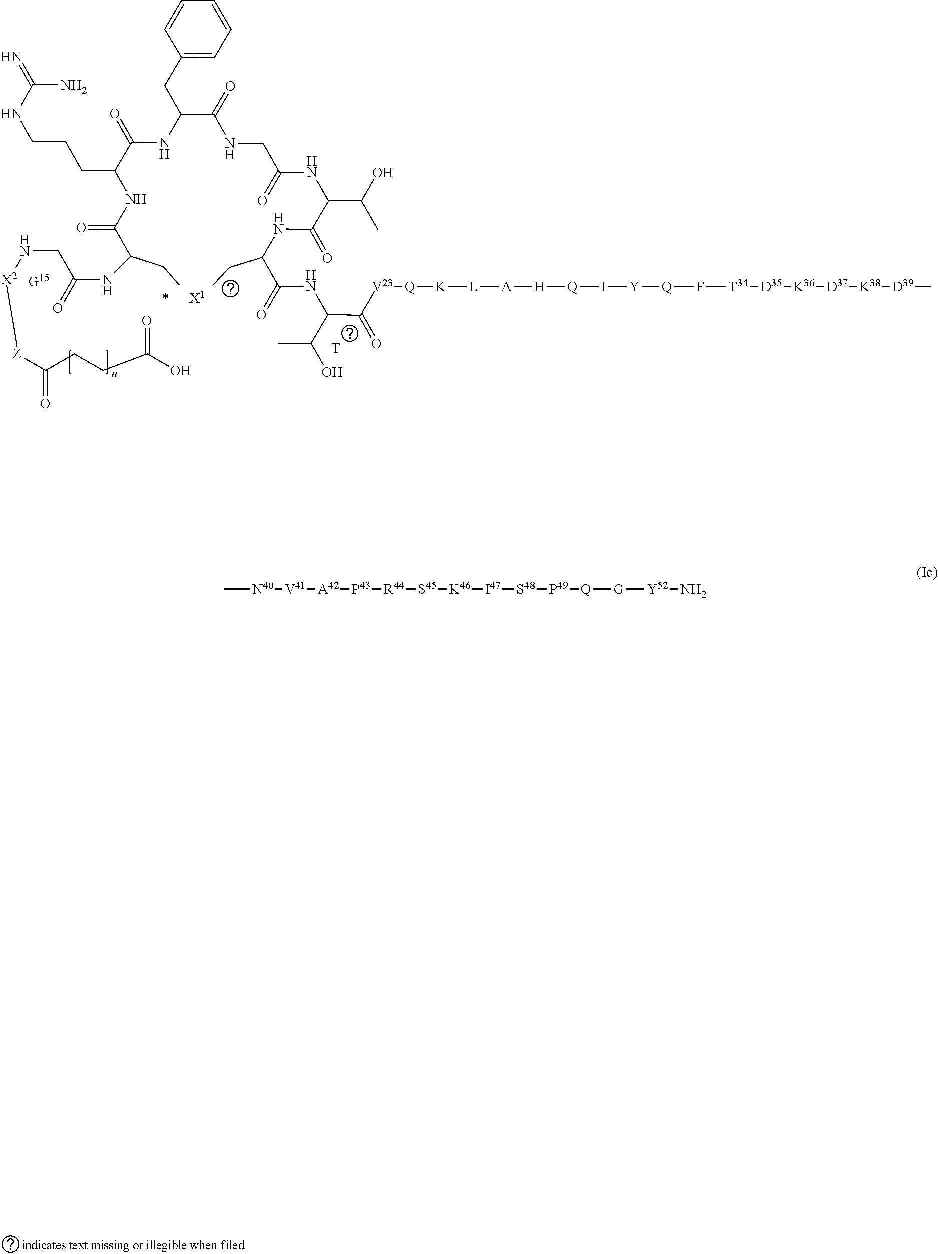

wherein n is 1 to 30 and X¹, X², Z are defined according to any one of the proceeding embodiments disclosed above or below,

a compound according to formula (If)

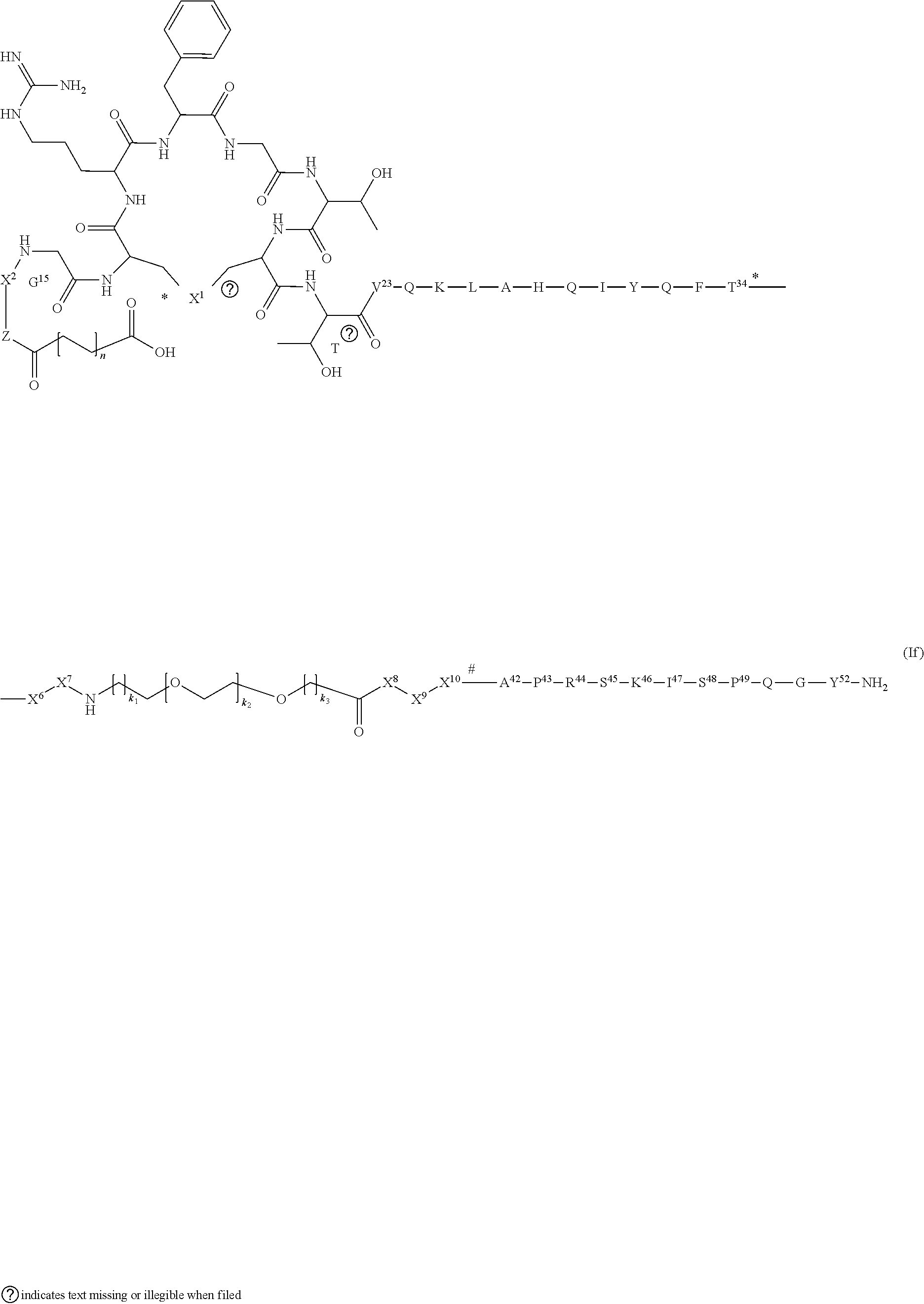

wherein n is 1 to 30 and X¹, X², Z, X⁶, X⁷, X⁸, X⁹, X¹⁰, k1, k2, k3 are defined according to any one of the proceeding embodiments disclosed above or below,

a compound according to formula (Ig)

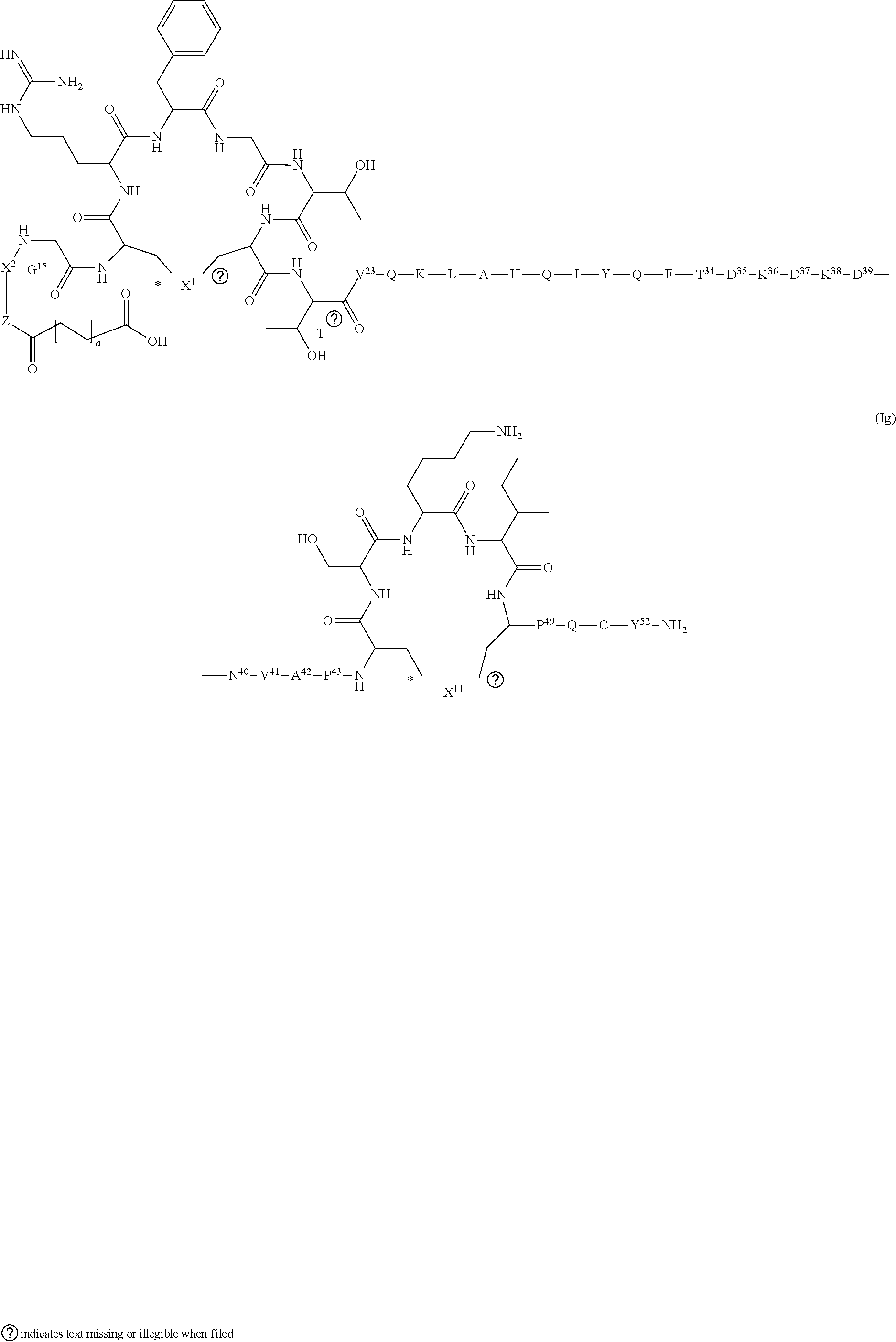

wherein n is 1 to 30 and X¹, X², Z and X¹¹ are defined according to any one of proceeding embodiments disclosed above or below,

a compound according to formula (Ih)

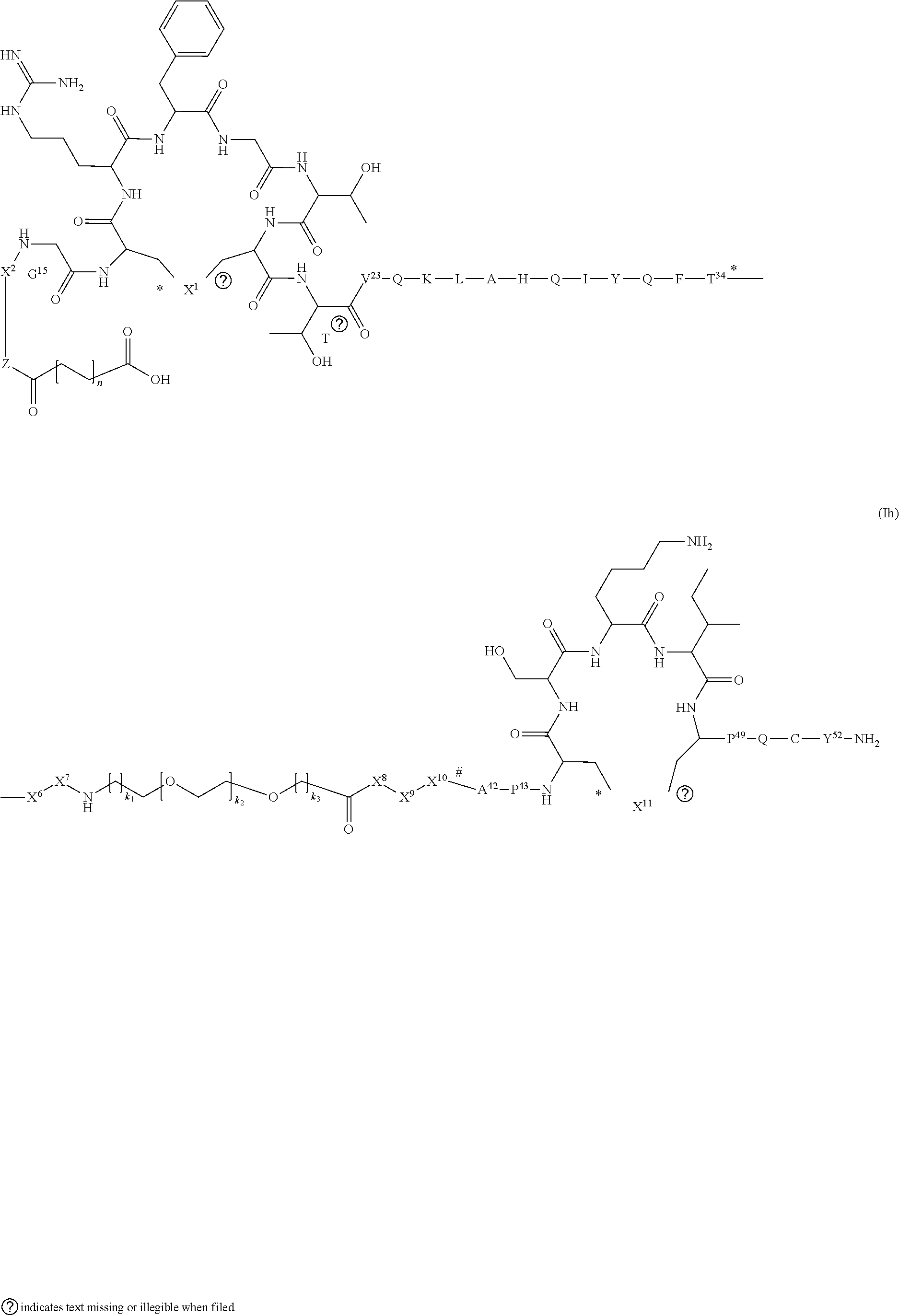

wherein n is 1 to 30 and X¹, X², Z, X⁶, X⁷, X⁸, X⁹, X¹⁰, k1, k2, k3 and X¹¹ are defined according to any one of the proceeding embodiments disclosed above or below.

In other words: In formula (Ia), X⁴ is a moiety according to formula (A) and X⁵ is the amino sequence *[R⁴⁴ S⁴⁵ K⁴⁶ I⁴⁷ S⁴⁸]g.

In formula (Ib), X⁴ is the amino sequence * [D³⁵ K³⁶ D³⁷ K³⁸ D³⁹ N⁴⁰ V⁴¹]# and X⁵ is a moiety according to formula (B).

In formula (Ic), X⁴ is a moiety according to formula (A) and X⁵ is a moiety according to formula (B).

In formula (Id), X⁴ is the amino sequence * [D³⁵ K³⁶ D³⁷ K³⁸ D³⁹ N⁴⁰ V⁴¹]# and X⁵ is the amino sequence *[R44 S⁴⁵ K⁴⁶ I⁴⁷ S⁴⁸]#.

In formula (Ie), X³ is a moiety according to formula (C), X⁴ is the amino sequence * [D³⁵ K³⁶ D³⁷ K³⁸ D³⁹ N⁴⁰ V⁴¹]# and X⁵ is the amino sequence * [R⁴⁴ S⁴⁵ K⁴⁶ I⁴⁷ S⁴⁸]#

In formula (If), X³ is a moiety according to formula (C), X⁴ is a moiety according to formula (A) and X⁵ is the amino sequence * [R⁴⁴ S⁴⁵ K⁴⁶ I⁴⁷ S⁴⁸]#

In formula (Ig), X³ is a moiety according to formula (C), X⁴ is the amino sequence * [D³⁵ K³⁶ D³⁷ K³⁸ D³⁹ N⁴⁰ V⁴¹]# and X⁵ is a moiety according to formula (B).

In formula (Ih), X³ is a moiety according to formula (C), X⁴ is a moiety according to formula (A) and X⁵ is a moiety according to formula (B).

According to an embodiment of the invention, the compounds of formula (Ia), a physiologically acceptable salt, a solvate or a solvate of a salt thereof are defined as follows:

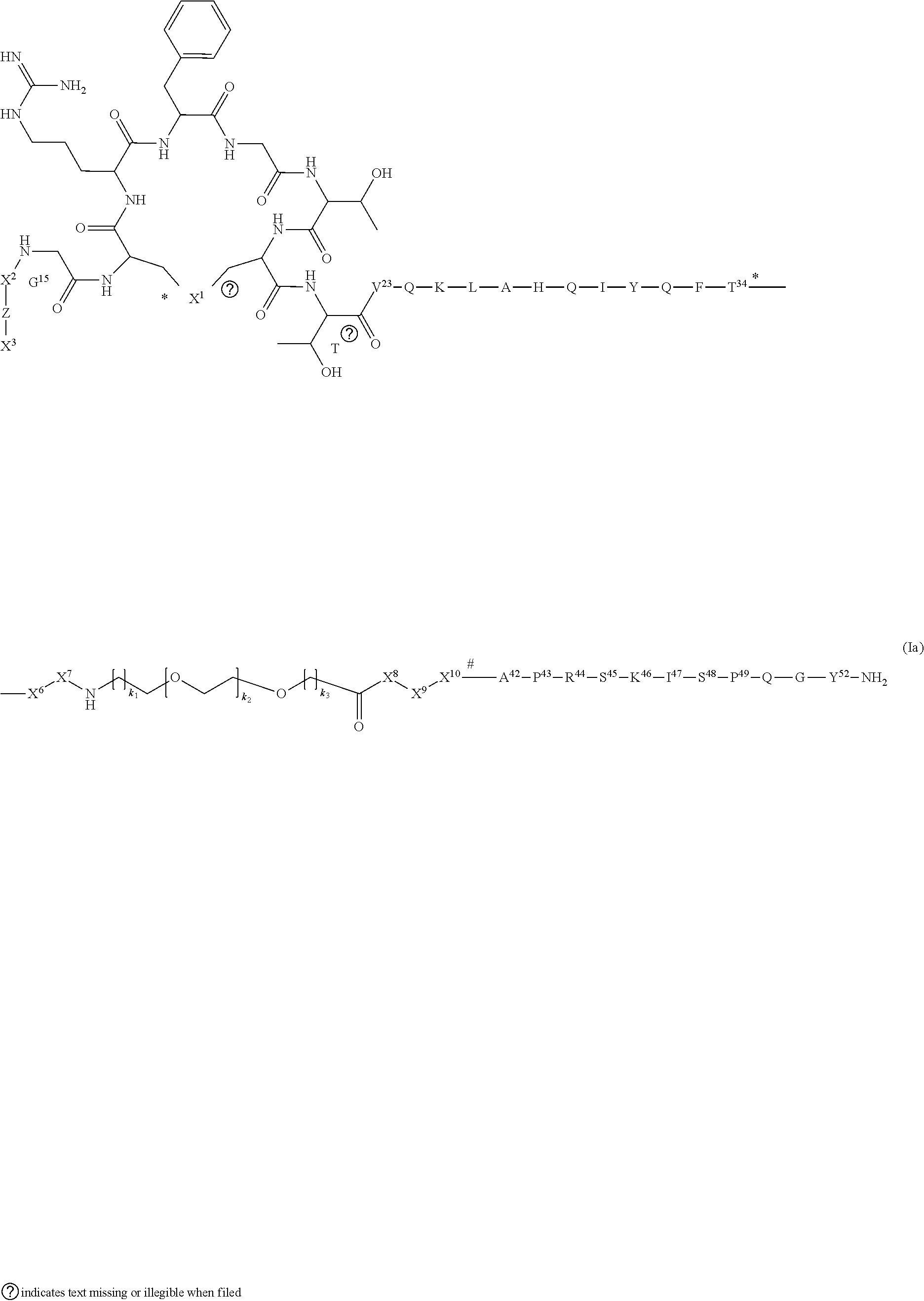

wherein

X¹ is selected from the group consisting of

*—(CH₂)ₘ₁—S—S—(CH₂)ₙ₁—^{#}, wherein m1 is 0-6, n1 is 0-6, with the proviso that m1+n1=0-6;

*—(CH₂)ₘ₆—CO—NH—(CH₂)ₙ₅—^{#}, wherein m6 is 0-4, and n5 is 0-4, with the proviso that m6+n5=0-6;

^{#}—(CH₂)ₘ₇—CO—NH—(CH₂)ₙ₆—*, wherein m7 is 0-4, and n6 is 0-4, with the proviso that m7+n6=0-6;

*—(CH₂)ₘ₃—^{#}, wherein m3 is 1-8;

wherein * and ^{#} reflect where X¹ is bound within the ring structure

X² is selected from the group consisting of G¹⁴, K¹⁴, which is covalently linked by an amide bond to the N-terminal G¹⁵ of the amino acid sequence of formula (I)

X³ is absent or is a linear or branched C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X2;

wherein if X³ is a linear or branched C14-C22 di-carboxylic acid, then X² is absent or is an amino acid or amino acid sequence as defined in any one of the proceeding claims above for X2,

X⁶, X⁷, X⁸ X⁹ and X¹⁰ are defined as in any one of the proceeding embodiments disclosed above or below,

wherein k1 is 1 or 2; wherein k2 is 0, 1, 2, 3, or 4; wherein k3 is 1 or 2.

In one embodiment according to the invention, the compounds according to any one according to formulae according to any of (Ia), (Ib), (Ic), (Id), (le), (If), (Ig) and/or (Ih) can independently from one another comprise at least one of the stereoisomeric forms, wherein all amino acids have the L-configuration, as depicted below:

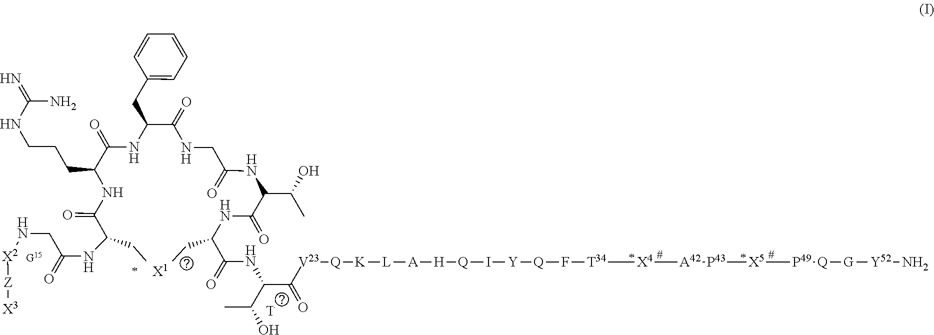

and/or wherein X⁵ is a moiety according to formula (B) as defined above, wherein all amino acids have the L-configuration:

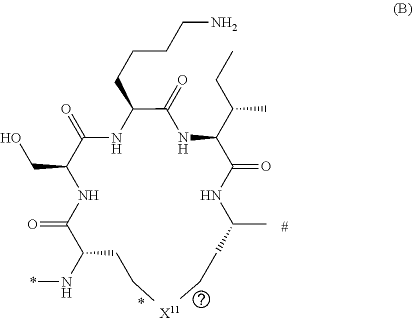

The present invention comprises all possible stereoisomeric forms, also in cases where no stereoisomerism is indicated.

In one embodiment, all amino acids comprised in any one of the formulae (I), (Ia), (Ib), (Ic), (Id), (le), (If), (Ig) and/or (Ih) are in L-configuration.

In one embodiment, the compound according to any of formulae (le), (If), (Ig) and/or (Ih) n is 7 to 9.

According to an embodiment of the invention, the compounds of formula (Ia), a physiologically acceptable salt, a solvate or a solvate of a salt thereof are defined as follows:

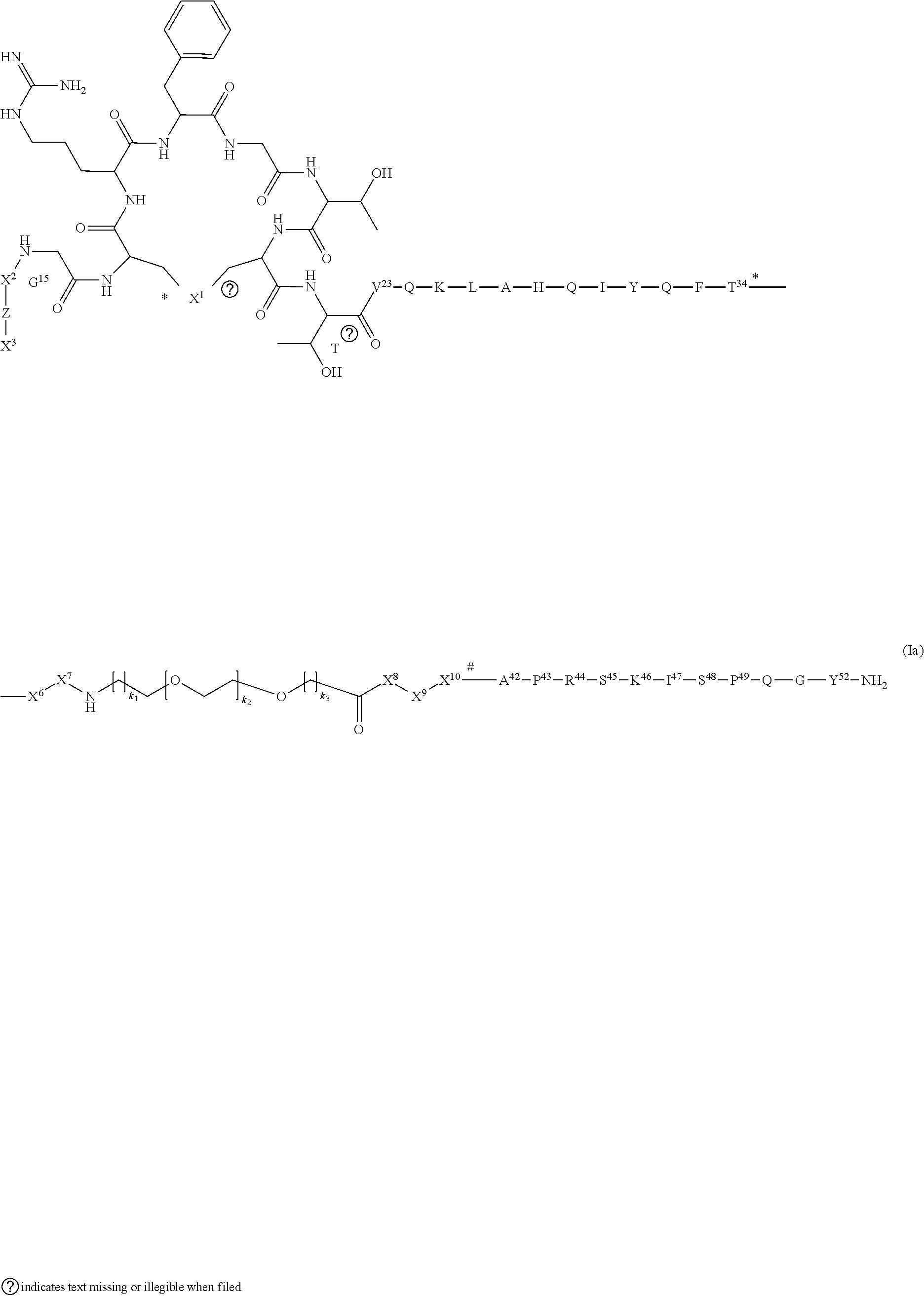

wherein

X¹ is selected from the group consisting of

*—(CH₂)ₘ₁—S—S—(CH₂)ₙ₁—^{#} wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ and Z are absent,

X⁶ is absent or selected from D, N, V;

X⁷ is absent or selected from D, N, V;

X⁸ is absent or selected from D, N, V;

X⁹ is absent or selected from D, N, V;

X¹⁰ is absent or selected from D, N, V;

wherein k1 is 1 or 2; wherein k2 is 0, 1, 2, 3, or 4; wherein k3 is 1 or 2.

According to an embodiment of the invention, the compounds of formula (Ib), a physiologically acceptable salt, a solvate or a solvate of a salt thereof are defined as follows:

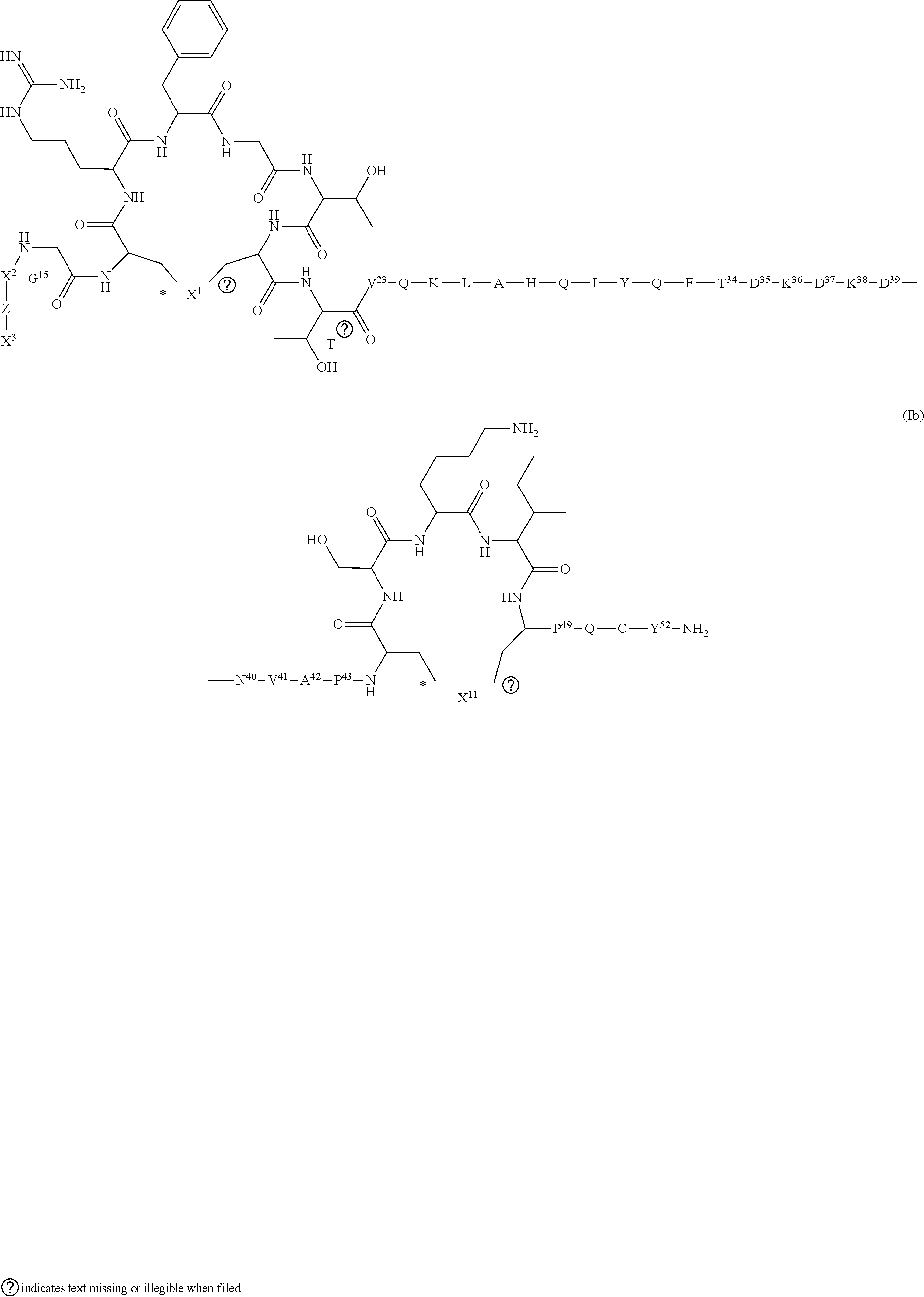

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ is absent or is a linear or branched C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X²;

wherein if X³ is a linear or branched C14-C22 di-carboxylic acid, then X² is absent or is an amino acid or amino acid sequence as defined in any one of the proceeding claims above for X²

X¹¹ is selected from

*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;

^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;

wherein * and ^{#} reflect where X¹¹ is bound within the ring structure.

According to an embodiment of the invention, the compounds of formula (Ib), a physiologically acceptable salt, a solvate or a solvate of a salt thereof are defined as follows:

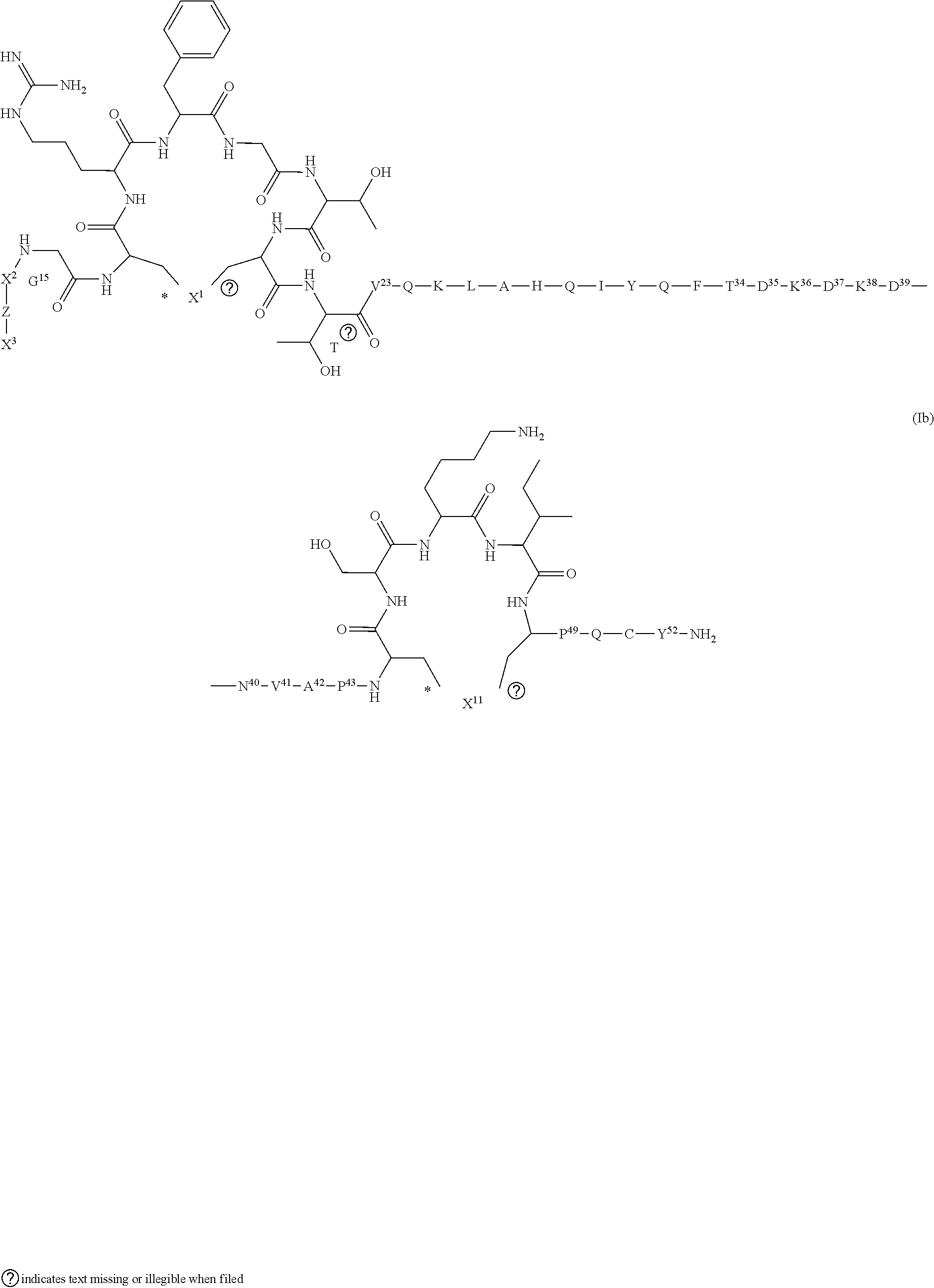

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ and Z are absent,

X¹¹ is selected from

*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;

^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;

wherein * and ^{#} reflect where X¹¹ is bound within the ring structure.

According to an embodiment of the invention, the compounds of formula (Ic), a physiologically acceptable salt, a solvate or a solvate of a salt thereof are defined as follows:

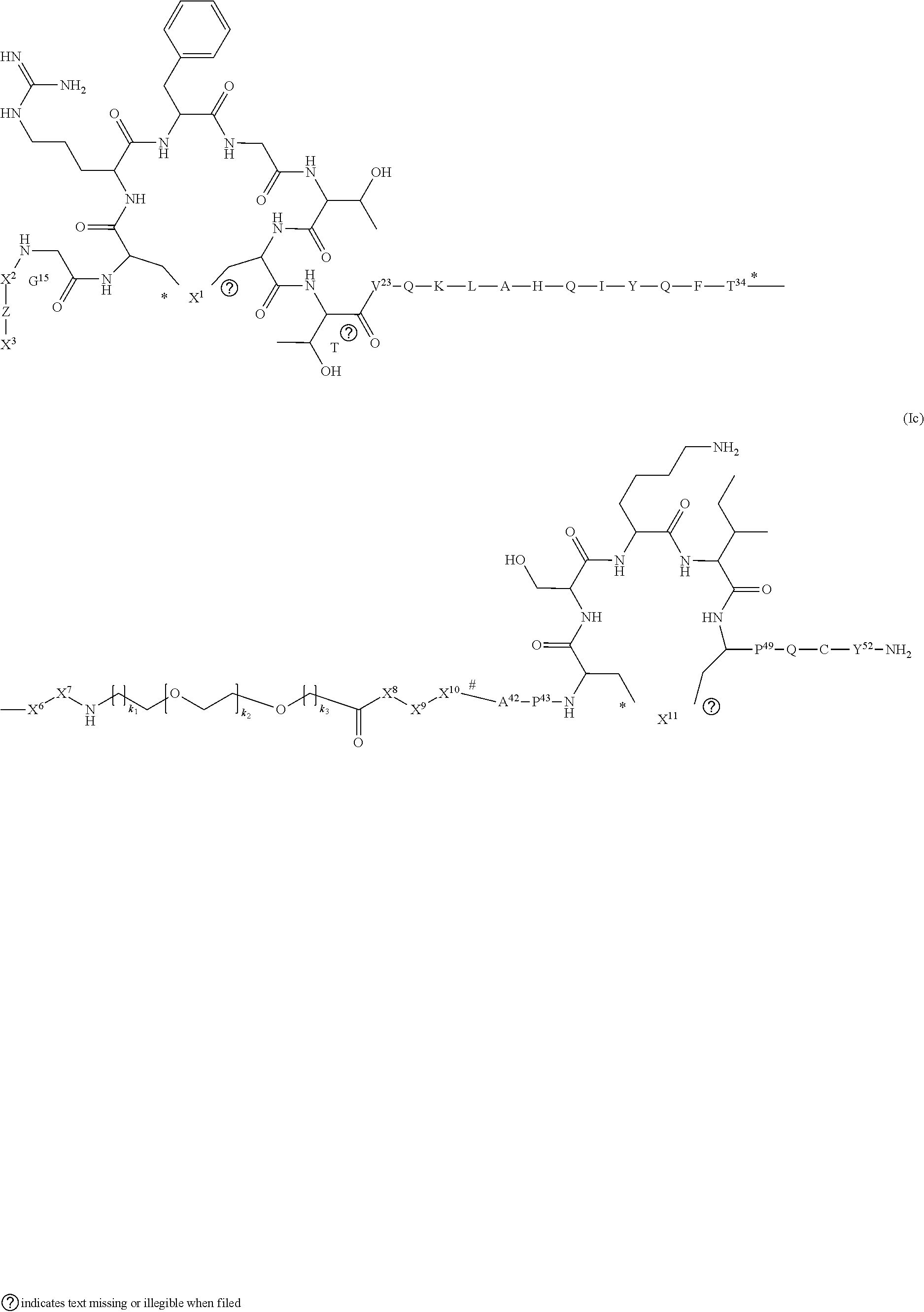

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ is absent or is a linear or branched C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X²;

wherein if X³ is a linear or branched C14-C22 di-carboxylic acid, then X² is absent or is an amino acid or amino acid sequence as defined in any one of the proceeding claims above for X²

X⁶ is absent or selected from D, N, V;

X⁷ is absent or selected from D, N, V;

X⁸ is absent or selected from D, N, V;

X⁹ is absent or selected from D, N, V;

X¹⁰ is absent or selected from D, N, V;

wherein k1 is 1 or 2; wherein k2 is 0, 1, 2, 3, or 4; wherein k3 is 1 or 2;

X¹¹ is selected from

*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;

^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;

wherein * and ^{#} reflect where X¹¹ is bound within the ring structure.

According to an embodiment of the invention, the compounds of formula (Ic), a physiologically acceptable salt, a solvate or a solvate of a salt thereof are defined as follows:

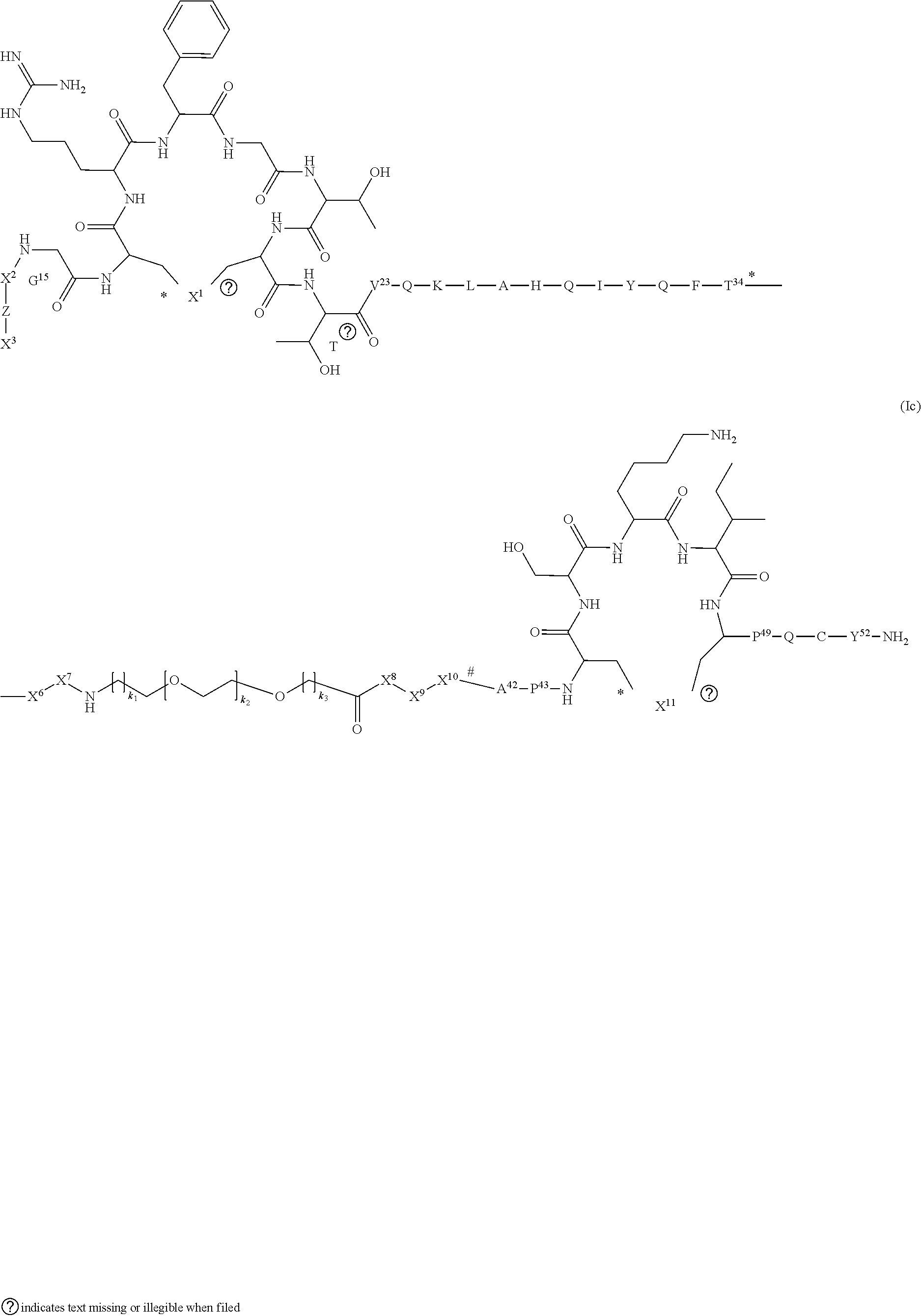

15 wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

20 wherein * and ^{#} reflect where X¹ is bound within the ring structure

X² is selected from the group consisting of G¹⁴ or K¹⁴;

X³ and Z are absent,

X⁶ is absent or selected from D, N, V;

X⁷ is absent or selected from D, N, V;

X⁸ is absent or selected from D, N, V;

X⁹ is absent or selected from D, N, V;

X¹⁰ is absent or selected from D, N, V;

wherein k1 is 1 or 2;

wherein k2 is 0, 1, 2, 3, or 4;

wherein k3 is 1 or 2;

X¹¹ is selected from

*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;

^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;

wherein * and ^{#} reflect where X¹¹ is bound within the ring structure.

According to an embodiment of the invention, the compounds of formula (Ic), a physiologically acceptable salt, a solvate or a solvate of a salt thereof are defined as follows:

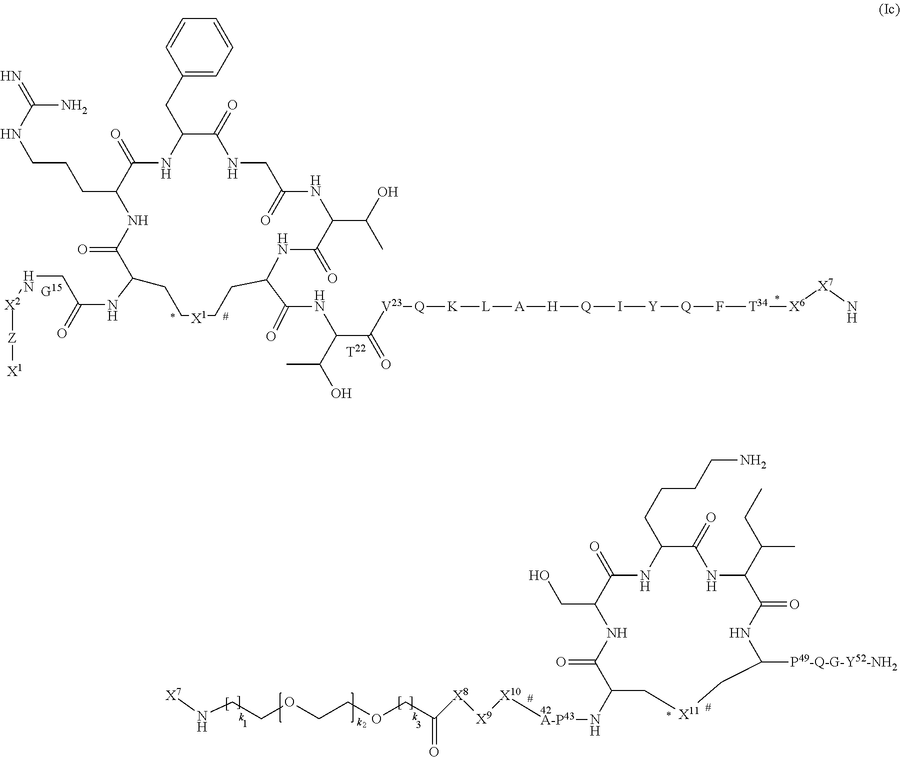

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ is absent or is a linear or branched C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X²;

wherein if X³ is a linear or branched C14-C22 di-carboxylic acid, then X² is absent or is an amino acid or amino acid sequence as defined in any one of the proceeding claims above for X²

X⁶ is absent or selected from D, N, V;

X⁷ is absent or selected from D, N, V;

X⁸ is absent or selected from D, N, V;

X⁹ is absent or selected from D, N, V;

X¹⁰ is absent or selected from D, N, V;

wherein k1 is 1 or 2;

wherein k2 is 0, 1 or 2;

wherein k3 is 1 or 2;

X¹¹ is selected from

*—(CH₂)ₚ₄—CO—NH—(CH₂)ᵣ₄—^{#}, wherein p4 is 0 or 1, r4 is 0, 1, 2 or 3, with the proviso that p4+r4=0-5;

^{#}—(CH₂)ₚ₅—CO—NH—(CH₂)ᵣ₅—*, wherein p5 is 0 or 1, r5 is 0, 1, 2 or 3, with the proviso that p5+r5=0-5;

wherein * and ^{#} reflect where X¹¹ is bound within the ring structure.

According to an embodiment of the invention, the compounds of formula (Ic), a physiologically acceptable salt, a solvate or a solvate of a salt thereof are defined as follows:

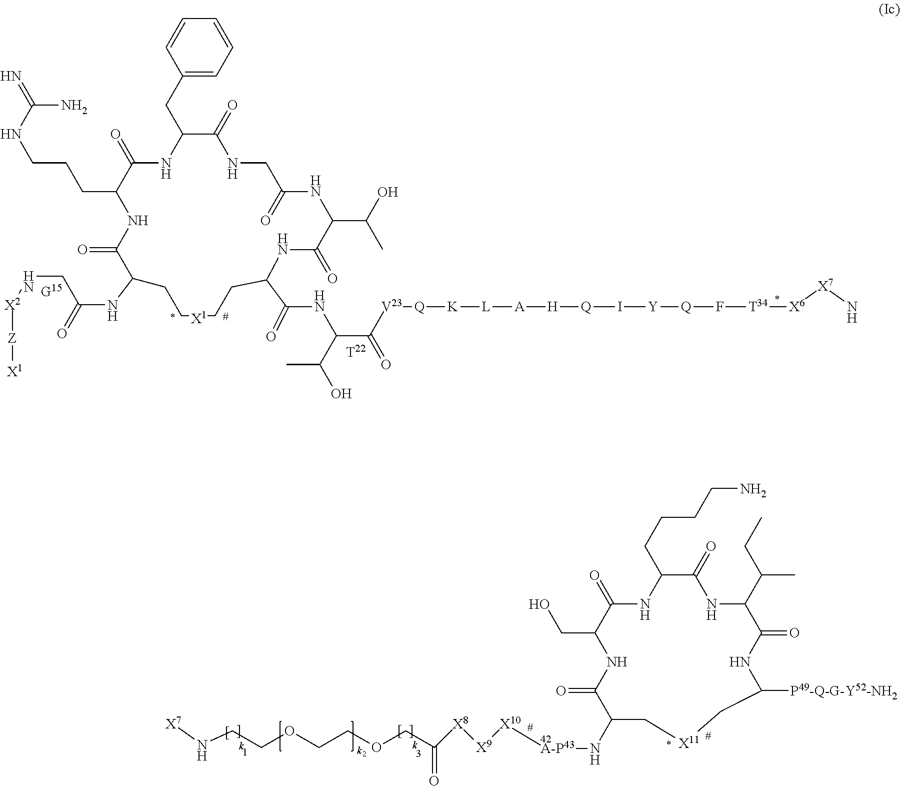

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ and Z are absent,

X⁶ is absent or selected from D, N, V;

X⁷ is absent or selected from D, N, V;

X⁸ is absent or selected from D, N, V;

X⁹ is absent or selected from D, N, V;

X¹⁰ is absent or selected from D, N, V;

wherein k1 is 1 or 2;

wherein k2 is 0, 1 or 2;

wherein k3 is 1 or 2;

X¹¹ is selected from

*—(CH₂)ₚ₄—CO—NH—(CH₂)ᵣ₄—^{#}, wherein p4 is 0 or 1, r4 is 0, 1, 2 or 3, with the proviso that p4+r4=0-5;

^{#}—(CH₂)ₚ₅—CO—NH—(CH₂)ᵣ₅—*, wherein p5 is 0 or 1, r5 is 0, 1, 2 or 3, with the proviso that p5+r5=0-5;

wherein * and ^{#} reflect where X¹¹ is bound within the ring structure.

According to an embodiment of the invention, the compounds of formula (Id), a physiologically acceptable salt, a solvate or a solvate of a salt thereof are defined as follows:

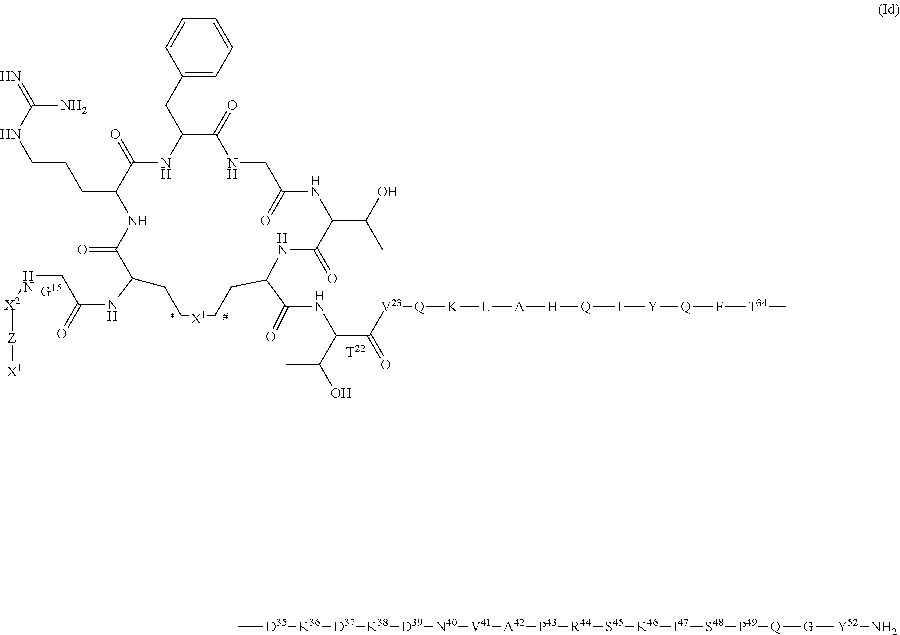

wherein

X¹ is selected from the group consisting of

*—(CH₂)ₘ₁—S—S—(CH₂)ₙ₁—^{#} wherein m1 is 0, 1 or 2, n1 is 0, 1 or 2;

*—(CH₂)ₘ₆—CO—NH—(CH₂)ₙ₅—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ is absent or is a linear or branched C14-C22 di-carboxylic acid,

Z is absent or is a cleavable linker covalently bound between the N terminus of any amino acid of X² or of G¹⁵ and X³ or between a functional group of the side chain of any amino acid of X² and X³

wherein if X³ is absent, then Z is also absent and X² is hydrogen or is an amino acid or amino acid sequence as defined in any one of the proceeding claims above for X²;

wherein if X³ is a linear or branched C14-C22 di-carboxylic acid, then X² is absent or is an amino acid or amino acid sequence as defined in any one of the proceeding claims above for X².

According to an embodiment of the invention, the compounds of formula (I), a physiologically acceptable salt, a solvate or a solvate of a salt thereof are a compound according to formula (Ie) as defined as follows:

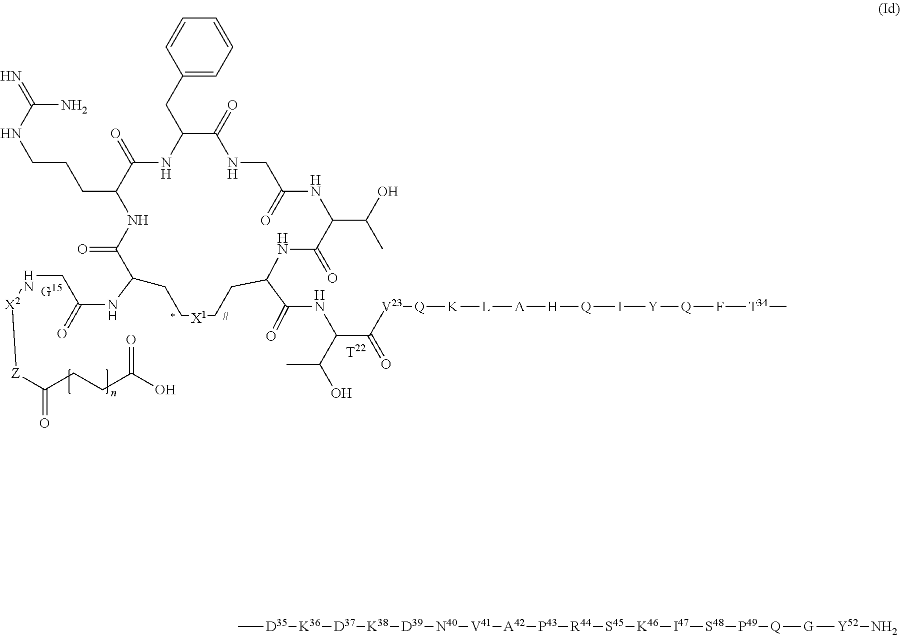

wherein n is 1 to 15, and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 2 to 11, and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 4 to 10, and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 6 to 9, preferably 7 to 9, and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 7 to 8, and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 6 and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 7 and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 8 and wherein X¹ and X² are as defined according to any one of the proceeding embodiments disclosed above or below.

According to an embodiment of the invention, the compounds of formula (Ie), a physiologically acceptable salt, a solvate or a solvate of a salt thereof are as defined as follows: wherein n is 9 and wherein X¹ and X² are as defined according to any one of the proceeding embodiments disclosed above or below.

According to an embodiment of the invention, the compounds of formula (I), a physiologically acceptable salt, a solvate or a solvate of a salt thereof are a compound according to formula (If), a physiologically acceptable salt, a solvate or a solvate of a salt thereof are as defined as follows:

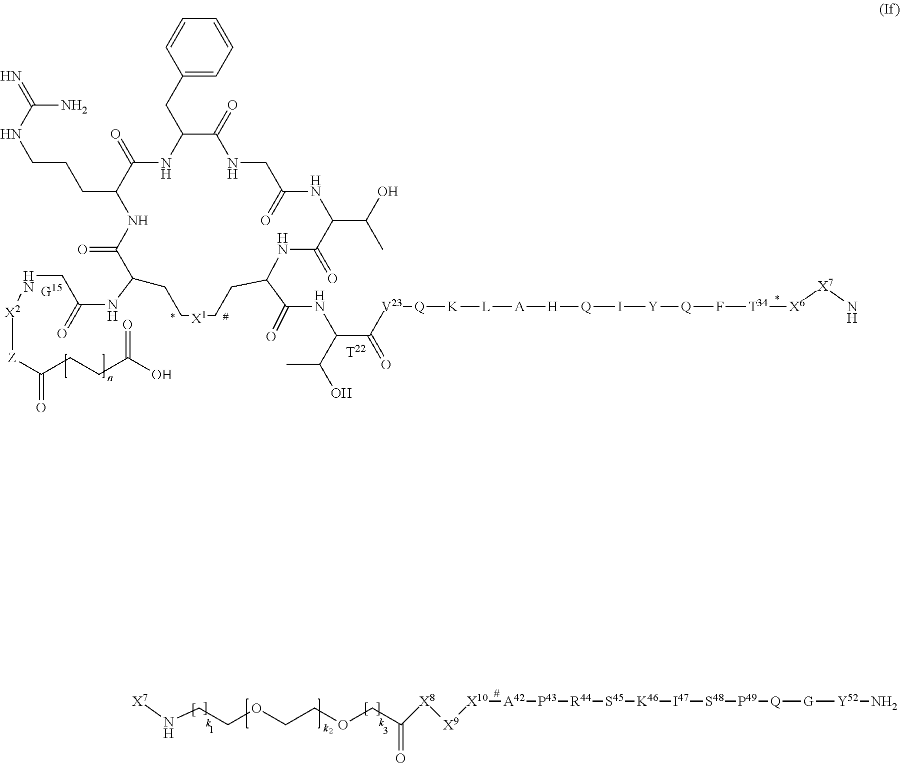

wherein n is 1 to 15, and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 2 to 11, and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 4 to 10, and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 6 to 9, preferably 7 to 9, and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 7 to 8, and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 6 and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 7 and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 8 and wherein X¹ and X² are as defined according to any one of the proceeding embodiments disclosed above or below.

According to an embodiment of the invention, the compounds of formula (If), a physiologically acceptable salt, a solvate or a solvate of a salt thereof are as defined as follows: wherein n is 9 and wherein X¹ and X² are as defined according to any one of the proceeding embodiments disclosed above or below.

According to an embodiment of the invention, the compounds of formula (I), a physiologically acceptable salt, a solvate or a solvate of a salt thereof are a compound according to formula (Ig), a physiologically acceptable salt, a solvate or a solvate of a salt thereof are as defined as follows:

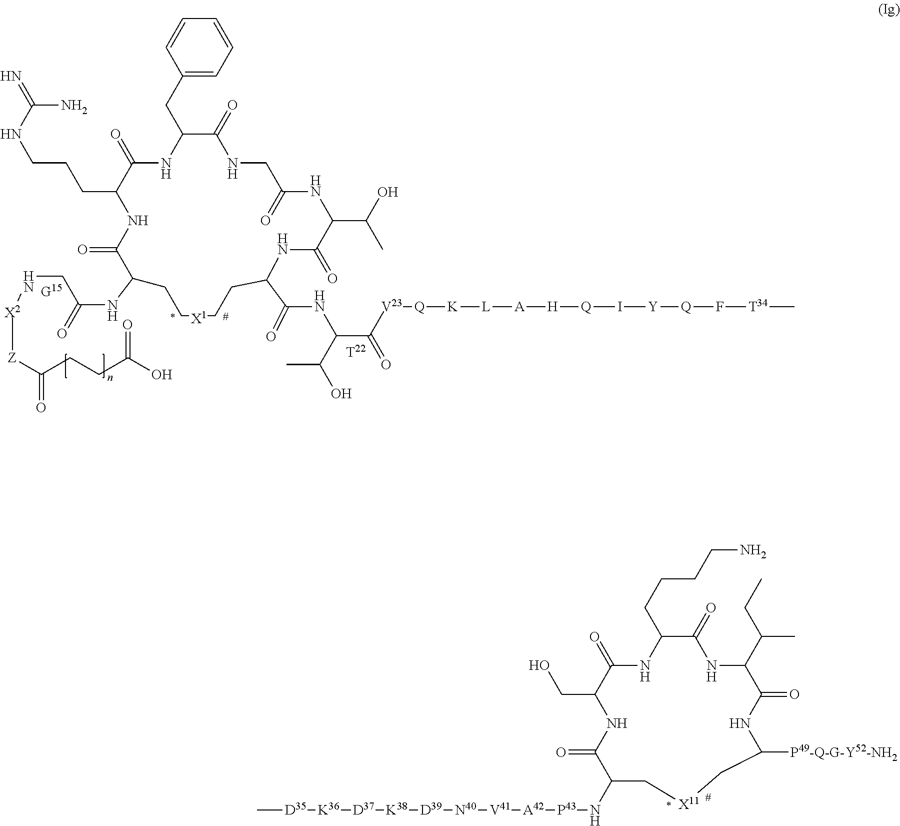

wherein n is 1 to 15, and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 2 to 11, and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 4 to 10, and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 6 to 9, preferably 7 to 9, and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 7 to 8, and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 6 and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 7 and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 8 and wherein X¹ and X² are as defined according to any one of the proceeding embodiments disclosed above or below.

According to an embodiment of the invention, the compounds of formula (Ig), a physiologically acceptable salt, a solvate or a solvate of a salt thereof are as defined as follows: wherein n is 9 and wherein X¹ and X² are as defined according to any one of the proceeding embodiments disclosed above or below.

According to an embodiment of the invention, the compounds of formula (I), a physiologically acceptable salt, a solvate or a solvate of a salt thereof are a compound according to formula (Ih), a physiologically acceptable salt, a solvate or a solvate of a salt thereof are as defined as follows:

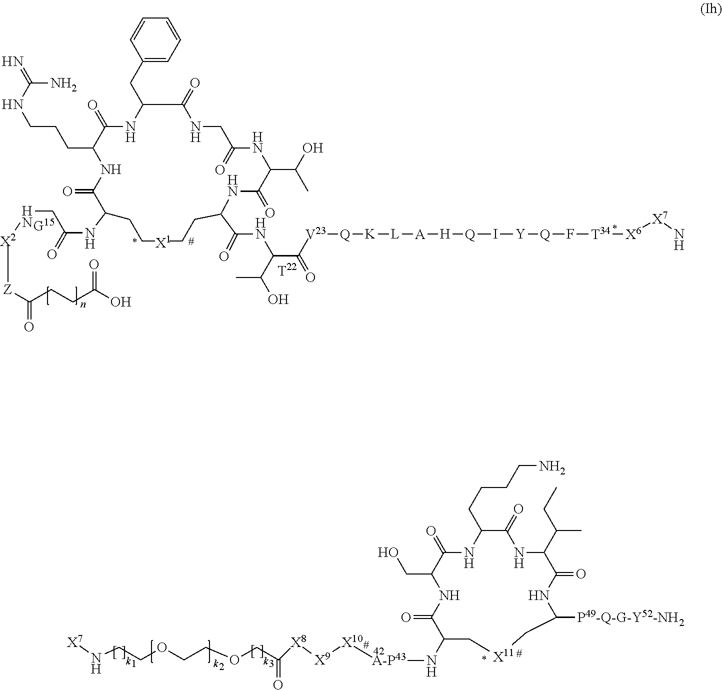

wherein n is 1 to 15, and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 2 to 11, and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 4 to 10, and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 6 to 9, preferably 7 to 9, and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 7 to 8, and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 6 and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 7 and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 8 and wherein X¹ and X² are as defined according to any one of the proceeding embodiments disclosed above or below.

According to an embodiment of the invention, the compounds of formula (Ih), a physiologically acceptable salt, a solvate or a solvate of a salt thereof are as defined as follows: wherein n is 9 and wherein X¹ and X² are as defined according to any one of the proceeding embodiments disclosed above or below.

According to an embodiment of the invention, the compounds of formula (I), a physiologically acceptable salt, a solvate or a solvate of a salt thereof are defined as follows: the compound is selected from

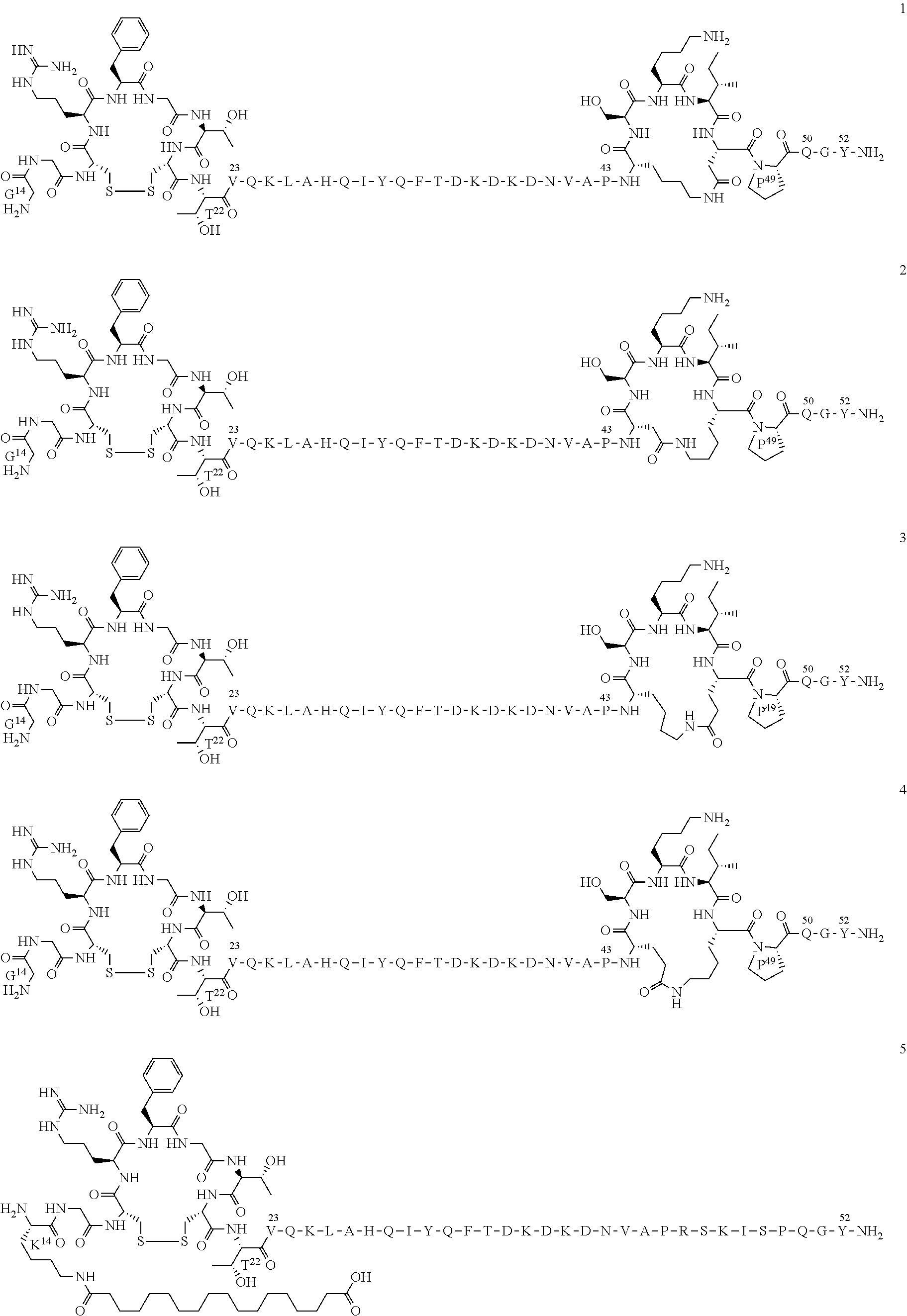

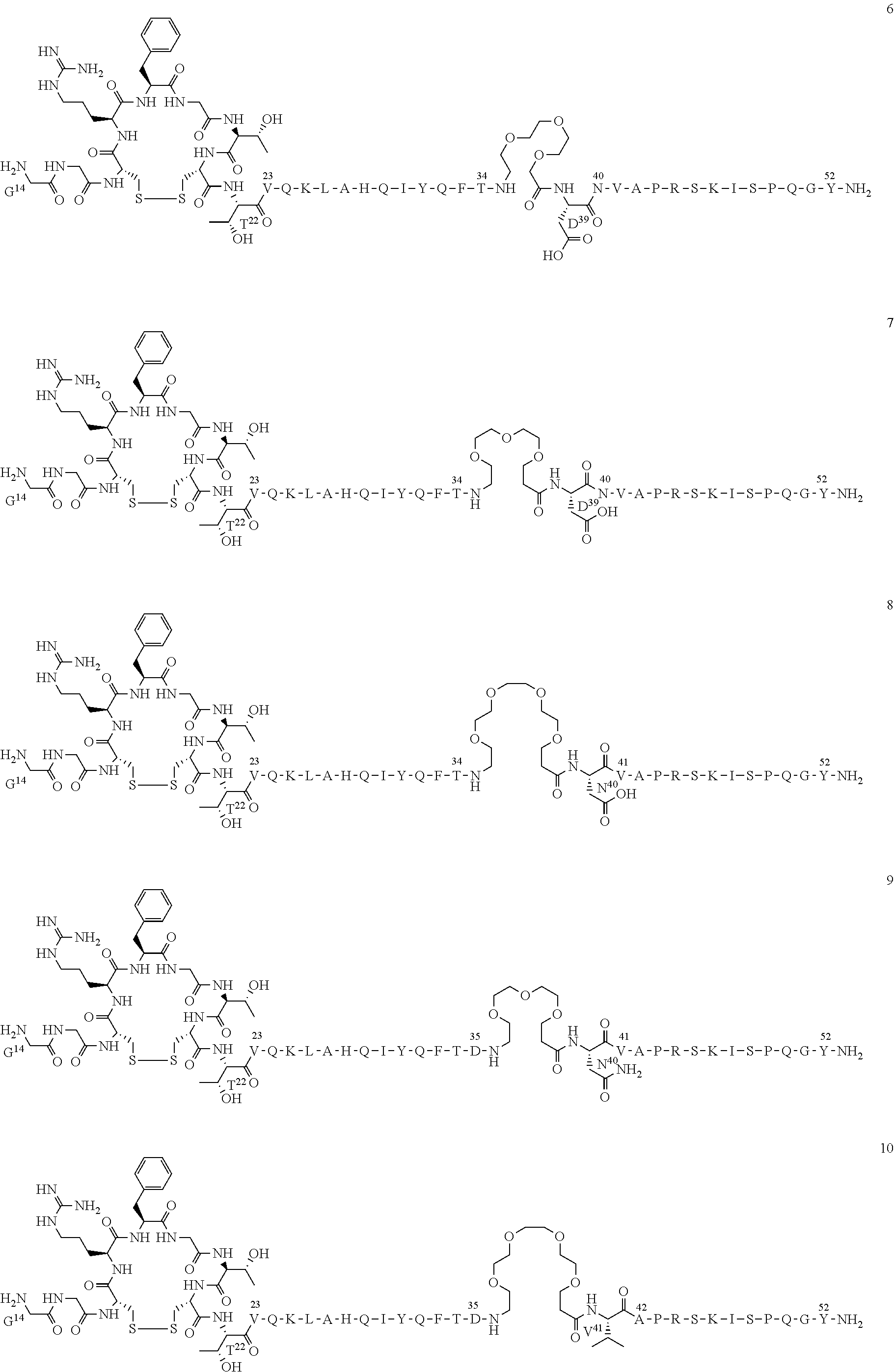

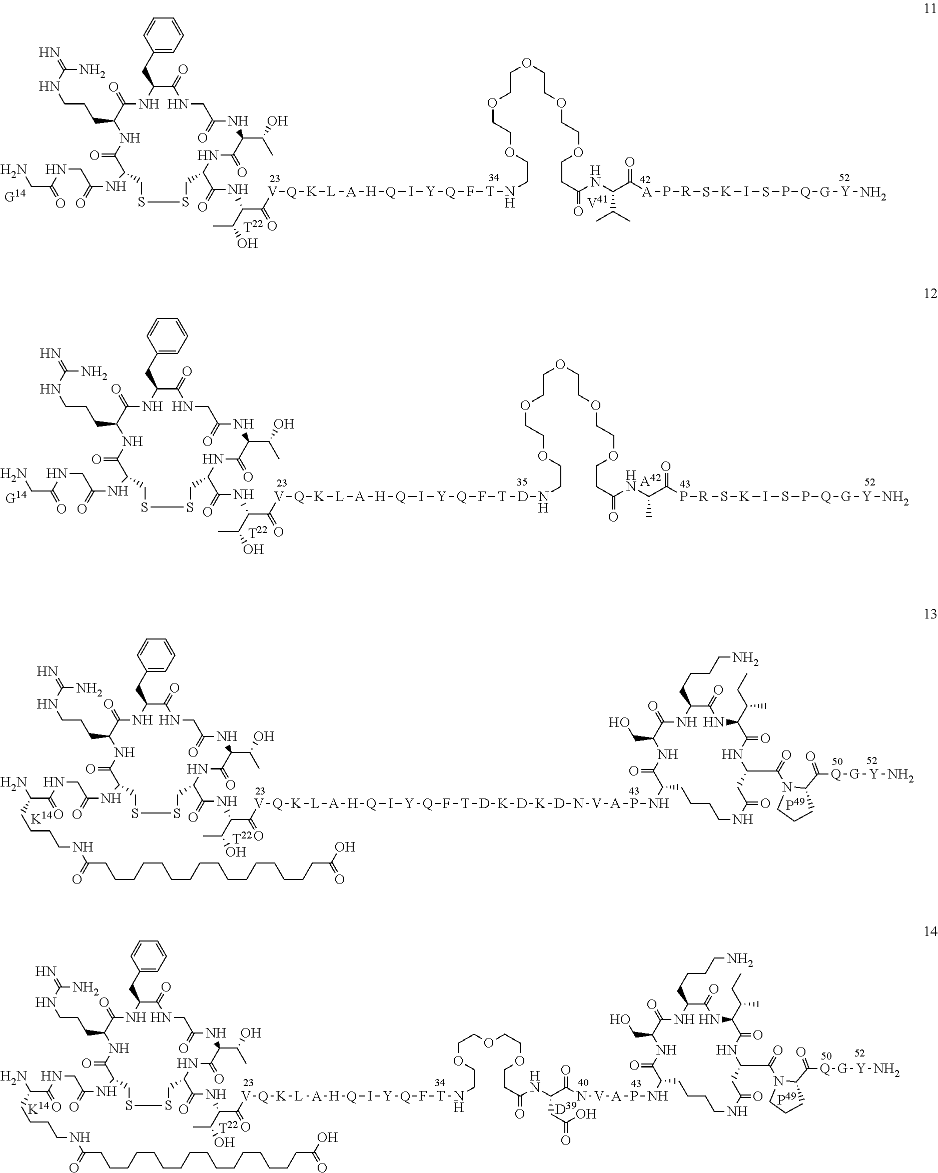

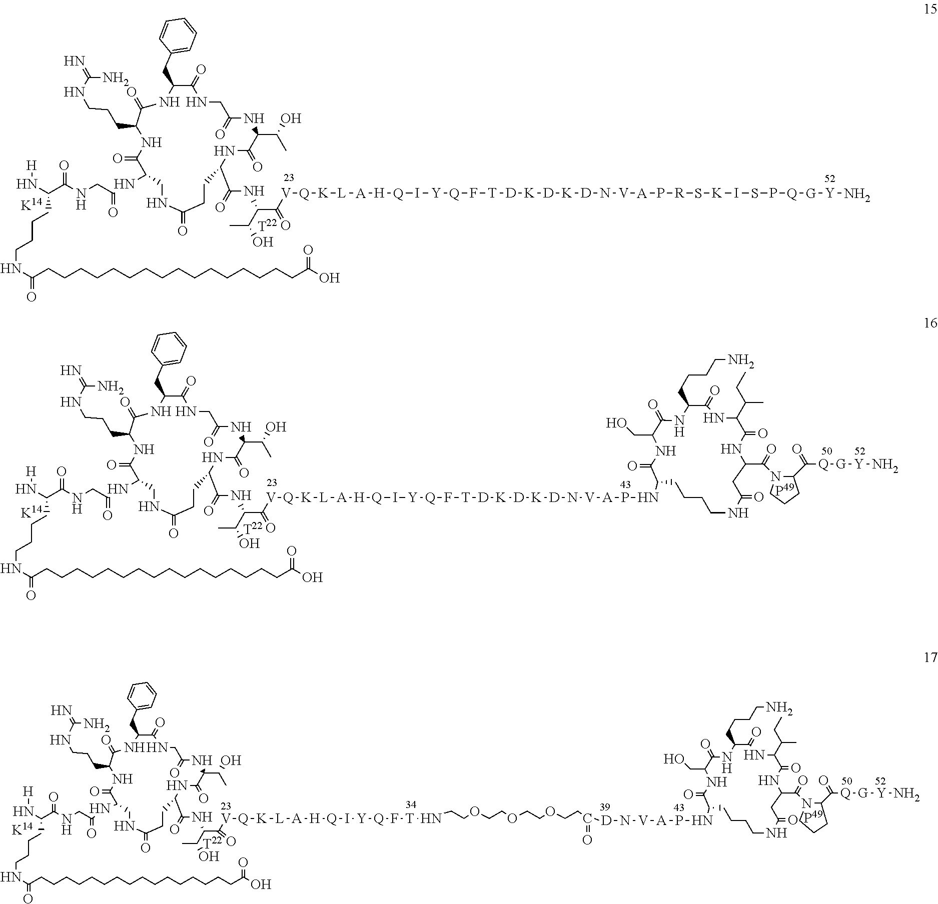

The compounds according to the invention show an unforeseeable useful spectrum of pharmacological activity.

Accordingly they are suitable for use as medicaments for treatment and/or prevention of diseases in humans and animals.

The present invention further provides for the use of the compounds according to the invention for treatment and/or prevention of disorders, especially of cardiovascular, edematous and/or inflammatory disorders.

For the present invention, the term “treatment” or “treating” includes inhibiting, delaying, relieving, mitigating, arresting, reducing, or causing the regression of a disease, disorder, condition, or state, the development and/or progression thereof, and/or the symptoms thereof. The term “prevention” or “preventing” includes reducing the risk of having, contracting, or experiencing, a disease, disorder, condition, or state, the development and/or progression thereof, and/or the symptoms thereof. The term prevention includes prophylaxis. Treatment or prevention of a disease, disorder, condition, or state may be partial or complete.

On the basis of their pharmacological properties, the compounds according to the invention can be employed for treatment and/or prevention of cardiovascular diseases, in particular heart failure, especially chronic and acute heart failure, worsening heart failure, diastolic and systolic (congestive) heart failure, acute decompensated heart failure, cardiac insufficiency, coronary heart disease, angina pectoris, myocardial infarction, ischemia reperfusion injury, ischemic and hemorrhagic stroke, arteriosclerosis, atherosclerosis, hypertension, especially essential hypertension, malignant essential hypertension, secondary hypertension, renovascular hypertension and hypertension secondary to renal and endocrine disorders, hypertensive heart disease, hypertensive renal disease, pulmonary hypertension, especially secondary pulmonary hypertension, pulmonary hypertension following pulmonary embolism with and without acute cor pulmonale, primary pulmonary hypertension, and peripheral arterial occlusive disease.

The compounds according to the invention are furthermore suitable for treatment and/or prevention of gestational [pregnancy-induced] edema and proteinuria with and without hypertension (pre-eclampsia).

The compounds according to the invention are furthermore suitable for treatment and/or prevention of pulmonary disorders, such as chronic obstructive pulmonary disease, asthma, acute and chronic pulmonary edema, allergic alveolitis and pneumonitis due to inhaled organic dust and particles of fungal, actinomycetic or other origin, acute chemical bronchitis, acute and chronic chemical pulmonary edema (e.g. after inhalation of phosgene, nitrogen oxide), neurogenic pulmonary edema, acute and chronic pulmonary manifestations due to radiation, acute and chronic interstitial lung disorders (such as but not restricted to drug-induced interstitial lung disorders, e.g. secondary to Bleomycin treatment), acute lung injury/acute respiratory distress syndrome (ALI/ARDS) in adult or child including newborn, ALI/ARDS secondary to pneumonia and sepsis, aspiration pneumonia and ALI/ARDS secondary to aspiration (such as but not restricted to aspiration pneumonia due to regurgitated gastric content), ALI/ARDS secondary to smoke gas inhalation, transfusion-related acute lung injury (TRALI), ALI/ARDS or acute pulmonary insufficiency following surgery, trauma or burns, ventilator induced lung injury (VILI), lung injury following meconium aspiration, pulmonary fibrosis, and mountain sickness.

The compounds according to the invention are furthermore suitable for treatment and/or prevention of chronic kidney diseases (stages 1-5), renal insufficiency, diabetic nephropathy, hypertensive chronic kidney disease, glomerulonephritis, rapidly progressive and chronic nephritic syndrome, unspecific nephritic syndrome, nephrotic syndrome, hereditary nephropathies, acute and chronic tubulo-interstitial nephritis, acute kidney injury, acute kidney failure, posttraumatic kidney failure, traumatic and postprocedural kidney injury, cardiorenal syndrome, and protection and functional improvement of kidney transplants.

The compounds are moreover suitable for treatment and/or prevention of diabetes mellitus and its consecutive symptoms, such as e.g. diabetic macro- and microangiopathy, diabetic nephropathy and neuropathy.

The compounds according to the invention can moreover be used for treatment and/or prevention of disorders of the central and peripheral nervous system such as viral and bacterial meningitis and encephalitis (e.g. Zoster encephalitis), traumatic and toxic brain injury, primary or secondary [metastasis] malignant neoplasm of the brain and spinal cord, radiculitis and polyradiculitis, Guillain-Barre syndrome [acute (post-)infective polyneuritis, Miller Fisher Syndrome], amyotrophic lateral sclerosis [progressive spinal muscle atrophy], Parkinson's disease, acute and chronic polyneuropathies, pain, cerebral edema, Alzheimer's disease, degenerative diseases of the nervous system and demyelinating diseases of the central nervous system such as but not restricted to multiple sclerosis.

The compounds according to the invention are furthermore suitable for treatment and/or prevention of portal hypertension and liver fibrosis [cirrhosis] and its sequelae such as esophageal varices and ascites, for the treatment and/or prevention of pleural effusions secondary to malignancies or inflammations and for the treatment and/or prevention of lymphedema and of edema secondary to varices.

The compounds according to the invention are furthermore suitable for treatment and/or prevention of inflammatory disorders of the gastrointestinal tract such as inflammatory bowel disease, Crohn's disease, ulcerative colitis, and toxic and vascular disorders of the intestine.

The compounds according to the invention are furthermore suitable for treatment and/or prevention of sepsis, septic shock, systemic inflammatory response syndrome (SIRS) of non-infectious origin, hemorrhagic shock, sepsis or SIRS with organ dysfunction or multi organ failure (MOF), traumatic shock, toxic shock, anaphylactic shock, urticaria, insect sting and bite-related allergies, angioneurotic edema [Giant urticaria, Quincke's edema], acute laryngitis and tracheitis, and acute obstructive laryngitis [croup] and epiglottitis.

The compounds are furthermore suitable for treatment and/or prevention of diseases of the rheumatic type and other disease forms to be counted as autoimmune diseases such as but not restricted to polyarthritis, lupus erythematodes, scleroderma, purpura and vasculitis.

The compounds according to the invention are furthermore suitable for treatment of edematous ocular disorders or ocular disorders associated with disturbed vascular function, including, but not being limited to, age-related macular degeneration (AMD), diabetic retinopathy, in particular diabetic macula edema (DME), subretinal edema, and intraretinal edema. In the context of the present invention, the term age-related macular degeneration (AMD) encompasses both wet (or exudative, neovascular) and dry (or non-exudative, non-neovascular) manifestations of AMD.

The compounds according to the invention are furthermore suitable for treatment of ocular hypertension (glaucoma).

The compounds according to the invention can moreover be used for treatment and/or prevention of operation-related states of ischemia and consecutive symptoms thereof after surgical interventions, in particular interventions on the heart using a heart-lung machine (e.g. bypass operations, heart valve implants), interventions on the carotid arteries, interventions on the aorta and interventions with instrumental opening or penetration of the skull cap.

The compounds are furthermore suitable for general treatment and/or prevention in the event of surgical interventions with the aim of accelerating wound healing and shortening the reconvalescence time. They are further suited for the promotion of wound healing.

The compounds are furthermore suitable for treatment and/or prevention of disorders of bone density and structure such as but not restricted to osteoporosis, osteomalacia and hyperparathyroidism-related bone disorders.

The compounds are furthermore suitable for treatment and/or prevention of sexual dysfunctions, in particular male erectile dysfunction.

Preferable the compounds are suitable for treatment and/or prevention of heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease and/or urticaria.

More preferable the compounds are suitable for treatment and/or prevention of heart failure, chronic heart failure, worsening heart failure, acute heart failure, acute decompensated heart failure, diastolic and systolic (congestive) heart failure, hypertension, pulmonary hypertension, asthma, acute and/or chronic chemical pulmonary edema, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, sepsis, septic shock, systemic inflammatory response syndrome (SIRS) of non-infectious origin, anaphylactic shock, inflammatory bowel disease and/or urticaria.

The present invention further provides for the use of the compounds according to the invention for treatment and/or prevention of disorders, in particular the disorders mentioned above.

The present invention further provides for the use of the compounds according to the invention for preparing a medicament for treatment and/or prevention of disorders, in particular the disorders mentioned above.

The present invention further provides a method for treatment and/or prevention of disorders, in particular the disorders mentioned above, using an active amount of the compounds according to the invention.

The invention further provides medicaments comprising a compound according to the invention and one or more further active ingredients, in particular for treatment and/or prevention of the disorders mentioned above. Exemplary and preferred active ingredient combinations are:

ACE inhibitors, angiotensin receptor antagonists, beta-2 receptor agonists, phosphodiesterase inhibitors, glucocorticoid receptor agonists, diuretics, or recombinant angiotensin converting enzyme-2 or acetylsalicylic acid (aspirin).

In a preferred embodiment of the invention, the compounds according to the invention are administered in combination with an ACE inhibitor, such as, by way of example and preferably, enalapril, quinapril, captopril, lisinopril, ramipril, delapril, fosinopril, perindopril, cilazapril, imidapril, benazepril, moexipnril, spirapril or trandopril.

In a preferred embodiment of the invention, the compounds according to the invention are administered in combination with an angiotensin receptor antagonist, such as, by way of example and preferably, losartan, candesartan, valsartan, telmisartan or embusartan.

In a preferred embodiment of the invention, the compounds according to the invention are administered in combination with a beta-2 receptor agonist, such as, by way of example and preferably, salbutamol, pirbuterol, salmeterol, terbutalin, fenoterol, tulobuterol, clenbuterol, reproterol or formoterol.

In a preferred embodiment of the invention, the compounds according to the invention are administered in combination with a phosphodiesterase (PDE) inhibitor, such as, by way of example and preferably, milrinone, amrinone, pimobendan, cilostazol, sildenafil, vardenafil or tadalafil.

In a preferred embodiment of the invention, the compounds according to the invention are administered in combination with a glucocorticoid receptor agonist, such as, by way of example and preferably, cortiosol, cortisone, hydrocortisone, prednisone, methyl-prednisolone, prednylidene, deflazacort, fluocortolone, triamcinolone, dexamethasone or betamethasone.

In a preferred embodiment of the invention, the compounds according to the invention are administered in combination with diuretics, such as, by way of example and preferably, furosemide, torasemide and hydrochlorothiazide.

In a preferred embodiment of the invention, the compounds according to the invention are administered in combination with natriuretic peptides, such as nesiritide (human B-type natriuretic peptide (hBNP)) and carperitide (alpha-human atrial natriuretic polypeptide (hANP)).

In a preferred embodiment of the invention, the compounds according to the invention are administered in combination with urodilatin, a derivative of ANP still under development for acute heart failure.

In a preferred embodiment of the invention, the compounds according to the invention are administered in combination with LCZ696 (Entresto), a neprilysin (enkephalinase, neutral endopeptidase, NEP, also involved in the metabolism of ADM) inhibitor.

The present invention further relates to medicaments which comprise at least one compound according to the invention, normally together with one or more inert, nontoxic, pharmaceutically suitable excipients and to the use thereof for the aforementioned purposes.

The compounds according to the invention can act systemically and/or locally. For this purpose, they can be administered in a suitable way, for example by the parenteral, pulmonary, nasal, sublingual, lingual, buccal, dermal, transdermal, conjunctival, optic route or as implant or stent.

The compounds according to the invention can be administered in administration forms suitable for these administration routes.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration include preparations for injection and infusion in the form of solutions, suspensions, emulsions, lyophilizates or sterile powders.

Suitable for the other administration routes are, for example, pharmaceutical forms for inhalation (including powder inhalers, nebulizers), nasal drops, eye drops, solutions or sprays; films/wafers or aqueous suspensions (lotions, shaking mixtures), lipophilic suspensions, ointments, creams, transdermal therapeutic systems (e.g. patches), milk, pastes, foams, dusting powders, implants or stents.

Parenteral administration is preferred, especially intravenous administration. Inhalative administration is also preferred, e.g. by using powder inhalers or nebulizers.

The compounds according to the invention can be converted into the stated administration forms. This can take place in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colors (e.g. inorganic pigments, for example iron oxides) and masking flavors and/or odors.

It has generally been found to be advantageous, in the case of parenteral administration, to administer amounts of about 0.001 to 5 mg/kg, preferably about 0.01 to 1 mg/kg, of body weight to achieve effective results.

It may nevertheless be necessary in some cases to deviate from the stated amounts; in particular as a function of the body weight, route of administration, individual response to the active ingredient, nature of the preparation and time or interval over which administration takes place. For instance, less than the aforementioned minimum amount may be sufficient in some cases, whereas in other cases the stated upper limit must be exceeded. In the case of administration of larger amounts, it may be advisable to divide these into a plurality of individual doses over the day.

The compounds of formula (I), a physiologically acceptable salt, a solvate or a solvate of a salt according to the invention can be used in a method for the treatment and/or prevention of cardiovascular, edematous and/or inflammatory disorders.

The compounds of formula (I), a physiologically acceptable salt, a solvate or a solvate of a salt according to the invention can be used in a method for the treatment and/or prevention of heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease, urticaria and/or edematous ocular disorders or ocular disorders associated with disturbed vascular function, including, age-related macular degeneration (AMD), diabetic retinopathy, in particular diabetic macula edema (DME), subretinal edema, and intraretinal edema.

Further disclosed is a medicament comprising a compound of formula (I), a physiologically acceptable salt, a solvate or a solvate of a salt according to the invention or to one of the embodiments disclosed herein in combination with an inert nontoxic pharmaceutically suitable excipient.

Disclosed is a medicament comprising a compound of formula (I), a physiologically acceptable salt, a solvate or a solvate of a salt according to the invention or to one of the embodiments disclosed herein in combination with a further active ingredient selected from the group consisting of ACE inhibitors, angiotensin receptor antagonists, beta-2 receptor agonists, phosphodiesterase (PDE) inhibitors, glucocorticoid receptor agonists, diuretics, recombinant angiotensin converting enzyme-2, acetylsalicylic acid, natriuretic peptides and derivatives thereof, and neprilysin inhibitors.

Disclosed is a medicament comprising a compound of formula (I), a physiologically acceptable salt, a solvate or a solvate of a salt according to the invention or to one of the embodiments disclosed herein for the treatment and/or prevention of cardiovascular, edematous and/or inflammatory disorders.

Further disclosed is a method for the treatment and/or prophylaxis of cardiovascular, edematous and/or inflammatory disorders in humans or animals using an effective amount of at least one a compound of formula (I), a physiologically acceptable salt, a solvate or a solvate of a salt according to the invention or to one of the embodiments disclosed herein or a medicament comprising a compound of formula (I), a physiologically acceptable salt, a solvate or a solvate of a salt according to the invention or to one of the embodiments disclosed herein.

The following working examples illustrate the invention. The invention is not restricted to the examples.

The percentages in the following tests and examples are, unless stated otherwise, percentages by weight; parts are parts by weight. Solvent ratios, dilution ratios and concentration data for the liquid/liquid solutions are each based on volume.

The invention further provides a process for preparing the compounds of the formula (I), or salts thereof, solvates thereof or the solvates of salts thereof.

The following clauses also form part of the disclosure herein:

### CLAUSES

1. A compound according to formula (I)

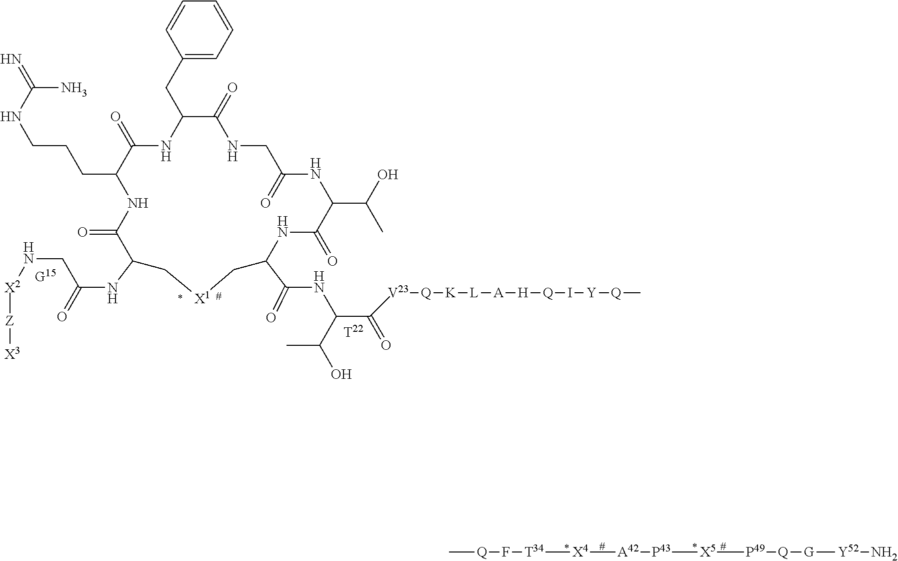

a physiologically acceptable salt, a solvate or a solvate of a salt thereof, wherein
X¹ is selected from the group consisting of
*—(CH₂)ₘ₁—S—S—(CH₂)ₙ₁—^{#}, wherein m1 is 0-6, n1 is 0-6, with the proviso that m1+n1=0-6;
*—(CH₂)ₘ₂—S—(CH₂)ₙ₂—^{#}, wherein m2 is 0-6, n2 is 0-6, with the proviso that m2+n2=0-6;
*—(CH₂)ₘ₃—^{#}, wherein m3 is 1-8;
*—(CH₂)ₘ₄—(CH₂═CH₂)—(CH₂)ₙ₃—^{#}, wherein m4 is 0-6, n3 is 0-6, with the proviso that m4+n3=0-6;
*—(CH₂)ₘ₅—(CH—CH)—(CH₂)ₙ₄—^{#}, wherein m5 is 0-6, and n4 is 0-6, with the proviso that m5+n4=0-6;
*—(CH₂)ₘ₆—CO—NH—(CH₂)ₙ₈—^{#} wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
^{#}—(CH₂)ₘ₈—SO—(CH₂)ₙ₇—*, wherein m8 is 0-4, and n7 is 0-4, with the proviso that m8+n7=0-6;
*^{#}—(CH₂)ₘ₉—SO₂—(CH₂)ₙ₈—*, wherein m9 is 0-4, and n8 is 0-4, with the proviso that m9+n8=0-6;
*-5-6 membered heteroaryl-^{#};
—(CH₂)ₘ₁₀—O—(CH₂)ₙ₉—^{#}, wherein m10 is 0-6, n9 is 0-6, with the proviso that m10+n9=0-6;
*—(CH₂)ₘ₁₈—NH—CO—CH₂—NH—CO—(CH₂)ₙ₈—^{#} wherein m18 is 0-3, and n5 is 0 or 1, with the proviso that m18+n5=0-3; ^{#}—(CH₂)ₘ₁₉—NH—CO—CH₂—NH—CO—(CH₂)ₙ₆—*, wherein m19 is 0-3, and n6 is 0 or 1, with the proviso that m19+n6=0-3;
*—(CH₂)ₘ₂₀—NH—CO—CH(CH₃)—NH—CO—(CH₂)ₙ₇—^{#}, wherein m20 is 0-3, and n7 is 0 or 1, with the proviso that m20+n7=0-3; ^{#}—(CH₂)ₘ₂₁—NH—CO—CH(CH₃)—NH—CO—(CH₂)ₙ₈—*, wherein m21 is 0-3, and n8 is 0 or 1, with the proviso that m21+n8=0-3;
*—(CH₂)ₘ₂₂—NH—CO—CH(CH₂—C(CH₃)₂)—NH—CO—(CH₂)ₙ₉—^{#}, wherein m22 is 0-3, and n9 is 0 or 1, with the proviso that m22+n9=0-3; ^{#}—(CH₂)ₘ₂₃—NH—CO—CH(CH₂—C(CH₃)₂)—NH—CO—(CH₂)ₙ₁₀—*, wherein m23 is 0-3, and n10 is 0 or 1, with the proviso that m23+n10=0-3;
*—(CH₂)ₘ₂₄—NH—CO—CH(CH(CH₃)C₂H₅)—NH—CO—(CH₂)ₙ₁—^{#}, wherein m24 is 0-3, and n11 is 0 or 1, with the proviso that m24+n11=0-3; ^{#}—(CH₂)ₘ₂—NH—CO—CH(CH(CH₃)C₂H₅)—NH—CO—(CH₂)ₙ₁₂—*, wherein m25 is 0-3, and n12 is 0 or 1, with the proviso that m25+n12=0-3;
*—(CH₂)ₘ₂₆—NH—CO—CH(CH₂(C₆H₅))—NH—CO—(CH₂)ₙ₁₃—^{#}, wherein m26 is 0-3, and n13 is 0 or 1, with the proviso that m26+n13=0-3; ^{#}—(CH₂)ₘ₂₇—NH—CO—CH(CH₂(C₆H₅))—NH—CO—(CH₂)ₙ₁₄—*, wherein m27 is 0-3, and n14 is 0 or 1, with the proviso that m27+n14=0-3;
*—(CH₂)ₘ₂₈—NH—CO—(CH₂)₃—NH—CO—(CH₂)ₙ₁₅—^{#}, wherein m28 is 0 or 1, and n15 is 0 or 1, with the proviso that m28+n15=0-1; ^{#}—(CH₂)ₘ₂₉—NH—CO—(CH₂)₃—NH—CO—(CH₂)ₙ₁₆—*, wherein m29 is 0 or 1, and n16 is 0 or 1, with the proviso that m29+n16=0-1;
*—(CH₂)ₘ₃₀—NH—CO—NH—(CH₂)ₙ₁₇—^{#}, wherein m30 is 0-5, and n17 is 0-5, with the proviso that m30+n17=0-5; ^{#}—(CH₂)ₘ₃₁—NH—CO—NH—(CH₂)ₙ₁₅—*, wherein m31 is 0-5, and n18 is 0-5, with the proviso that m31+n18=0-5;
*—(CH₂)ₘ₃₂—O—CO—NH—(CH₂)ₙ₁₉—^{#}, wherein m32 is 0-5, and n19 is 0-5, with the proviso that m32+n19=0-5; ^{#}—(CH₂)ₘ₃₃—O—CO—NH—(CH₂)ₙ₂₀—*, wherein m33 is 0-5, and n20 is 0-5, with the proviso that m33+n20=0-5;
*—(CH₂)ₘ₃₄—O—CO—O—(CH₂)ₙ₂₁—^{#}, wherein m34 is 0-5, and n21 is 0-5, with the proviso that m34+n21=0-5;
*—(CH₂)ₘ₃₅—NH—CO—(CH₂)ₙ₂₂—NH—(CH₂)ₚ₁—^{#}, wherein m35 is 0-4, n22 is 0-4, and p1 is 0-4, with the proviso that m35+n22+p1=0-4; and
*—(CH₂)ₘ₃₆—NH—CO—(CH═CH)—CO—NH—(CH₂)ₙ₂₃—^{#}, wherein m36 is 0-2, and n23 is 0-2, with the proviso that m36+n23=0-2;
wherein * and ^{#} reflect where X¹ is bound within the ring structure; and
X² is absent, is hydrogen, or is an amino acid or an amino acid sequence selected from the group consisting of
G¹⁴, K¹⁴, F¹⁴, SEQ ID NO:1 [Y¹RQSMNNFQGLRSF¹⁴], SEQ ID NO:2 [R²QSMNNFQGLRSF¹⁴], SEQ ID NO:3 [Q³SMNNFQGLRSF¹⁴], SEQ ID NO:4 [S⁴MNNFQGLRSF¹⁴], SEQ ID NO:5 [M⁵NNFQGLRSF¹⁴], SEQ ID NO:6 [N⁶NFQGLRSF¹⁴], SEQ ID NO:7 [N⁷FQGLRSF¹⁴], SEQ ID NO:8 [F⁸QGLRSF¹⁴], SEQ ID NO:9 [Q⁹GLRSF¹⁴], SEQ ID NO:10 [G¹⁰LRSF¹⁴], SEQ ID NO:11 [L¹¹RSF¹⁴], SEQ ID NO: 12 [R¹²SF¹⁴], and SEQ ID NO: 13 [S¹³F¹⁴], wherein any one of the SEQ ID NO:1 to SEQ ID NO: 13 is covalently linked between F¹⁴ of said sequences by an amide bond to the N-terminal G¹⁵ of the amino acid sequence of formula (I), wherein any amino acid of X² can optionally be replaced by a natural or unnatural amino acid;
wherein A is L-Alanine; R is L-Arginine; N is L-Asparagine; D is L-Aspartic acid; Q is L-Glutamine; G is L-Glycine; H is L-Histidine; I is L-Isoleucine; L is L-Leucine; K is L-Lysine; M is L-Methionine; F is L-Phenylalanine; P is L-Proline; S is L-Serine; T is L-Threonine; Y is L-Tyrosine; V is L-Valine;
X³ is absent or is a heterologous moiety which is covalently linked to the N-terminus or to a functional group of the side chain of any amino acid of X², to the N-terminus of G¹⁵ or to Z;
Z is absent or is a cleavable linker covalently bound between the N terminus of any amino acid of X² or of G¹⁵ and X³ or between a functional group of the side chain of any amino acid of X² and X³ wherein if X³ is absent, then Z is also absent and X² is hydrogen or is an amino acid or amino acid sequence as defined above for X²;
wherein if X³ is a heterologous moiety, then X² is absent or is an amino acid or amino acid sequence as defined above for X²;
X⁴ is the amino sequence * [D³⁵ K³⁶ D³⁷ K³⁸ D³⁹ N⁴⁰ V⁴¹]#, wherein at least one amino acid of said sequence can optionally be replaced by a natural or unnatural amino acid and wherein * indicates the binding site to T³⁴ and # indicates the binding site to A⁴², or X⁴ is a moiety according to formula (A), wherein * indicates the binding site to T³⁴ and # indicates the binding site to A⁴²

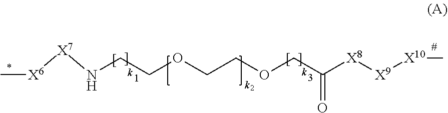

wherein X⁶, X⁷, X⁸, X⁹ and X¹⁰ are independently from another absent or an amino acid selected from L-Alanine; L-Arginine; L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or L-Valine,
wherein k1 is 1, 2, 3 or 4,
wherein k2 is 0, 1, 2, 3, 4, 5, 6, 7 or 8,
wherein k3 is 1, 2, 3 or 4,

X⁵ is the amino sequence * [R⁴⁴ S⁴⁵ K⁴⁶ I⁴⁷ S⁴⁸]#, wherein the sequence can optionally comprise at least one amino acid replaced by a natural or unnatural amino acid and wherein * indicates the binding site to P⁴³ and # indicates the binding site to P⁴⁹, or X⁵ is a moiety according to formula (B), wherein * and ^{#} reflect where X⁵ is bound within the amino acid chain and wherein * indicates the binding site of X⁵ to P⁴³ and # indicates the binding site to P⁴⁹,

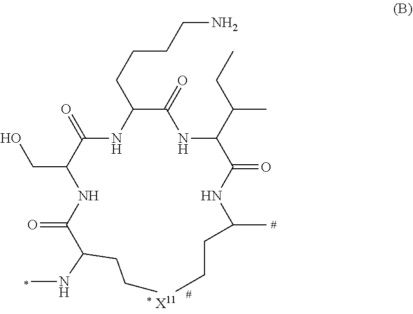

wherein X¹¹ is selected from the group consisting of
*—(CH₂)ₚ₁—S—(CH₂)ᵣ₁—^{#}, wherein p1 is 0-6; r1 is 0-6 with the proviso that p1+r1=0-6;
*—(CH₂)ₚ₂—O—(CH₂)ᵣ₂—^{#}, wherein p2 is 0-6; r2 is 0-6 with the proviso that p1+r2=0-6;
*—(CH₂)ₚ₃—^{#}, wherein p3 is 1-8;
*—(CH₂)ₚ₄—CO—NH—(CH₂)ᵣ₄—^{#}, wherein p4 is 0-4, and r4 is 0-4, with the proviso that p4+r4=0-6;
^{#}—(CH₂)ₚ₅—CO—NH—(CH₂)ᵣ₅*, wherein p5 is 0-4, and r5 is 0-4, with the proviso that p5+r5=0-6;
wherein * and ^{#} reflect where X¹¹ is bound within the ring structure;
wherein the numbering of amino acids in formula (I) refers to the corresponding human adrenomedullin (ADM) sequence;
wherein if X³ is not a di-carboxylic acid, then at least X⁴ is a moiety according to formula (A) as defined above and/or X⁵ is a moiety according to formula (B) as defined above.
2. The compound according to formula (I) according to any one of the proceeding clauses, wherein

X¹ is selected from
*—(CH₂)ₘ₁—S—S—(CH₂)ₙ₁—^{#}, wherein m1 is 0-6, n1 is 0-6, with the proviso that m1+n1=0-6;
*—(CH₂)ₘ₂—S—(CH₂)ₙ₂—^{#}, wherein m2 is 0-6, n2 is 0-6, with the proviso that m2+n2=0-6;
*—(CH₂)ₘ₃—^{#}, wherein m3 is 1-8;
*—(CH₂)ₘ₆—CO—NH—(CH₂)ₙ₈—^{#} wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
*—(CH₂)ₘ₁₀—O—(CH₂)ₙ₉ #, wherein m10 is 0-6, n9 is 0-6, with the proviso that m10+n9=0-6,
wherein * and ^{#} reflect where X¹ is bound within the ring structure.
3. The compound according to formula (I) according to any one of the proceeding clauses, wherein the compound has the following stereoisomerism L-configuration

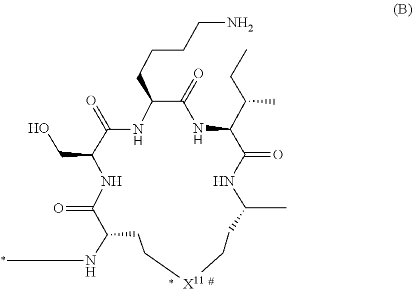

4. The compound according to formula (I) according to any one of the proceeding clauses, wherein X⁵ is a moiety according to formula (B), the compound has the following stereoisomerism L-configuration

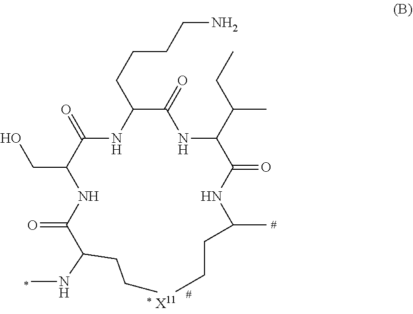

5. The compound according to formula (I) according to any one of the proceeding clauses, wherein X¹ is selected from
   *—(CH₂)ₘ₁—S—S—(CH₂)ₙ₁—^{#}, wherein m1 is 0, 1 or 2, n1 is 0, 1 or 2, with the proviso that m1+n1=0-6;
   *—(CH₂)ₘ₂—S—(CH₂)ₙ₂—^{#}, wherein m2 is 0, 1 or 2, n2 is 0, 1 or 2, with the proviso that m2+n2=0-6;
   *—(CH₂)ₘ₃—^{#}, wherein m3 is 0, 1 or 2;
   *—(CH₂)ₘ₆—CO—NH—(CH₂)ₙ₈—^{#}, wherein m6 is 0, 1 or 2, and n5 is 0, 1 or 2, with the proviso that m6+n5=0-6;
   *^{#}—(CH₂)ₘ₇—CO—NH—(CH₂)ₙ₆—*, wherein m7 is 0, 1 or 2, and n6 is 0, 1 or 2, with the proviso that m7+n6=0-6;
   *—(CH₂)ₘ₁₀—O—(CH₂)ₙ₉ #, wherein m10 is 0, 1 or 2, n9 is 0, 1 or 2, with the proviso that m10+n9=0-6,
   wherein * and ^{#} reflect where X¹ is bound within the ring structure.
6. The compound according to formula (I) according to any one of the proceeding clauses, wherein X¹ is *—(CH₂)ₘ₁—S—S—(CH₂)ₙ₁—^{#}, wherein m1 is 0-6, n1 is 0-6 with the proviso that m1+n1=0-6, and wherein * and ^{#} reflect where X¹ is bound within the ring structure.
7. The compound according to formula (I) according to any one of the proceeding clauses, wherein X¹ is ^{#}—(CH₂)ₘ₁—S—S—(CH₂)ₙ₁—*, wherein m1 is 0, 1 or 2 and n1 is 0, 1 or 2, and wherein * and ^{#} reflect where X¹ is bound within the ring structure.
8. The compound according to formula (I) according to any one of the proceeding clauses, wherein X¹ is ^{#}—(CH₂)ₘ₁—S—S—(CH₂)ₙ₁—*, wherein m1 is 0, 1 or 2 and n1 is 0 or 1, and wherein * and ^{#} reflect where X¹ is bound within the ring structure.
9. The compound according to formula (I) according to any one of the proceeding clauses, wherein X¹ is ^{#}—(CH₂)ₘ₁—S—S—(CH₂)ₙ₁—*, wherein m1 is 0, 1 or 2 and n1 is 0, and wherein * and ^{#} reflect where X¹ is bound within the ring structure.
10. The compound according to formula (I) according to any one of the proceeding clauses, wherein X¹ is ^{#}—(CH₂)ₘ₁—S—S—(CH₂)ₙ₁—*, wherein m1 is 0, 1 or 2 and n1 is 1, and wherein * and ^{#} reflect where X¹ is bound within the ring structure.
11. The compound according to formula (I) according to any one of the proceeding clauses, wherein X¹ is ^{#}—(CH₂)ₘ₁—S—S—(CH₂)ₙ₁—*, wherein m1 is 0, or 1 and n1 is 0, 1 or 2, and wherein * and ^{#} reflect where X¹ is bound within the ring structure.
12. The compound according to formula (I) according to any one of the proceeding clauses, wherein X¹ is ^{#}—(CH₂)ₘ₁—S—S—(CH₂)ₙ₁—*, wherein m1 is 0, or 1 and n1 is 0 or 1, and wherein * and ^{#} reflect where X¹ is bound within the ring structure.
13. The compound according to formula (I) according to any one of the proceeding clauses, wherein X¹ is ^{#}—(CH₂)ₘ₁—S—S—(CH₂)ₙ₁—*, wherein m1 is 0, or 1 and n1 is 0, and wherein * and ^{#} reflect where X¹ is bound within the ring structure.
14. The compound according to formula (I) according to any one of the proceeding clauses, wherein X¹ is ^{#}—(CH₂)ₘ₁—S—S—(CH₂)ₙ₁—*, wherein m1 is 0, or 1 and n1 is 1, and wherein * and ^{#} reflect where X¹ is bound within the ring structure.
15. The compound according to formula (I) according to any one of the proceeding clauses, wherein X¹ is ^{#}—(CH₂)ₘ₁—S—S—(CH₂)ₙ₁—*, wherein m1 is 0 and n1 is 0, 1 or 2, and wherein * and ^{#} reflect where X¹ is bound within the ring structure.
16. The compound according to formula (I) according to any one of the proceeding clauses, wherein X¹ is ^{#}—(CH₂)ₘ₁—S—S—(CH₂)ₙ₁—*, wherein m1 is 0 and n1 is 0, or 1, and wherein * and ^{#} reflect where X¹ is bound within the ring structure.
17. The compound according to formula (I) according to any one of the proceeding clauses, wherein X¹ is ^{#}—(CH₂)ₘ₁—S—S—(CH₂)ₙ₁—*, wherein m1 is 0 and n1 is 1, and wherein * and ^{#} reflect where X¹ is bound within the ring structure.
18. The compound according to formula (I) according to any one of the proceeding clauses, wherein X¹ is ^{#}—(CH₂)ₘ₁—S—S—(CH₂)ₙ₁—*, wherein m1 is 0 and n1 is 0, and wherein * and ^{#} reflect where X¹ is bound within the ring structure.
19. The compound according to formula (I) according to any one of the proceeding clauses, wherein X¹ is ^{#}—(CH₂)ₘ₁—S—S—(CH₂)ₙ₁—*, wherein m1 is 1, or 2 and n1 is 0, 1 or 2, and wherein * and ^{#} reflect where X¹ is bound within the ring structure.
20. The compound according to formula (I) according to any one of the proceeding clauses, wherein X¹ is ^{#}—(CH₂)ₘ₁—S—S—(CH₂)ₙ₁—*, wherein m1 is 1, or 2 and n1 is 0 or 1, and wherein * and ^{#} reflect where X¹ is bound within the ring structure.
21. The compound according to formula (I) according to any one of the proceeding clauses, wherein X¹ is ^{#}—(CH₂)ₘ₁—S—S—(CH₂)ₙ₁—*, wherein m1 is 1 and n1 is 0, 1 or 2, and wherein * and ^{#} reflect where X¹ is bound within the ring structure.
22. The compound according to formula (I) according to any one of the proceeding clauses, wherein X¹ is ^{#}—(CH₂)ₘ₁—S—S—(CH₂)ₙ₁—*, wherein m1 is 1 and n1 is 0, or 1, and wherein * and ^{#} reflect where X¹ is bound within the ring structure.
23. The compound according to formula (I) according to any one of the proceeding clauses, wherein X¹ is ^{#}—(CH₂)ₘ₁—S—S—(CH₂)ₙ₁—*, wherein m1 is 1 and n1 is 1, and wherein * and ^{#} reflect where X¹ is bound within the ring structure.
24. The compound according to formula (I) according to any one of the proceeding clauses, wherein X¹ is ^{#}—(CH₂)ₘ₁—S—S—(CH₂)ₙ₁—*, wherein m1 is 1 and n1 is 0, and wherein * and ^{#} reflect where X¹ is bound within the ring structure.
25. The compound according to formula (I) according to any one of the proceeding clauses, wherein X¹ is *—(CH₂)ₘ₆—CO—NH—(CH₂)ₙ₈—^{#}, wherein m6 is 0-6, n5 is 0-6 with the proviso that m6+n5=0-6, and wherein * and ^{#} reflect where X¹ is bound within the ring structure.
26. The compound according to formula (I) according to any one of the proceeding clauses, wherein X¹ is ^{#}—(CH₂)ₘ₆—CO—NH—(CH₂)ₙ₅—*, wherein m6 is 0, 1 or 2 and n5 is 0, 1 or 2, and wherein * and #reflect where X¹ is bound within the ring structure.
27. The compound according to formula (I) according to any one of the proceeding clauses, wherein X¹ is ^{#}—(CH₂)ₘ₆—CO—NH—(CH₂)ₙ₅—*, wherein m6 is 0, 1 or 2 and n5 is 0 or 1, and wherein * and #reflect where X¹ is bound within the ring structure.
28. The compound according to formula (I) according to any one of the proceeding clauses, wherein X¹ is ^{#}—(CH₂)ₘ₆—CO—NH—(CH₂)ₙ₈—*, wherein m6 is 0, 1 or 2 and n5 is 0, and wherein * and ^{#} reflect where X¹ is bound within the ring structure.
29. The compound according to formula (I) according to any one of the proceeding clauses, wherein X¹ is ^{#}—(CH₂)ₘ₆—CO—NH—(CH₂)ₙ₈—*, wherein m6 is 0, 1 or 2 and n5 is 1, and wherein * and ^{#} reflect where X¹ is bound within the ring structure.
30. The compound according to formula (I) according to any one of the proceeding clauses, wherein X¹ is ^{#}—(CH₂)ₘ₆—CO—NH—(CH₂)ₙ₅—*, wherein m6 is 0, or 1 and n5 is 0, 1 or 2, and wherein * and ^{#} reflect where X¹ is bound within the ring structure.
31. The compound according to formula (I) according to any one of the proceeding clauses, wherein X¹ is ^{#}—(CH₂)ₘ₆—CO—NH—(CH₂)ₙ₅—*, wherein m6 is 0, or 1 and n5 is 0 or 1, and wherein * and ^{#} reflect where X¹ is bound within the ring structure.
32. The compound according to formula (I) according to any one of the proceeding clauses, wherein X¹ is ^{#}—(CH₂)ₘ₆—CO—NH—(CH₂)ₙ₈—*, wherein m6 is 0, or 1 and n5 is 0, and wherein * and ^{#} reflect where X¹ is bound within the ring structure.
33. The compound according to formula (I) according to any one of the proceeding clauses, wherein X¹ is ^{#}—(CH₂)ₘ₆—CO—NH—(CH₂)ₙ₈—*, wherein m6 is 0, or 1 and n5 is 1, and wherein * and ^{#} reflect where X¹ is bound within the ring structure.
34. The compound according to formula (I) according to any one of the proceeding clauses, wherein X¹ is ^{#}—(CH₂)ₘ₆—CO—NH—(CH₂)ₙ₈—*, wherein m6 is 0 and n5 is 0, 1 or 2, and wherein * and ^{#} reflect where X¹ is bound within the ring structure.
35. The compound according to formula (I) according to any one of the proceeding clauses, wherein X¹ is ^{#}—(CH₂)ₘ₆—CO—NH—(CH₂)ₙ₈—*, wherein m6 is 0 and n5 is 0, or 1, and wherein * and ^{#} reflect where X¹ is bound within the ring structure.
36. The compound according to formula (I) according to any one of the proceeding clauses, wherein X¹ is ^{#}—(CH₂)ₘ₆—CO—NH—(CH₂)ₙ₈—*, wherein m6 is 0 and n5 is 1, and wherein * and ^{#} reflect where X¹ is bound within the ring structure.
37. The compound according to formula (I) according to any one of the proceeding clauses, wherein X¹ is ^{#}—(CH₂)ₘ₆—CO—NH—(CH₂)ₙ₈—*, wherein m6 is 0 and n5 is 0, and wherein * and ^{#} reflect where X¹ is bound within the ring structure.
38. The compound according to formula (I) according to any one of the proceeding clauses, wherein X¹ is ^{#}—(CH₂)ₘ₆—CO—NH—(CH₂)ₙ₅—*, wherein m6 is 1, or 2 and n5 is 0, 1 or 2, and wherein * and ^{#} reflect where X¹ is bound within the ring structure.
39. The compound according to formula (I) according to any one of the proceeding clauses, wherein X¹ is ^{#}—(CH₂)ₘ₆—CO—NH—(CH₂)ₙ₅—*, wherein m6 is 1, or 2 and n5 is 0 or 1, and wherein * and ^{#} reflect where X¹ is bound within the ring structure.
40. The compound according to formula (I) according to any one of the proceeding clauses, wherein X¹ is ^{#}—(CH₂)ₘ₆—CO—NH—(CH₂)ₙ₈—*, wherein m6 is 1 and n5 is 0, 1 or 2, and wherein * and ^{#} reflect where X¹ is bound within the ring structure.
41. The compound according to formula (I) according to any one of the proceeding clauses, wherein X¹ is ^{#}—(CH₂)ₘ₆—CO—NH—(CH₂)ₙ₈—*, wherein m6 is 1 and n5 is 0, or 1, and wherein * and ^{#} reflect where X¹ is bound within the ring structure.
42. The compound according to formula (I) according to any one of the proceeding clauses, wherein X¹ is ^{#}—(CH₂)ₘ₆—CO—NH—(CH₂)ₙ₈—*, wherein m6 is 1 and n5 is 1, and wherein * and ^{#} reflect where X¹ is bound within the ring structure.
43. The compound according to formula (I) according to any one of the proceeding clauses, wherein X¹ is ^{#}—(CH₂)ₘ₆—CO—NH—(CH₂)ₙ₈—*, wherein m6 is 1 and n5 is 0, and wherein * and ^{#} reflect where X¹ is bound within the ring structure.
44. The compound according to formula (I) according to any one of the proceeding clauses, wherein X¹ is ^{#}—(CH₂)ₘ₇—CO—NH—(CH₂)ₙ₆—*, wherein m7 is 0-6, n6 is 0-6 with the proviso that m7+n6=0-6, and wherein * and ^{#} reflect where X¹ is bound within the ring structure.
45. The compound according to formula (I) according to any one of the proceeding clauses, wherein X¹ is ^{#}—(CH₂)ₘ₇—CO—NH—(CH₂)ₙ₆—*, wherein m7 is 0, 1 or 2 and n6 is 0, 1 or 2, and wherein * and ^{#} reflect where X¹ is bound within the ring structure.
46. The compound according to formula (I) according to any one of the proceeding clauses, wherein X¹ is ^{#}—(CH₂)ₘ₇—CO—NH—(CH₂)ₙ₆—*, wherein m7 is 0, 1 or 2 and n6 is 0 or 1, and wherein * and ^{#} reflect where X¹ is bound within the ring structure.
47. The compound according to formula (I) according to any one of the proceeding clauses, wherein X¹ is ^{#}—(CH₂)ₘ₇—CO—NH—(CH₂)ₙ₆—*, wherein m7 is 0, 1 or 2 and n6 is 0, and wherein * and ^{#} reflect where X¹ is bound within the ring structure.
48. The compound according to formula (I) according to any one of the proceeding clauses, wherein X¹ is ^{#}—(CH₂)ₘ₇—CO—NH—(CH₂)ₙ₆—*, wherein m7 is 0, 1 or 2 and n6 is 1, and wherein * and ^{#} reflect where X¹ is bound within the ring structure.
49. The compound according to formula (I) according to any one of the proceeding clauses, wherein X¹ is ^{#}—(CH₂)ₘ₇—CO—NH—(CH₂)ₙ₆—*, wherein m7 is 0, or 1 and n6 is 0, 1 or 2, and wherein * and ^{#} reflect where X¹ is bound within the ring structure.
50. The compound according to formula (I) according to any one of the proceeding clauses, wherein X¹ is ^{#}—(CH₂)ₘ₇—CO—NH—(CH₂)ₙ₆—*, wherein m7 is 0, or 1 and n6 is 0 or 1, and wherein * and ^{#} reflect where X¹ is bound within the ring structure.
51. The compound according to formula (I) according to any one of the proceeding clauses, wherein X¹ is ^{#}—(CH₂)ₘ₇—CO—NH—(CH₂)ₙ₆—*, wherein m7 is 0, or 1 and n6 is 0, and wherein * and ^{#} reflect where X¹ is bound within the ring structure.
52. The compound according to formula (I) according to any one of the proceeding clauses, wherein X¹ is ^{#}—(CH₂)ₘ₇—CO—NH—(CH₂)ₙ₆—*, wherein m7 is 0, or 1 and n6 is 1, and wherein * and ^{#} reflect where X¹ is bound within the ring structure.
53. The compound according to formula (I) according to any one of the proceeding clauses, wherein X¹ is ^{#}—(CH₂)ₘ₇—CO—NH—(CH₂)ₙ₆—*, wherein m7 is 0 and n6 is 0, 1 or 2, and wherein * and ^{#} reflect where X¹ is bound within the ring structure.
54. The compound according to formula (I) according to any one of the proceeding clauses, wherein X¹ is ^{#}—(CH₂)ₘ₇—CO—NH—(CH₂)ₙ₆—*, wherein m7 is 0 and n6 is 0, or 1, and wherein * and ^{#} reflect where X¹ is bound within the ring structure.
55. The compound according to formula (I) according to any one of the proceeding clauses, wherein X¹ is ^{#}—(CH₂)ₘ₇—CO—NH—(CH₂)ₙ₆—*, wherein m7 is 0 and n6 is 1, and wherein * and ^{#} reflect where X¹ is bound within the ring structure.
56. The compound according to formula (I) according to any one of the proceeding clauses, wherein X¹ is ^{#}—(CH₂)ₘ₇—CO—NH—(CH₂)ₙ₆—*, wherein m7 is 0 and n6 is 0, and wherein * and ^{#} reflect where X¹ is bound within the ring structure.
57. The compound according to formula (I) according to any one of the proceeding clauses, wherein X¹ is ^{#}—(CH₂)ₘ₇—CO—NH—(CH₂)ₙ₆—*, wherein m7 is 1, or 2 and n6 is 0, 1 or 2, and wherein * and ^{#} reflect where X¹ is bound within the ring structure.
58. The compound according to formula (I) according to any one of the proceeding clauses, wherein X¹ is ^{#}—(CH₂)ₘ₇—CO—NH—(CH₂)ₙ₆—*, wherein m7 is 1, or 2 and n6 is 0 or 1, and wherein * and ^{#} reflect where X¹ is bound within the ring structure.
59. The compound according to formula (I) according to any one of the proceeding clauses, wherein X¹ is ^{#}—(CH₂)ₘ₇—CO—NH—(CH₂)ₙ₆—*, wherein m7 is 1 and n6 is 0, 1 or 2, and wherein * and ^{#} reflect where X¹ is bound within the ring structure.
60. The compound according to formula (I) according to any one of the proceeding clauses, wherein X¹ is ^{#}—(CH₂)ₘ₇—CO—NH—(CH₂)ₙ₆—*, wherein m7 is 1 and n6 is 0, or 1, and wherein * and ^{#} reflect where X¹ is bound within the ring structure.
61. The compound according to formula (I) according to any one of the proceeding clauses, wherein X¹ is ^{#}—(CH₂)ₘ₇—CO—NH—(CH₂)ₙ₆—*, wherein m7 is 1 and n6 is 1, and wherein * and ^{#} reflect where X¹ is bound within the ring structure.
62. The compound according to formula (I) according to any one of the proceeding clauses, wherein X¹ is ^{#}—(CH₂)ₘ₇—CO—NH—(CH₂)ₙ₆—*, wherein m7 is 1 and n6 is 0, and wherein * and ^{#} reflect where X¹ is bound within the ring structure.
63. The compound according to formula (I) according to any one of the proceeding clauses, wherein X² is absent, is hydrogen, or is an amino acid or an amino acid sequence selected from the group consisting of G¹⁴ and K¹⁴, which is covalently linked by an amide bond to the N-terminal G¹⁵ of the compound of formula (I).
64. The compound according to formula (I) according to any one of the proceeding clauses, wherein X² is G¹⁴ or K¹⁴, which is covalently linked by an amide bond to the N-terminal G¹⁵ of the compound of formula (I).
65. The compound of formula (I) according to any one of the proceeding clauses, wherein X³ is a heterologous moiety selected from the group consisting of a polymer, a Fc, a FcRn binding ligand, albumin and an albumin-binding ligand; or a physiologically acceptable salt, a solvate or a solvate of a salt thereof.
66. The compound of formula (I) according to any one of the proceeding clauses, wherein X³ is a polymer and the polymer is selected from the group consisting of linear or branched C1-C100 carboxylic acids and carboxylic di-acids, preferably C4-C30 carboxylic acids and carboxylic di-acids, optionally substituted with halo, hydroxy, alkoxy, amino, alkylamino, dialky lamino, sulfate, or phosphate, and which may be saturated, or mono- or di-unsaturated, a PEG moiety, a PPG moiety, a PAS moiety and a HES moiety; or a physiologically acceptable salt, a solvate or a solvate of a salt thereof.
67. The compound according to formula (I) according to any one of the proceeding clauses, wherein X³ is selected from the group consisting of linear or branched C3-C30 carboxylic acid or di carboxylic acid, preferably C4-C20 carboxylic acids or di carboxylic acid, more preferably C16-C18 carboxylic acid or di-carboxylic acid, most preferably C16-C18 di carboxylic acid, optionally substituted with halo, hydroxy, alkoxy, amino, alkylamino, dialkylamino, sulfate, or phosphate, and which may be saturated, or mono- or di-unsaturated, a PEG moiety, a PPG moiety, a PAS moiety and a HES moiety.
68. The compound of formula (I) according to any one of the proceeding clauses, wherein the carboxylic acid is selected from the group consisting of arachidic acid, arachidonic acid, behenic acid, capric acid, caproic acid, caprylic acid, ceroplastic acid, cerotic acid, docosahexaenoic acid, eicosapentaenoic acid, elaidic acid, enanthic acid, erucic acid, geddic acid, hen atriacontylic acid, heneicosylic acid, heptacosylic acid, hexatriacontylic acid, lacceroic acid, lauric acid, lignoceric acid, linoelaidic acid, linoleic acid, margaric acid, melissic acid, montanic acid, myristic acid, myristoleic acid, nona cosylic acid, nonadecylic acid, oleic acid, palmitic acid, palmitoleic acid, pantothenic acid, pelargonic acid, penta cosylic acid, pentadecylic acid, psyllic acid, sapienic acid, stearic acid, tricosylic acid, tridecylic acid, undecylic acid, vaccenic acid, valeric acid, a—linolenic acid, C14-C22 carboxylic di-acids and derivatives thereof; or a physiologically acceptable salt, a solvate or a solvate of a salt thereof.
69. The compound of formula (I) according to any one of the proceeding clauses, wherein X³ is a carboxylic di-acid, preferably a C14-C22 carboxylic di-acid, more preferably a C14-C18 carboxylic di-acid or derivatives thereof.
70. The compound of formula (I) according to any one of the proceeding clauses, wherein X³ is a C-14 carboxylic di-acid or derivatives thereof
71. The compound of formula (I) according to any one of the proceeding clauses, wherein X³ is a C-16 carboxylic di-acid or derivatives thereof.
72. The compound of formula (I) according to any one of the proceeding clauses, wherein X³ is a C-18 carboxylic di-acid or derivatives thereof.
73. The compound of formula (I) according to any one of the proceeding clauses, wherein X³ is a moiety according to Formula (C)

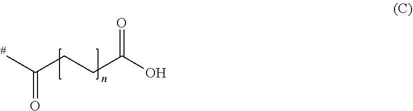

wherein n is 1 to 15, and wherein X¹, X², X⁴ and X⁵ and are as defined according to any one of the proceeding clauses and wherein # indicates the binding site to Z, wherein, if Z is absent, # indicates the binding site to X².
74. The compound of formula (I) according to any one of the proceeding clauses, wherein X³ is a moiety according to Formula (C), wherein n is 2 to 11, and wherein X¹, X², X⁴ and X⁵ are as defined according to any one of the proceeding clauses and wherein # indicates the binding site to Z, wherein, if Z is absent, # indicates the binding site to X².
75. The compound of formula (I) according to any one of the proceeding clauses, wherein X³ is a moiety according to Formula (C), wherein n is 4 to 10, and wherein X¹, X², X⁴ and X⁵ are as defined according to any one of the proceeding clauses and wherein # indicates the binding site to Z, wherein, if Z is absent, # indicates the binding site to X².
76. The compound of formula (I) according to any one of the proceeding clauses, wherein X³ is a moiety according to Formula (C), wherein n is 6 to 9, preferably 7 to 9, and wherein X¹, X², X⁴ and X⁵ are as defined according to any one of the proceeding clauses and wherein # indicates the binding site to Z, wherein, if Z is absent, # indicates the binding site to X².
77. The compound of formula (I) according to any one of the proceeding clauses, wherein X³ is a moiety according to Formula (C), wherein n is 7 to 8, and wherein X¹, X², X⁴ and X⁵ are as defined according to any one of the proceeding clauses and wherein # indicates the binding site to Z, wherein, if Z is absent, # indicates the binding site to X².
78. The compound of formula (I) according to any one of the proceeding clauses, wherein X³ is a moiety according to Formula (C), wherein n is 6 and wherein X¹, X², X⁴ and X⁵ are as defined according to any one of the proceeding clauses and wherein # indicates the binding site to Z, wherein, if Z is absent, # indicates the binding site to X².
79. The compound of formula (I) according to any one of the proceeding clauses, wherein X³ is a moiety according to Formula (C), wherein n is 7 and wherein X¹, X², X⁴ and X⁵ are as defined according to any one of the proceeding clauses and wherein # indicates the binding site to Z, wherein, if Z is absent, # indicates the binding site to X².
80. The compound of formula (I) according to any one of the proceeding clauses, wherein X³ is a moiety according to Formula (C), wherein n is 8 and wherein X¹, X², X⁴ and X⁵ are as defined according to any one of the proceeding clauses and wherein # indicates the binding site to Z, wherein, if Z is absent, # indicates the binding site to X².
81. The compound of formula (I) according to any one of the proceeding clauses, wherein X³ is a moiety according to Formula (C), wherein n is 9 and wherein X¹, X², X⁴ and X⁵ are as defined according to any one of the proceeding clauses and wherein # indicates the binding site to Z, wherein, if Z is absent, # indicates the binding site to X².
82. The compound according to formula (I) according to any one of the proceeding clauses, wherein X² is G¹⁴ or K¹⁴, which is covalently linked by an amide bond to the N-terminal G¹⁵ of the compound of formula (I);
   X³ is absent or is a heterologous moiety, which is covalently linked to the N-terminus of G¹⁴ or K¹⁴ or to a functional group of the side chain of K¹⁴, or to Z;
   Z is absent or is a cleavable linker covalently bound between the N terminus of G¹⁴ or K¹⁴ and X³, or between a functional group of the side chain of K¹⁴ and X³;
   wherein if X³ is absent, then Z is also absent;
   wherein if X³ is a heterologous moiety, then Z is absent or is a cleavable linker covalently bound between the N terminus of G¹⁴ or K¹⁴ and X³, or between a functional group of the side chain of K¹⁴ and X³.
83. The compound according to formula (I) according to any one of the proceeding clauses, wherein X³ is absent or X³ is a dicarboxylic acid.
84. The compound according to formula (I) according to any one of the proceeding clauses, wherein
   X² is G¹⁴ or K¹⁴, which is covalently linked by an amide bond to the N-terminal G¹⁵ of the compound of formula (I);
   X³ is absent;
   Z is absent;
   or a physiologically acceptable salt, a solvate or a solvate of a salt thereof.
85. The compound according to formula (I) according to any one of the proceeding clauses, wherein

X⁴ is the amino sequence * [D³⁵ K³⁶ D³⁷ K³⁸ D³⁹ N⁴⁰ V⁴¹]#, wherein * indicates the binding site to T³⁴ and # indicates the binding site to A⁴².
86. The compound according to formula (I) according to any one of the proceeding clauses, wherein X⁴ is the following amino sequence * [D³⁵ K³⁶ D³⁷ K³⁸ D³⁹ N⁴⁰ V⁴¹]#, wherein * indicates the binding site to T³⁴ and # indicates the binding site to A⁴², wherein one or more of the amino acids of said sequence is substituted by a natural or unnatural amino acid.
87. The compound according to formula (I) according to any one of the proceeding clauses, wherein X⁴ is the following amino sequence * [D³⁵ K³⁶ D³⁷ K³⁸ D³⁹ N⁴⁰ V⁴¹]#, wherein * indicates the binding site to T³⁴ and # indicates the binding site to A⁴², wherein V⁴1 is substituted by a natural or unnatural amino acids and/or A⁴² is substituted by natural or unnatural amino acids.
88. The compound according to formula (I) according to any one of the proceeding clauses, wherein X⁴ is a moiety according to formula (A)

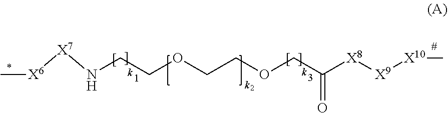

wherein X⁶, X⁷, X⁸, X⁹ and X¹⁰ are independently from another absent or an amino acid selected from L-Alanine; is L-Arginine; is L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or V is L-Valine,
wherein k1 is 1 or 2;
wherein k2 is 0, 1, 2, 3, or 4;
wherein k3 is 1 or 2,
* and ^{#} reflect where X⁴ is bound within the amino acid chain, wherein * indicates the binding site to T³⁴ and # indicates the binding site to A⁴²
89. The compound according to formula (I) according to any one of the proceeding clauses, wherein X⁴ is a moiety according to formula (A)
   wherein X⁶ is absent or selected from the group consisting of D, N and V;
   wherein X⁷ is absent or is selected from the group consisting of D, N and V;
   wherein X⁸ is absent or is selected from the group consisting of D, N and V;
   wherein X⁹ is absent or is selected from the group consisting of D, N and;
   and wherein X¹⁰ is absent or is selected from the group consisting of D, N and V;
   wherein k1 is 1 or 2;
   wherein k2 is 0, 1, 2, 3, or 4;
   wherein k3 is 1 or 2,
   * and ^{#} reflect where X⁴ is bound within the amino acid chain, wherein * indicates the binding site to T³⁴ and # indicates the binding site to A⁴².
90. The compound according to formula (I) according to any one of the proceeding clauses, wherein X⁴ is a moiety according to formula (A)
   wherein k1 is 1;
   wherein k2 is 2, 3, or 4;
   wherein k3 is 1 or 2,
   wherein * and ^{#} reflect where X⁴ is bound within the amino acid chain, wherein * indicates the binding site to T³⁴ and # indicates the binding site to A⁴²
91. The compound according to formula (I) according to any one of the proceeding clauses, wherein X⁴ is a moiety according to formula (A)
   wherein k1 is 1;
   wherein k2 is 2;
   wherein k3 is 1,
   wherein * and ^{#} reflect where X⁴ is bound within the amino acid chain, wherein * indicates the binding site to T³⁴ and # indicates the binding site to A⁴²
92. The compound according to formula (I) according to any one of the proceeding clauses, wherein X⁴ is a moiety according to formula (A)
   wherein k1 is 1;
   wherein k2 is 3;
   wherein k3 is 2,
   wherein * and ^{#} reflect where X⁴ is bound within the amino acid chain, wherein * indicates the binding site to T³⁴ and # indicates the binding site to A⁴²
93. The compound according to formula (I) according to any one of the proceeding clauses, wherein X⁴ is a moiety according to formula (A)
   wherein k1 is 1;
   wherein k2 is 4;
   wherein k3 is 2,
   wherein * and ^{#} reflect where X⁴ is bound within the amino acid chain, wherein * indicates the binding site to T³⁴ and # indicates the binding site to A⁴².
94. The compound according to formula (I) according to any one of the proceeding clauses, wherein X⁴ is a moiety according to formula (A)
   wherein X⁶ is absent or selected from the group consisting of D, N and V;
   wherein X⁷ is absent or is selected from the group consisting of D, N and V;
   wherein * and ^{#} reflect where X⁴ is bound within the amino acid chain, wherein * indicates the binding site to T³⁴ and # indicates the binding site to A⁴²
95. The compound according to formula (I) according to any one of the proceeding clauses, wherein
   X⁴ is a moiety according to formula (A), wherein
   X⁸ is absent or is selected from the group consisting of D, N and V;
   wherein X⁹ is absent or is selected from the group consisting of D, N and;
   and wherein X¹⁰ is absent or is selected from the group consisting of D, N and V;
   * and ^{#} reflect where X⁴ is bound within the amino acid chain, wherein * indicates the binding site to T³⁴ and # indicates the binding site to A⁴²
96. The compound according to formula (I) according to any one of the proceeding clauses, wherein X⁵ is the amino sequence * [R⁴⁴ S⁴⁵ K⁴⁶ I⁴⁷ S⁴⁸]#, wherein * indicates the binding site to P⁴³ and # indicates the binding site to P⁴⁹.
97. The compound according to formula (I) according to any one of the proceeding clauses, wherein X⁵ is the amino sequence * [R⁴⁴ S⁴⁵ K⁴⁶ I⁴⁷ S⁴⁸]#, wherein * indicates the binding site to P⁴³ and #indicates the binding site to P⁴⁹, wherein at least one amino acid of said sequence is substituted by a natural or unnatural amino acid.
98. The compound according to formula (I) according to any one of the proceeding clauses, wherein X⁵ is the amino sequence * [R⁴⁴ S⁴⁵ K⁴⁶ I⁴⁷ S⁴⁸]#, wherein * indicates the binding site to P⁴³ and #indicates the binding site to P⁴⁹, wherein S⁴⁵ and/or S^{4U} are independently substituted by a natural or unnatural amino acid.
99. The compound according to formula (I) according to any one of the proceeding clauses, wherein X⁵ is the moiety according to formula (B),

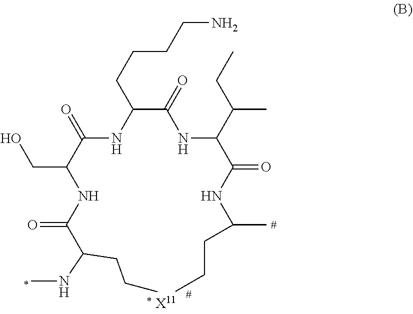

wherein * and ^{#} reflect where X⁵ is bound within the amino acid chain and wherein * indicates the binding site of X⁵ to P⁴³ and # indicates the binding site to P⁴⁹,
wherein X¹¹ is selected from the group consisting of
*—(CH₂)ₚ₁—S—(CH₂)ᵣ₁^{#}, wherein p1 is 0-4 and r1 is 0 or 1;
^{#}—(CH₂)ₚ₂—S—(CH₂)ᵣ₂*, wherein p2 is 0-4 and r2 is 0 or 1;
*—(CH₂)ₚ₃—^{#}, wherein p3 is 1-4;
*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;
^{#}—(CH₂)ₚ₅—CO—NH—(CH₂)ᵣ₅*, wherein p5 is 0, 1, 2 or 3, and r5 is 0, 1, 2 or 3, with the proviso that p5+r5=0-4;
wherein * and ^{#} reflect where X¹¹ is bound within the ring structure.
100. The compound according to formula (I) according to any one of the proceeding clauses, wherein X⁵ is the moiety according to formula (B),

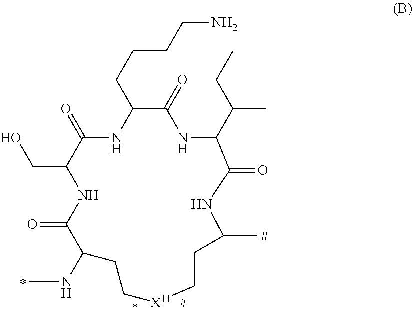

wherein * and ^{#} reflect where X⁵ is bound within the amino acid chain and wherein * indicates the binding site of X⁵ to P⁴³ and # indicates the binding site to P⁴⁹
wherein X¹ is selected from the group consisting of
*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;
^{#}—(CH₂)ₚ₅—CO—NH—(CH₂)ᵣ₅*, wherein p5 is 0, 1, 2 or 3, and r5 is 0, 1, 2 or 3, with the proviso that p5+r5=0-4;
wherein * and ^{#} reflect where X¹ is bound within the ring structure.
101. The compound according to formula (I) according to any one of the proceeding clauses, wherein X⁵ is the moiety according to formula (B),

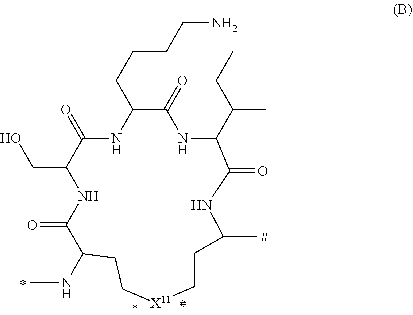

wherein * and ^{#} reflect where X⁵ is bound within the amino acid chain, wherein * indicates the binding site to P⁴³ and # indicates the binding site to P⁴⁹ wherein X¹ is selected from the group consisting of
*—(CH₂)ₚ₄—CO—NH—(CH₂)ᵣ₄—^{#}, wherein p4 is 0 or 1 or, and r4 is 0, 1, 2 or 3, with the proviso that p4+r4=0-4;
^{#}—(CH₂)ₚ₅—CO—NH—(CH₂)ᵣ₅—*, wherein p5 is 0 or 1, and r5 is 0, 1, 2 or 3, with the proviso that p5+r5=0-4;
wherein * and ^{#} reflect where X¹ is bound within the ring structure.
102. The compound according to formula (I) according to any one of the proceeding clauses, wherein X⁵ is the moiety according to formula (B),

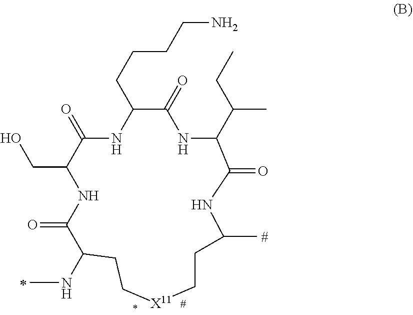

wherein * and ^{#} reflect where X⁵ is bound within the amino acid chain, wherein * indicates the binding site to P⁴³ and # indicates the binding site to P⁴⁹
wherein X¹¹ is selected from the group consisting of
*—(CH₂)ₚ₄—CO—NH—(CH₂)ᵣ₄—^{#}, wherein p4 is 0 or 1 or, and r4 is 1, with the proviso that p4+r4=0-4;
^{#}—(CH₂)ₚ₅—CO—NH—(CH₂)ᵣ₅*, wherein p5 is 0 or 1, and r5 is 1, with the proviso that p5+r5=0-4;
wherein * and ^{#} reflect where X¹¹ is bound within the ring structure.
103. The compound according to formula (I) according to any one of the proceeding clauses, wherein X⁵ is the moiety according to formula (B),

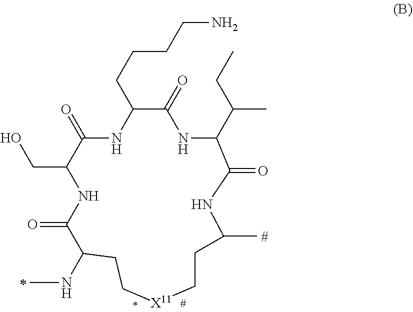

wherein * and ^{#} reflect where X⁵ is bound within the amino acid chain, wherein * indicates the binding site to P⁴³ and # indicates the binding site to P⁴⁹
wherein X¹¹ is selected from the group consisting of
*—(CH₂)ₚ₄—CO—NH—(CH₂)ᵣ₄—^{#}, wherein p4 is 0 or 1 or, and r4 is 2, with the proviso that p4+r4=0-4;
^{#}—(CH₂)ₚ₅—CO—NH—(CH₂)ᵣ₅—*, wherein p5 is 0 or 1, and r5 is 2, with the proviso that p5+r5=0-4;
wherein * and ^{#} reflect where X¹ is bound within the ring structure.
104. The compound according to formula (I) according to any one of the proceeding clauses, wherein X⁵ is the moiety according to formula (B),

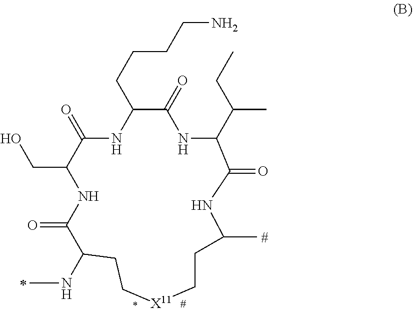

wherein * and ^{#} reflect where X⁵ is bound within the amino acid chain, wherein * indicates the binding site to P⁴³ and # indicates the binding site to P⁴⁹ wherein X¹ is selected from the group consisting of
*—(CH₂)ₚ₄—CO—NH—(CH₂)ᵣ₄—^{#}, wherein p4 is 0 or 1 or, and r4 is 3, with the proviso that p4+r4=0-4;
^{#}—(CH₂)ₚ₅—CO—NH—(CH₂)ᵣ₅*, wherein p5 is 0 or 1, and r5 is 3, with the proviso that p5+r5=0-4;
wherein * and ^{#} reflect where X¹ is bound within the ring structure.
105. The compound according to formula (I) according to any one of the proceeding clauses, wherein
   X¹ is selected from the group consisting of
   *—(CH₂)ₘ₁—S—S—(CH₂)ₙ₁—^{#}, wherein m1 is 0, 1 or 2, n1 is 0, 1 or 2;
   *—(CH₂)ₘ₆—CO—NH—(CH₂)ₙ₈—^{#}, wherein m6 is 0, 1 or 2, and n5 is 0, 1 or 2;
   ^{#}—(CH₂)ₘ₇—CO—NH—(CH₂)ₙ₆—*, wherein m7 is 0, 1 or 2, and n6 is 0, 1 or 2;
   wherein * and ^{#} reflect where X¹ is bound within the ring structure;
   X² is selected from the group consisting of G¹⁴, K¹⁴, which is covalently linked by an amide bond to the N-terminal G¹⁵ of the amino acid sequence of formula (I)
   X³ is absent or is a C14-C22 di-carboxylic acid,
   Z is absent or is a cleavable linker covalently bound between the N terminus of any amino acid of X² or of G¹⁵ and X³ or between a functional group of the side chain of any amino acid of X² and X³
   wherein if X³ is absent, then Z is also absent and X² is hydrogen or is an amino acid or amino acid sequence as defined in any one of the proceeding clauses above for X²;
   wherein if X³ is a C14-C22 di-carboxylic acid, then X² is absent or is an amino acid or amino acid sequence as defined in any one of the proceeding clauses above for X²;
   X⁴ is the amino sequence * [D³⁵ K³⁶ D³⁷ K³⁸ D³⁹ N⁴⁰ V⁴¹]#, wherein * indicates the binding site to T³⁴ and # indicates the binding site to A⁴², or X⁴ is a moiety according to formula (A), wherein * indicates the binding site to T³⁴ and # indicates the binding site to A⁴²

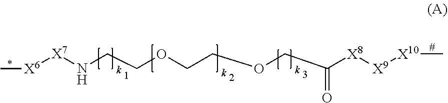

wherein
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein
k1 is 1 or 2;
k2 is 0, 1, 2, 3, or 4;
k3 is 1 or 2.
X⁵ is the amino sequence *[R⁴⁴ S⁴⁵ K⁴⁶ I⁴⁷ S⁴⁸]#, wherein * indicates the binding site to P⁴³ and #indicates the binding site to P⁴⁹, or X⁵ is a moiety according to formula (B), wherein * and ^{#} reflect where X⁵ is bound within the amino acid chain and wherein * indicates the binding site to P⁴³ and # indicates the binding site to P⁴⁹,

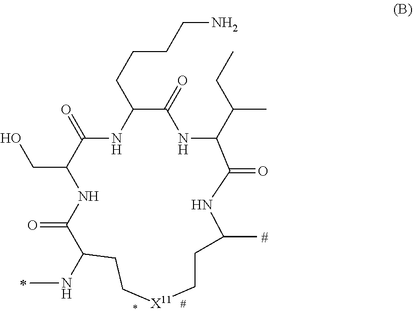

wherein X¹¹ is selected from the group consisting of
*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;
^{#}—(CH₂)ₚ₅—CO—NH—(CH₂)ᵣ₅*, wherein p5 is 0, 1, 2 or 3, and r5 is 0, 1, 2 or 3, with the proviso that p5+r5=0-4
wherein * and ^{#} reflect where X¹¹ is bound within the ring structure.
106. The compound according to formula (I) according to any one of the proceeding clauses, wherein the compound is a compound according to formula (Ia),

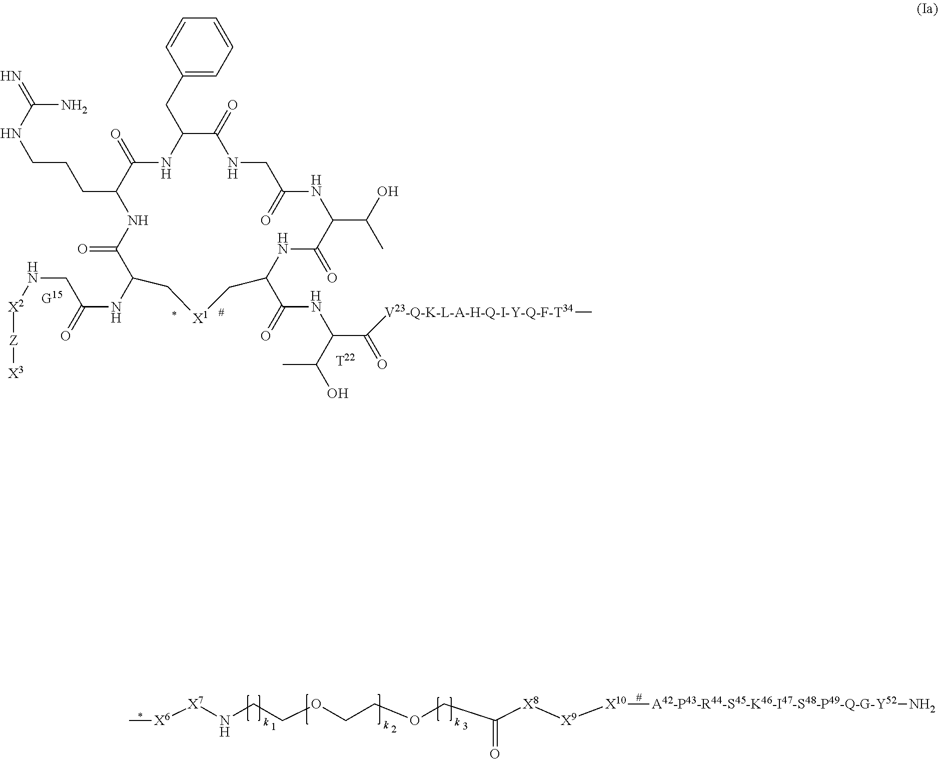

wherein X¹, X², X³, X⁶, X⁷, X⁸, X⁹, X¹⁰, k1, k2, and k3 are defined according to any one of the proceeding clauses;
a compound according to formula (Ib)

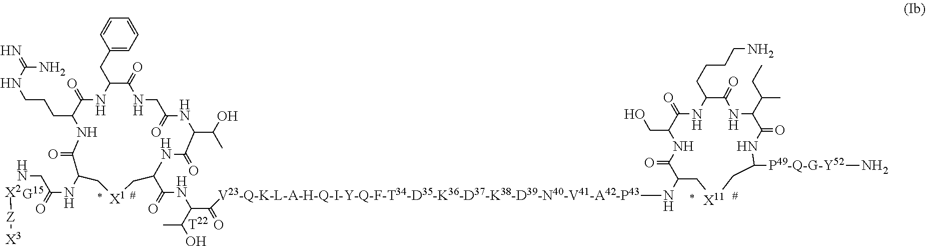

wherein X¹, X², X³ and X¹¹ are defined according to any one of the proceeding clauses;
a compound according to formula (Ic)

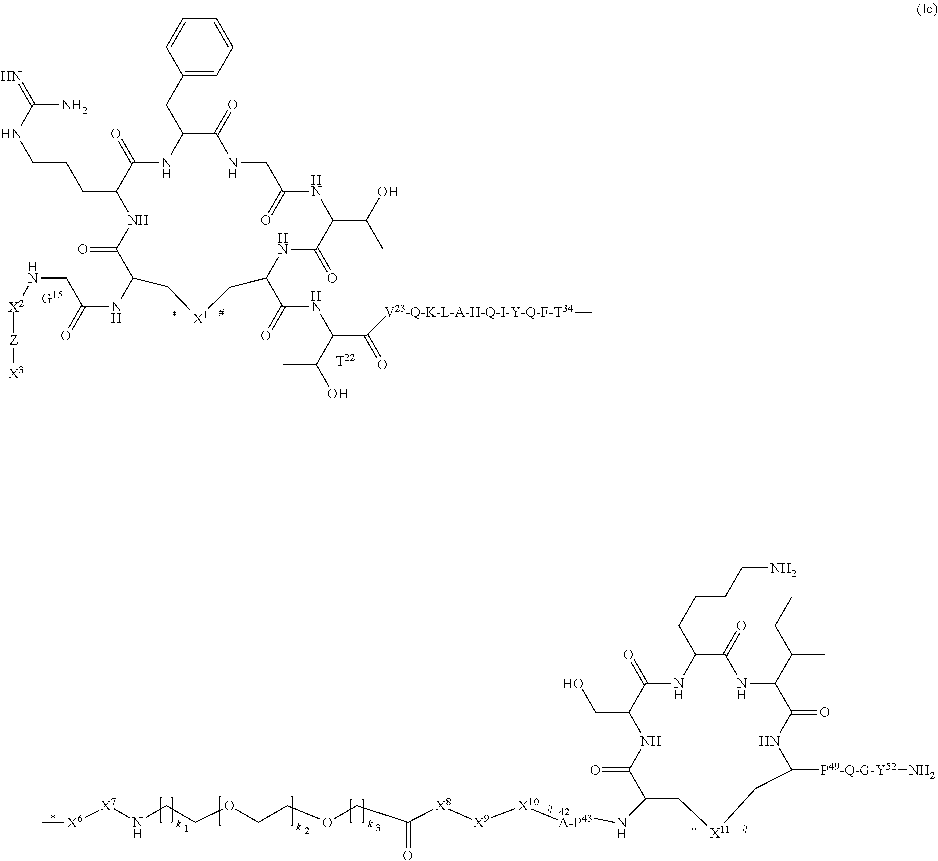

wherein X¹, X², X³, Z, X⁶, X⁷, X⁸, X⁹, X¹⁰, k1, k2, k3 and X¹¹ are defined according to any one of the proceeding clauses;

a compound according to formula (Id)

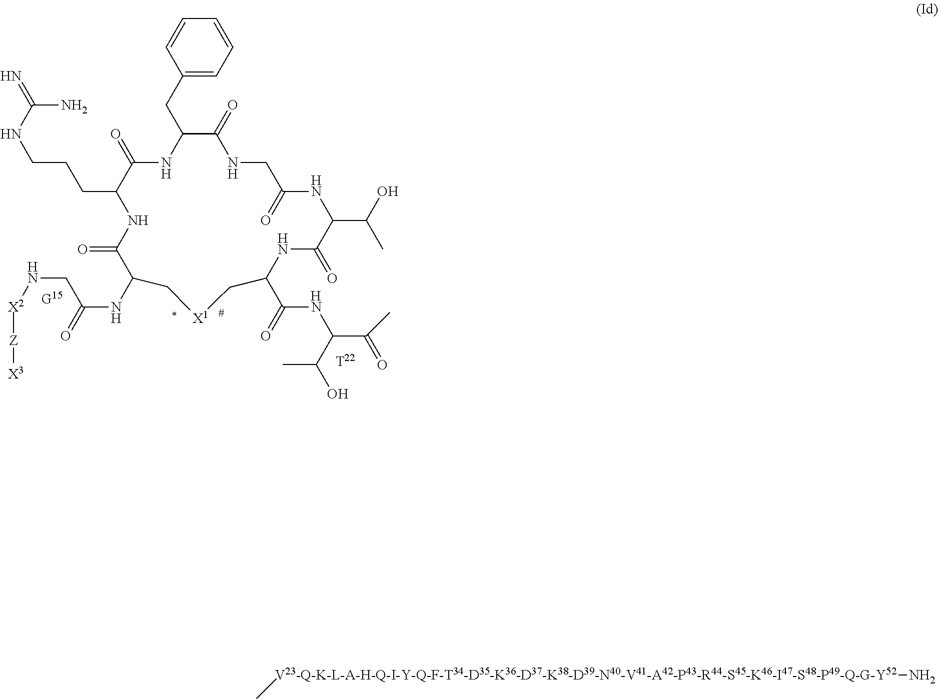

wherein X³ is a di-carboxylic acid, and X¹, X², Z, X⁶, X⁷, X⁸, X⁹, X¹⁰, k1, k2, k3 and X¹¹ are defined 10 according to any one of the proceeding clauses;
a compound according to formula (Ie) according to any one of the proceeding clauses,

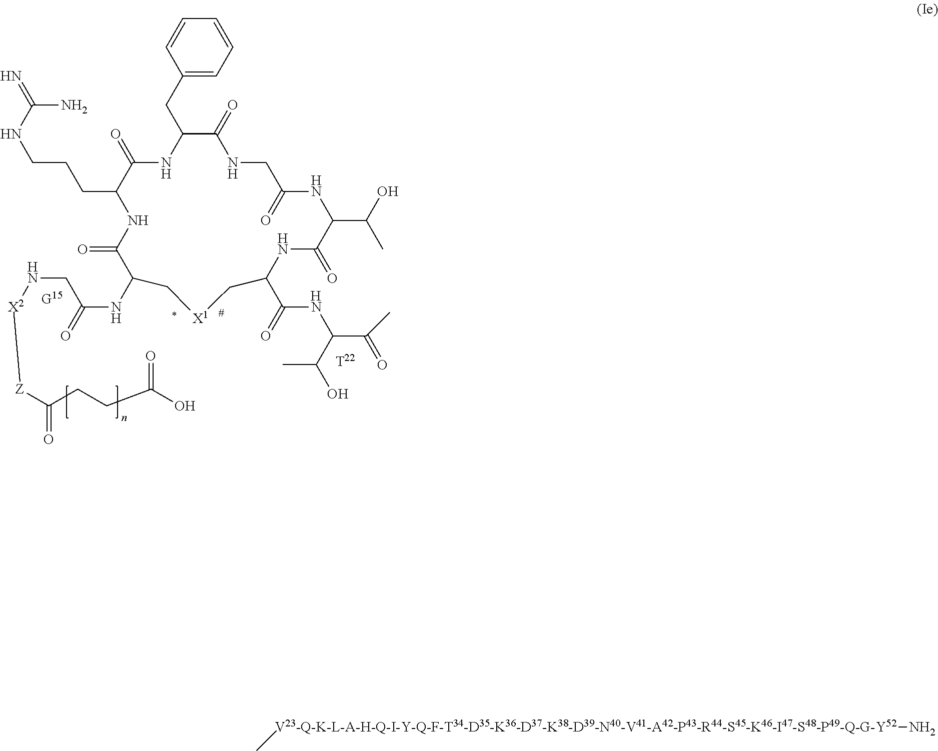

wherein n is 1 to 30 and X¹, X², Z are defined according to any one of the proceeding clauses;
a compound according to formula (If)

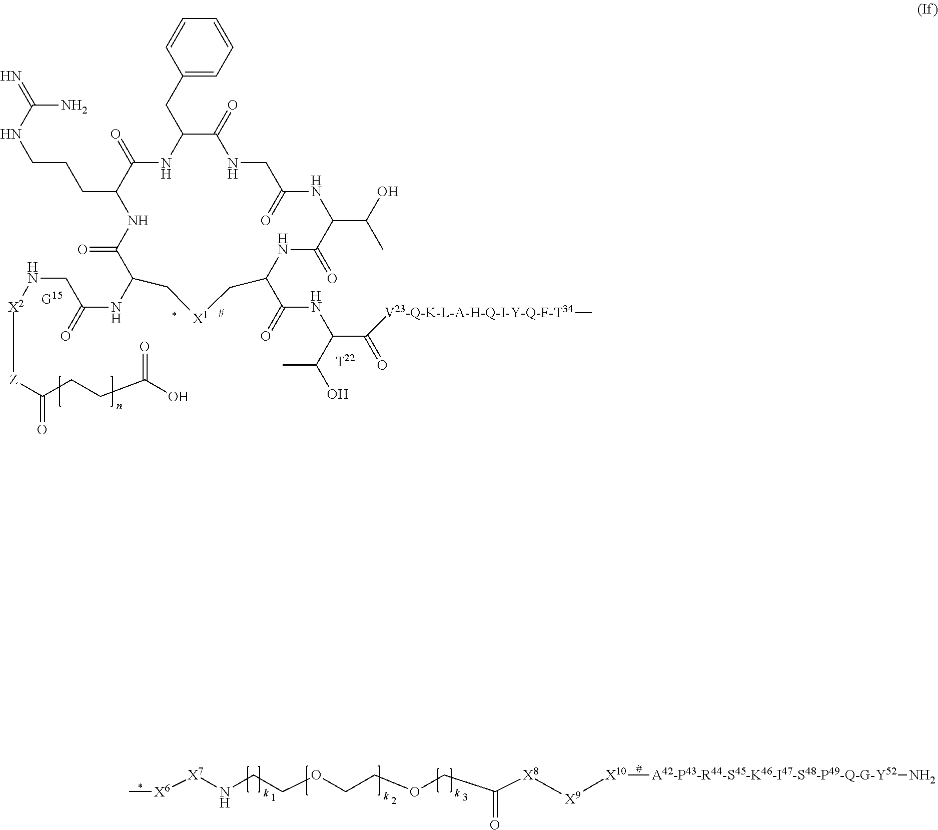

wherein n is 1 to 30 and X¹, X², Z, X⁶, X⁷, X⁸, X⁹, X¹⁰, k1, k2, k3 are defined according to any one of the proceeding clauses;
a compound according to formula (Ig)

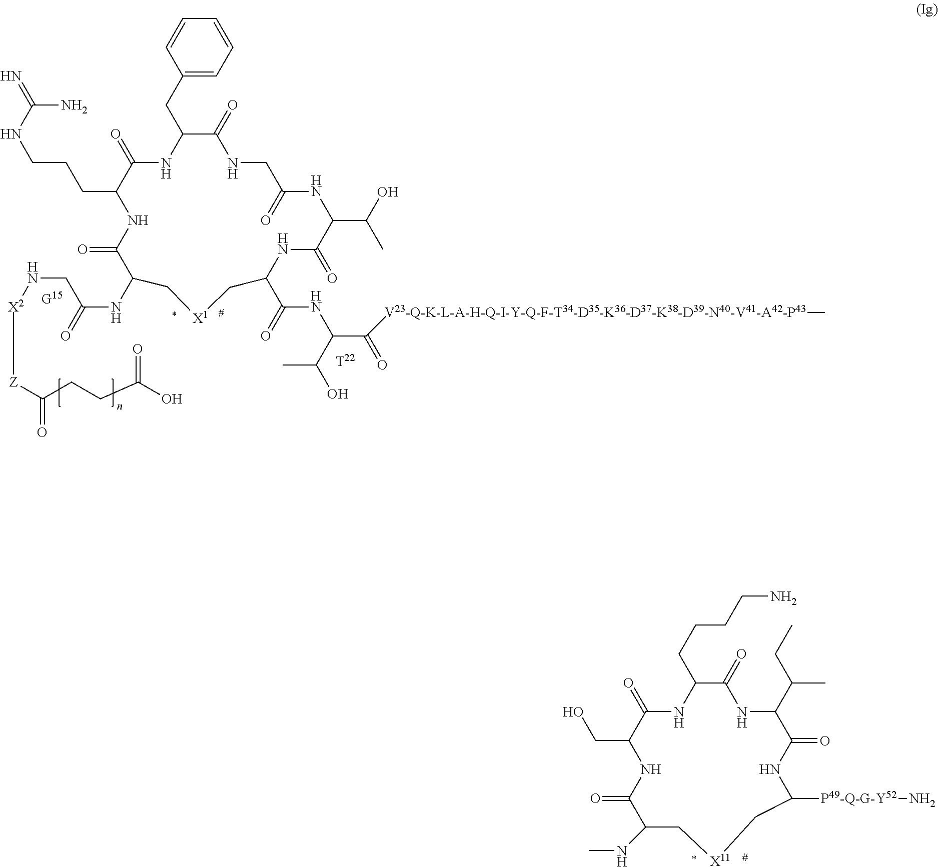

wherein n is 1 to 30 and X¹, X², Z and X¹¹ are defined according to any one of proceeding clauses;
a compound according to formula (Ih)

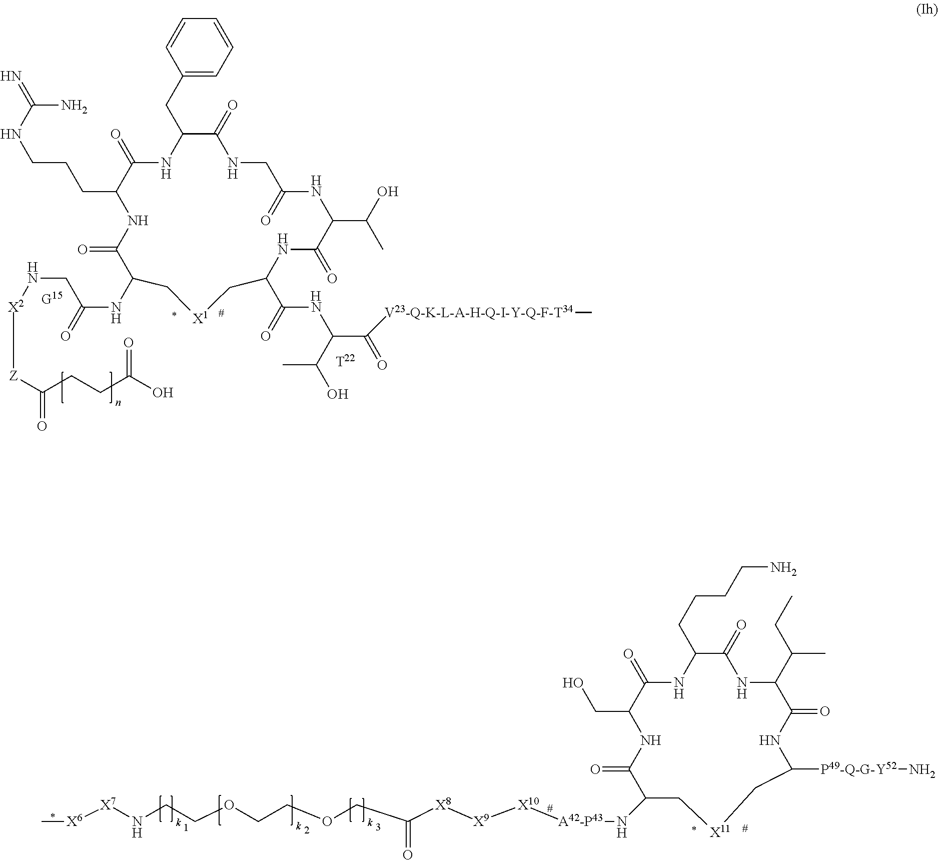

wherein n is 1 to 30 and X¹, X², Z, X⁶, X⁷, X⁸, X⁹, X¹⁰, k1, k2, k3 and X¹¹ are defined according to any one of the proceeding clauses.
107. The compound according to formula (I) or formula (Ia) according to any one of the proceeding clauses,

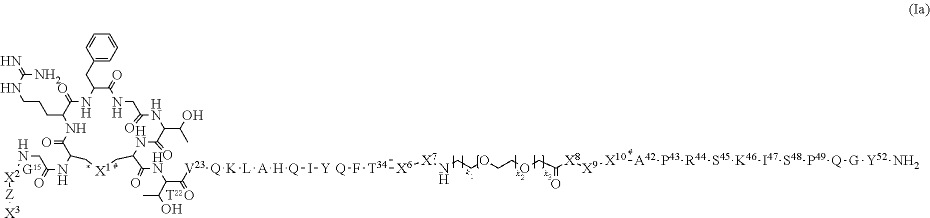

wherein
X¹ is selected from the group consisting of
*—(CH₂)ₘ₁—S—S—(CH₂)ₙ₁—^{#} wherein m1 is 0-6, n1 is 0-6, with the proviso that m1+n1=0-6;
*—(CH₂)ₘ₆—CO—NH—(CH₂)ₙ₈—^{#}, wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
*—(CH₂)ₘ₃—^{#}, wherein m3 is 1-8;
wherein * and ^{#} reflect where X¹ is bound within the ring structure
X² is selected from the group consisting of G¹⁴, K¹⁴, which is covalently linked by an amide bond to the N-terminal G¹⁵ of the amino acid sequence of formula (I)
X³ is absent or is a linear or branched C14-C22 di-carboxylic acid,
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined in any one of the proceeding clauses above for X²;
wherein if X³ is a linear or branched C14-C22 di-carboxylic acid, then X² is absent or is an amino acid or amino acid sequence as defined in any one of the proceeding clauses above for X²,
X⁶, X⁷, X⁸ X⁹ and X¹⁰ are defined as in any one of the proceeding clauses,
wherein k1 is 1 or 2;
wherein k2 is 0, 1, 2, 3, or 4;
wherein k3 is 1 or 2.
108. The compound according to formula (I) or formula (Ia) according to any one of the proceeding clauses,

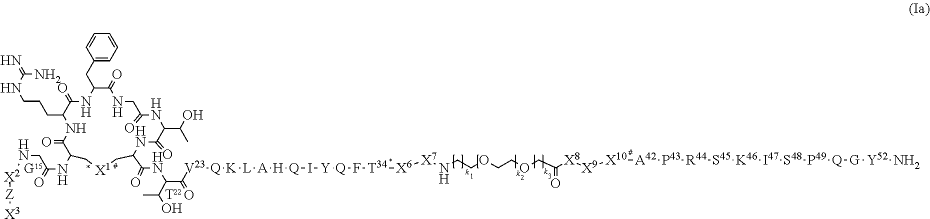

wherein
X¹ is selected from the group consisting of
*—(CH₂)ₘ₁—S—S—(CH₂)ₙ₁—^{#} wherein m1 is 0, 1 or 2, n1 is 0, 1 or 2;
*—(CH₂)ₘ₆—CO—NH—(CH₂)ₙ₈—^{#} wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure,
X² is selected from the group consisting of G¹⁴ or K¹⁴;
X³ and Z are absent,
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein k1 is 1 or 2;
wherein k2 is 0, 1, 2, 3, or 4;
wherein k3 is 1 or 2.
109. The compound according to formula (I) or formula (Ib) according to any one of the proceeding clauses,

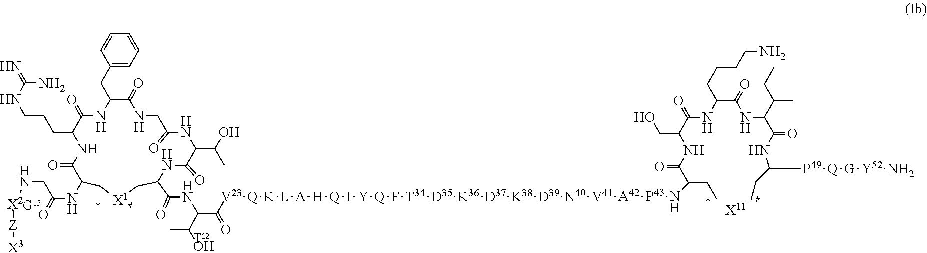

wherein
X¹ is selected from the group consisting of
*—(CH₂)ₘ₁—S—S—(CH₂)ₙ₁—^{#}, wherein m1 is 0, 1 or 2, n1 is 0, 1 or 2;
*—(CH₂)ₘ₆—CO—NH—(CH₂)ₙ₈—^{#}, wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure,
X² is selected from the group consisting of G¹⁴ or K¹⁴;
X³ is absent or is a linear or branched C14-C22 di-carboxylic acid,
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined in any one of the proceeding clauses above for X²;
wherein if X³ is a linear or branched C14-C22 di-carboxylic acid, then X² is absent or is an amino acid or amino acid sequence as defined in any one of the proceeding clauses above for X²
X¹¹ is selected from
*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;
^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;
   wherein * and ^{#} reflect where X¹¹ is bound within the ring structure.
110. The compound according to formula (I) or formula (Ib) according to any one of the proceeding clauses,

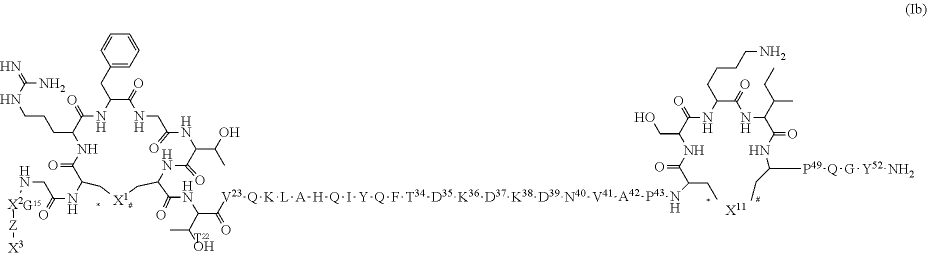

wherein

X¹ is selected from the group consisting of

*—(CH₂)ₘ₁—S—S—(CH₂)ₙ₁—^{#}, wherein m1 is 0, 1 or 2, n1 is 0, 1 or 2;

*—(CH₂)ₘ₆—CO—NH—(CH₂)ₙ₈—^{#}, wherein m6 is 0, 1 or 2, and n5 is 0, 1 or 2;

^{#}—(CH₂)ₘ₇—CO—NH—(CH₂)ₙ₆—*, wherein m7 is 0, 1 or 2, and n6 is 0, 1 or 2;

wherein * and ^{#} reflect where X¹ is bound within the ring structure

X² is selected from the group consisting of G¹⁴ or K¹⁴;

X³ and Z are absent,

X¹¹ is selected from

*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;

^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;
wherein * and ^{#} reflect where X¹¹ is bound within the ring structure.
111. The compound according to formula (I) or formula (Ic) according to any one of the proceeding clauses,

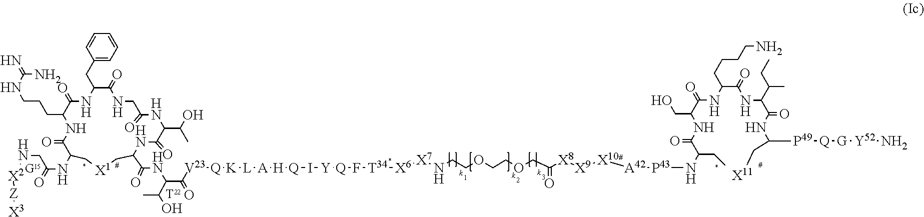

wherein
X¹ is selected from the group consisting of
*—(CH₂)ₘ₁—S—S—(CH₂)ₙ₁—^{#}, wherein m1 is 0, 1 or 2, n1 is 0, 1 or 2;
*—(CH₂)ₘ₆—CO—NH—(CH₂)ₙ₈—^{#} wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure
X² is selected from the group consisting of G¹⁴ or K⁴;
X³ is absent or is a linear or branched C14-C22 di-carboxylic acid,
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined in any one of the proceeding clauses above for X²;
wherein if X³ is a linear or branched C14-C22 di-carboxylic acid, then X² is absent or is an amino acid or amino acid sequence as defined in any one of the proceeding clauses above for X²
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein k1 is 1 or 2;
wherein k2 is 0, 1, 2, 3, or 4;
wherein k3 is 1 or 2;
X¹¹ is selected from
*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;
^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;
   wherein * and ^{#} reflect where X¹¹ is bound within the ring structure.
112. The compound according to formula (I) or formula (Ic) according to any one of the proceeding clauses,

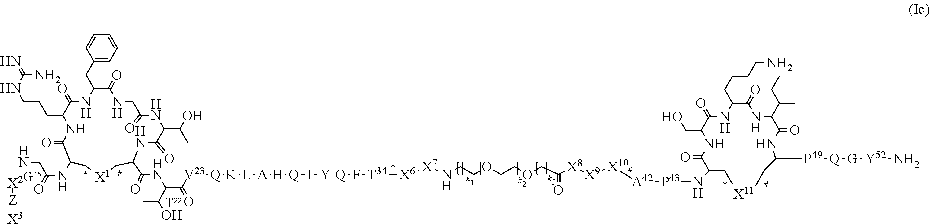

wherein
X¹ is selected from the group consisting of
*—(CH₂)ₘ₁—S—S—(CH₂)ₙ₁—^{#}, wherein m1 is 0, 1 or 2, n1 is 0, 1 or 2;
*—(CH₂)ₘ₆—CO—NH—(CH₂)ₙ₈—^{#}, wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure
X² is selected from the group consisting of G¹⁴ or K¹⁴;
X³ and Z are absent,
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein k1 is 1 or 2;
wherein k2 is 0, 1, 2, 3, or 4;
wherein k3 is 1 or 2;
X¹¹ is selected from
*—(CH₂)ₚ₄—CO—NH—(CH₂)ᵣ₄—^{#}, wherein p4 is 0, 1, 2 or 3, r4 is 0, 1, 2 or 3, with the proviso that p4+r4=0-5;
^{#}—(CH₂)ₚ₅—CO—NH—(CH₂)ᵣ₅—*, wherein p5 is 0, 1, 2 or 3, r5 is 0, 1, 2 or 3, with the proviso that p5+r5=0-5;
   wherein * and ^{#} reflect where X¹¹ is bound within the ring structure.
113. The compound according to formula (I) or formula (Ic) according to any one of the proceeding clauses,

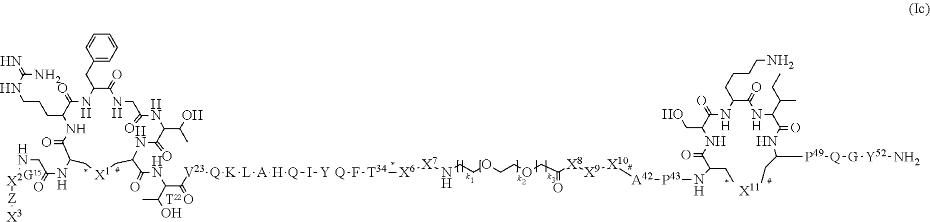

wherein
X¹ is selected from the group consisting of
*—(CH₂)ₘ₁—S—S—(CH₂)ₙ₁—^{#} wherein m1 is 0, 1 or 2, n1 is 0, 1 or 2;
*—(CH₂)ₘ₆—CO—NH—(CH₂)ₙ₈—^{#}, wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure
X² is selected from the group consisting of G¹⁴ or K¹⁴;
X³ is absent or is a linear or branched C14-C22 di-carboxylic acid,
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined in any one of the proceeding clauses above for X²;
wherein if X³ is a linear or branched C14-C22 di-carboxylic acid, then X² is absent or is an amino acid or amino acid sequence as defined in any one of the proceeding clauses above for X²
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein k1 is 1 or 2;
wherein k2 is 0, 1 or 2;
wherein k3 is 1 or 2;
X¹¹ is selected from
*—(CH₂)ₚ₄—CO—NH—(CH₂)ᵣ₄—^{#}, wherein p4 is 0 or 1, r4 is 0, 1, 2 or 3, with the proviso that p4+r4=0-5;
^{#}—(CH₂)ₚ₅—CO—NH—(CH₂)ᵣ₅—*, wherein p5 is 0 or 1, r5 is 0, 1, 2 or 3, with the proviso that p5+r5=0-5;
wherein * and ^{#} reflect where X¹¹ is bound within the ring structure.
114. The compound according to formula (I) or formula (Ic) according to any one of the proceeding clauses,

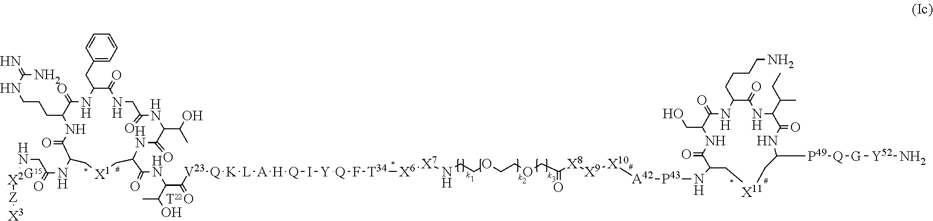

wherein
X¹ is selected from the group consisting of
*—(CH₂)ₘ₁—S—S—(CH₂)ₙ₁—^{#}, wherein m1 is 0, 1 or 2, n1 is 0, 1 or 2;
*—(CH₂)ₘ₆—CO—NH—(CH₂)ₙ₈—^{#}, wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure
X² is selected from the group consisting of G¹⁴ or K¹⁴;
X³ and Z are absent,
X⁶ is absent or selected from D, N, V;
X⁷ is absent or selected from D, N, V;
X⁸ is absent or selected from D, N, V;
X⁹ is absent or selected from D, N, V;
X¹⁰ is absent or selected from D, N, V;
wherein k1 is 1 or 2;
wherein k2 is 0, 1 or 2;
wherein k3 is 1 or 2,
X¹¹ is selected from
*—(CH₂)ₚ₄—CO—NH—(CH₂)ᵣ₄—^{#}, wherein p4 is 0 or 1, r4 is 0, 1, 2 or 3, with the proviso that p4+r4=0-5;
^{#}—(CH₂)ₚs-CO—NH—(CH₂)ᵣ₅—*, wherein p5 is 0 or 1, r5 is 0, 1, 2 or 3, with the proviso that p5+r5=0-5;
wherein * and ^{#} reflect where X¹¹ is bound within the ring structure.
115. The compound according to formula (I) or formula (Id) according to any one of the proceeding clauses,

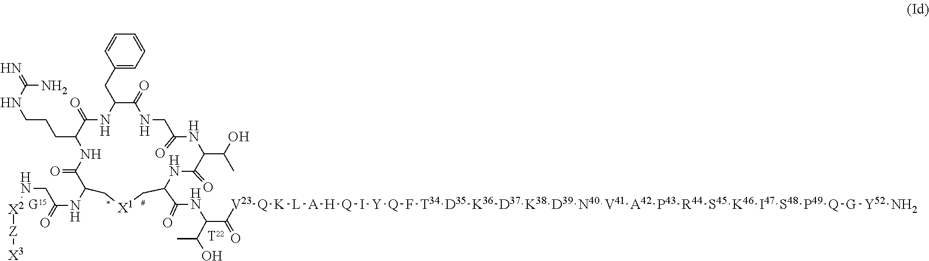

wherein
X¹ is selected from the group consisting of
*—(CH₂)ₘ₁—S—S—(CH₂)ₙ₁—^{#}, wherein m1 is 0, 1 or 2, n1 is 0, 1 or 2;
*—(CH₂)ₘ₆—CO—NH—(CH₂)ₙ₈—^{#}, wherein m6 is 0, 1 or 2, and n5 is 0, 1 or 2;
^{#}—(CH₂)ₘ₇—CO—NH—(CH₂)ₙ₆—*, wherein m7 is 0, 1 or 2, and n6 is 0, 1 or 2;
wherein * and ^{#} reflect where X¹ is bound within the ring structure
X² is selected from the group consisting of G¹⁴ or K⁴;
X³ is absent or is a linear or branched C14-C22 di-carboxylic acid,
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined in any one of the proceeding clauses above for X²;
wherein if X³ is a linear or branched C14-C22 di-carboxylic acid, then X² is absent or is an amino acid or amino acid sequence as defined in any one of the proceeding clauses above for X².
116. The compound according to formula (Ie) according to any one of the proceeding clauses,

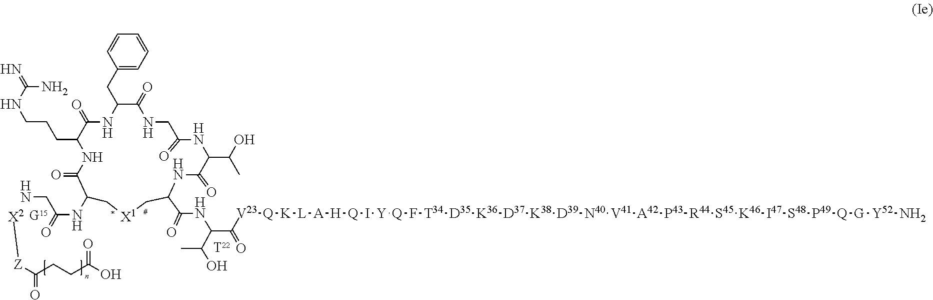

wherein n is 1 to 15, and wherein X¹ and X² are as defined according to any one of the proceeding clauses.
117. The compound according to formula (Ie) according to any one of the proceeding clauses, wherein n is 2 to 11, and wherein X¹ and X² are as defined according to any one of the proceeding clauses.
118. The compound according to formula (Ie) according to any one of the proceeding clauses, wherein n is 4 to 10, and wherein X¹ and X² are as defined according to any one of the proceeding clauses.
119. The compound according to formula (Ie) according to any one of the proceeding clauses, wherein n is 6 to 9, preferably 7 to 9, and wherein X¹ and X² are as defined according to any one of the proceeding clauses.
120. The compound according to formula (Ie) according to any one of the proceeding clauses, wherein n is 7 to 8, and wherein X¹ and X² are as defined according to any one of the proceeding clauses.
121. The compound according to formula (Ie) according to any one of the proceeding clauses, wherein n is 6 and wherein X¹ and X² are as defined according to any one of the proceeding clauses.
122. The compound according to formula (Ie) according to any one of the proceeding clauses, wherein n is 7 and wherein X¹ and X² are as defined according to any one of the proceeding clauses.
123. The compound according to formula (Ie) according to any one of the proceeding clauses, wherein n is 8 and wherein X¹ and X² are as defined according to any one of the proceeding clauses.
124. The compound according to formula (Ie) according to any one of the proceeding clauses, wherein n is 9 and wherein X¹ and X² are as defined according to any one of the proceeding clauses.
125. The compound according to formula (If) according to any one of the proceeding clauses, wherein

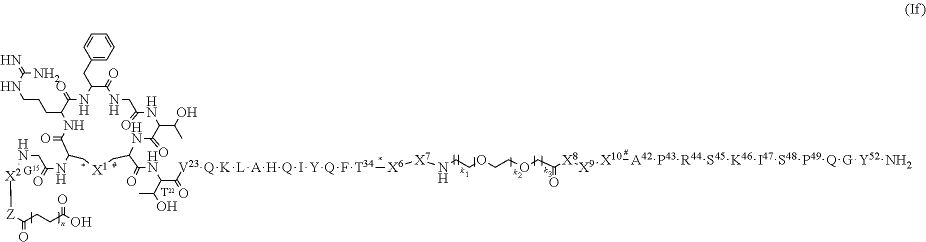

wherein n is 1 to 15, and wherein X¹ and X² are as defined according to any one of the proceeding clauses.
126. The compound according to formula (If) according to any one of the proceeding clauses, wherein n is 2 to 11, and wherein X¹ and X² are as defined according to any one of the proceeding clauses.
127. The compound according to formula (If) according to any one of the proceeding clauses, wherein n is 4 to 10, and wherein X¹ and X² are as defined according to any one of the proceeding clauses.
128. The compound according to formula (If) according to any one of the proceeding clauses, wherein n is 6 to 9, preferably 7 to 9, and wherein X¹ and X² are as defined according to any one of the proceeding clauses.
129. The compound according to formula (If) according to any one of the proceeding clauses, wherein n is 7 to 8, and wherein X¹ and X² are as defined according to any one of the proceeding clauses.
130. The compound according to formula (If) according to any one of the proceeding clauses, wherein n is 6 and wherein X¹ and X² are as defined according to any one of the proceeding clauses.
131. The compound according to formula (If) according to any one of the proceeding clauses, wherein n is 7 and wherein X¹ and X² are as defined according to any one of the proceeding clauses.
132. The compound according to formula (If) according to any one of the proceeding clauses, wherein n is 8 and wherein X¹ and X² are as defined according to any one of the proceeding clauses.
133. The compound according to formula (If) according to any one of the proceeding clauses, wherein n is 9 and wherein X¹ and X² are as defined according to any one of the proceeding clauses.
134. The compound according to formula (Ig) according to any one of the proceeding clauses,

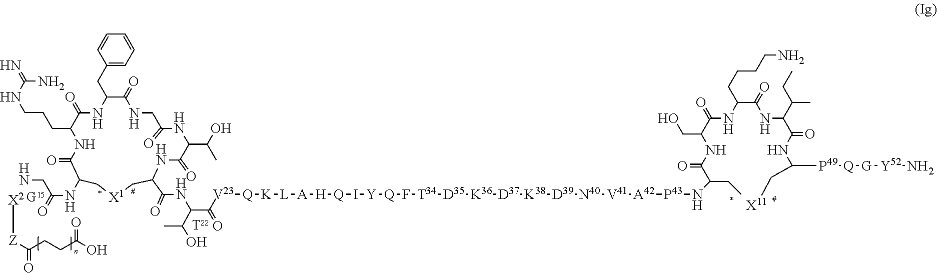

wherein n is 1 to 15, and wherein X¹ and X² are as defined according to any one of the proceeding clauses.
135. The compound according to formula (Ig) according to any one of the proceeding clauses, wherein n is 2 to 11, and wherein X¹ and X² are as defined according to any one of the proceeding clauses.
136. The compound according to formula (Ig) according to any one of the proceeding clauses, wherein n is 4 to 10, and wherein X¹ and X² are as defined according to any one of the proceeding clauses.
137. The compound according to formula (Ig) according to any one of the proceeding clauses, wherein n is 6 to 9, preferably 7 to 9, and wherein X¹ and X² are as defined according to any one of the proceeding clauses.
138. The compound according to formula (Ig) according to any one of the proceeding clauses, wherein n is 7 to 8, and wherein X¹ and X² are as defined according to any one of the proceeding clauses.
139. The compound according to formula (Ig) according to any one of the proceeding clauses, wherein n is 6 and wherein X¹ and X² are as defined according to any one of the proceeding clauses.
140. The compound according to formula (Ig) according to any one of the proceeding clauses, wherein n is 7 and wherein X¹ and X² are as defined according to any one of the proceeding clauses.
141. The compound according to formula (Ig) according to any one of the proceeding clauses, wherein n is 8 and wherein X¹ and X² are as defined according to any one of the proceeding clauses.
142. The compound according to formula (Ig) according to any one of the proceeding clauses, wherein n is 9 and wherein X¹ and X² are as defined according to any one of the proceeding clauses.
143. The compound according to formula (Ih) according to any one of the proceeding clauses,

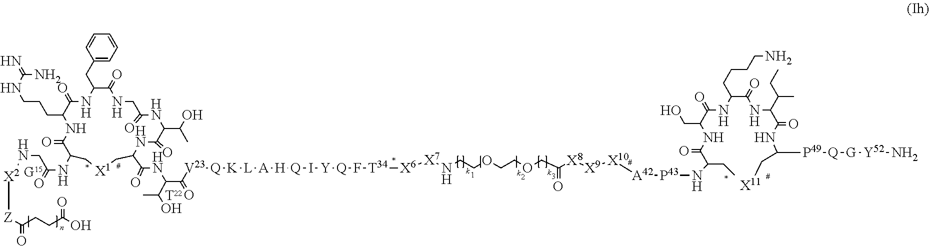

wherein n is 1 to 15, and wherein X¹ and X² are as defined according to any one of the proceeding clauses.
144. The compound according to formula (Ih) according to any one of the proceeding clauses, wherein n is 2 to 11, and wherein X¹ and X² are as defined according to any one of the proceeding clauses.
145. The compound according to formula (Ih) according to any one of the proceeding clauses, wherein n is 4 to 10, and wherein X¹ and X² are as defined according to any one of the proceeding clauses.
146. The compound according to formula (Ih) according to any one of the proceeding clauses, wherein n is 6 to 9, preferably 7 to 9, and wherein X¹ and X² are as defined according to any one of the proceeding clauses.
147. The compound according to formula (Ih) according to any one of the proceeding clauses, wherein n is 7 to 8, and wherein X¹ and X² are as defined according to any one of the proceeding clauses.
148. The compound according to formula (Ih) according to any one of the proceeding clauses, wherein n is 6 and wherein X¹ and X² are as defined according to any one of the proceeding clauses.
149. The compound according to formula (Ih) according to any one of the proceeding clauses, wherein n is 7 and wherein X¹ and X² are as defined according to any one of the proceeding clauses.
150. The compound according to formula (Ih) according to any one of the proceeding clauses, wherein n is 8 and wherein X¹ and X² are as defined according to any one of the proceeding clauses.
151. The compound according to formula (Ih) according to any one of the proceeding clauses, wherein n is 9 and wherein X¹ and X² are as defined according to any one of the proceeding clauses.
152. The compound according to any one of formulae (I), (Ia), (Ib), (Ic), (Id), (le), (If), (Ig) and/or (Ih) according to any one of the proceeding clauses, wherein the compound is selected from

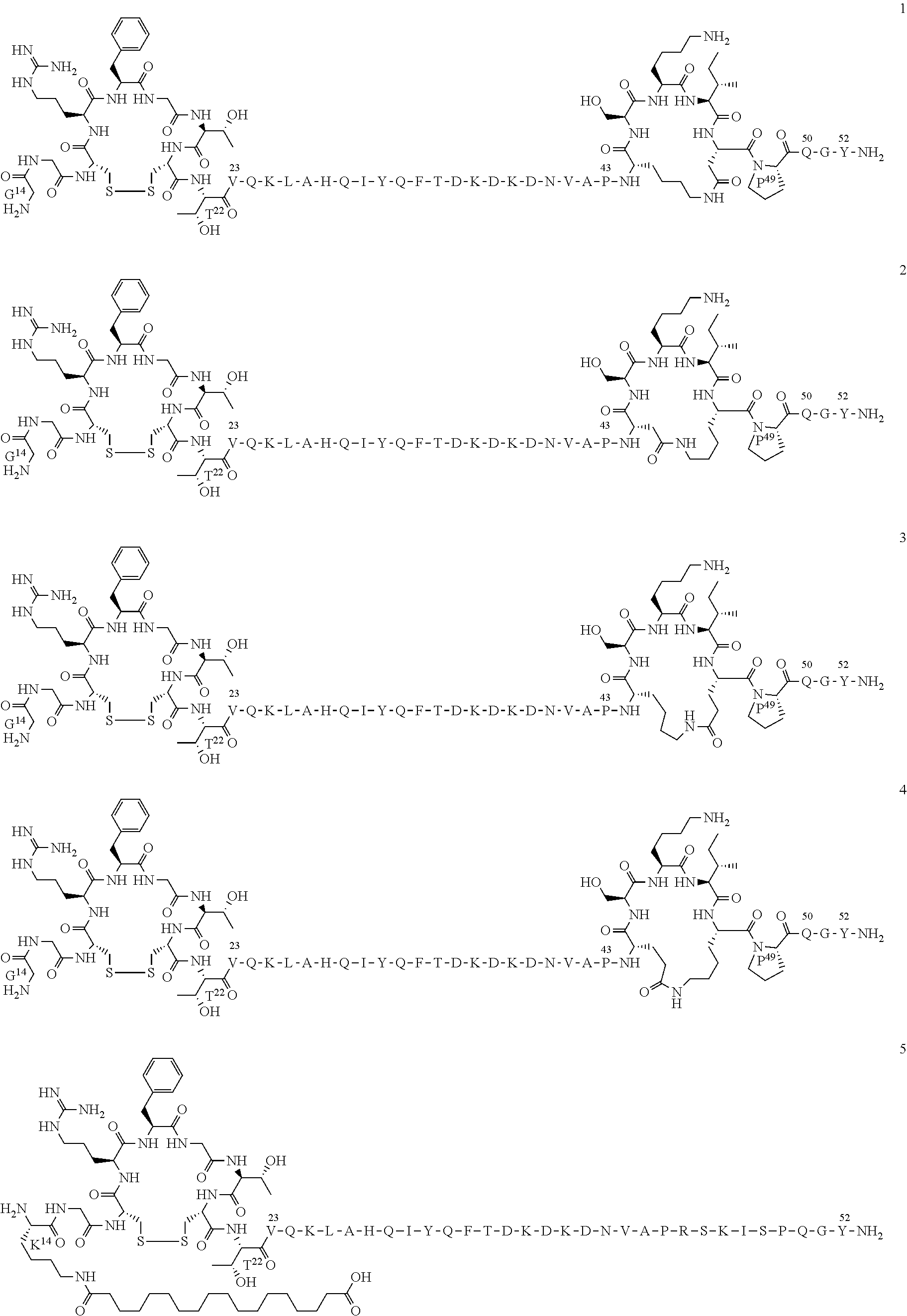

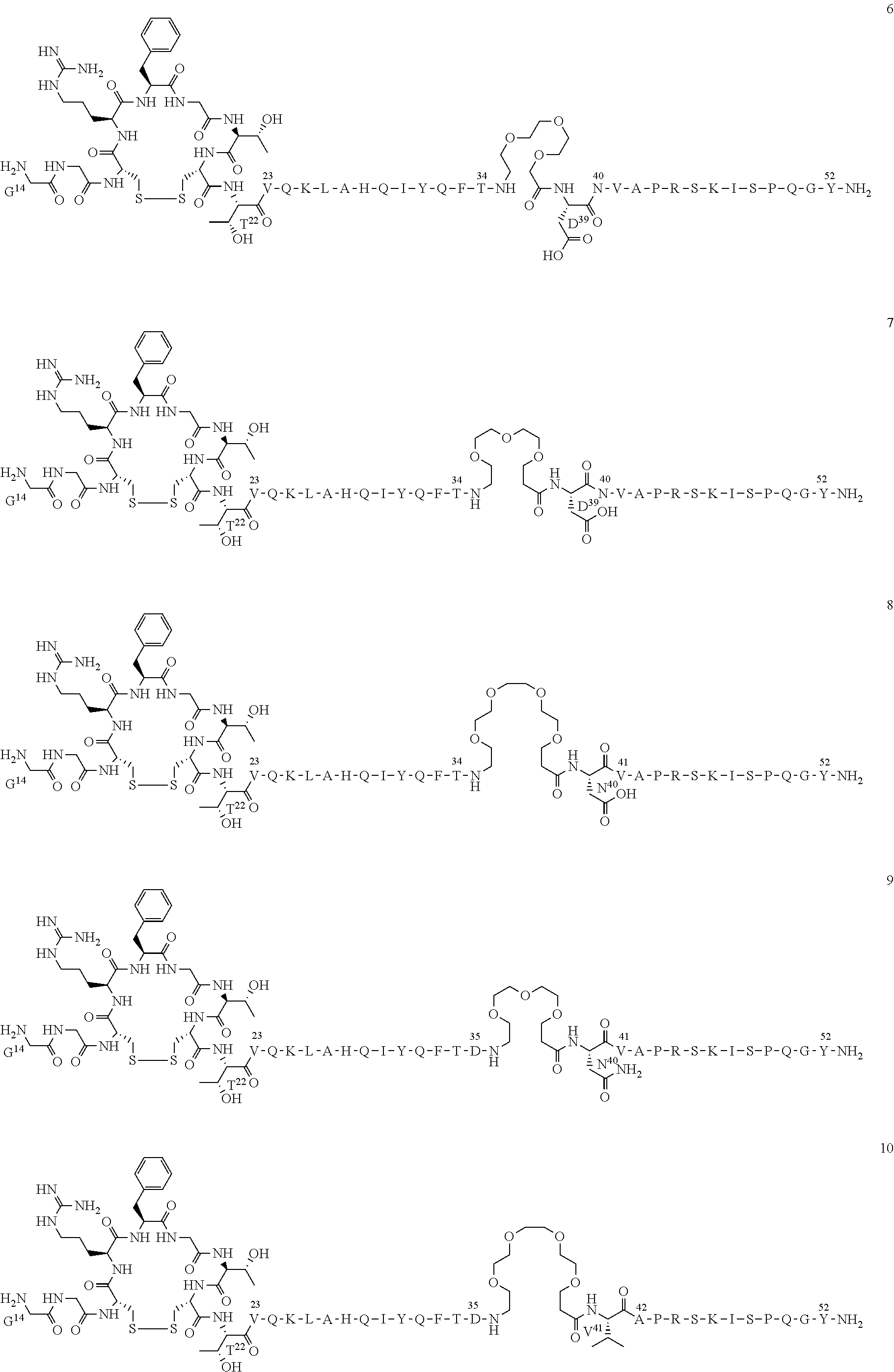

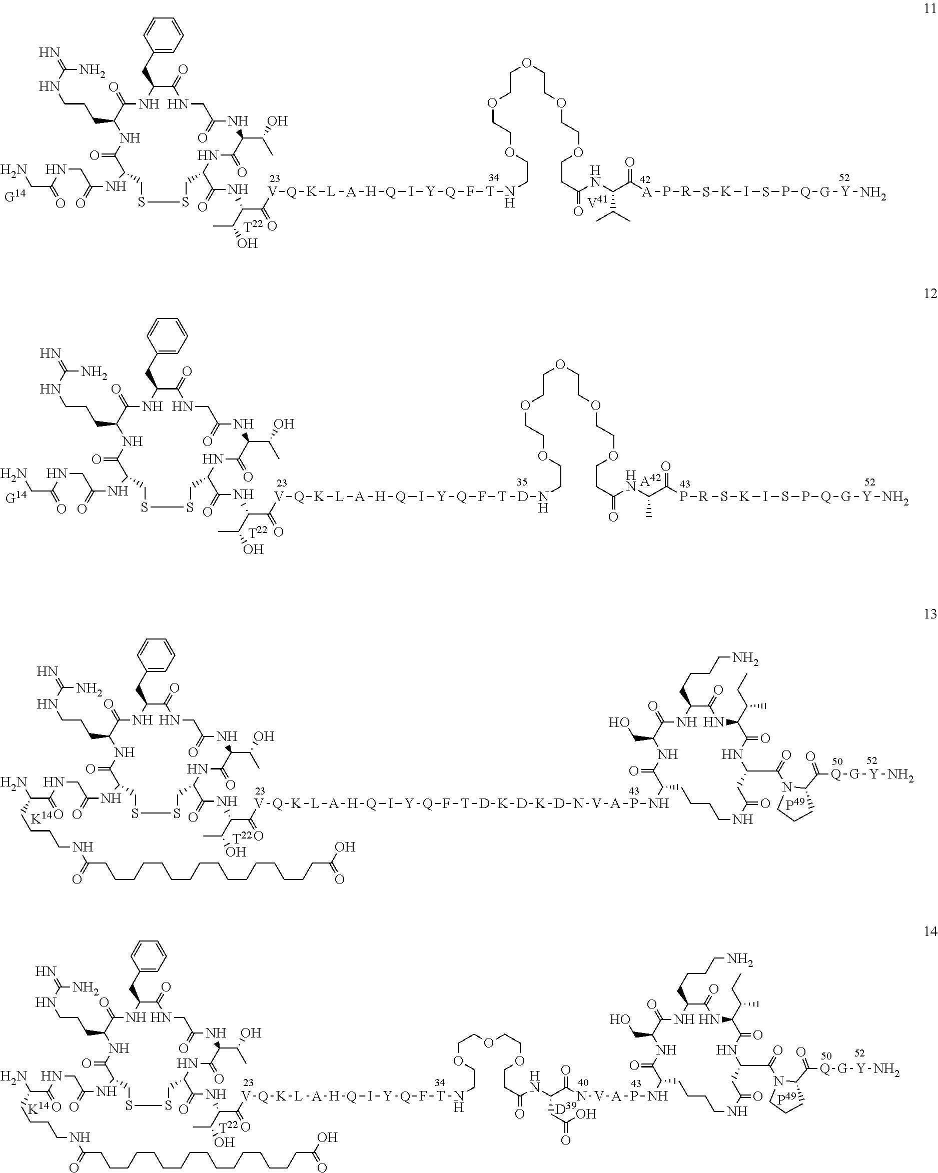

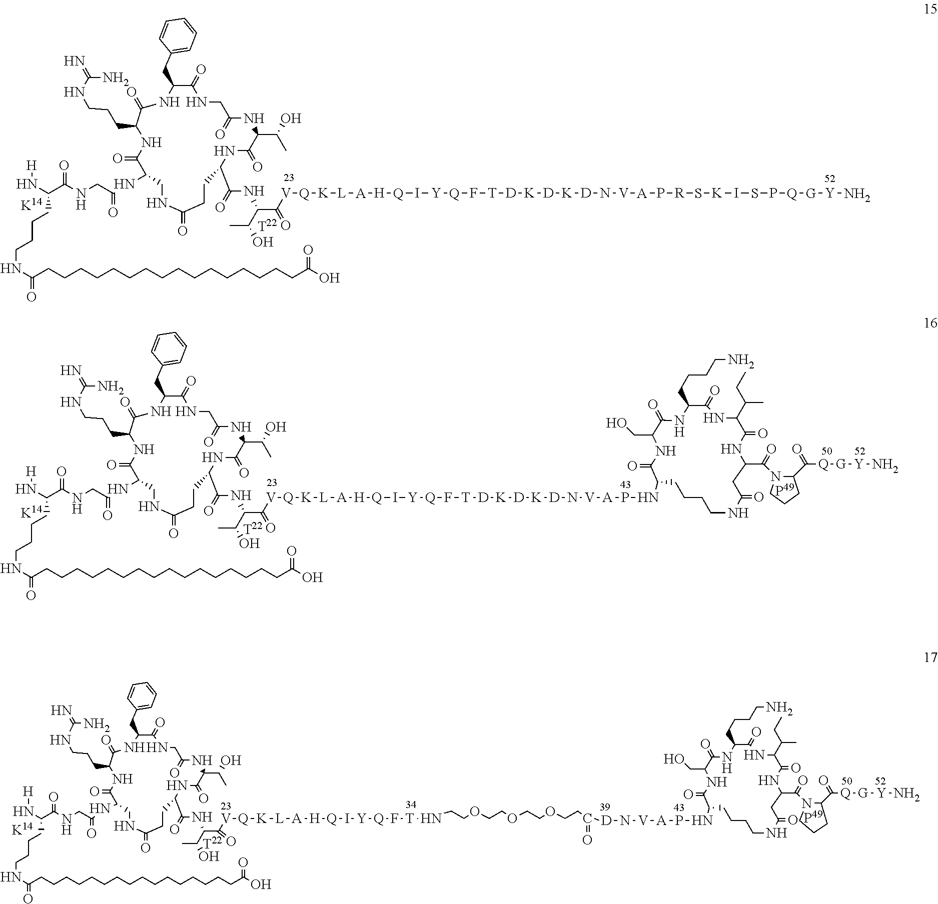

153. A compound as claimed in any of the proceeding clauses 1 to 152 for use in a method for the treatment and/or prevention of cardiovascular, edematous and/or inflammatory disorders.
154. The compound as claimed in any of clauses 1 to 152 for use in a method for the treatment and/or prevention of heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease, urticaria and/or edematous ocular disorders or ocular disorders associated with disturbed vascular function, including, age-related macular degeneration (AMD), diabetic retinopathy, in particular diabetic macula edema (DME), subretinal edema, and intraretinal edema.
155. A medicament comprising a compound as claimed in any of clauses 1 to 152 in combination with an inert nontoxic pharmaceutically suitable excipient.
156. A medicament comprising a compound as claimed in any of clauses 1 to 152 in combination with a further active ingredient selected from the group consisting of ACE inhibitors, angiotensin receptor antagonists, beta-2 receptor agonists, phosphodiesterase (PDE) inhibitors, glucocorticoid receptor agonists, diuretics, recombinant angiotensin converting enzyme-2, acetylsalicylic acid, natriuretic peptides and derivatives thereof, and neprilysin inhibitors.
157. The medicament as claimed in clause 155 or 156 for the treatment and/or prevention of cardiovascular, edematous and/or inflammatory disorders.
158. Method for the treatment and/or prophylaxis of cardiovascular, edematous and/or inflammatory disorders in humans or animals using an effective amount of at least one compound as claimed in any of clauses 1 to 152 or a medicament as defined in any of clauses 154 to 156.

### EXPLANATION OF THE FIGURES

FIG. 1 Stability of ADM analogues in human blood plasma.

FIG. 2A 24 hour profiles of mean arterial blood pressure (MABP) recorded from telemeterd normotensive female Wistar rats after subcutaneous administration of 100 μg/kg of example 13 (compound 13) (filled squares, solid line) or vehicle (open circles, dotted line). Data points were plotted as means of averaged 30 min intervals from 11 control animals and 5 treated animals, respectively.

FIG. 2B 24 hour profiles of mean arterial blood pressure (MABP) recorded from telemeterd normotensive female Wistar rats after subcutaneous administration of 100 μg/kg of example 16 (compound 16) (filled squares, solid line) or vehicle (open circles, dotted line). Data points were plotted as means±SEM of averaged 30 min intervals from 6 control animals and 4 treated animals, respectively.

FIG. 2C 24 hour profiles of mean arterial blood pressure (MABP) recorded from telemeterd normotensive female Wistar rats after subcutaneous administration of 100 μg/kg of example 17 (compound 17) filled squares, solid line) or vehicle (open circles, dotted line). Data points were plotted as means±SEM of averaged 30 min intervals from 6 control animals and 4 treated animals, respectively.

FIG. 3A depicts the general formula (Ia).

FIG. 3B depicts the general formula (Ib).

FIG. 3C depicts the general formula (Ic).

FIG. 3D depicts the general formula (Id).

FIG. 3E depicts the general formula (Ie).

FIG. 3F depicts the general formula (If).

FIG. 3G depicts the general formula (Ig).

FIG. 3H depicts the general formula (Ihi).

Methods

A. Synthesis of Adrenomedullin Analogues

Abbreviations

| | |
|---|---|
| AA | amino acid |
| ACN | acetonitrile |
| AcOH | acetic acid |
| ADM | Adrenomedullin (human) |
| approx. | approximately |
| Boc | tert-butyloxycarbonyl |
| DCM | dichloromethane |
| Dde | N-γ-(4,4-dimethy1-2,6-dioxocyclohex-1-ylidene)ethyl |
| DIC | N,N′-diisopropylcarbodiimide |
| DIPEA | N,N-diisopropyldiethylamine |
| DMF | N,N-dimethylformamide |
| Dpr | L-diaminopropionic acid |
| EDT | ethane-1,2-dithiol |
| equiv. | equivalent(s) |
| ESI | electrospray ionization (in MS) |
| Fmoc | N-[(9H-fluoren-9-ylmethoxy)carbonyl |
| HATU | O-(7-Azabenzotriazol-1-y1)-N,N,N′,N′-tetramethyluronium- |
| | hexafluorphosphat |
| HCl | hydrochloric acid |
| HOBt | 1-hydroxybenzotriazole |
| HPLC | high pressure, high performance liquid chromatography |
| MALDI- | matrix-assisted laser desorption/ionization-time |
| ToF | of flight (in MS) |
| Mmt | methoxytrityl |
| MS | mass spectrometry |
| NaCl | sodium chloride |
| NaOH | sodium hydroxyide |
| ODD | octadecanedioic acid |
| OEG | oligoethylene glycol |
| OPp | 2-phenylisopropyl |
| Oxyma | ethyl 2-cyano-2-(hydroxyimino)acetate |
| Pbf | 2,2,4,6,7-pentamethyldihydrobenzofuran-5-sulfonyl |
| RP | reversed phase (in HPLC) |
| TA | thioanisole |
| TAM/6- | 6-carboxytetramethylrhodamine |
| TAMRA | |
| TBS | tris buffered saline |
| tBu | tert-butyl |
| TFA | trifluoroacetic acid |
| TIS | triisopropylsilane |
| Trt | trityl |

Nomenclature of amino acids and peptide sequences is according to:

International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

| Trivial Name | Symbol | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Suppliers

| | |
|---|---|
| Acetic acid | Grüssing |
| ACN | Biosolve |
| Boc-Gly-OH | Iris Bitotech |
| Boc-Lys(Fmoc)-OH | Iris Bitotech |
| DCM | Biosolve |
| DIC | Iris Bitotech |
| DIPEA | Roth |
| DMF | Biosolve |
| EDT | Sigma-Aldirch |
| Fmoc-Ala-OH | OPC Orpegen |
| Fmoc-Arg(Pbf)-OH | OPC Orpegen |
| Fmoc-Asn(Trt)-OH | OPC Orpegen |
| Fmoc-Asp(OPp)-OH | Merck |
| Fmoc-Asp(tBu)-OH | OPC Orpegen |
| Fmoc-Cys(Trt)-OH | OPC Orpegen |
| Fmoc-Dpr(Mtt)-OH | Iris Bitotech |
| Fmoc-Gln(Trt)-OH | OPC Orpegen |
| Fmoc-Glu(OPp)-OH | Merck |
| Fmoc-Gly-OH | OPC Orpegen |
| Fmoc-His(Trt)-OH | OPC Orpegen |
| Fmoc-Ile-OH | OPC Orpegen |
| Fmoc-Leu-OH | OPC Orpegen |
| Fmoc-Lys(Boc)-OH | OPC Orpegen |
| Fmoc-Lys(Mmt)-OH | Iris Bitotech |
| Fmoc-Phe-OH | OPC Orpegen |
| Fmoc-Pro-OH | OPC Orpegen |
| Fmoc-Ser(tBu)-OH | OPC Orpegen |
| Fmoc-Thr(tBU)-OH | OPC Orpegen |
| Fmoc-Tyr(tBu)-OH | OPC Orpegen |
| Fmoc-Val-OH | OPC Orpegen |
| HOBt | Merck |
| NaOH | Grüssing |
| NovaSyn ® TGR R resin | Novabiochem |
| Octadecanedioic acid mono-tert-butyl ester | Apollo Scientific |
| Oxyma | Iris Biotech |
| TA | Sigma-Aldric |
| TFA | Merck |
| TIS | Merck |

### Example/Compound List

| Example/ | | |
|---|---|---|
| Compound | Code | Sequence |
| 1 | [G¹⁴, (K⁴⁴, D⁴⁸)_{lac}] | H- |
| | ADM(14-52) | GGC*RFGTC*TVQKLAHQIYQFTDKDKDNVAPK+SKID+PQGY- |
| | | NH₂ |
| 2 | [G¹⁴, (D⁴⁴, K⁴⁸)_{lac}] | H- |
| | ADM(l4-52) | GGC*RFGTC*TVQKLAHQIYQFTDKDKDNVAPD+SKIK+PQGY- |
| | | NH₂ |
| 3 | [G¹⁴, (K⁴⁴, E⁴⁸)_{lac}] | H- |
| | ADM(14-52) | GGC*RFGTC*TVQKLAHQIYQFTDKDKDNVAPK+SKIE+PQGY- |
| | | NH₂ |
| 4 | [G¹⁴, (E⁴⁴, K⁴⁸)_{lac}] | H- |
| | ADM(14-52) | GGC*RFGTC*TVQKLAHQIYQFTDKDKDNVAPE+SKIK+PQGY- |
| | | NH₂ |
| 5 | [K¹⁴(ODD)] ADM(14-52) | H-K(ODD)- |
| | | GC*RFGTC*TVQKLAHQIYQFTDKDKDNVAPRSKISPQGY-NH₂ |
| 6 | [G¹⁴, OEG(12)^{[35-38]}] | H-GGC*RFGTC*TVQKLAHQIYQFT-OEG(12)- |
| | ADM(14-52) | DNVAPRSKISPQGY-NH₂ |
| 7 | [G¹⁴, OEG(13)^{[35-38]}] | H-GGC*RFGTC*TVQKLAHQIYQFT-OEG(13)- |
| | ADM(14-52) | DNVAPRSKISPQGY-NH₂ |
| 8 | [G¹⁴, OEG(16)^{[35-39]}] | H-GGC*RFGTC*TVQKLAHQIYQFT-OEG(16)- |
| | ADM(14-52) | NVAPRSKISPQGY-NH₂ |
| 9 | [G¹⁴, OEG(13)^{[35-39]}] | H-GGC*RFGTC*TVQKLAHQIYQFTD-OEG(13)- |
| | ADM(14-52) | NVAPRSKISPQGY-NH₂ |
| 10 | [G¹⁴, OEG(16)^{[36-40]}] | H-GGC*RFGTC*TVQKLAHQIYQFTD-OEG(16)- |
| | ADM(14-52) | VAPRSKISPQGY-NH₂ |
| 11 | [G¹⁴, OEG(19)^{[35-40]}] | H-GGC*RFGTC*TVQKLAHQIYQFT-OEG(19)-VAPRSKISPQGY- |
| | ADM(14-52) | NH₂ |
| 12 | [G¹⁴, OEG(19)^{[36-41]}] | H-GGC*RFGTC*TVQKLAHQIYQFTD-OEG(19)-APRSKISPQGY- |
| | ADM(14-52) | NH₂ |
| 13 | [K¹⁴(ODD), | H-K(ODD)- |
| | (K⁴⁴, D⁴⁸)_{lac}] | GC*RFGTC*TVQKLAHQIYQFTDKDKDNVAPK+SKID+PQGY- |
| | ADM(14-52) | NH₂ |
| 14 | [K¹⁴(ODD), | H-K(ODD)-GC*RFGTC*TVQKLAHQIYQFT-OEG(13)- |
| | OEG(13)^{[35-38]}, | DNVAPK+SKID+PQGY-NH₂ |
| | (K⁴⁴, D⁴⁸)_{lac,}] | |
| | ADM(14-52) | |
| 15 | [K¹⁴(ODD), | H-K(ODD)-G-Dpr+- |
| | (Dpr¹⁶, E²¹)_{lac}] | RFGTE+TVQKLAHQIYQFTDKDKDNVAPRSKISPQGY-NH₂ |
| | ADM(14-52) | |
| 16 | [K¹⁴(ODD), | H-K(ODD)-G-Dpr+¹- |
| | (Dpr¹⁶, E²¹)_{lac}, | RFGTE+¹TVQKLAHQIYQFTDKDKDNVAPK+²SKID+²PQGY- |
| | (K⁴⁴, D⁴⁸)_{lac}] | |
| | ADM(14-52) | |
| 17 | [K¹⁴(ODD), | H-K(ODD)-G-Dpr+¹-RFGTE+¹TVQKLAHQIYQFT-OEG(13)- |
| | (Dpr¹⁶, E²¹)_{lac}, | DNVAPK+²SKID+²PQGY-NH₂ |
| | OEG(13)^{[35-38]}, | |
| | (K⁴⁴, D⁴⁸)_{lac}] | |
| | ADM(14-52) | |

amino acids in brackets separated with commas ( . . . )_{lac} indicate a lactam-bridge between the side chains of the corresponding amino acids; in sequence these lactam-bridged amino acids are marked with +; in case of two lactams, superscripted numbers reflect connected amino acids
* indicates disulfide bond
OEG(x)^{[y-z]} indicates that amino acids in pos y to z were replaced by a OEG-linker consisting of x atoms
(ODD) indicates the attachment of octadecanedioic acid to the side chain of the corresponding amino acid
Dpr is diamino propionic acid

Synthesis

All reactions and procedures were performed at room temperature if not indicated otherwise. After each coupling and deprotection step, the resins were repeatedly washed with DMF and DCM to remove excess of reagents.

General Method for Peptide Synthesis:

ADM analogues were synthesized stepwise on a NovaSyn®TGR R resin (Novabiochem) with an automated peptide synthesizer (SYRO I, MultiSynTech). The reaction vessels were loaded with 15 μmol NovaSyn®TGR R resin. Each amino acid and the reagents Oxyma and DIC were added in 8-fold molar excess (120 μmol). If not indicated otherwise, the amino acids were N-α-Fmoc-protected; the protecting groups indicated in brackets were used for side chain functionalities. All reactions were performed in DMF. Each coupling step was performed twice with a reaction time of 40 min. Cleavage of the Fmoc protecting group was achieved using 40% piperidine in DMF (v/v) for 3 min and 20% piperidine in DMF (v/v) for 10 min after each coupling step.

Lactam-Bridged Adrenomedullin-Analogues 1-4

Synthesis:

The syntheses of compounds 1-4 were performed using automated peptide synthesis as described in the general method. The amino acids used in coupling cycles 1-38 were N-α-Fmoc-protected, while Boc-Gly-OH was used as N-terminal amino acid in coupling cycle 39.

The coupling sequences were as follows:

| | | | | | AA of |
|---|---|---|---|---|---|
| Coupling | compound | compound | compound | compound | human |
| Cycle | 1 | 2 | 3 | 4 | ADM |
| 1. | Tyr(tBu) | | | | 52 |
| 2. | Gly | | | | 51 |
| 3. | Gln(Trt) | | | | 50 |
| 4. | Pro | | | | 49 |
| 5. | Asp(OPp) | Lys(Mmt) | Glu(OPp) | Lys(Mmt) | 48 |
| 6. | Ile | | | | 47 |
| 7. | Lys(Boc) | | | | 46 |
| 8. | Ser(tBu) | | | | 45 |
| 9. | Lys(Mmt) | Asp(OPp) | Lys(Mmt) | Glu(OPp) | 44 |

| | | |
|---|---|---|
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 37 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |
| 32. | Cys(Trt) | 21 |
| 33. | Thr(tBu) | 20 |
| 34. | Gly | 19 |
| 35. | Phe | 18 |
| 36. | Arg(Pbf) | 17 |
| 37. | Cys(Trt) | 16 |
| 38. | Gly | 15 |
| 39. | Boc-Gly | 14 |

For the simultaneous removal of Mmt/OPp protecting groups, the resins were treated with TFA/TIS/DCM (3:5:92, v/v/v) (15×2 min, 1 mL). Subsequently, the resins were washed with 2% DIPEA in DMF (v/v) for 10 min twice (1 mL).

Cyclization was performed using a 15-fold molar excess of HOBt and DIC in DMF as solvent for approx. 24 h.

Cleavage of the peptides from the resin and simultaneous side chain deprotection was achieved with TFA/TA/EDT (90:7:3, v/v/v) for approx. 3 h. The peptides were precipitated and washed with ice-cold diethyl ether and subsequently dried under reduced pressure.

Oxidation of the disulfide bond was achieved by solving the peptide in 10 m1 ACN/H₂O/TBS (1:4:5, v/v/v), adjusting the pH to 7.6-7.8 (1 M NaOH) and subsequent shaking for 12 h. Upon completion of the oxidation, the pH was adjusted to 3-4 using 1 M HCl.

Purification of the crude peptide was performed using preparative RP-HPLC on an Aeris PEPTIDE 5 μm XB-C18 LC column (Phenomenex, 250 mm×21.2 mm, 5 m, 100 Å). A linear gradient of 10% to 60% eluent B in A over 40 min was applied (Eluent A=0.1% TFA in water; Eluent B=0.08% TFA in 15 ACN). The flow rate was 15 mL/min, UV detection was measured at λ=220 nm.

Analytics:

The identity of the peptides was confirmed via MALDI-MS (UltraflexIII, Bruker) and ESI-MS (HCT, Bruker). The purities were analyzed using analytical RP-HPLC.

Compound 1: [G¹⁴, (K⁴⁴, D⁴⁸)_{lac}]ADM(14-52)

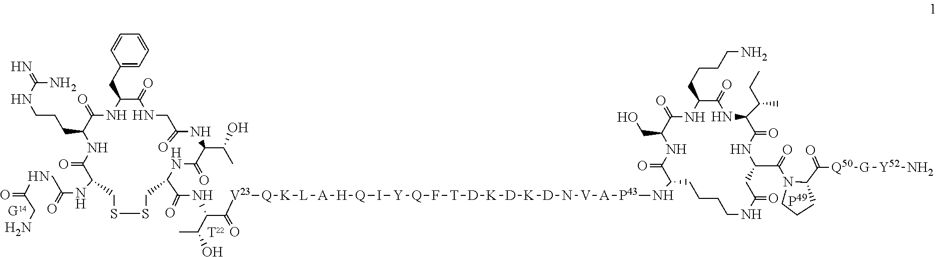

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-43)-((2S,5S,8S,11S,18S)-5-(4-aminobutyl)-18-(λ²-azaneyl)-8-((S)-sec-butyl)-2-(hydroxymethyl)-3,6,9,13,19-pentaoxo-1,4,7,10,14-pentaazacyclononadecane-11-carbonyl)-L-prolyl-ADM(50-52)

Chemical Formula: C₁₉₁H₂₉₅N₅₅O₅₇S₂

Exact Mass: 4335.13 Da

Molecular Weight: 4337.91 g/mol

Compound 1 was synthesized in a 15 μmol scale. The yield was 5.1 mg (8% f theory).

Compound 1 was analyzed via analytical RP-HPLC using a Kinetex® 5 μm C18 100 Å LC column (Phenomenex, 250 mm×4.6 mm, 5 m, 100 Å) applying a linear gradient of 10% to 60% eluent B in A over 40 min (Eluent A=0.1% TFA in water; Eluent B=0.08% TFA in ACN; flow rate=1.55 mL/min; λ=220 nm). Rt=19.2 min, purity≥95%.

In addition, a Jupiter® 4 μm Proteo 90 Å LC column (Phenomenex, 250 mm×4.6 mm, 4 m, 90 Å) was used, applying a linear gradient of 10% to 60% eluent B in A over 40 min (Eluent A=0.1% TFA in water; Eluent B=0.08% TFA in ACN; flow rate=1.0 mL/min; λ=220 nm). Rt=19.5 min, purity≥95%.

The observed mass was in accordance with the calculated mass.

ESI Ion-Trap: m/z=1085.3 [M+4H]⁴⁺, 868.5 [M+5H]⁵⁺, 723.9 [M+6H]⁶⁺, 620.6 [M+7H]⁷⁺.

MALDI-ToF: m/z=4336.1 [M+H]⁺, 2168.5 [M+2H]²⁺, 1446.7 [M+H]³⁺.

Compound 2: [G¹⁴, (D⁴⁴, K⁴⁸)_{lac}]ADM(14-52)

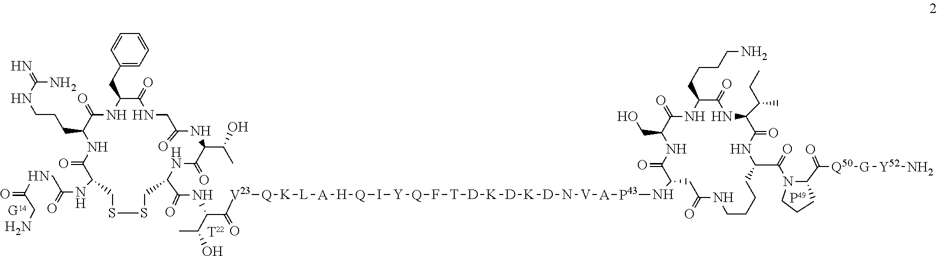

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-43)-((3S,6S,9S,12S,20S)-6-(4-aminobutyl)-12-(λ²-azaneyl)-3-((S)-sec-butyl)-9-(hydroxymethyl)-2,5,8,11,14-pentaoxo-1,4,7,10,15-pentaazacycloicosane-20-carbonyl)-L-prolyl-ADM(50-52)

Chemical Formula: C₁₉₁H₂₉₅N₅₅O₅₇S₂

Exact Mass: 4335.13 Da

Molecular Weight: 4337.91 g/mol

Compound 2 was synthesized in a 15 μmol scale. The yield was 3.9 mg (6% f theory).

Compound 2 was analyzed via analytical RP-HPLC using a Kinetex® 5 μm C_{18 100} Å LC column (Phenomenex, 250 mm×4.6 mm, 5 m, 100 Å) applying a linear gradient of 10% to 60% eluent B in A over 40 min (Eluent A=0.1% TFA in water; Eluent B=0.08% TFA in ACN; flow rate=1.55 mL/min; λ=220 nm). Rt=19.3 min, purity≥95%.

In addition, a Jupiter® 4 μm Proteo 90 Å LC column (Phenomenex, 250 mm×4.6 mm, 4 m, 90 Å) was used, applying a linear gradient of 10% to 60% eluent B in A over 40 min (Eluent A=0.1% TFA in water; Eluent B=0.08% TFA in ACN; flow rate=1.0 mL/min; λ=220 nm). Rt=19.6 min, purity≥95%.

The observed mass was in accordance with the calculated mass.

ESI Ion-Trap: m/z=1085.4 [M+4H]⁴⁺, 868.5 [M+5H]⁵⁺, 723.9 [M+6H]⁶⁺, 620.6 [M+7H]⁷⁺.

MALDI-ToF: m/z=4336.1 [M+H]⁺, 2168.5 [M+2H]²⁺, 1446.7 [M+H]³⁺.

Compound 3: [G¹⁴, (K⁴⁴, E⁴⁸)_{lac}]ADM(14-52)

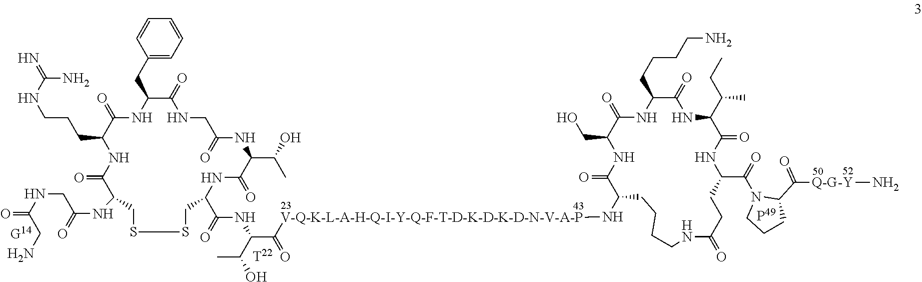

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-43)-((2S,5S,8S,11S,20S)-5-(4-aminobutyl)-20-(λ²-azaneyl)-8-((S)-sec-butyl)-2-(hydroxymethyl)-3,6,9,14,21-pentaoxo-1,4,7,10,15-pentaazacyclohenicosane-11-carbonyl)-L-prolyl-ADM(50-52)

Chemical Formula: C₁₉₂H₂₉₇N₅₅O₅₇S₂

Exact Mass: 4349.15 Da

Molecular Weight: 4351.94 g/mol

Compound 3 was synthesized in a 15 μmol scale. The yield was 4.5 mg (7% f theory).

Compound 3 was analyzed via analytical RP-HPLC using a Kinetex® 5 μm C18 100 Å LC column (Phenomenex, 250 mm×4.6 mm, 5 m, 100 Å) applying a linear gradient of 10% to 60% eluent B in A over 40 min (Eluent A=0.1% TFA in water; Eluent B=0.08% TFA in ACN; flow rate=1.55 mL/min; λ=220 nm). Rt=19.1 min, purity≥95%.

In addition, a Jupiter® 4 μm Proteo 90 Å LC column (Phenomenex, 250 mm×4.6 mm, 4 m, 90 Å) was used, applying a linear gradient of 10% to 60% eluent B in A over 40 min (Eluent A=0.1% TFA in water; Eluent B=0.08% TFA in ACN; flow rate=1.0 mL/min; λ=220 nm). Rt=19.4 min, purity≥95%.

The observed mass was in accordance with the calculated mass.

ESI Ion-Trap: m/z=1088.9 [M+4H]⁴⁺, 871.3 [M+5H]⁵⁺, 726.2 [M+6H]⁶⁺, 622.6 [M+7H]⁷⁺.

MALDI-ToF: m/z=4350.1 [M+H]⁺, 2175.5 [M+2H]²⁺, 1451.3 [M+H]³⁺.

Compound 4: [G¹⁴, (E⁴⁴, K⁴⁸)_{lac}]ADM(14-52)

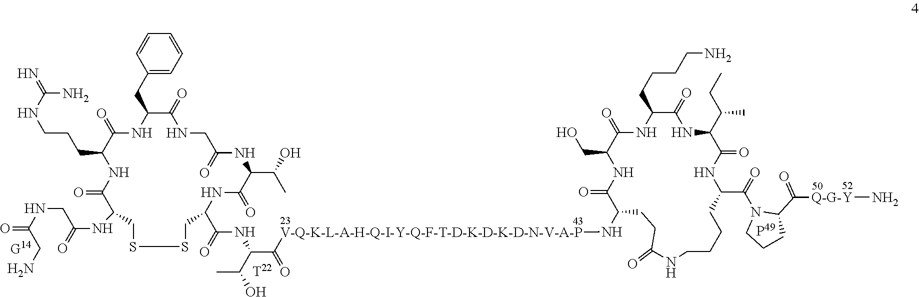

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-43)-((2S,5S,8S,11S,20S)-5-(4-aminobutyl)-20-(λ²-azaneyl)-8-((S)-sec-butyl)-2-(hydroxymethyl)-3,6,9,17,21-pentaoxo-1,4,7,10,16-pentaazacyclohenicosane-11-carbonyl)-L-prolyl-ADM(50-52) Chemical Formula: C₁₉₂H₂₉₇N₅₅O₅₇S₂

Exact Mass: 4349.15 Da

Molecular Weight: 4351.94 g/mol

Compound 4 was synthesized in a 15 μmol scale. The yield was 6.0 mg (9% f theory).

Compound 4 was analyzed via analytical RP-HPLC using a Kinetex® 5 μm C18 100 Å LC column (Phenomenex, 250 mm×4.6 mm, 5 m, 100 Å) applying a linear gradient of 10% to 60% eluent B in A over 40 min (Eluent A=0.1% TFA in water; Eluent B=0.08% TFA in ACN; flow rate=1.55 mL/min; λ=220 nm). Rt=19.2 min, purity≥95%.

In addition, a Jupiter® 4 μm Proteo 90 Å LC column (Phenomenex, 250 mm×4.6 mm, 4 m, 90 Å) was used, applying a linear gradient of 10% to 60% eluent B in A over 40 min (Eluent A=0.1% TFA in water; Eluent B=0.08% TFA in ACN; flow rate=1.0 mL/min; λ=220 nm). Rt=19.4 min, purity≥95%.

The observed mass was in accordance with the calculated mass.

ESI Ion-Trap: m/z=1088.9 [M+4H]⁴⁺, 871.3 [M+5H]⁵⁺, 726.2 [M+6H]⁶⁺, 622.6 [M+7H]⁷⁺.

MALDI-ToF: m/z=4350.1 [M+H]⁺, 2175.5 [M+2H]²⁺, 1450.3 [M+H]³⁺.

ODD-Modified Adrenomedullin Analogue 5

Synthesis:

The synthesis of compound 5 was performed using automated peptide synthesis of the sequence ADM(15-52) as described in the general method.

The coupling sequences were as follows:

| Coupling Cycle | compound 5 | AA of human ADM |
|---|---|---|
| 1. | Tyr(tBu) | 52 |
| 2. | Gly | 51 |
| 3. | Gln(Trt) | 50 |
| 4. | Pro | 49 |
| 5. | Ser(tBu) | 48 |
| 6. | Ile | 47 |
| 7. | Lys(Boc) | 46 |
| 8. | Ser(tBu) | 45 |
| 9. | Arg(Pbf) | 44 |
| 10. | Pro | 43 |
| 11. | Ala | 42 |
| 12. | Val | 41 |
| 13. | Asn(Trt) | 40 |
| 14. | Asp(tBu) | 39 |
| 15. | Lys(Boc) | 38 |
| 16. | Asp(tBu) | 37 |
| 17. | Lys(Boc) | 36 |
| 18. | Asp(tBu) | 35 |
| 19. | Thr(tBu) | 34 |
| 20. | Phe | 33 |
| 21. | Gln(Trt) | 32 |
| 22. | Tyr(tBu) | 31 |
| 23. | Ile | 30 |
| 24. | Gln(Trt) | 29 |
| 25. | His(Trt) | 28 |
| 26. | Ala | 27 |
| 27. | Leu | 26 |
| 28. | Lys(Boc) | 25 |
| 29. | Gln(Trt) | 24 |
| 30. | Val | 23 |
| 31. | Thr(tBu) | 22 |
| 32. | Cys(Trt) | 21 |
| 33. | Thr(tBu) | 20 |
| 34. | Gly | 19 |
| 35. | Phe | 18 |
| 36. | Arg(Pbf) | 17 |
| 37. | Cys(Trt) | 16 |
| 38. | Gly | 15 |

After automated synthesis of the sequence ADM(15-52), the N-terminal amino acid Boc-Lys(Fmoc)-OH was coupled manually with HOBt and DIC in 5-fold molar excess. The reaction was performed in DMF as solvent for 24 h.

Subsequently, the Fmoc protecting group was removed from the N-terminal amino acid using 20% piperidine in DMF (v/v) for 10 min twice.

Coupling of ODD to the free lysine side chain was achieved using a 5-fold excess (75 μmol) of octadecanedioic acid mono-tert-butyl ester, HOBt and DIC in 400 μDMF/DCM (3:1, v/v) as solvent for approx. 24 h.

Cleavage of the peptide from the resin and simultaneous side chain deprotection was achieved with TFA/TA/EDT (90:7:3, v/v/v) for approx. 3 h. The peptide was precipitated, washed with ice-cold diethyl ether and dried under reduced pressure.

Oxidation of the disulfide bond was achieved by solving the peptide in 10 m1 ACN/H₂O/TBS (2:3:5, v/v/v), adjusting the pH to 7.6-7.8 (1 M NaOH) and subsequent shaking for 12 h. Upon completion of the oxidation, the pH was adjusted to 3-4 using 1 M HCl.

Purification of the crude peptide was performed using preparative RP-HPLC on an Aeris PEPTIDE 5 μm XB-C18 LC column (Phenomenex, 250 mm×21.2 mm, 5 m, 100 Å). A linear gradient of 10% to 60% eluent B in A over 40 min was applied (Eluent A=0.1% TFA in water; Eluent B=0.08% TFA in ACN). The flow rate was 15 mL/min, UV detection was measured at λ=220 nm.

Analytics:

The identity of the peptides was confirmed via MALDI-MS (UltraflexIII, Bruker) and ESI-MS (HCT, Bruker). The purities were analyzed using analytical RP-HPLC.

Compound 5: [K¹⁴(ODD)]ADM(14-52)

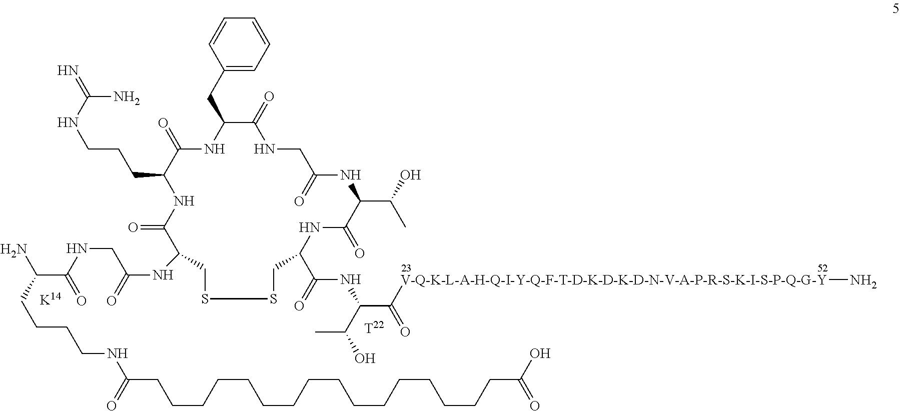

### ((4R,7S,13S,16S,19R)-19-(2-((S)-2-amino-6-(17-carboxyoctadecanamido)hexanamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-52)

Chemical Formula: C₂₁₂H₃₃₅N₅₈O₆₀S₂

Exact Mass: 4720.46 Da

Molecular Weight: 4723.50 g/mol

Compound 5 was synthesized in a 15 μmol scale. The yield was 3.8 mg (5% f theory).

Compound 5 was analyzed via analytical RP-HPLC using a Kinetex® 5 μm Biphenyl 100 Å LC column (Phenomenex, 250 mm×4.6 mm, 5 m, 100 Å) applying a linear gradient of 20% to 70% eluent B in A over 40 min (Eluent A=0.1% TFA in water; Eluent B=0.08% TFA in ACN; flow rate=1.55 mL/min; λ=220 nm). Rt=14.3 min, purity≥95%.

In addition, a Jupiter® 4 μm Proteo 90 Å LC column (Phenomenex, 250 mm×4.6 mm, 4 m, 90 Å) was used, applying a linear gradient of 20% to 70% eluent B in A over 40 min (Eluent A=0.1% TFA in water; Eluent B=0.08% TFA in ACN; flow rate=1.0 mL/min; λ=220 nm). Rt=17.8 min, purity≥95%.

The observed mass was in accordance with the calculated mass.

ESI Ion-Trap: m/z=1181.6 [M+4H]⁴⁺, 945.5 [M+5H]⁵⁺, 788.1 [M+6H]⁶⁺, 675.6 [M+7H]⁷⁺, 591.3 [M+8H]⁸⁺.

MALDI-ToF: m/z=4721.47 [M+H]⁺, 2361.09 [M+2H]²⁺.

Adrenomedullin Analogues 6-12 Containing Different Length OEG-Linkers

For the synthesis of compounds 6-12, different-length oligo ethylene glycol linker blocks herein termed OEG-linker building blocks A, B, C and D (shown below) were used as replacements for four, five or six amino acids of ADM positions 35 to 41. Sometimes the term polyethylene glycole PEG is used also for very low repetition numbers, it is considered synonymous under these circumstances. The building blocks were N-α-Fmoc-protected; Here, nomenclature is defined as Fmoc-NH-OEG(x)-OH, where x describes the number of atoms in the linker upon incorporation.

Structures of Fmoc-NH-OEG(x)-OH building blocks:

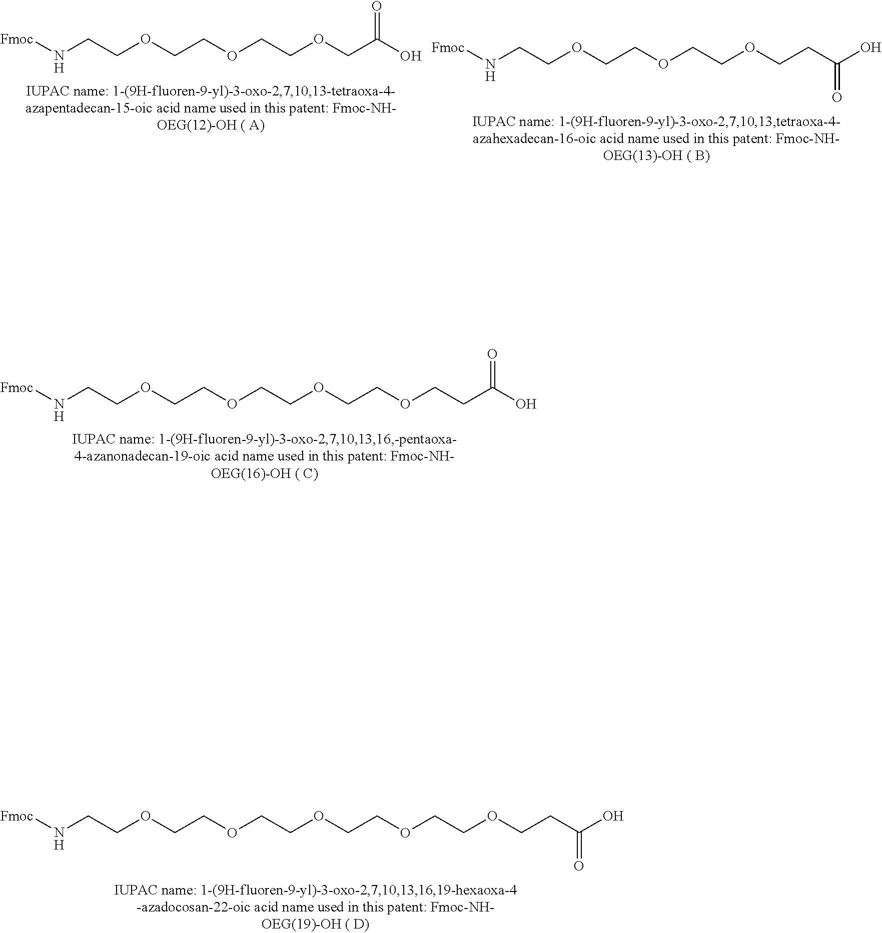

A: Fmoc-NH-OEG(12)-OH; CAS #: 139338-72-0; The compound was purchased from ChemPep Inc.

B: Fmoc-NH-OEG(13)-OH; CAS #: 867062-95-1; The compound was purchased from Iris Biochem.

C: Fmoc-NH-OEG(16)-OH; CAS #: 557756-85-1; The compound was purchased from Iris Biochem.

D: Fmoc-NH-OEG(19)-OH; CAS #: 882847-32-7; The compound was purchased from Iris Biochem.

Structures of —NH—OEG(x)-OH Building Blocks

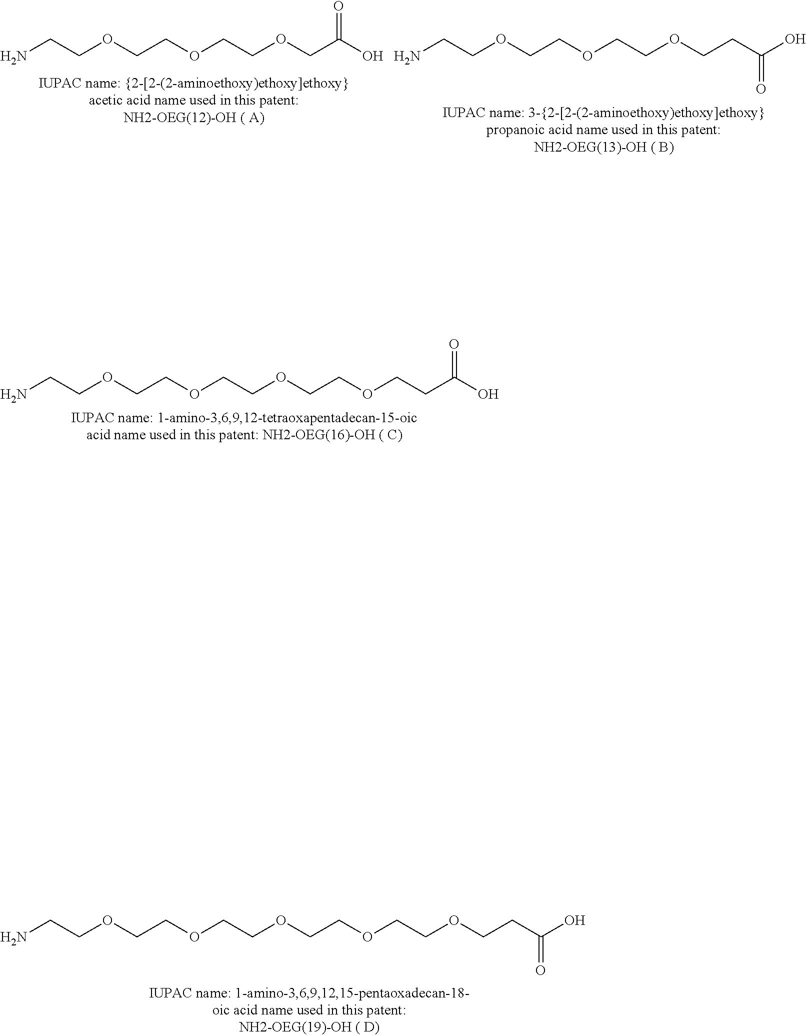 [SMILES: NCCOCCOCCOCC(=O)O ; NCCOCCOCCOCCC(=O)O ; NCCOCCOCCOCCOCCC(=O)O ; NCCOCCOCCOCCOCCOCCC(=O)O]

Synthesis:

The syntheses of compounds 6-12 were performed using automated peptide synthesis as described in the general method.

The coupling sequences were as follows:

| | | | | | | | | AA of |
|---|---|---|---|---|---|---|---|---|
| | comp. | comp. | comp. | comp. | comp. | comp. | comp. | human |
| Cycle | 6 | 7 | 8 | 11 | 9 | 10 | 23 | ADM |

| 1. | Tyr(tBu) | | | | | | 52 |
|---|---|---|---|---|---|---|---|
| 2. | Gly | | | | | | 51 |
| 3. | Gln(Trt) | | | | | | 50 |
| 4. | Pro | | | | | | 49 |
| 5. | Ser(tBu) | | | | | | 48 |
| 6. | Ile | | | | | | 47 |
| 7. | Lys(Boc) | | | | | | 46 |
| 8. | Ser(tBu) | | | | | | 45 |
| 9. | Arg(Pbf) | | | | | | 44 |
| 10. | Pro | | | | | | 43 |
| 11. | Ala | | | | | | 42 |
| 12. | Val | | | | | — | 41 |
| 13. | Asn(Trt) | | | — | — | — | 40 |
| 14. | Asp(tBu) | — | — | — | — | — | 39 |

After automated synthesis of the C-terminal sequences, Fmoc-NH-OEG(x)-OH building block A (6), B (7, 9), C (8, 10) or D (11, 12) was coupled manually with HOBt and DIC in 5-fold molar excess. The reaction was performed in DMF as solvent for 24 h.

Subsequently, the Fmoc protecting group was removed using 20% piperidine in DMF (v/v) for 10 min twice and Fmoc-Thr(tBu)-OH (6, 7, 8, 11) or Fmoc-Asp(tBu)-OH (9, 10, 12) was coupled manually with HOBt and DIC in 5-fold molar excess. The reaction was performed in DMF as solvent for 24 h.

Elongation of the peptide chain was performed using the general method for automated peptide synthesis described above. The elongation amino acids were N-α-Fmoc-protected, while Boc-Gly-OH was used as N-terminal amino acid.

The coupling sequences were as follows:

| | | | | | | | | AA of |
|---|---|---|---|---|---|---|---|---|
| | comp. | comp. | comp. | comp. | comp. | comp. | comp. | human |
| Cycle | 6 | 7 | 8 | 11 | 9 | 10 | 23 | ADM |

| | | | |
|---|---|---|---|
| 1 | Phe | Thr(tBu) | 33/34 |
| 2 | Gln(Trt) | Phe | 32/33 |
| 3 | Tyr(tBu) | Gln(Trt) | 31/32 |
| 4 | Ile | Tyr(tBu) | 30/31 |
| 5 | Gln(Trt) | Ile | 29/30 |
| 6 | Hi s (Trt) | Gln(Trt) | 28/29 |
| 7 | Ala | His(Trt) | 27/28 |
| 8 | Leu | Ala | 26/27 |
| 9 | Lys(Boc) | Leu | 25/26 |
| 10 | Gln(Trt) | Lys(Boc) | 24/25 |
| 11 | Val | Gln(Trt) | 23/24 |
| 12 | Thr(tBu) | Val | 22/23 |
| 13 | Cys (Trt) | Thr(tBu) | 21/22 |
| 14 | Thr(tBu) | Cys(Trt) | 20/21 |
| 15 | Gly | Thr(tBu) | 19/20 |
| 16 | Phe | Gly | 18/19 |
| 17 | Arg(Pbf) | Phe | 17/18 |
| 18 | Cys(Trt) | Arg(Pbf) | 16/17 |
| 19 | Gly | Cys(Trt) | 15/16 |
| 20 | Boc-Gly | Gly | 14/15 |
| 21 | — | Boc-Gly | −/14 |

Cleavage of the peptides from the resin and simultaneous side chain deprotection was achieved with PGP-124T, TFA/TA/EDT (90:7:3, v/v/v) for approx. 3 h. The peptides were precipitated and washed with ice-cold diethyl ether and subsequently dried under reduced pressure.

Oxidation of the disulfide bond was achieved by solving the peptide in ACN/H₂O/TBS (1:4:5, v/v/v), adjusting the pH to 7.6-7.8 (1 M NaOH) and subsequent shaking for 12 h. Upon completion of the oxidation, the pH was adjusted to 3-4 using 1 M HCl.

Purification of the crude peptide was performed using preparative RP-HPLC on an Aeris PEPTIDE 5 μm XB-C18 LC column (Phenomenex, 250 mm×21.2 mm, 5 m, 100 Å). A linear gradient of 10% to 50% eluent B in A over 30 min was applied (Eluent A=0.1% TFA in water; Eluent B=0.08% TFA in ACN). The flow rate was 15 mL/min, UV detection was measured at λ=220 nm.

Analytics:

The identity of the peptides was confirmed by MALDI-MS (UltraflexIII, Bruker) and ESI-MS (HCT, Bruker). The purities were analyzed using analytical RP-HPLC.

Compound 6: [G¹⁴, OEG(12)^{[35-38]}]ADM(14-52)

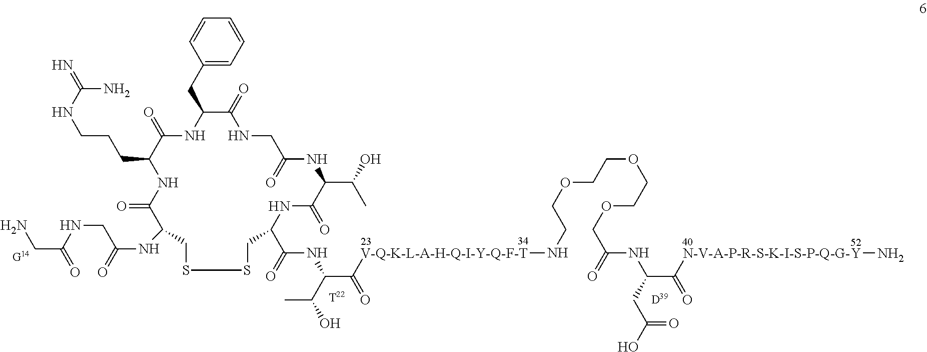

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-34)-(2-(2-(2-(2-(k2-azaneyl)ethoxy)ethoxy)ethoxy)acetyl)-L-aspartyl-ADM(40-52)

Chemical Formula: C₁₇₈H₂₇₈N₅₂O₅₃S₂

Exact Mass: 4056.01 Da

Molecular Weight: 4058.61 g/mol

Compound 6 was synthesized in a 15 μmol scale. The yield was 1.8 mg (3% f theory).

Compound 6 was analyzed by analytical RP-HPLC using a Kinetex® 5 μm Biphenyl 100 Å LC column (Phenomenex, 250 mm×4.6 mm, 5 m, 100 Å) applying a linear gradient of 10% to 60% eluent B in A over 40 min (Eluent A=0.1% TFA in water; Eluent B=0.08% TFA in ACN; flow rate=1.55 mL/min; λ=220 nm). Rt=15.6 min, purity≥95%.

In addition, a Jupiter® 4 μm Proteo 90 Å LC column (Phenomenex, 250 mm×4.6 mm, 4 m, 90 Å) was used, applying a linear gradient of 10% to 60% eluent B in A over 40 min (Eluent A=0.1% TFA in water; Eluent B=0.08% TFA in ACN; flow rate=1.0 mL/min; λ=220 nm). Rt=18.7 min, purity≥95%.

The observed mass was in accordance with the calculated mass.

ESI Ion-Trap: m/z=1015.4 [M+4H]⁴⁺, 812.6 [M+5H]⁵⁺, 677.3 [M+6H]⁶⁺.

MALDI-ToF: m/z=4057.0 [M+H]⁺, 2029.0 [M+2H]²⁺.

Compound 7: [G¹⁴, OEG(13)^{[35-38]}]ADM(14-52)

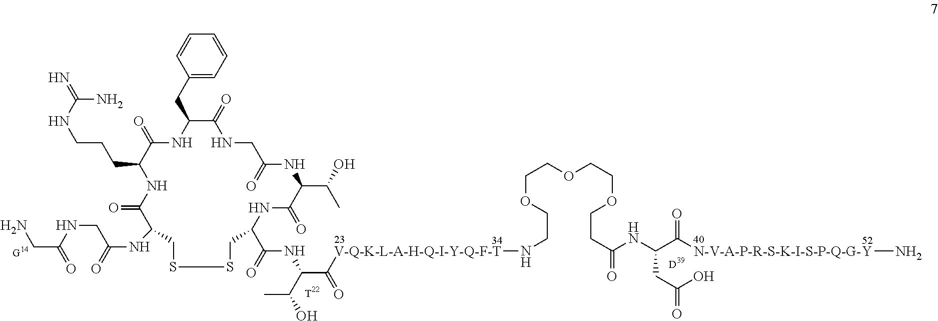

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-34)-(3-(2-(2-(2-(λ²-azaneyl)ethoxy)ethoxy)ethoxy)propanoyl)-L-aspartyl-ADM(40-52)

Chemical Formula: C₁₇₉H₂₈₀N₅₂O₅₃S₂

Exact Mass: 4070.03 Da

Molecular Weight: 4072.64 g/mol

Compound 7 was synthesized in a 7.5 μmol scale. The yield was 6.0 mg (20% f theory).

Compound 7 was analyzed by analytical RP-HPLC using a Kinetex® 5 μm Biphenyl 100 Å LC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=21.2 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=23.4 min, purity≥95%.

The observed mass was in accordance with the calculated mass.

ESI Ion-Trap: m/z=1018.8 [M+4H]⁴⁺, 815.4 [M+5H]⁵⁺, 679.6 [M+6H]⁶⁺, 582.6 [M+7H]⁷⁺.

MALDI-ToF: m/z=4071.0 [M+H]⁺, 2036.0 [M+2H]²⁺.

Compound 8: [G¹⁴, OEG(16)^{[35-39]}]ADM(14-52)

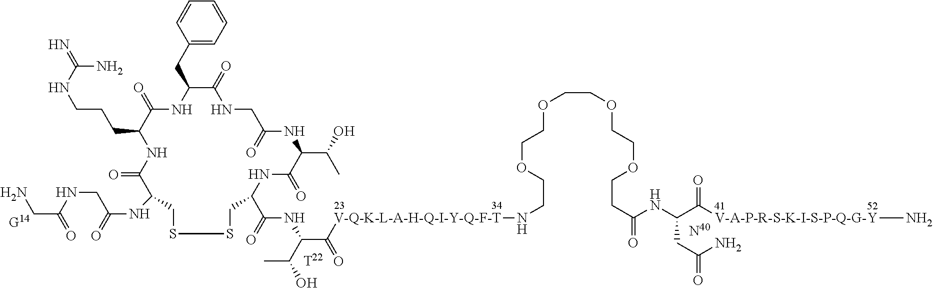

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-34)-(1-(λ²-azaneyl)-3,6,9,12-tetraoxapentadecan-15-oyl)-L-asparagyl-ADM(41-52)

Chemical Formula: C₁₇₇H₂₇₉N₅₁O₅₁S₂

Exact Mass: 3999.02 Da

Molecular Weight: 4001.61 g/mol

Compound 8 was synthesized in a 7.5 μmol scale. The yield was 5.4 mg (18% f theory).

Compound 8 was analyzed by analytical RP-HPLC using a Kinetex® 5 μm Biphenyl 100 Å LC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=21.4 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=23.5 min, purity≥95%.

The observed mass was in accordance with the calculated mass.

ESI Ion-Trap: m/z=1001.2 [M+4H]⁴⁺, 801.1 [M+5H]⁵⁺, 667.8 [M+6H]⁶⁺, 572.5 [M+7H]⁷⁺.

MALDI-ToF: m/z=4000.0 [M+H]⁺, 2000.5 [M+2H]²⁺, 1334.0 [M+3H]³⁺.

Compound 9: [G¹⁴, OEG(13)^{[36-39]}]ADM(14-52)

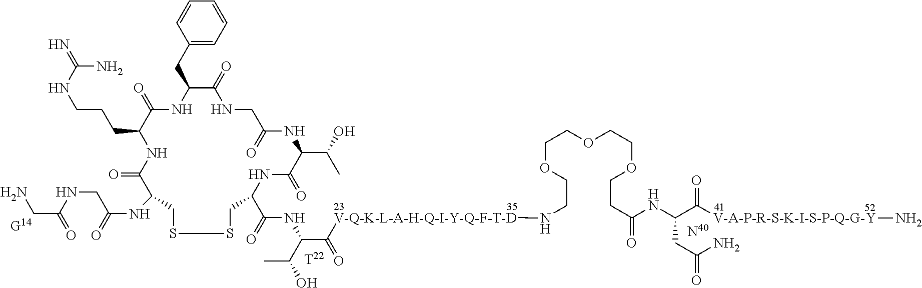

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-35)-(3-(2-(2-(2-(λ²-azaneyl)ethoxy)ethoxy)ethoxy)propanoyl)-L-asparagyl-ADM(41-52)

Chemical Formula: C₁₇₉H₂₈₀N₅₂O₅₃S₂

Exact Mass: 4070.03 Da

Molecular Weight: 4072.64 g/mol

Compound 9 was synthesized in a 7.5 μmol scale. The yield was 2.4 mg (8% f theory).

Compound 9 was analyzed by analytical RP-HPLC using a Kinetex® 5 μm Biphenyl 100 Å LC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=21.6 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=23.7 min, purity≥95%.

The observed mass was in accordance with the calculated mass.

ESI Ion-Trap: m/z=1358.3 [M+3H]³⁺, 1018.9 [M+4H]⁴⁺, 815.4 [M+5H]⁵⁺, 679.7 [M+6H]⁶⁺.

MALDI-ToF: m/z=4071.0 [M+H]⁺, 2036.0 [M+2H]²⁺.

Compound 10: [G¹⁴, OEG(16)^{[36-40]}]ADM(14-52)

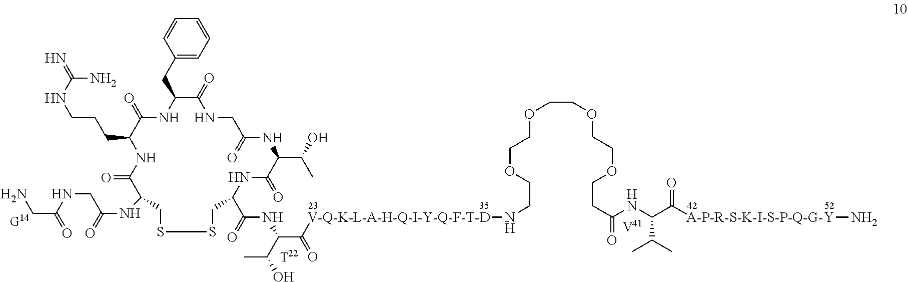

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-35)-(1-(λ²-azaneyl)-3,6,9,12-tetraoxapentadecan-15-oyl)-L-valyl-ADM(42-52)

Chemical Formula: C₁₇₇H₂₇₈N₅₀O₅₂S₂

Exact Mass: 4000.01 Da

Molecular Weight: 4002.59 g/mol

Compound 10 was synthesized in a 7.5 μmol scale. The yield was 3.6 mg (12% f theory).

Compound 10 was analyzed by analytical RP-HPLC using a Kinetex® 5 μm Biphenyl 100 ALC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=22.2 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=24.3 min, purity≥95%.

The observed mass was in accordance with the calculated mass.

ESI Ion-Trap: m/z=1001.6 [M+4H]⁴⁺, 801.3 [M+5H]⁵⁺, 668.0 [M+6H]⁶⁺, 572.7 [M+7H]⁷⁺.

MALDI-ToF: m/z=4001.0 [M+H]⁺, 2001.0 [M+2H]²⁺, 1334.3 [M+3H]³⁺.

Compound 11: [G¹⁴, OEG(19)[35-40]]ADM(14-52)

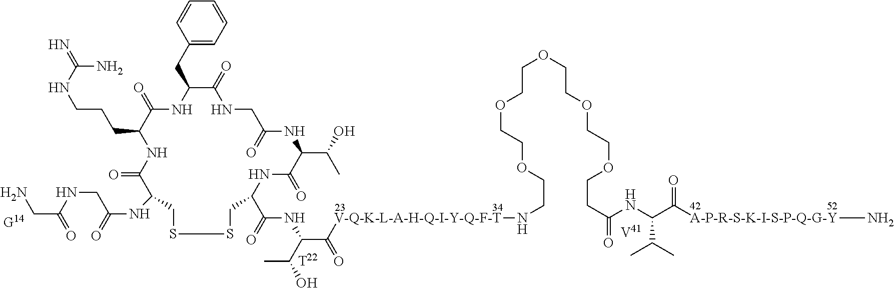

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-34)-(1-(λ²-azaneyl)-3,6,9,12,15-pentaoxaoctadecan-18-oyl)-L-valyl-ADM(42-52)

Chemical Formula: C₁₇₅H₂₇₇N₄₉O₅₀S₂

Exact Mass: 3929.01 Da

Molecular Weight: 3931.55 g/mol

Compound 11 was synthesized in a 7.5 μmol scale. The yield was 6.9 mg (23% f theory).

Compound 11 was analyzed by analytical RP-HPLC using a Kinetex® 5 μm Biphenyl 100 ALC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=22.1 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=24.1 min, purity≥95%.

The observed mass was in accordance with the calculated mass.

ESI Ion-Trap: m/z=983.5 [M+4H]^{4, 787.2} [M+5H]⁵⁺, 656.1 [M+6H]⁶⁺, 562.5 [M+7H]⁷⁺.

MALDI-ToF: m/z=3930.1 [M+H]⁺, 1965.5 [M+2H]²⁺.

Compound 12: [G¹⁴, OEG(19)^{[36}-^{41]}]ADM(14-52)

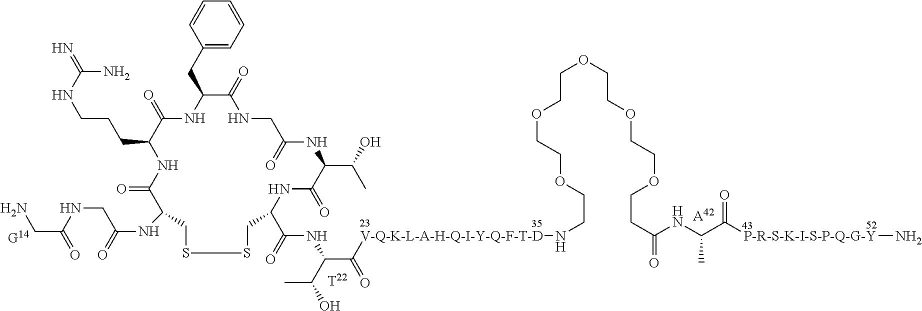

### ((4R,7S,13S,16S,19R)-19-(2-(2-aminoacetamido)acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-35)-(1-(λ²-azaneyl)-3,6,9,12,15-pentaoxaoctadecan-18-oyl)-L-alanyl-ADM(43-52)

Chemical Formula: C₁₇₄H₂₇₃N₄₉O₅₂S₂

Exact Mass: 3944.97 Da

Molecular Weight: 3947.51 g/mol

Compound 12 was synthesized in a 7.5 μmol scale. The yield was 0.7 mg (2% f theory).

Compound 12 was analyzed by analytical RP-HPLC using a Kinetex® 5 μm Biphenyl 100 ALC column (Phenomenex, 250 mm×4.6 mm, 5 m, 100 Å) applying a linear gradient of 10% to 50% eluent B in A over 40 min (Eluent A=0.1% TFA in water; Eluent B=0.08% TFA in ACN; flow rate=1.55 mL/min; λ=220 nm). Rt=21.8 min, purity≥95%.

In addition, a Jupiter® 4 μm Proteo 90 Å LC column (Phenomenex, 250 mm×4.6 mm, 4 m, 90 Å) was used, applying a linear gradient of 10% to 50% eluent B in A over 40 min (Eluent A=0.1% TFA in 15 water; Eluent B=0.08% TFA in ACN; flow rate=1.0 mL/min; λ=220 nm). Rt=24.0 min, purity≥95%.

The observed mass was in accordance with the calculated mass.

ESI Ion-Trap: m/z=987.6 [M+4H]⁴⁺, 790.3 [M+5H]⁵⁺, 658.8 [M+6H]⁶⁺, 564.8 [M+7H]⁷⁺.

MALDI-ToF: m/z=3946.0 [M+H]⁺, 1973.5 [M+2H]²⁺.

Double Modified Adrenomedullin-Analogues 13

Synthesis:

The synthesis of compound 13 was performed using automated peptide synthesis as described in the general method.

The coupling sequence was as follows:

| Coupling Cycle | compound 13 | AA of human ADM |
|---|---|---|
| 1 | Tyr(tBu) | 52 |
| 2 | Gly | 51 |
| 3 | Gln(Trt) | 50 |
| 4 | Pro | 49 |
| 5 | Asp(OPp) | 48 |
| 6 | Ile | 47 |
| 7 | Lys(Boc) | 46 |
| 8 | Ser(tBu) | 45 |
| 9 | Lys(Mmt) | 44 |
| 10 | Pro | 43 |
| 11 | Ala | 42 |
| 12 | Val | 41 |
| 13 | Asn(Trt) | 40 |
| 14 | Asp(tBu) | 39 |
| 15 | Lys(Mmt) | 38 |
| 16 | Asp(tBu) | 37 |
| 17 | Lys(Mmt) | 36 |
| 18 | Asp(tBu) | 35 |
| 19 | Thr(tBu) | 34 |
| 20 | Phe | 33 |
| 21 | Gln(Trt) | 32 |
| 22 | Tyr(tBu) | 31 |
| 23 | Ile | 30 |
| 24 | Gln(Trt) | 29 |
| 25 | His(Trt) | 28 |
| 26 | Ala | 27 |
| 27 | Leu | 26 |
| 28 | Lys(Boc) | 25 |
| 29 | Gln(Trt) | 24 |
| 30 | Val | 23 |
| 31 | Thr(tBu) | 22 |
| 32 | Cys(Trt) | 21 |
| 33 | Thr(tBu) | 20 |
| 34 | Gly | 19 |
| 35 | Phe | 18 |
| 36 | Arg(Pbf) | 17 |
| 37 | Cys(Trt) | 16 |
| 38 | Gly | 15 |

After automated synthesis, the N-terminal amino acid Boc-Lys(Fmoc)-OH was coupled manually with HOBt and DIC in 5-fold molar excess. The reaction was performed in DMF as solvent for 24 h.

Subsequently, the Fmoc protecting group was removed from the N-terminal amino acid using 20% piperidine in DMF (v/v) for 10 min twice.

Coupling of ODD to the free lysine side chain was achieved using a 5-fold excess (75 μmol) of octadecanedioic acid mono-tert-butyl ester, HOBt and DIC in 400 μDMF/DCM (3:1, v/v) as solvent for approx. 24 h.

For the simultaneous removal of Mmt/OPp protecting groups the resin was treated with TFA/TIS/DCM (3:5:92, v/v/v) (10×2 min, 1 mL). Subsequently, the resin was washed with 2.5% DIPEA in DMF (v/v) for 10 min twice (1 mL).

Cyclization was performed using a 30-fold molar excess of HOBt and DIC in DMF as solvent for approx. 24 h at T=40° C.

Cleavage of the peptides from the resin and simultaneous side chain deprotection was achieved with TFA/TA/EDT (90:7:3, v/v/v) for approx. 3 h. The peptides were precipitated and washed with ice-cold diethyl ether/n-hexane (3:1, v/v) and dried under reduced pressure.

Oxidation of the disulfide bond was achieved by solving the peptide in 20 m1 ACN/H₂O/TBS (2:3:5, v/v/v), adjusting the pH to 7.6-7.8 (1 M NaOH) and subsequent shaking for 12 h. Upon completion of the oxidation, the pH was adjusted to 3-4 using 1 M HCl.

Purification of the crude peptide was performed using preparative RP-HPLC on an Aeris PEPTIDE 5 μm 20 XB-C18 LC column (Phenomenex, 250 mm×21.2 mm, 5 m, 100 Å). A linear gradient of 20% to 60% eluent B in A over 40 min was applied (Eluent A=0.1% TFA in water; Eluent B=0.08% TFA in ACN). The flow rate was 20 mL/min, UV detection was measured at λ=220 nm.

Analytics:

The identity of the peptides was confirmed via MALDI-MS (UltraflexIII, Bruker) and ESI-Orbitrap-MS (Orbitrap Elite™, Thermo Scientific). The purities were analyzed using analytical RP-HPLC.

Compound 13: [K¹⁴(ODD), (K⁴⁴, D⁴⁸)_{lac}] ADM(14-52)

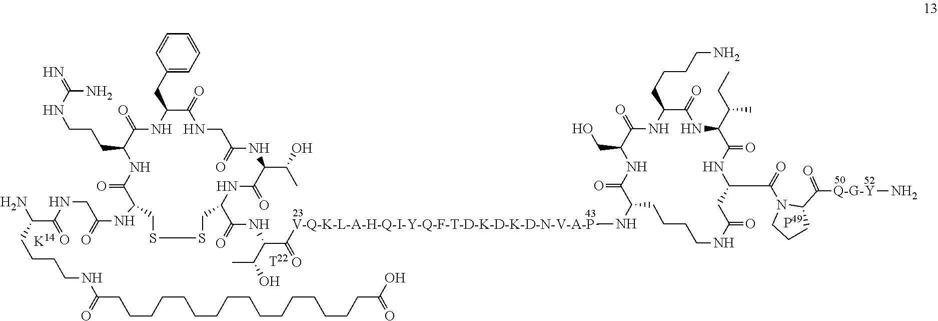

### ((4R,7S,13S,16S,19R)-19-(2-((S)-2-amino-6-(17-carboxyoctadecanamido)hexanamido) acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-43)-((2S,5S,8S,11S,18S)-5-(4-aminobutyl)-18-(λ²-azaneyl)-8-((S)-sec-butyl)-2-(hydroxymethyl)-3,6,9,13,19-pentaoxo-1,4,7,10,14-pentaazacyclononadecane-11-carbonyl)-L-prolyl-ADM(50-52)

Chemical Formula: C₂₁₃H₃₃₆N₅₆O₆₀S₂

Exact Mass: 4702.44 Da

Molecular Weight: 4705.48 g/mol

Compound 13 was synthesized in a 15 μmol scale. The yield was 1.0 mg (1% f theory).

Compound 13 was analyzed via analytical RP-HPLC using a Kinetex® 5 μm C18 100 Å LC column (Phenomenex, 250 mm×4.6 mm, 5 m, 100 Å) applying a linear gradient of 20% to 70% eluent B in A over 30 min (Eluent A=0.1% TFA in water; Eluent B=0.08% TFA in ACN; flow rate=1.55 mL/min; λ=220 nm). Rt=15.1 min, purity≥95%.

In addition, a Jupiter® 4 μm Proteo 90 Å LC column (Phenomenex, 250 mm×4.6 mm, 4 m, 90 Å) was used, applying a linear gradient of 20% to 70% eluent B in A over 40 min (Eluent A=0.1% TFA in water; Eluent B=0.08% TFA in ACN; flow rate=1.0 mL/min; λ=220 nm). Rt=18.3 min, purity≥95%.

The observed mass was in accordance with the calculated mass.

ESI Orbitrap: m/z=1177.4 [M+4H]⁴⁺, 942.1 [M+5H]⁵⁺, 785.1 [M+6H]⁶⁺, 673.2 [M+7H]⁷⁺.

MALDI-ToF: m/z=4703.4 [M+H]⁺, 2352.1 [M+2H]²⁺.

Triple modified Adrenomedullin-Analogue 14

Synthesis:

The synthesis of compound 14 was performed using automated peptide synthesis as described in the general method. In coupling cycle 15, Fmoc-NH—OEG(13)-OH was used as amino acid.

The coupling sequence was as follows:

| | | AA of |
|---|---|---|
| | | human |
| Coupling Cycle | compound 14 | ADM |
| 1 | Tyr(tBu) | 52 |
| 2 | Gly | 51 |
| 3 | Gln(Trt) | 50 |
| 4 | Pro | 49 |
| 5 | Asp(OPp) | 48 |
| 6 | Ile | 47 |
| 7 | Lys(Boc) | 46 |
| 8 | Ser(tBu) | 45 |
| 9 | Lys(Mmt) | 44 |
| 10 | Pro | 43 |
| 11 | Ala | 42 |
| 12 | Val | 41 |
| 13 | Asn(Trt) | 40 |
| 14 | Asp(tBu) | 39 |
| 15 | OEG(13) | 35-38 |
| 16 | Thr(tBu) | 34 |
| 17 | Phe | 33 |
| 18 | Gln(Trt) | 32 |
| 19 | Tyr(tBu) | 31 |
| 20 | Ile | 30 |
| 21 | Gln(Trt) | 29 |
| 22 | His(Trt) | 28 |
| 23 | Ala | 27 |
| 24 | Leu | 26 |
| 25 | Lys(Boc) | 25 |
| 26 | Gln(Trt) | 24 |
| 27 | Val | 23 |
| 28 | Thr(tBu) | 22 |
| 29 | Cys(Trt) | 21 |
| 30 | Thr(tBu) | 20 |
| 31 | Gly | 19 |
| 32 | Phe | 18 |
| 33 | Arg(Pbf) | 17 |
| 34 | Cys(Trt) | 16 |
| 35 | Gly | 15 |

After automated synthesis, the N-terminal amino acid Boc-Lys(Fmoc)-OH was coupled manually with HOBt and DIC in 5-fold molar excess. The reaction was performed in DMF for 24 h.

Subsequently, the Fmoc protecting group was removed from the N-terminal amino acid using 20% piperidine in DMF (v/v) for 10 min twice.

Coupling of ODD to the free lysine side chain was achieved using a 5-fold excess (75 μmol) of octadecanedioic acid mono-tert-butyl ester, HOBt and DIC in 400 μDMF/DCM (3:1, v/v) as solvent for approx. 24 h.

For simultaneous removal of the Mmt/OPp protecting groups, the resin was treated with TFA/TIS/DCM (3:5:92, v/v/v) (10×2 min, 1 mL). Subsequently, the resin was washed with 2.5% DIPEA in DMF (v/v) for 10 min twice (1 mL).

Cyclization was performed using a 30-fold molar excess of HOBt and DIC in DMF for approx. 24 h at T=40° C.

Cleavage of the peptides from the resin and simultaneous side chain deprotection was achieved with TFA/TA/EDT (90:7:3, v/v/v) for approx. 3 h. The peptides were precipitated and washed with ice-cold diethyl ether/n-hexane (3:1, v/v) and dried under reduced pressure.

Oxidation of the disulfide bond was achieved by solving the peptide in 20 m1 ACN/H₂O/TBS (2:3:5, v/v/v), adjusting the pH to 7.6-7.8 (1 M NaOH) and subsequent shaking for 12 h. Upon completion of the oxidation, the pH was adjusted to 3-4 using 1 M HCl.

Purification of the crude peptide was performed using preparative RP-HPLC on an Aeris PEPTIDE 5 μm XB-C18 LC column (Phenomenex, 250 mm×21.2 mm, 5 m, 100 Å). A linear gradient of 20% to 60% eluent B in A over 40 min was applied (Eluent A=0.1% TFA in water; Eluent B=0.08% TFA in ACN). The flow rate was 20 mL/min, UV detection was measured at, =220 nm.

Analytics:

The identity of the peptides was confirmed via MALDI-MS (UltraflexIII, Bruker) and ESI-Orbitrap-MS (Orbitrap Elite™, Thermo Scientific). The purities were analyzed using analytical RP-HPLC.

Compound 14: [K¹⁴(ODD), OEG(13)^{[35-38]}, (K⁴⁴, D⁴⁸)_{lac}]ADM(14-52)

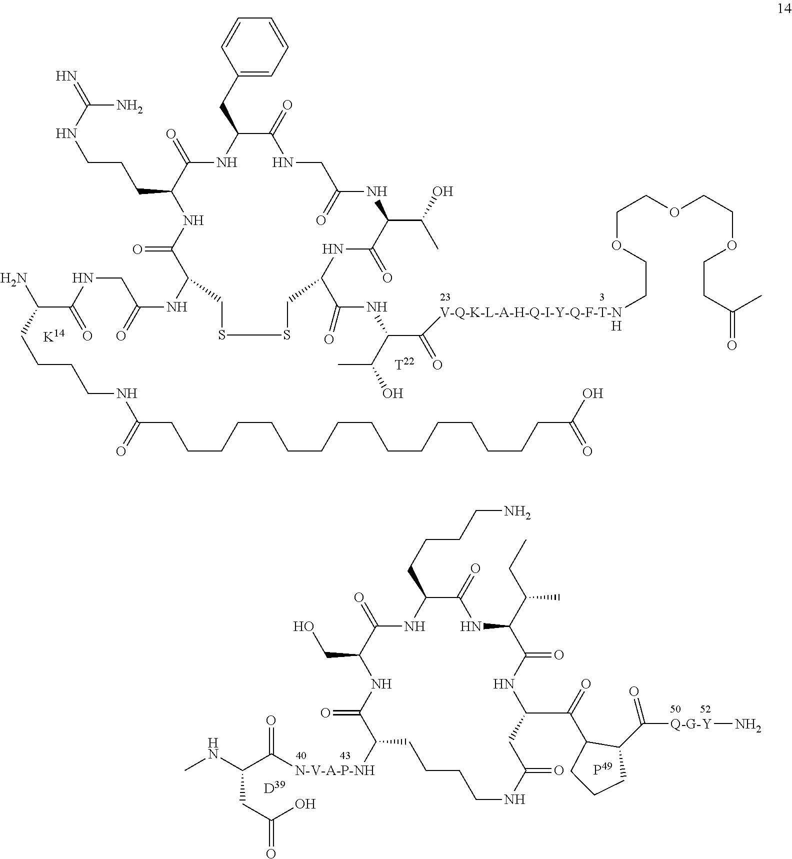

### ((4R,7S,13S,16S,19R)-19-(2-((S)-2-amino-6-(17-carboxyoctadecanamido)hexanamido) acetamido)-13-benzyl-16-(3-guanidinopropyl)-7-((R)-1-hydroxyethyl)-6,9,12,15,18-pentaoxo-1,2-dithia-5,8,11,14,17-pentaazacycloicosane-4-carbonyl)-L-threonyl-ADM(23-34)-(3-(2-(2-(2-(λ²-azaneyl)ethoxy)ethoxy)ethoxy)propanoyl)-L-aspartyl-ADM(40-43)-((2S,5S,8S,11S,18S)-5-(4-aminobutyl)-18-(λ²-azaneyl)-8-((S)-sec-butyl)-2-(hydroxymethyl)-3,6,9,13,19-pentaoxo-1,4,7,10,14-pentaazacyclononadecane-11-carbonyl)-L-prolyl-ADM(50-52)

Chemical Formula: C₂₀₂H₃₁₉N₅₁O₅₆S₂

Exact Mass: 4419.31 Da

Molecular Weight: 4422.20 g/mol

Compound 14 was synthesized in a 15 μmol scale. The yield was 1.7 mg (3% of theory).

Compound 14 was analyzed via analytical RP-HPLC using a Kinetex® 5 μm C18 100 Å LC column (Phenomenex, 250 mm×4.6 mm, 5 m, 100 Å) applying a linear gradient of 20% to 70% eluent B in A over 30 min (Eluent A=0.1% TFA in water; Eluent B=0.08% TFA in ACN; flow rate=1.55 mL/min; λ=220 nm). Rt=15.8 min, purity≥95%.

In addition, a Jupiter® 4 μm Proteo 90 Å LC column (Phenomenex, 250 mm×4.6 mm, 4 m, 90 Å) was used, applying a linear gradient of 20% to 70% eluent B in A over 40 min (Eluent A=0.1% TFA in water; Eluent B=0.08% TFA in ACN; flow rate=1.0 mL/min; λ=220 nm). Rt=19.4 min, purity≥95%.

The observed mass was in accordance with the calculated mass.

ESI Orbitrap: m/z=1474.8 [M+3H]³⁺, 1106.3 [M+4H]⁴⁺, 885.3 [M+5H]⁵⁺.

MALDI-ToF: m/z=4420.3 [M+H]⁺, 2210.6 [M+2H]²⁺.

N-terminal Lactam-bridged Adrenomedullin-Analogues 15-17

Synthesis:

The synthesis of compound 15-17 was performed using automated peptide synthesis as described in the general method.

The coupling sequence was as follows:

| | | | | AA of |
|---|---|---|---|---|
| Coupling | compound | compound | compound | human |
| Cycle | 15 | 16 | 17 | ADM |

| 1 | Tyr(tBu) | | 52 |
|---|---|---|---|
| 2 | Gly | | 51 |
| 3 | Gln(Trt) | | 50 |
| 4 | Pro | | 49 |
| 5 | Ser(tBu) | Asp(OPp) | 48 |
| 6 | Ile | | 47 |
| 7 | Lys(Boc) | | 46 |
| 8 | Ser(tBu) | | 45 |
| 9 | Arg(Pbf) | Lys(Mmt) | 44 |

| 10 | Pro | | 43 |
|---|---|---|---|
| 11 | Ala | | 42 |
| 12 | Val | | 41 |
| 13 | Asn(Trt) | | 40 |
| 14 | Asp(tBu) | | 39 |
| 15 | Lys(Boc) | OEG(13) | 38 |
| 16 | Asp(tBu) | — | 37 |
| 17 | Lys(Boc) | — | 36 |
| 18 | Asp(tBu) | — | 35 |

| | | |
|---|---|---|
| 19 | Thr(tBu) | 34 |
| 20 | Phe | 33 |
| 21 | Gln(Trt) | 32 |
| 22 | Tyr(tBu) | 31 |
| 23 | Ile | 30 |
| 24 | Gln(Trt) | 29 |
| 25 | His(Trt) | 28 |
| 26 | Ala | 27 |
| 27 | Leu | 26 |
| 28 | Lys(Boc) | 25 |
| 29 | Gln(Trt) | 24 |
| 30 | Val | 23 |
| 31 | Thr(tBu) | 22 |
| 32 | Cys(Trt) | 21 |
| 33 | Thr(tBu) | 20 |
| 34 | Gly | 19 |
| 35 | Phe | 18 |
| 36 | Arg(Pbf) | 17 |
| 37 | Cys(Trt) | 16 |
| 38 | Gly | 15 |
| 39 | Boc-Lys(Fmoc) | 14 |

Coupling of ODD to the free lysine side chain was achieved using a 5-fold excess (75 μmol) of octadecanedioic acid mono-tert-butyl ester, HOBt and DIC in 400 μl DMF/DCM (3:1, v/v) as solvent for approx. 24 h.

The side chain protection groups OPp, Mint and Mtt were removed simultaneously by incubating the resin 15-times in TFA/TIS/DCM (2:5:93, v/v/v) for 1 min. Subsequently, the resin was neutralized two using 2.5% DIPEA/DMF (v/v) for 10 min Lactam cyclisation was achieved using 30 eq. HOBt and 30 eq. DIC in DMF, 40° C. for 12 h.

Cleavage of the peptide from the resin and simultaneous side chain deprotection was achieved with TFA/TA/EDT (90:7:3, v/v/v) for 3 h. The peptide was precipitated and washed with ice-cold diethyl ether.

Purification of the peptides was performed using preparative RP-HPLC on a Kinetex® 5 m Biphenyl 100A LC column (Phenomenex, 250 mm×4.6 mm, 5 μm, 100 Å). A linear gradient of 20% to 50% eluent B in 30 min was applied (Eluent A=0.1% TFA in water; Eluent B=0.08% TFA in ACN). The flow rate was 20 mL/min, UV detection was measured at λ=220 nm.

Analytics:

The identity of the peptide was confirmed via analytical RP-HPLC and ESI-Orbitrap (Orbitrap Elite™, Thermo Fischer). The purities were analyzed using analytical RP-HPLC.

Compound 15: [K¹⁴(ODD), (Dpr¹⁶, E²¹)_{lac}]ADM₁₄₋₅₂

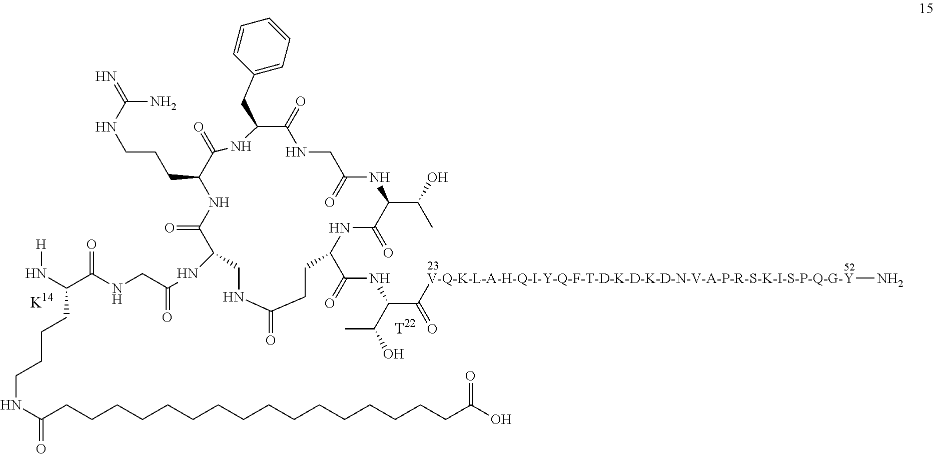

### ((3S,9S,12S,15S,21S)-15-(2-((S)-2-amino-6-(17-carboxyoctadecanamido)hexanamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-ADM(23-52)

Chemical Formula: C₂₁₄H₃₄₁N₅₉O₆₁

Exact Mass: 4713.54 Da

Molecular Weight: 4716.43 g/mol

Compound 15 was synthesized in a 15 μmol scale. The yield was 1.4 mg (1.6% f theory).

Compound 15 was analyzes via analytical RP-HPLC using a Kinetex® 5 μm Biphenyl 100 ALC column (Phenomenex, 250 mm×4.6 mm, 5 μm, 100 Å) applying a linear gradient of 20% to 70% eluent B in A over 40 min (Eluent A=0.1% TFA in water; Eluent B=0.08% TFA in ACN; flow rate=1.55 mL/min; λ=220 nm). Rt=16.4 min, purity≥95%.

In addition, an Aeris Peptide 3.6 μm XB-C18 100A (Phenomenex, 250 mm×4.6 mm, 3.6 μm, 100A) was used, applying a linear gradient of 20% to 70% eluent B in A over 40 min (Eluent A=0.1% TFA in water; Eluent B=0.08% TFA in ACN; flow rate=1.55 mL/min; λ=220 nm). Rt=17.6 min, purity≥95%.

The observed mass was in accordance with the calculated mass.

ESI Orbitrap: m/z=1179.9 [M+4H]⁴⁺; m/z=944.1 [M+5H]⁵⁺; m/z=786.9 [M+6H]⁶⁺; m/z=674.7 [M+7H]⁷⁺; m/z=590.5 [M+8H]⁸⁺.

Compound 16: [K¹⁴(ODD), (Dpr¹⁶, E²¹)_{lac}, (K⁴⁴, D⁴⁸)_{lac}]ADM₁₄₋₅₂

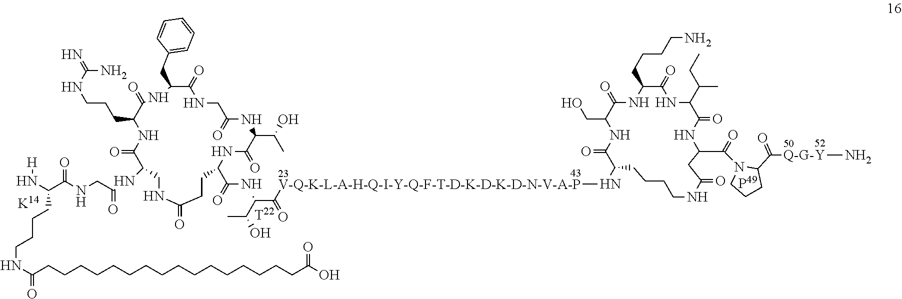

### ((3S,9S,12S,15S,21S)-15-(2-((S)-2-amino-6-(17-carboxyoctadecanamido)hexanamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-ADM(23-43)-((2S,5S,8S,11S,18S)-5-(4-aminobutyl)-18-(λ²-azaneyl)-8-((S)-sec-butyl)-2-(hydroxymethyl)-3,6,9,13,19-pentaoxo-1,4,7,10,14-pentaazacyclononadecane-11-carbonyl)-L-prolyl-ADM(50-52)

Chemical Formula: C₂₁₅H₃₃₉N₅₇O₆₁

Exact Mass: 4695.52 Da

Molecular Weight: 4698.42 g/mol

Compound 16 was synthesized in a 2×15 μmol scale. The yield was 2.1 mg (1.2% f theory).

Compound 16 was analyzes via analytical RP-HPLC using a Kinetex® 5 μm Biphenyl 100 ALC column (Phenomenex, 250 mm×4.6 mm, 5 μm, 100 Å) applying a linear gradient of 20% to 70% eluent B in A over 40 min (Eluent A=0.1% TFA in water; Eluent B=0.08% TFA in ACN; flow rate=1.55 mL/min; λ=220 nm). Rt=16.7 min, purity≥95%.

In addition, an Aeris Peptide 3.6 μm XB-C18 100A (Phenomenex, 250 mm×4.6 mm, 3.6 μm, 100A) was used, applying a linear gradient of 20% to 70% eluent B in A over 40 min (Eluent A=0.1% TFA in water; Eluent B=0.08% TFA in ACN; flow rate=1.55 mL/min; λ=220 nm). Rt=18.5 min, purity≥95%.

The observed mass was in accordance with the calculated mass.

ESI Orbitrap: m/z=1175.4 [M+4H]⁴⁺; m/z=940.5 [M+5H]⁵⁺; m/z=783.9 [M+6H]⁶⁺; m/z=672.1 [M+7H]⁷⁺.

Compound 17: [K¹⁴(ODD), (Dpr¹⁶, E²¹)_{lac}, OEG(13)³⁵⁻³⁸, (K⁴⁴, D⁴⁸)_{lac}]ADM₁₄₋₅₂

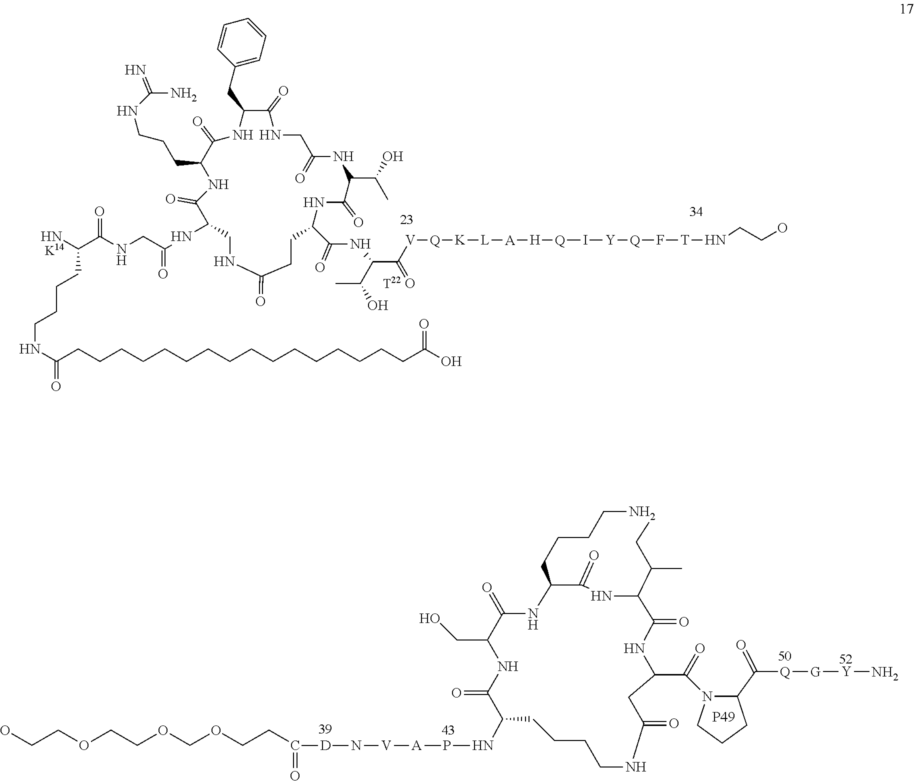

### ((3S,9S,12S,15S,21S)-15-(2-((S)-2-amino-6-(17-carboxyoctadecanamido)hexanamido)acetamido)-9-benzyl-12-(3-guanidinopropyl)-3-((R)-1-hydroxyethyl)-2,5,8,11,14,18-hexaoxo-1,4,7,10,13,17-hexaazacyclohenicosane-21-carbonyl)-L-threonyl-ADM(23-34)-(3-(2-(2-(2-(λ²-azaneyl)ethoxy)ethoxy)ethoxy)propanoyl)-L-aspartyl-ADM(40-43)-((2S,5S,8S,11S,18S)-5-(4-aminobutyl)-18-(λ²-azaneyl)-8-((S)-sec-butyl)-2-(hydroxymethyl)-3,6,9,13,19-pentaoxo-1,4,7,10,14-pentaazacyclononadecane-11-carbonyl)-L-prolyl-ADM(50-52)

Chemical Formula: C₂₄₀H₃₂₂N₅₂O₅₇

Exact Mass: 4412.39 Da

Molecular Weight: 4415.13 g/mol

Compound 17 was synthesized in a 2×15 μmol scale. The yield was 2.1 mg (1.2% f theory).

Compound 17 was analyzes via analytical RP-HPLC using a Kinetex® 5 μm Biphenyl 100 ALC column (Phenomenex, 250 mm×4.6 mm, 5 μm, 100 Å) applying a linear gradient of 20% to 70% eluent B in A over 40 min (Eluent A=0.1% TFA in water; Eluent B=0.08% TFA in ACN; flow rate=1.55 mL/min; λ=220 nm). Rt=13.9 min, purity≥94%.

In addition, a Jupiter® 4 μm Proteo 90 Å LC column (Phenomenex, 250 mm×4.6 mm, 4 μm, 90 Å) was used, applying a linear gradient of 20% to 70% eluent B in A over 40 min (Eluent A=0.1% TFA in water; Eluent B=0.08% TFA in ACN; flow rate=0.6 mL/min; λ=220 nm). Rt=24.1 min, purity≥94%.

The observed mass was in accordance with the calculated mass.

ESI Orbitrap: m/z=1472.5 [M+3H]³⁺; m/z=1104.6 [M+4H]⁴⁺; m/z=883.9 [M+5H]⁵⁺; m/z=736.7 [M+6H]⁶⁺.

Synthesis of fluorescently labelled analogues for in vitro stability determination Abbreviations

| | |
|---|---|
| Dde | N-γ-(4,4-dimethyl-2,6-dioxocyclohex-1-ylidene)ethyl |
| DIPEA | N,N-diisopropylethylamine |
| HATU | O-(7-Azabenzotriazol-1-yl)-N,N,N′,N′-tetramethyluronium- |
| | hexafluorphosphat |
| TAM | 6-Tamra |

Suppliers

| | |
|---|---|
| 6-TAMRA | EMP Biotech |
| Dde-Lys(Fmoc)-OH | Iris Bitotech |
| DIPEA | Roth |
| HATU | Merck |

Fluorescently labelled versions of analogues 5 and 13-15 were synthesized by incorporation of 6-TAMRA at the N-terminus of the peptides.

| compound | analogue |
|---|---|
| TAM-5 | TAM-[K¹⁴(ODD)]ADM(14-52) |
| TAM-13 | TAM-[K¹⁴(ODD), (K⁴⁴, D⁴⁸)_{lac}]ADM(14-52) |
| TAM-14 | TAM-[K¹⁴(ODD), OEG(13)^{[35-38]}, (K⁴⁴, D⁴⁸)_{lac}]ADM(14-52) |
| TAM-15 | TAM-[K¹⁴(ODD), (Dpr¹⁶, E²¹)_{lac}]ADM(14-52) |
| TAM-16 | TAM-[K¹⁴(ODD), (Dpr¹⁶, E²¹)_{lac}, (K⁴⁴, D⁴⁸)_{lac}]ADM(14-52) |
| TAM-17 | TAM-[K¹⁴(ODD), (Dpr¹⁶, E²¹)_{lac}, OEG(13)^{[35-38]}, (K⁴⁴, D⁴⁸)_{lac}]ADM(14-52) |

The synthesis was carried out as described above. Notably, Dde-Lys(Fmoc)-OH was coupled as N-terminal amino acid (ADM pos. 14) instead of Boc-Lys(Fmoc).

Before cleavage from the resin, the Dde protecting group was removed from the N-terminal amino acid using 1 ml 3% hydrazine in DMF (v/v) (10 times, 10 min each) and coupling of 6-TAMRA (3 equiv.) was carried out with 2.5 equiv. HATU and 3 equiv. DIPEA in DMF for approximately 24h.

The identity of the peptides was confirmed by mass spectrometry with a MALDI-ToF-MS (UltraflexIII, Bruker) and an ESI-MS (HCT, Bruker). The observed masses were in accordance with the calculated masses. Purity>95% f all analogues was demonstrated by analytical RP-HPLC.

B. Assessment of Pharmacological Activity

The suitability of the compounds according to the invention for treatment of diseases can be demonstrated using the following assay systems:

(1) Test Descriptions (In Vitro)

(1a) Activity Determination of Adrenomedullin Analogues in vitro

Abbreviations

| | |
|---|---|
| CLR | calcitonin receptor-like receptor |
| CRE | cAMP response element |
| DMEM | Dulbecco's Modified Eagle Medium |
| DPBS | Dulbecco's Phosphate-Buffered Saline |
| ECFP | enhanced cyan fluorescent protein |
| EYFP | enhanced yellow fluorescent protein |
| FCS | fetal calf serum |
| RAMP2 | receptor activity-modifying protein 2 |

Suppliers

| | |
|---|---|
| DMEM | Lonza |
| DPBS | Lonza |
| FCS | Biochrom |
| Ham's F-12 | Fluka |
| Metafectene ® Pro | Biontex |
| ONE Glo ™ Luciferase Assay System | Promega |
| poly-D-lysine hydrobromide | Sigma-Alrich |

Cell Culture HEK-293 cells (human embryonic kidney cells) were cultured in Ham's F-12/DMEM (1/1; v/v) containing 15% FCS under humidified atmosphere at 37° C. and 5% CO₂ in 75 cm² cell culture flasks.

Transient Co-Transfection of HEK293 Cells

Cells were cultured in 75 cm² flasks to 70-80% confluency. 45 μl Metafectene® Pro was diluted in 900 ρl Ham's F-12/DMEM (1/1; v/v) and incubated for 20 min at room temperature. 9 μg plasmid containing DNA of CLR fused to EYFP and 3 μg plasmid containing DNA of RAMP2 fused to ECFP were dissolved in 900 μl Ham's F-12/DMEM (1/1; v/v). The plasmid solution was mixed with the Metafectene® Pro solution and incubated for 25 min at room temperature. Medium was removed from the cells and replaced by 6 m1 Ham's F-12/DMEM (1/1; v/v) containing 15% FCS. After addition of transfection solution the cells were incubated for 3 h under humidified atmosphere at 37° C. and 5% CO₂. For the second transfection 45 μl Metafectene® Pro was diluted in 900 μl Ham's F-12/DMEM (1/1; v/v) and incubated for 20 min at room temperature. 12 μg of pGL4.29[Luc2P/CRE/Hygro] plasmid containing DNA for the luciferase reporter gene luc2P (with CRE promotor region) were dissolved in 900 μl Ham's F-12/DMEM (1/1; v/v). The plasmid solution was mixed with the Metafectene® Pro solution and incubated for 25 min at room temperature. Medium was removed from the cells and replaced by 6 m1 Ham's F-12/DMEM (1/1; v/v) containing 15% FCS. After addition of transfection solution, the cells were incubated under humidified atmosphere at 37° C. and 5% CO₂ over night.

cAMP-Assay

Seeding of Transiently Transfected Cells in 96-Well-Plates.

For coating of 96-well-plates, 50 μl of a poly-D-lysine solution in DPBS (0.1 mg/ml) were pipetted in each well and incubated for 40 min. After removal of poly-D-lysine, each well was washed with 50 μl DPBS. Transiently transfected cells were detached from the cell culture flask by removal of the medium, 2-fold washing with 5 m1 DPBS and resuspending in 40 m1 Ham's F-12/DMEM (1/1; v/v) containing 15% FCS. 90,000 to 120,000 cells in 150 μl Ham's F-12/DMEM (1/1; v/v) containing 15% FCS were seeded per well and the plates were incubated under humidified atmosphere at 37° C. and 5% CO₂ over night.

Cell Stimulation

For each ligand, a serial dilution with eight different concentrations was prepared using Ham's F¹²/DMEM (1/1; v/v). Before stimulation, the medium on the cells was replaced by 100 μl Ham's F¹²/DMEM (1/1; v/v) and the plates were incubated for 1 h under humidified atmosphere at 37° C. and 5% CO₂. For stimulation, the medium was removed and the cells were incubated for 3 h in 80 μl of ligand-solution under humidified atmosphere at 37° C. and 5% CO₂. In addition 80 μl of a 5 μM forskolin solution in Ham's F-12/DMEM (1/1; v/v) was used as a positive control and 80 μl of Ham's F-12/DMEM (1/1; v/v) as a negative control. Each concentration and the controls were tested as triplicates.

Luminescence Measurement

After 3 h of stimulation, the solutions were removed and the cells were washed with 50 μl of Ham's F-12/DMEM (1/1; v/v) perwell. After 10 m incubation in 30 μl of Ham's F-12/DMEM (1/1; v/v) at room temperature, 30 μl of luciferase-solution (ONE-Glo™ Luciferase Assay System) was added and the luminescence was directly measured using an Infinite M200 (Tecan).

Data Analysis

Data analysis of the luminescence measurement was carried out with GraphPad Prism 5. Therefore, the measured luminescence values of each plate were first corrected on the base of the respective average of forskolin stimulation. Afterwards they were normalized to [G14]ADM(14-52), which was used as standard peptide in every assay. After correction and normalization, data was analyzed using non-linear regression giving dose-response curves for each tested ligand.

Representative EC₅₀ values for the embodiment examples are given in the following Table 1 below.

| | | AM₁R activity data | | |
|---|---|---|---|---|
| | | EC₅₀ | Eₘₐₓ ± SEM | |
| Example | Example | [nM] | [%] | n |
| Reference | [G¹⁴]ADM(14-52) | 3.5 | 99 ± 1 | 38 |
| 1 | [G¹⁴, (K⁴⁴, D⁴⁸)_{lac}]ADM(14-52) | 3.9 | 102 ± 3 | 2 |
| 2 | [G¹⁴, (D⁴⁴, K⁴⁸)_{lac}]ADM(14-52) | 4.8 | 91 ± 1 | 2 |
| 3 | [G¹⁴, (K⁴⁴, E⁴⁸)_{lac}]ADM(14-52) | 16 | 93 ± 2 | 2 |
| 4 | [G¹⁴, (E⁴⁴, K⁴⁸)_{lac}]ADM(14-52) | | | |
| 5 | [K¹⁴(ODD)]ADM(14-52) | 17 | 105 ± 5 | 2 |
| 6 | [G¹⁴, OEG(12)^{[35-38]}]ADM(14-52) | 21 | 95 ± 7 | 2 |
| 7 | [G¹⁴, OEG(13)^{[35-38]}]ADM(14-52) | 13 | 103 ± 4 | 6 |
| 8 | [G¹⁴, OEG(16)^{[35-39]}]ADM(14-52) | 16 | 95 ± 3 | 3 |
| 9 | [G¹⁴, OEG(13)^{[36-39]}]ADM(14-52) | 49 | 87 ± 4 | 3 |
| 10 | [G¹⁴, OEG(16)^{[36-40]}]ADM(14-52) | 81 | 77 ± 3 | 3 |
| 11 | [G¹⁴, OEG(19)^{[35-40]}]ADM(14-52) | 39 | 94 ± 2 | 2 |
| 12 | [G¹⁴, OEG(19)^{[36-41]}]ADM(14-52) | 431 | 79 ± 5 | 3 |
| 13 | [K¹⁴(ODD), (K⁴⁴, D⁴⁸)_{lac}]ADM(14-52) | 2.6 | 94 ± 7 | 2 |
| 14 | [K¹⁴(ODD), OEG(13)^{[35-38]},(K⁴⁴, D⁴⁸)_{lac}] ADM(14-52) | 6.6 | 92 ± 5 | 2 |
| 15 | [K¹⁴(ODD), (Dpr¹⁶, E²¹)_{lac}]ADM(14-52) | 3.4 | 89 ± 2 | 2 |
| 16 | [K¹⁴(ODD), (Dpr¹⁶, E²¹)_{lac}, (K⁴⁴, D⁴⁸)_{lac}]ADM(14-52) | 1.4 | 100 ± 5 | 3 |
| 17 | [K¹⁴(ODD), (Dpr¹⁶, E²¹)_{lac}, OEG(13)^{[35-38]}, | 5.0 | 77 ± 3 | 3 |
| | (K⁴⁴, D⁴⁸)_{lac}]ADM(14-52) | | | |

(1b) Stability Determination of Adrenomedullin Analogues in Human Blood Plasma Suppliers

| | |
|---|---|
| ACN | Biosolve |
| Ethanol (absolute for molecular biology) | ITW Reagents |
| Human blood plasma | Haema |

The stability of the peptides was investigated using fluorescently labeled analogues, which were prepared as described above.

The TAMRA-labeled analogues were dissolved in 1.5 m1 of human blood plasma to a concentration of 10⁻⁵ M and incubated at 37° C. under constant shaking. Samples of 150 μl were taken at different time points and precipitated with 300 μl Ethanol/ACN (1:1) for at least 1 h at −20° C. After centrifugation for 30 s at 12000 rpm, the supernatant was collected and incubated at −20° C. for at least 3 h. Subsequently, it was transferred into Costar® Spin-X® Centrifuge Tube Filters (0.22 μm) and centrifuged for 1 h at 12000 rpm. The samples were analyzed by RP-HPLC using a VariTide RPC column (Agilent Technologies, 250 mm×4.6 mm, 6 m, 200 Å) with linear gradients of 0.1% TFA in water and 0.08% TFA in ACN; fluorescence measurement (λₑₓ=525 nm; λₑₘ=572 nm) was used for the detection of the analogues and their N-terminal fragments. The percentage of intact peptide was determined by peak integration. The values of peaks containing additional cleavage fragments were corrected by comparing intensities of cleavage fragments and intact peptide using MALDI-MS analysis (UltraflexIII, Bruker). The stability of the peptides was calculated with GraphPad Prism 5 (GraphPad Software) using a two phase exponential decay function for the determination of slow-decay phase half-lives (ln(2)/K_{slow}; K_{slow}: rate constant of slow part of exponential decay).

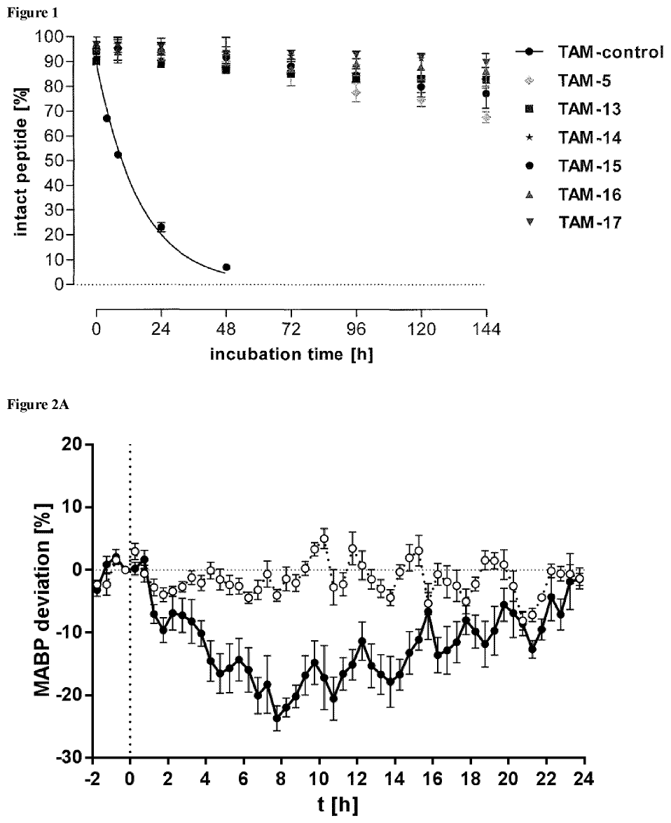

The stability of ADM analogues in human blood plasma are shown in Table 2 and FIG. 1.

TABLE 2
stability in human blood plasma

| compound | analogue | t_{1/2} (slow) |
|---|---|---|
| TAM-control | TAM-[G¹⁴]ADM(14-52) | 12 h |
| TAM-5 | TAM-[K¹⁴(ODD)]ADM(14-52) | 149 h |
| TAM-13 | TAM-[K¹⁴(ODD), (K⁴⁴, D⁴⁸)_{lac}]ADM(14-52) | >>144 h |
| TAM-14 | TAM-[K¹⁴(ODD), OEG(13)^{[35-38]}, (K⁴⁴, D⁴⁸)_{lac},]ADM(14-52) | >>144 h |
| TAM-15 | TAM-[K¹⁴(ODD), (Dpr¹⁶, E²¹)_{lac}]ADM(14-52) | >>144 h |
| TAM-16 | TAM-[K¹⁴(ODD), (Dpr¹⁶, E²¹)_{lac}, (K⁴⁴, D⁴⁸)_{lac}]ADM(14-52) | >>144 h |
| TAM-17 | TAM-[K¹⁴(ODD), (Dpr¹⁶, E²¹)_{lac}, OEG(13)^{[35-38]}(K⁴⁴, D⁴⁸)_{lac}]ADM(14-52) | >>144 h |

1c) Tests on a Recombinant Adrenomedullin-Receptor Reporter Cell

The activity of the compounds according to the invention is quantified with the aid of a recombinant Chinese hamster ovary (CHO) cell line that carries the human adrenomedullin-receptor. Activation of the receptor by ligands can be measured by aequorin luminescence. Construction of the cell line and measurement procedure has been described in detail [Wunder F., Rebmann A., Geerts A, and Kalthof B., Mol Pharmacol, 73, 1235-1243 (2008)]. In brief: Cells are seeded on opaque 384-well microtiter plates at a density of 4000 cells/well and are grown for 24 h. After removal of culture medium, cells are loaded for 3 h with 0.6 μg/ml coelenterazine in Ca²⁺-free Tyrode solution (130 mM sodium chloride, 5 mM potassium chloride, 20 mM HEPES (4-(2-hydroxyethyl)-1-piperazineethanesulfonic acid), 1 mM magnesium chloride, and 4.8 mM sodium hydrogen carbonate, pH 7.4) supplemented with 0.2 mM 3-Isobutyl-1-methylxanthine (IBMX) in a cell culture incubator. Compounds are added for 6 min in calcium²⁺-free Tyrode solution containing 0.1% bovine serum albumin. Immediately before adding calcium²⁺ to a final concentration of 3 mM measurement of the aequorin luminescence is started by use of a suitable luminometer. Luminescence is measured for 60 s. In a typical experiment compounds are tested in a concentration range of 1×10⁻¹³ to 3×10⁻⁶ M.

1d) Transcellular Electrical Resistance Assays in Endothelial Cells

The activity of the compounds according to the invention is characterized in in vitro-permeability assays in human umbilical venous cells (HUVEC, Lonza). By use of an xCELLigence® apparatus (ACEA Biosciences, Inc.; San Diego, Calif.) changes of transendothelial electrical resistance (TEER) over an endothelial monolayer are continuously measured by use of a small gold electrodes on which the cells have been seeded. HUVEC are grown on the 96-well sensor electrode plates (OMNI Life Science, 2801035) to confluent monolayers and hyperpermeability can be induced by inflammatory stimuli such as Thrombin, TNF-α, IL-1β, VEGF, Histamine and hydrogen peroxide which all have been demonstrated to cause break down of endothelial cell contacts and reduction of TEER. Test compounds are added before or after addition of thrombin. In a typical experiment compounds are tested in a concentration range of 1×10⁻¹⁰ to 1×10⁻⁶ M.

1c) In Vitro-Permeability Assays in Endothelial Cells

In another in vitro model of endothelial hyperpermeability the activity of compounds according to the invention is examined with respect to modulation of macromolecular permeability. Human umbilical vein endothelial cells (HUVECS) are grown to confluency on fibronectin-coated Transwell® filter membranes (24-well plates, 6.5 mm-inserts with 0.4 μM polycarbonate membrane; Costar #3413) which separate an upper from a lower tissue culture chamber with endothelial cells growing on the bottom of the upper chamber. The medium of the upper chamber is supplemented with 250 μg/ml of 40 kDa FITC-Dextran (Invitrogen, D1844). Hyperpermeability of the monolayer is induced by addition of thrombin. Medium samples are collected from the lower chamber every 30 min and relative fluorescence as a parameter for changes of macromolecular permeability over time is measured in a suitable fluorimeter. Thrombin challenge typically induces a significant increase of FITC-dextran transition across the endothelial monolayers. In a typical experiment compounds are tested in a concentration range of 1×10⁻¹⁰ to 1×10⁻⁶ M.

(2) Test Descriptions (In Vivo)

2a) Measurement of Blood Pressure and Heart Rate in Telemetered, Normotensive Wistar Rats

The cardiovascular effects induced by compounds according to the invention are investigated in freely moving conscious female Wistar rats (body weight>200 g) by radiotelemetric measurement of blood pressure and heart rate. Briefly, the telemetric system (DSI Data Science International, MN, USA) is composed on 3 basic elements: implantable transmitters (PhysioTel HD-S10), receivers (PhisioTel RPC-1 with PhisioTel MX2 Data Exchange Matrix) and a computer-based acquisition software (Dataquest™ A.R.T for Windows). Rats are instrumented with pressure implants for chronic use at least 14 days prior to the experiments. During catheter implantation rats are anesthetized with pentobabital (Nembutal, Sanofi: 50 mg/kg i.p.). After shaving the abdominal skin, a midline abdominal incision is made, and the fluid-filled sensor catheter is inserted upstream into the exposed descending aorta between the iliac bifurcation and the renal arteries. The catheter is tied several times at the stopper. The tip of the telemetric catheter is located just caudal to the renal arteries and secured by tissue adhesive. The transmitter body is affixed to the inner peritoneal wall before closure of abdomen. A two-layer closure of the abdominal incision is used, with individual suturing of the peritoneum and the muscle wall followed by closure of the outer skin. For postsurgical protection against infections and pain a single dosage of an antibiotic (Oxytetracyclin® 10%, 60 mg/kg s.c., 0.06 m1/100 g body weight, Beta-Pharma GmbH & Co, Germany) and analgesic were injected (Rimadyl®, 4 mg/kg s.c., Pfizer, Germany). The hardware configuration is equipped for 24 animals. Each rat cage is positioned on top of an individual receiver platform. After activation of the implanted transmitters, an on-line data acquisition system, samples data and converts telemetric pressure signals to mm Hg. A barometric pressure reference allows for relation of absolute pressure (relative to vacuum) to ambient atmospheric pressure. Data acquisition software is predefined to sample hemodynamic data for 10-s intervals every 5 minutes and are displayed as average values every 30 minutes. Data collection to file is started 2 hours before administration of test compounds and finished after completion of 24-h cycles. In a typical experiment test compounds are administered as bolus either subcutaneously or intravenously at doses of 1 to 1000 μg/kg body weight (as referred to the peptide component).

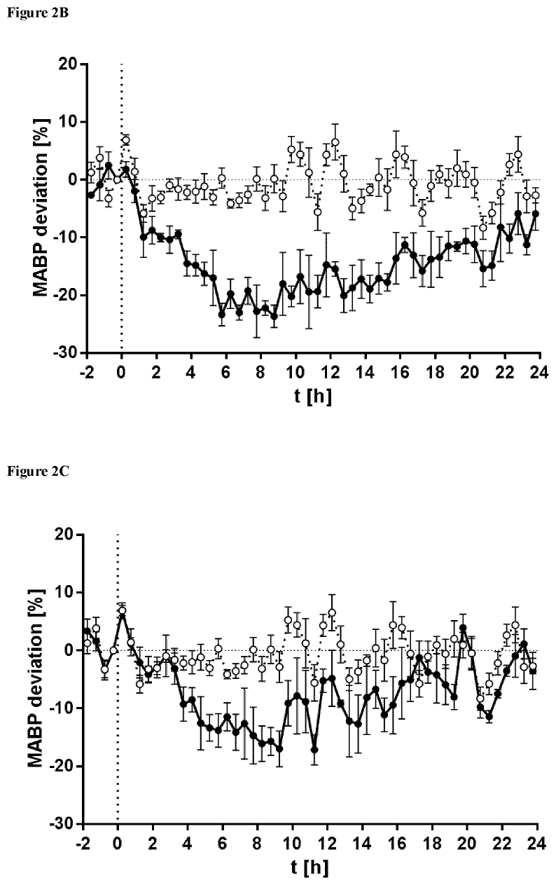

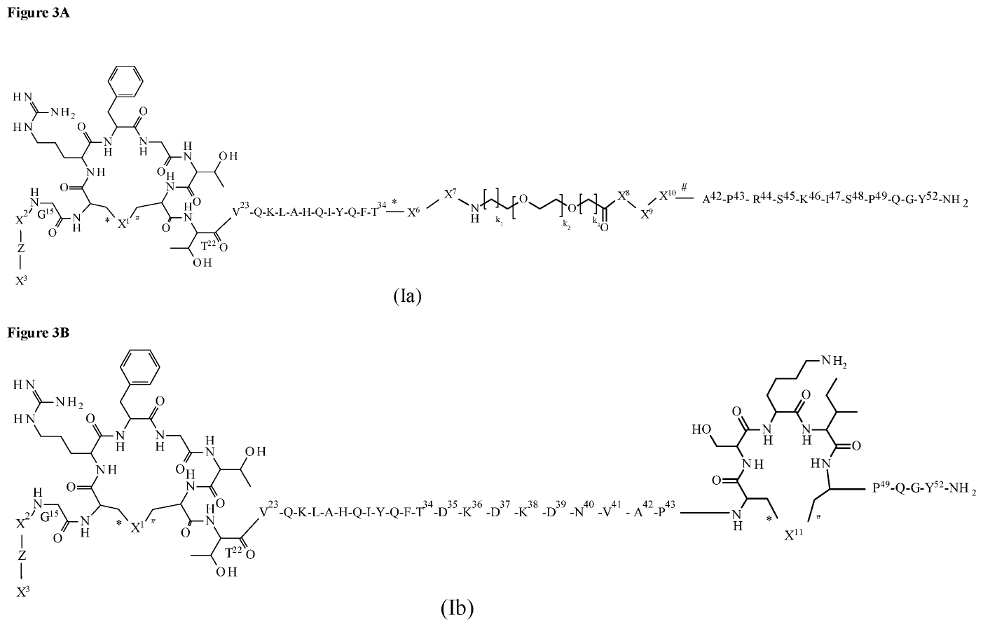

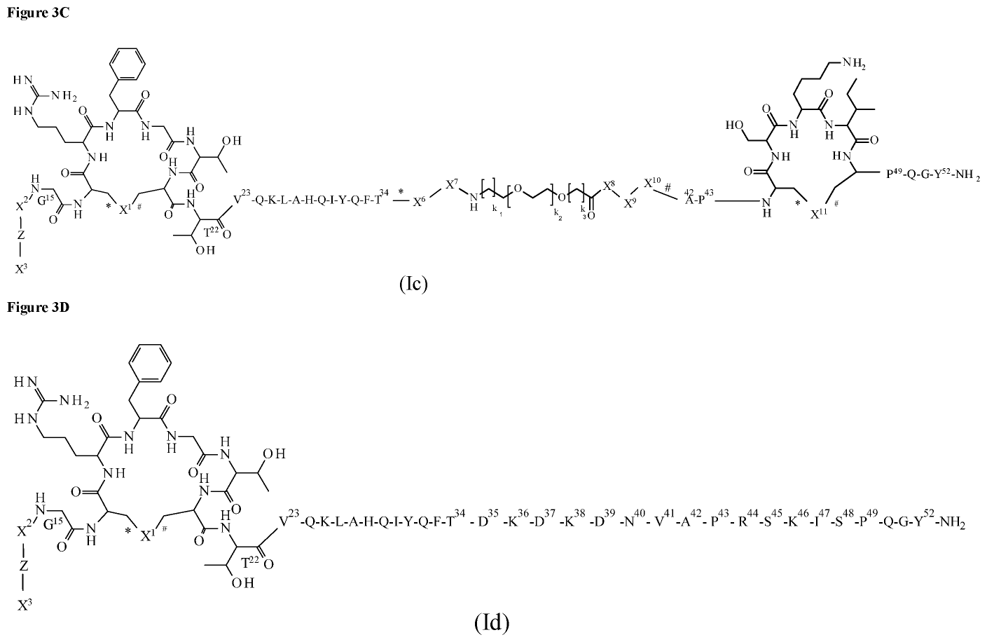

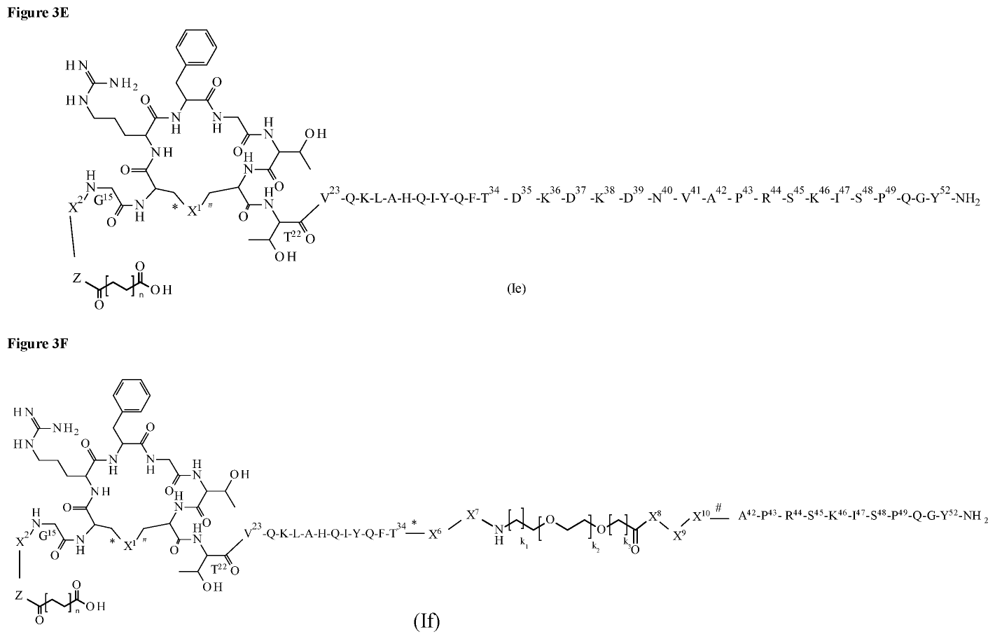

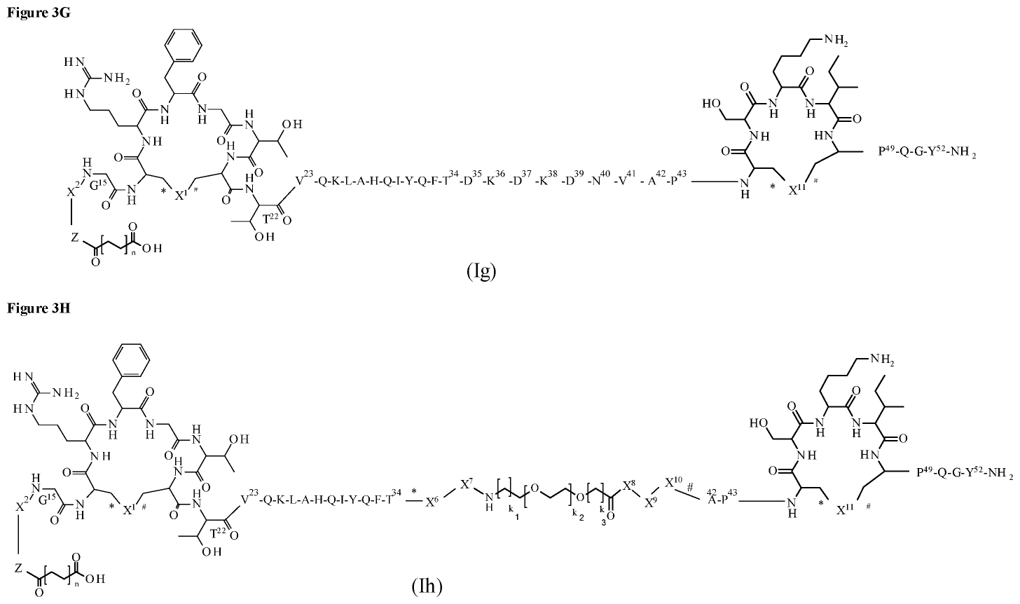

In this test single dose application of substances according to the present invention induce long lasting blood pressure reduction at doses of <500 μg/kg body weight [FIGS. 2A, 2B, 2C].

1: A compound according to formula (I) 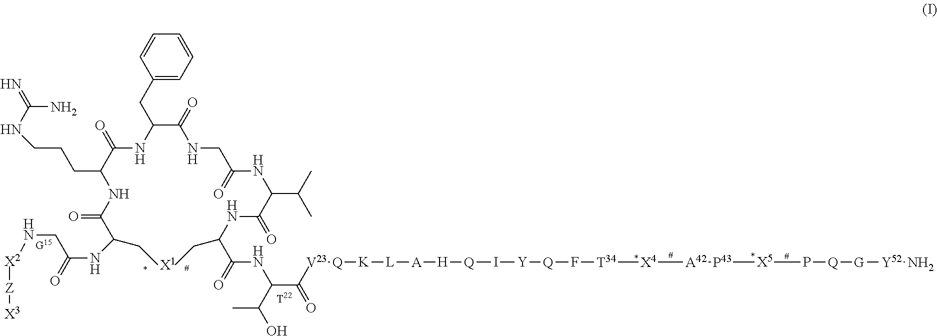
a physiologically acceptable salt, a solvate or a solvate of a salt thereof, wherein
X¹ is selected from the group consisting of
*—(CH₂)ₘ₁—S—S—(CH₂)ₙ₁—^{#}, wherein m1 is 0-6, n1 is 0-6, with the proviso that m1+n1=0-6;
*—(CH₂)ₘ₂—S—(CH₂)ₙ₂—^{#}, wherein m2 is 0-6, n2 is 0-6, with the proviso that m2+n2=0-6;
*—(CH₂)ₘ₃—^{#}, wherein m3 is 1-8;
*—(CH₂)ₘ₄—(CH₂═CH₂)—(CH₂)ₙ₃—^{#}, wherein m4 is 0-6, n3 is 0-6, with the proviso that m4+n3=0-6;
*—(CH₂)ₘ₅—(CH—CH)—(CH₂)ₙ₄—^{#}, wherein m5 is 0-6, and n4 is 0-6, with the proviso that m5+n4=0-6;
*—(CH₂)ₘ₆—CO—NH—(CH₂)ₙ₅—^{#}, wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
^{#}—(CH₂)ₘ₈—SO—(CH₂)ₙ₇—*, wherein m8 is 0-4, and n7 is 0-4, with the proviso that m8+n7=0-6;
^{#}—(CH₂)ₘ₉—SO₂—(CH₂)ₙ₈—*, wherein m9 is 0-4, and n8 is 0-4, with the proviso that m9+n8=0-6;
*-5-6 membered heteroaryl-^{#};
*—(CH₂)ₘ₁₀—O—(CH₂)ₙ₉—^{#}, wherein m10 is 0-6, n9 is 0-6, with the proviso that m10+n9=0-6;
*—(CH₂)ₘ₁₈—NH—CO—CH₂—NH—CO—(CH₂)ₙ—^{#}, wherein m18 is 0-3, and n5 is 0 or 1, with the proviso that m18+n5=0-3; #—(CH₂)ₘ₉—NH—CO—CH₂—NH—CO—(CH₂)ₙ₆—*, wherein m19 is 0-3, and n6 is 0 or 1, with the proviso that m19+n6=0-3;
*—(CH₂)ₘ₂₀—NH—CO—CH(CH₃)—NH—CO—(CH₂)ₙ₇—^{#}, wherein m20 is 0-3, and n7 is 0 or 1, with the proviso that m20+n7=0-3; #—(CH₂)ₘ₂₁—NH—CO—CH(CH₃)—NH—CO—(CH₂)ₙ₈—*, wherein m21 is 0-3, and n8 is 0 or 1, with the proviso that m21+n8=0-3;
*—(CH₂)ₘ₂₂—NH—CO—CH(CH₂—C(CH₃)₂)—NH—CO—(CH₂)ₙ₉—^{#}, wherein m22 is 0-3, and n9 is 0 or 1, with the proviso that m22+n9=0-3; #—(CH₂)ₘ₂₃—NH—CO—CH(CH₂—C(CH₃)₂)—NH—CO—(CH₂)ₙ₁₀—*, wherein m23 is 0-3, and n10 is 0 or 1, with the proviso that m23+n10=0-3;
*—(CH₂)ₘ₂₄—NH—CO—CH(CH(CH₃)C₂H₅)—NH—CO—(CH₂)ₙ₁₁—^{#}, wherein m24 is 0-3, and n11 is 0 or 1, with the proviso that m24+n11=0-3; #—(CH₂)ₘ₂₅—NH—CO—CH(CH(CH₃)C₂H)—NH—CO—(CH₂)ₙ₁₂—*, wherein m25 is 0-3, and n12 is 0 or 1, with the proviso that m25+n12=0-3;
*—(CH₂)ₘ₂₆—NH—CO—CH(CH₂(C₆H₅))—NH—CO—(CH₂)ₙ₁₃—^{#}, wherein m26 is 0-3, and n13 is 0 or 1, with the proviso that m26+n13=0-3; #—(CH₂)ₘ₂₇—NH—CO—CH(CH₂(C₆H₅))—NH—CO—(CH₂)ₙ₁₄—*, wherein m27 is 0-3, and n14 is 0 or 1, with the proviso that m27+n14=0-3;
—(CH₂)ₘ₂₈—NH—CO—(CH₂)₃—NH—CO—(CH₂)ₙ₁₅—^{#}, wherein m28 is 0 or 1, and n15 is 0 or 1, with the proviso that m28+n15=0-1; #—(CH₂)ₘ₂₉—NH—CO—(CH₂)₃—NH—CO—(CH₂)ₙ₁₆—*, wherein m29 is 0 or 1, and n16 is 0 or 1, with the proviso that m29+n16=0-1;
*—(CH₂)ₘ₃₀—NH—CO—NH—(CH₂)ₙ₁₇—^{#}, wherein m30 is 0-5, and n17 is 0-5, with the proviso that m30+n17=0-5; ^{#}—(CH₂)ₘ₃₁—NH—CO—NH—(CH₂)ₙ₁₅—*, wherein m31 is 0-5, and n18 is 0-5, with the proviso that m31+n18=0-5;
*—(CH₂)ₘ₃₂—O—CO—NH—(CH₂)ₙ₁₉—^{#}, wherein m32 is 0-5, and n19 is 0-5, with the proviso that m32+n19=0-5; ^{#}—(CH₂)ₘ₃₃—O—CO—NH—(CH₂)ₙ₂₀—*, wherein m33 is 0-5, and n20 is 0-5, with the proviso that m33+n20=0-5;
*—(CH₂)ₘ₃₄—O—CO—O—(CH₂)ₙ₂₁—^{#}, wherein m34 is 0-5, and n21 is 0-5, with the proviso that m34+n21=0-5;
*—(CH₂)ₘ₃₅—NH—CO—(CH₂)ₙ₂₂—NH—(CH₂)ₚ₁—, wherein m35 is 0-4, n22 is 0-4, and p1 is 0-4, with the proviso that m35+n22+p1=0-4; and
*—(CH₂)ₘ₃₆—NH—CO—(CH═CH)—CO—NH—(CH₂)ₙ₂₃—^{#}, wherein m36 is 0-2, and n23 is 0-2, with the proviso that m36+n23=0-2;
wherein * and ^{#} reflect where X¹ is bound within the ring structure; and
X² is absent, is hydrogen, or is an amino acid or an amino acid sequence selected from the group consisting of
G¹⁴, K¹⁴, F¹⁴, SEQ ID NO:1 [Y¹RQSMNNFQGLRSF¹⁴], SEQ ID NO:2 [R²QSMNNFQGLRSF¹⁴], SEQ ID NO:3 [Q³SMNNFQGLRSF¹⁴], SEQ ID NO:4 [S⁴MNNFQGLRSF¹⁴], SEQ ID NO:5 [M⁵NNFQGLRSF¹⁴], SEQ ID NO:6 [N⁶NFQGLRSF¹⁴], SEQ ID NO:7 [N⁷FQGLRSF¹⁴], SEQ ID NO:8 [F⁸QGLRSF¹⁴], SEQ ID NO:9 [Q⁹GLRSF¹⁴], SEQ ID NO:10 [G¹⁰LRSF¹⁴], SEQ ID NO:11 [L¹¹RSF¹⁴], SEQ ID NO:12 [R¹²SF¹⁴], and SEQ ID NO:13 [S¹³F¹⁴], wherein any one of the SEQ ID NO:1 to SEQ ID NO:13 is covalently linked between F¹⁴ of said sequences by an amide bond to the N-terminal G¹⁵ of the amino acid sequence of formula (I), wherein any amino acid of X² can optionally be replaced by a natural or unnatural amino acid;
wherein A is L-Alanine; R is L-Arginine; N is L-Asparagine; D is L-Aspartic acid; Q is L-Glutamine; G is L-Glycine; H is L-Histidine; I is L-Isoleucine; L is L-Leucine; K is L-Lysine; M is L-Methionine; F is L-Phenylalanine; P is L-Proline; S is L-Serine; T is L-Threonine; Y is L-Tyrosine; V is L-Valine;
X³ is absent or is a heterologous moiety which is covalently linked to the N-terminus or to a functional group of the side chain of any amino acid of X², to the N-terminus of G¹⁵ or to Z;
Z is absent or is a cleavable linker covalently bound between the N terminus of any amino acid of X² or of G¹⁵ and X³ or between a functional group of the side chain of any amino acid of X² and X³
wherein if X³ is absent, then Z is also absent and X² is hydrogen or is an amino acid or amino acid sequence as defined above for X²;
wherein if X³ is a heterologous moiety, then X² is absent or is an amino acid or amino acid sequence as defined above for X²;
X⁴ is the amino sequence *[D³⁵ K³⁶ D³⁷ K³⁸ D³⁹ N⁴⁰ V⁴¹]#, wherein at least one amino acid of said sequence can optionally be replaced by a natural or unnatural amino acid and wherein * indicates the binding site to T³⁴ and # indicates the binding site to A⁴², or X⁴ is a moiety according to formula (A), wherein * indicates the binding site to T³⁴ and # indicates the binding site to A⁴² 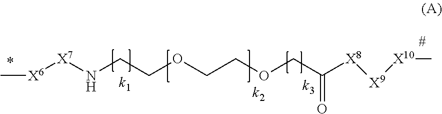
wherein X⁶, X⁷, X⁸, X⁹ and X¹⁰ are independently from another absent or an amino acid selected from L-Alanine; L-Arginine; L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or L-Valine,
wherein k1 is 1, 2, 3 or 4,
wherein k2 is 0, 1, 2, 3, 4, 5, 6, 7 or 8,
wherein k3 is 1, 2, 3 or 4,
X⁵ is the amino sequence *[R⁴⁴ S⁴⁵ K⁴⁶ I⁴⁷ S⁴⁸]#, wherein the sequence can optionally comprise at least one amino acid replaced by a natural or unnatural amino acid and wherein * indicates the binding site to P⁴³ and # indicates the binding site to P⁴⁹, or X⁵ is a moiety according to formula (B), wherein * and ^{#} reflect where X⁵ is bound within the amino acid chain and wherein * indicates the binding site of X⁵ to P⁴³ and # indicates the binding site to P⁴⁹, 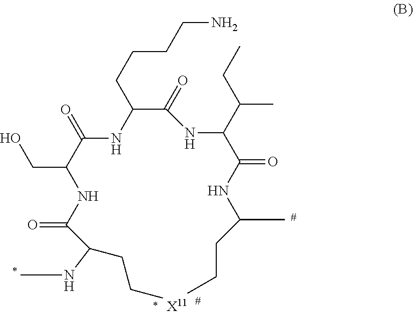
wherein X¹¹ is selected from the group consisting of
*—(CH₂)ₚ₁—S—(CH₂)ᵣ₁—^{#}, wherein p1 is 0-6; r1 is 0-6 with the proviso that p1+r1=0-6;
*—(CH₂)ₚ₂—O—(CH₂)ᵣ₂—^{#}, wherein p2 is 0-6; r2 is 0-6 with the proviso that p1+r2=0-6;
*—(CH₂)ₚ₃—^{#}, wherein p3 is 1-8;
*—(CH₂)ₚ₄—CO—NH—(CH₂)ᵣ₄—^{#}, wherein p4 is 0-4, and r4 is 0-4, with the proviso that p4+r4=0-6;
^{#}—(CH₂)ₚ₅—CO—NH—(CH₂)ᵣ₅—*, wherein p5 is 0-4, and r5 is 0-4, with the proviso that p5+r5=0-6;
wherein * and ^{#} reflect where X¹¹ is bound within the ring structure;
wherein the numbering of amino acids in formula (I) refers to the corresponding human adrenomedullin (ADM) sequence;
wherein if X³ is not a di-carboxylic acid, then at least X⁴ is a moiety according to formula (A) as defined above and/or X⁵ is a moiety according to formula (B) as defined above.

2: The compound according to formula (I) according to claim 1, wherein X¹ is selected from
*—(CH₂)ₘ₁—S—S—(CH₂)ₙ₁—^{#}, wherein m1 is 0-6, n1 is 0-6, with the proviso that m1+n1=0-6;
*—(CH₂)ₘ₂—S—(CH₂)ₙ₂—^{#}, wherein m2 is 0-6, n2 is 0-6, with the proviso that m2+n2=0-6;
*—(CH₂)ₘ₃—^{#}, wherein m3 is 1-8;
*—(CH₂)ₘ₆—CO—NH—(CH₂)ₙ₅—^{#}, wherein m6 is 0-4, and n5 is 0-4, with the proviso that m6+n5=0-6;
^{#}—(CH₂)ₘ₇—CO—NH—(CH₂)ₙ₆—*, wherein m7 is 0-4, and n6 is 0-4, with the proviso that m7+n6=0-6;
*—(CH₂)ₘ₁₀—O—(CH₂)ₙ₉—^{#}, wherein m10 is 0-6, n9 is 0-6, with the proviso that m10+n9=0-6,
wherein * and ^{#} reflect where X¹ is bound within the ring structure.

3: The compound according to formula (I) according to claim 1, wherein X¹ is *—(CH₂)ₘ₁—S—S—(CH₂)ₙ₁—^{#}, wherein m1 is 0-6, n1 is 0-6 with the proviso that m1+n1=0-6, and wherein * and ^{#} reflect where X¹ is bound within the ring structure, or wherein X¹ is
*—(CH₂)ₘ₆—CO—NH—(CH₂)ₙ₅—^{#}, wherein m6 is 0-6, n5 is 0-6 with the proviso that m6+n5=0-6, and wherein * and ^{#} reflect where X¹ is bound within the ring structure.

4: The compound according to formula (I) according to claim 1, wherein X² is absent, is hydrogen, or is an amino acid.

5: The compound according to formula (I) according claim 1, wherein X² is G¹⁴ or K¹⁴, which is covalently linked by an amide bond to the N-terminal G¹⁵ of the compound of formula (I).

6: The compound of formula (I) according to claim 1, wherein
X³ is a heterologous moiety selected from the group consisting of a polymer, a Fc, a FcRn binding ligand, albumin and an albumin-binding ligand; or a physiologically acceptable salt, a solvate or a solvate of a salt thereof, or wherein X³ is a polymer and the polymer is selected from the group consisting of linear or branched C1-C100 carboxylic acids and carboxylic di-acids, preferably C4-C30 carboxylic acids and carboxylic di-acids, optionally substituted with halo, hydroxy, alkoxy, amino, alkylamino, dialky lamino, sulfate, or phosphate, and which may be saturated, or mono- or di-unsaturated, a PEG moiety, a PPG moiety, a PAS moiety and a HES moiety; or a physiologically acceptable salt, a solvate or a solvate of a salt thereof; or
wherein X³ is a carboxylic di-acid, preferably a C14-C22 carboxylic di-acid, more preferably a C14-C18 carboxylic di-acid gr derivatives thereof, or wherein X³ is a moiety according to Formula (C) 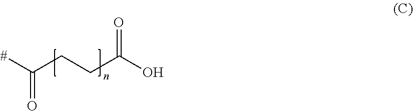
wherein n is 1 to 15, and wherein X¹, X², X⁴ and X⁵ and are as defined according to claim 1 and wherein # indicates the binding site to Z, wherein, if Z is absent, #indicates the binding site to X2.

7: The compound according to formula (I) according to claim 1, wherein X⁴ is a moiety according to formula (A) 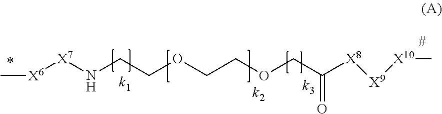
wherein X⁶, X⁷, X⁸, X⁹ and X¹⁰ are independently from another absent or an amino acid selected from L-Alanine; is L-Arginine; is L-Asparagine; L-Aspartic acid; L-Glutamine; L-Glycine; L-Histidine; L-Isoleucine; L-Leucine; L-Lysine; L-Methionine; L-Phenylalanine; L-Proline; L-Serine; L-Threonine; L-Tyrosine; or V is L-Valine,
wherein k1 is 1 or 2; wherein k2 is 0, 1, 2, 3, or 4; wherein k3 is 1 or 2,
and ^{#} reflect where X⁴ is bound within the amino acid chain, wherein * indicates the binding site to T³⁴ and # indicates the binding site to A⁴²; or
wherein X⁴ is a moiety according to formula (A), wherein X⁶ is absent or selected from the group consisting of D, N and V; wherein X⁷ is absent or is selected from the group consisting of D, N and V; wherein X⁸ is absent or is selected from the group consisting of D, N and V; wherein X⁹ is absent or is selected from the group consisting of D, N and; wherein X¹⁰ is absent or is selected from the group consisting of D, N and V; wherein k1 is 1 or 2; wherein k2 is 0, 1, 2, 3, or 4; wherein k3 is 1 or 2,
and ^{#} reflect where X⁴ is bound within the amino acid chain, wherein * indicates the binding site to T³⁴ and # indicates the binding site to A⁴².

8: The compound according to formula (I) according to claim 1, wherein X⁵ is the moiety according to formula (B), 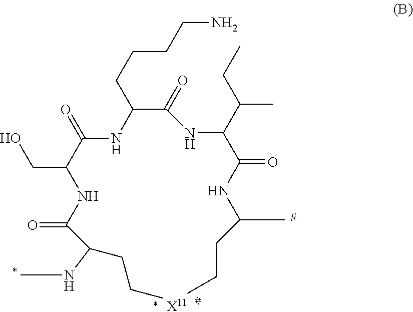
wherein * and ^{#} reflect where X⁵ is bound within the amino acid chain and wherein * indicates the binding site of X⁵ to P⁴³ and # indicates the binding site to P⁴⁹,
wherein X¹¹ is selected from the group consisting of
*—(CH₂)ₚ₁—S—(CH₂)ᵣ₁^{#}, wherein p1 is 0-4 and r1 is 0 or 1;
^{#}—(CH₂)ₚ₂—S—(CH₂)ᵣ₂*, wherein p2 is 0-4 and r2 is 0 or 1;
*—(CH₂)ₚ₃—^{#}, wherein p3 is 1-4;
*—(CH₂)ₚ₄—CO—NH—(CH₂)ᵣ₄—^{#}, wherein p4 is 0, 1, 2 or 3 or, and r4 is 0, 1, 2 or 3, with the proviso that p4+r4=0-4;
^{#}—(CH₂)ₚ₅—CO—NH—(CH₂)ᵣ₅—*, wherein p5 is 0, 1, 2 or 3, and r5 is 0, 1, 2 or 3, with the proviso that p5+r5=0-4;
wherein * and ^{#} reflect where X¹¹ is bound within the ring structure.

9: The compound according to formula (I) according claim 1, wherein the compound is a compound according to formula (Ia) 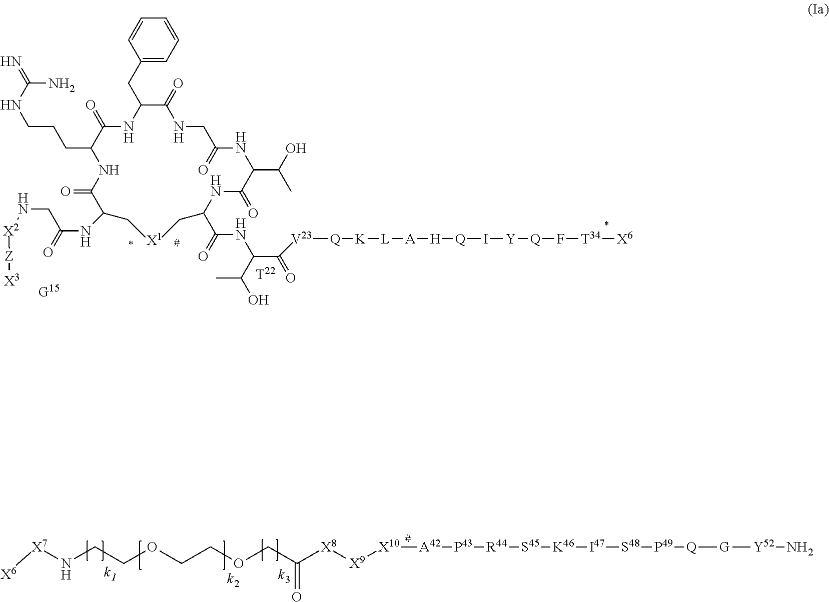
wherein X¹, X², X³, X⁶, X⁷, X⁸, X⁹, X¹⁰, k1, k2, and k3 are defined according to claim 1;
a compound according to formula (Ib) 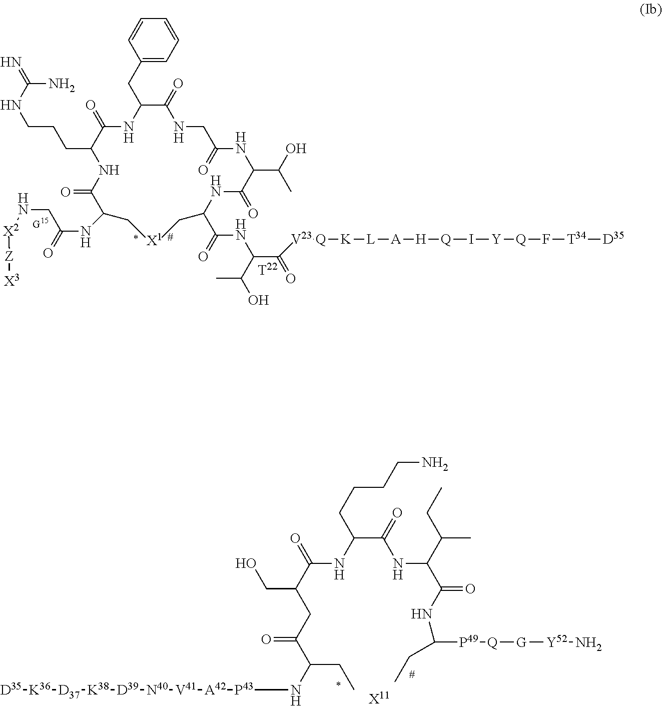
wherein X¹, X², X³ and X¹¹ are defined according to claim 1;
a compound according to formula (Ic) 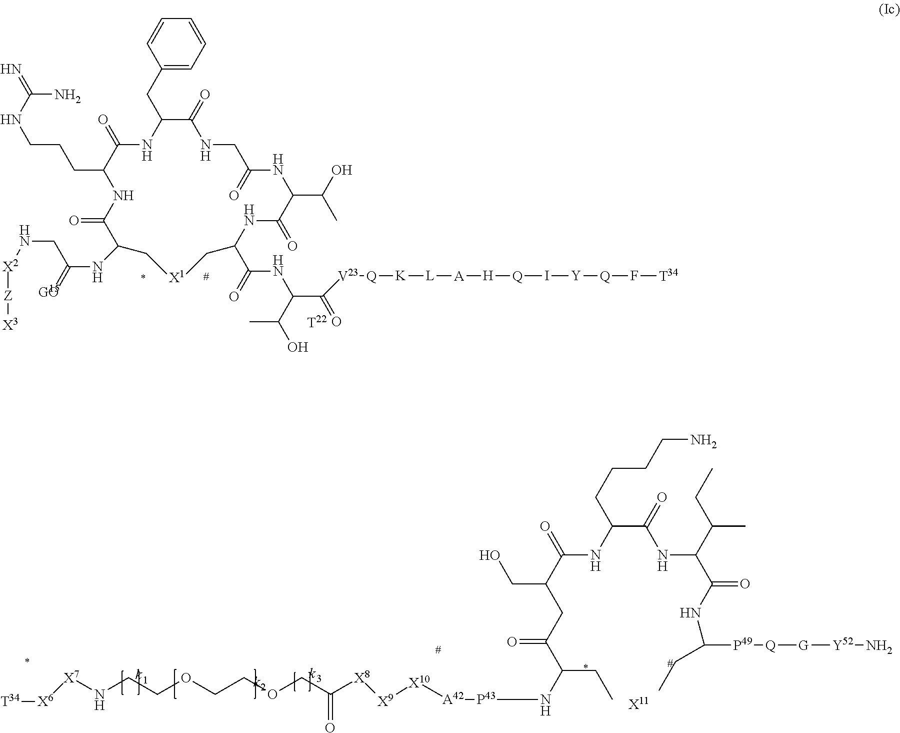
wherein X¹, X², X³, Z, X⁶, X⁷, X⁸, X⁹, X¹⁰, k1, k2, k3 and X¹¹ are defined according to claim 1;
a compound according to formula (Id) 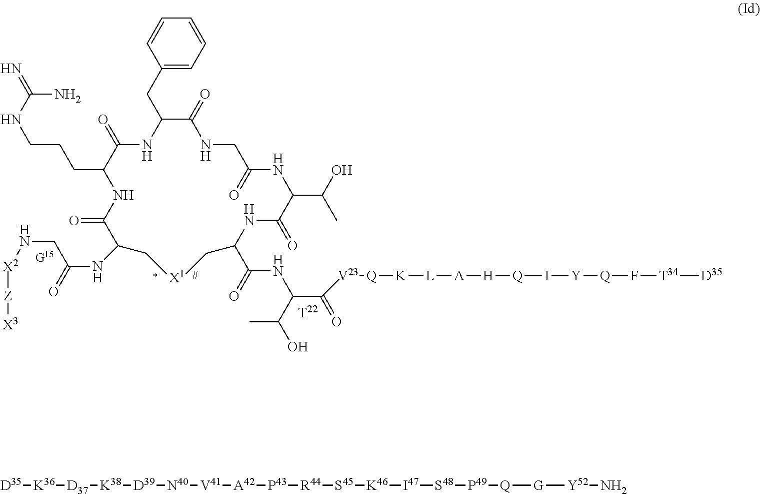
wherein X³ is a di-carboxylic acid, and X¹, X², Z, X⁶, X⁷, X⁸, X⁹, X¹⁰, k1, k2, k3 and X¹¹ are defined according claim 1;
a compound according to formula (Ie) according to claim 1, 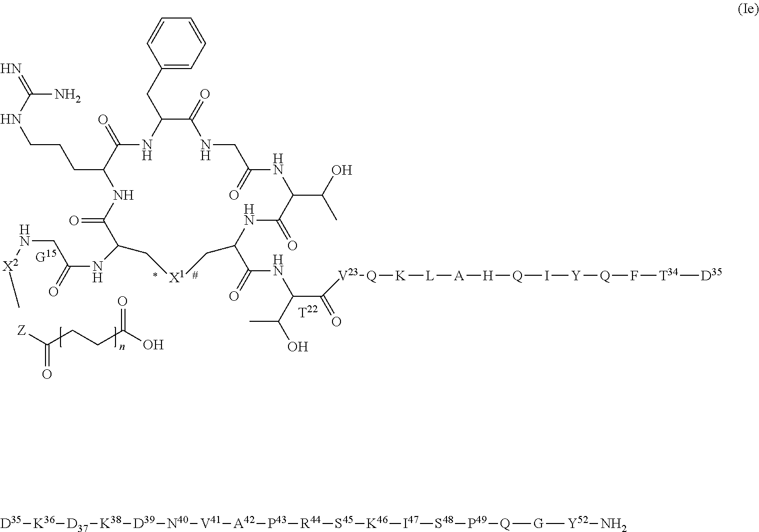
wherein n is 1 to 30 and X¹, X², Z are defined according to claim 1,
a compound according to formula (If) 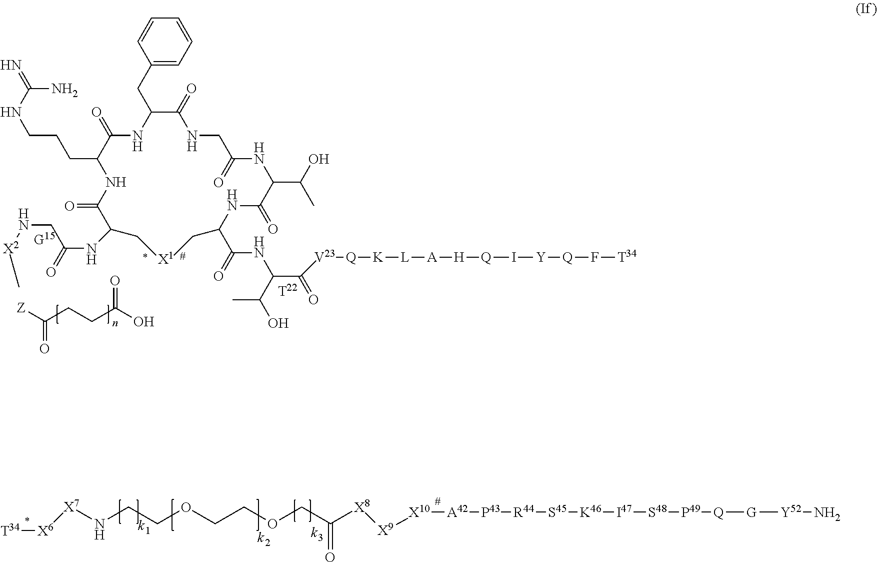
wherein n is 1 to 30 and X¹, X², Z, X⁶, X⁷, X⁸, X⁹, X¹⁰, k1, k2, k3 are defined according to claim 1;
a compound according to formula (Ig) 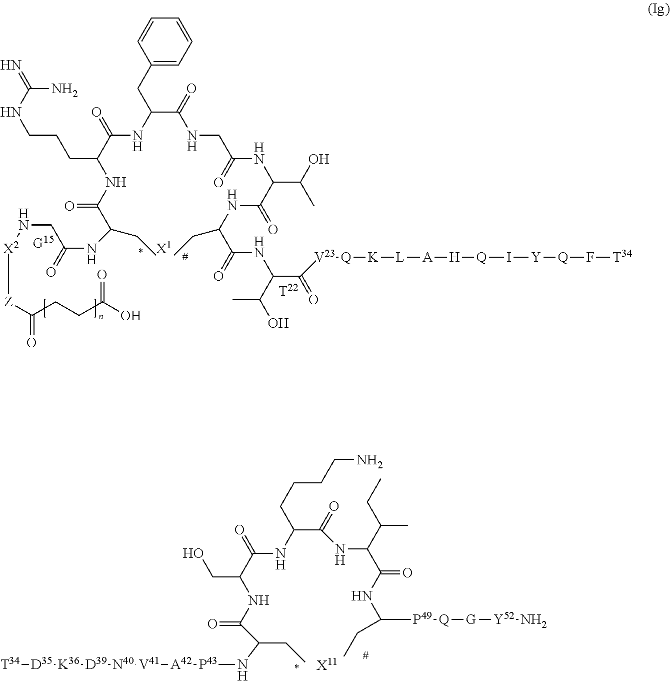
wherein n is 1 to 30 and X¹, X², Z and X¹¹ are defined according to claim 1;
a compound according to formula (Ih) 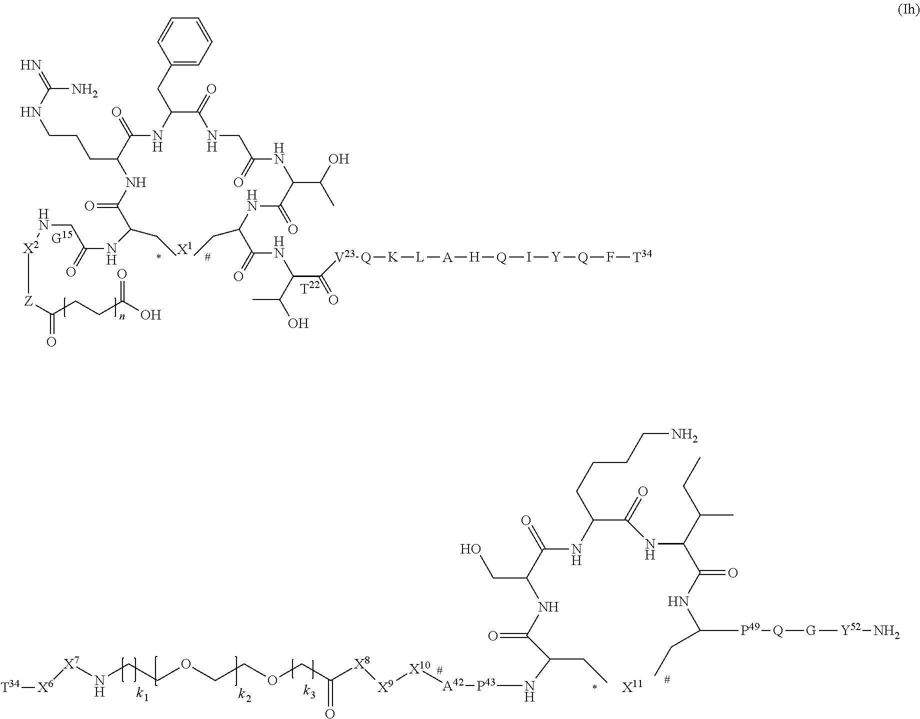
wherein n is 1 to 30 and X¹, X², Z, X⁶, X⁷, X⁸, X⁹, X¹⁰, k1, k2, k3 and X¹¹ are defined according to claim 1.

10: The compound of formulae (I), (Ia), (Ib), (Ic), (Id), (le), (If), (Ig) and/or (Ih) according to claim 1, wherein the compound is selected from

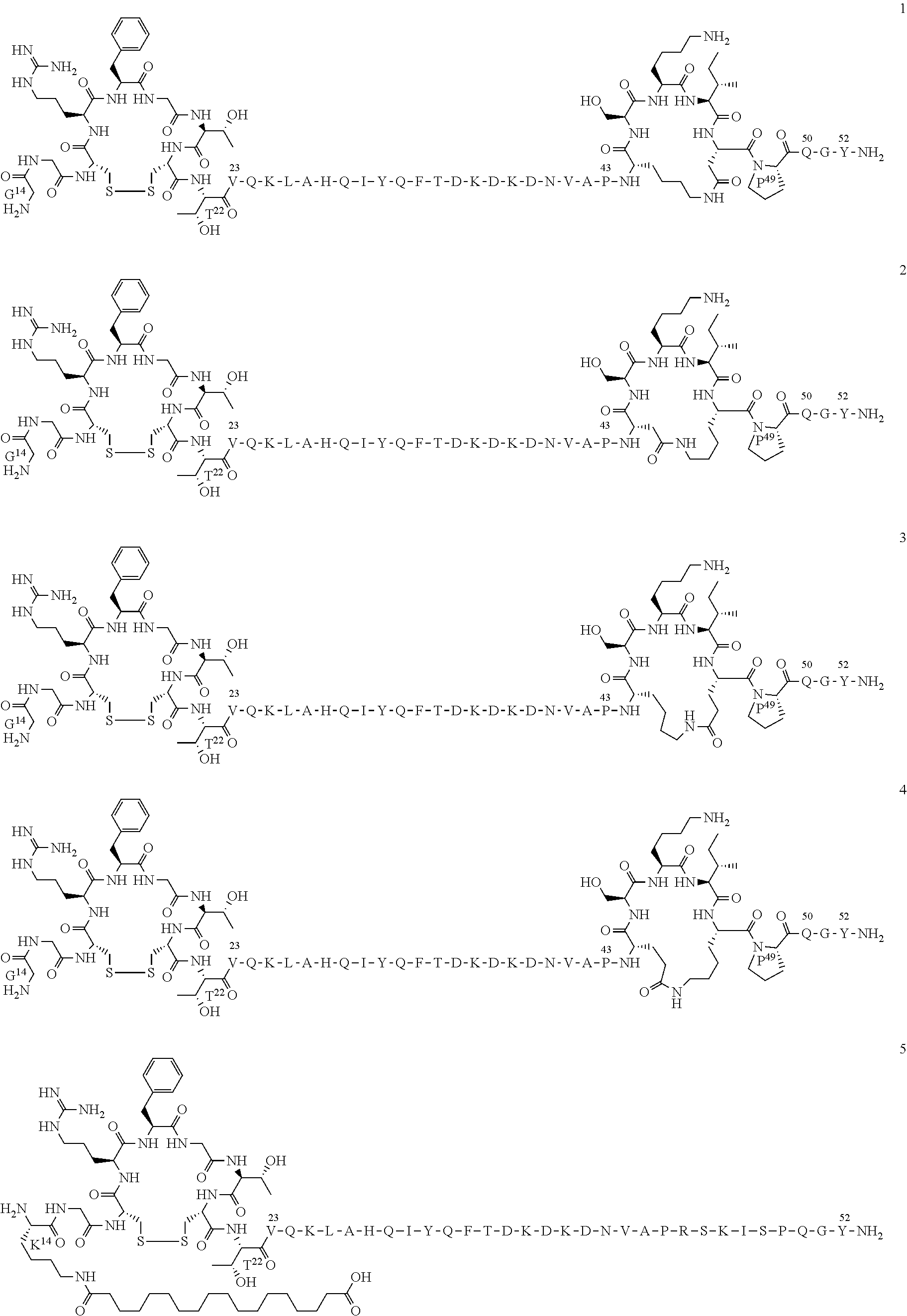

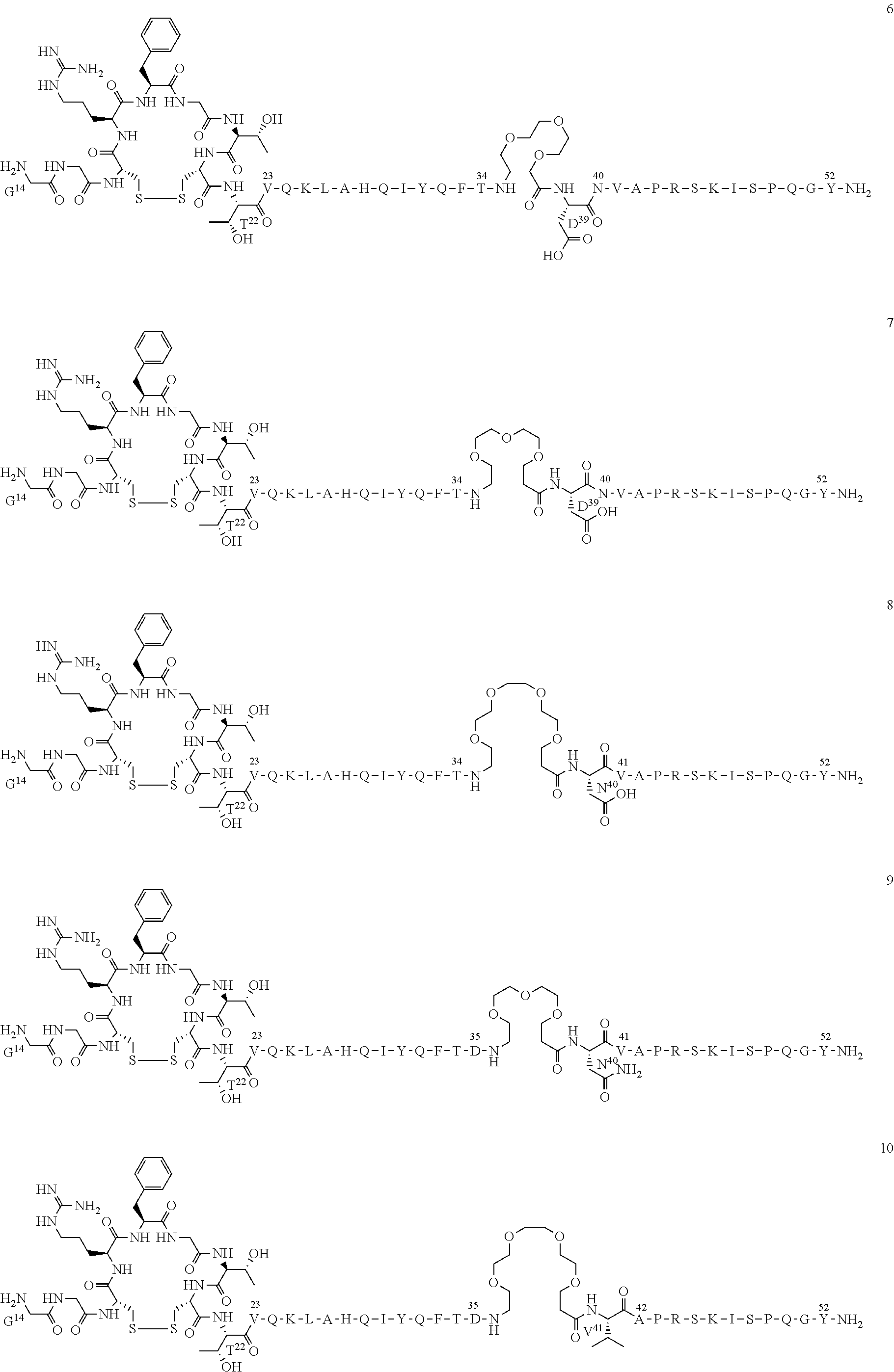

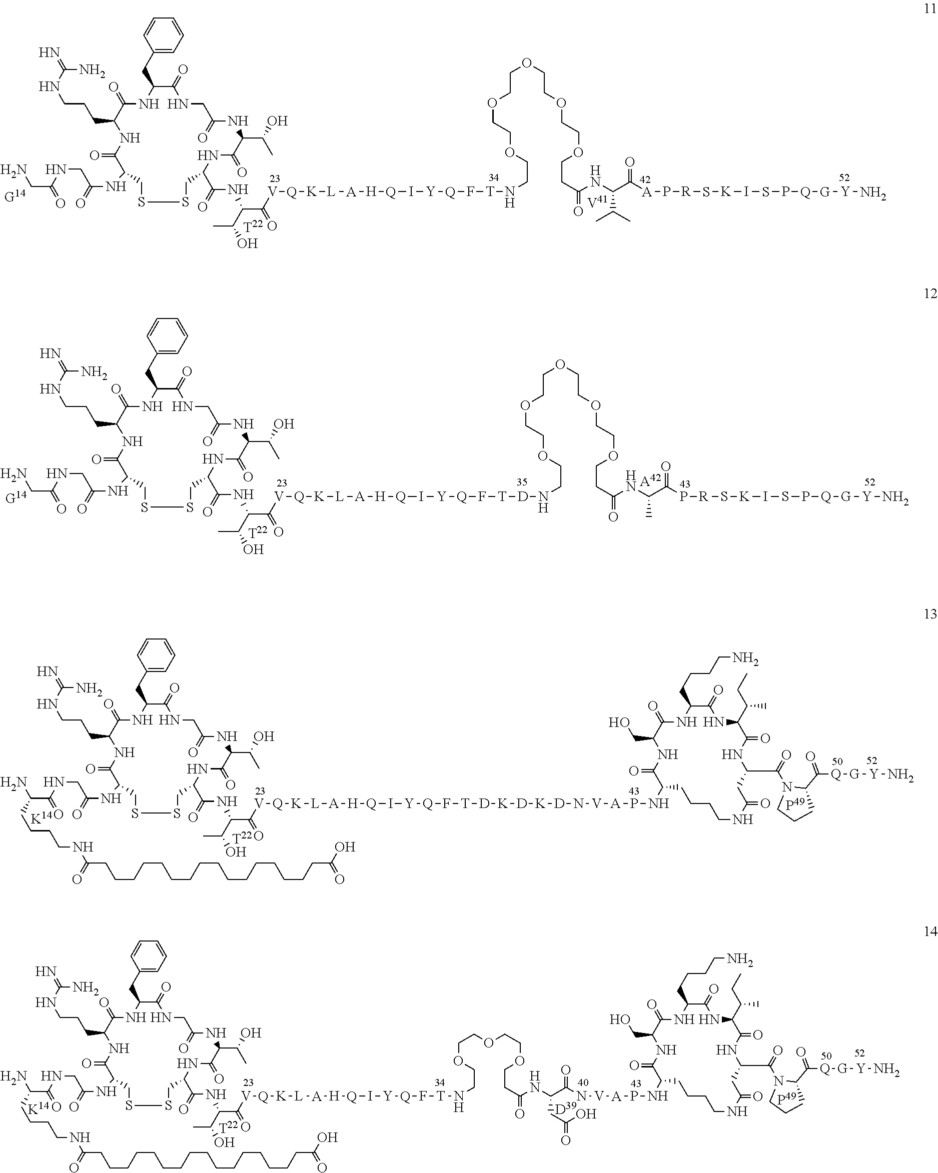

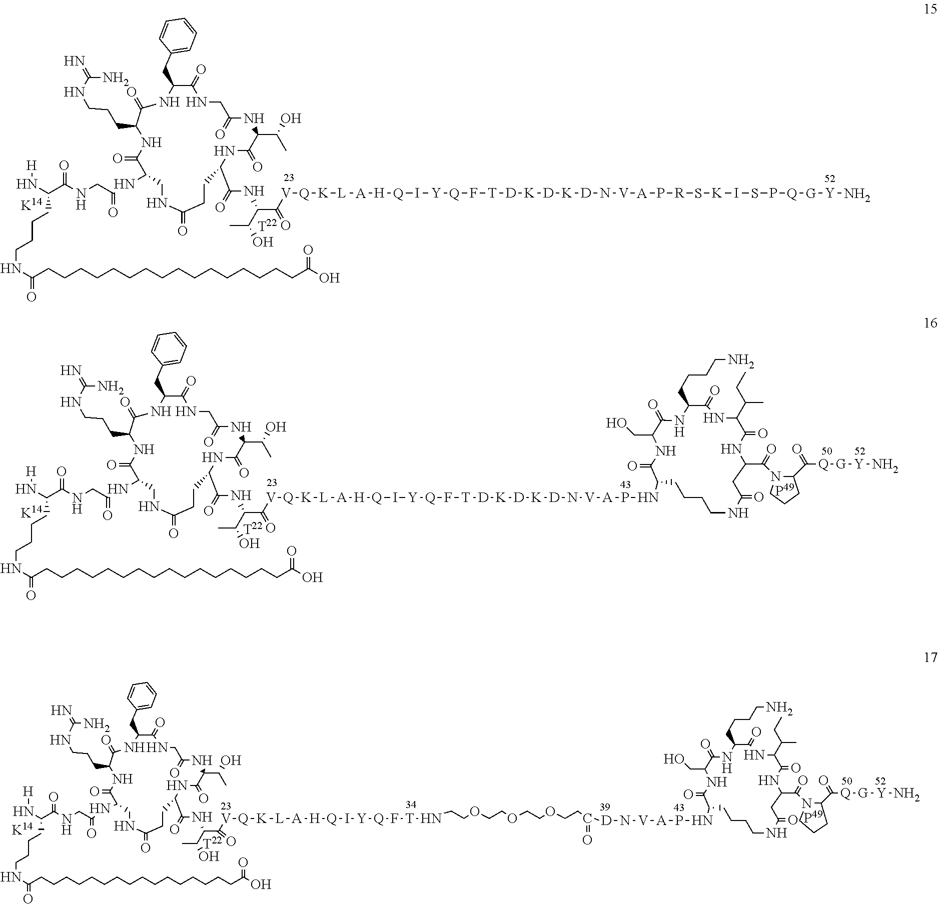

11: A method for the treatment and/or prevention of a cardiovascular, edematous and/or inflammatory disorder comprising administering the compound of claim 1 to a subject with the disorder.

12. A method for the treatment and/or prevention of a condition or disorder selected from heart failure, chronic heart failure, worsening heart failure, acute heart failure, acute decompensated heart failure, diastolic and systolic (congestive) heart failure, coronary heart disease, ischemic and/or hemorrhagic stroke, hypertension, pulmonary hypertension, peripheral arterial occlusive disease, pre-eclampsia, chronic obstructive pulmonary disease, asthma, acute and/or chronic pulmonary edema, allergic alveolitis and/or pneumonitis due to inhaled organic dust and particles of fungal, actinomycetic or other origin, and/or acute chemical bronchitis, acute and/or chronic chemical pulmonary edema, neurogenic pulmonary edema, acute and/or chronic pulmonary manifestations due to radiation, acute and/or chronic interstitial lung disorders, acute lung injury/acute respiratory distress syndrome (ALI/ARDS) in adult or child including newborn, ALI/ARDS secondary to pneumonia and sepsis, aspiration pneumonia and ALI/ARDS secondary to aspiration, ALI/ARDS secondary to smoke gas inhalation, transfusion-related acute lung injury (TRALI), ALI/ARDS and/or acute pulmonary insufficiency following surgery, trauma and/or burns, and/or ventilator induced lung injury (VILI), lung injury following meconium aspiration, pulmonary fibrosis, mountain sickness, chronic kidney diseases, glomerulonephritis, acute kidney injury, cardiorenal syndrome, lymphedema, inflammatory bowel disease, sepsis, septic shock, systemic inflammatory response syndrome (SIRS) of non-infectious origin, anaphylactic shock, inflammatory bowel disease, urticaria and/or edematous ocular disorders or ocular disorders associated with disturbed vascular function, including, age-related macular degeneration (AMD), diabetic retinopathy, in particular diabetic macula edema (DME), subretinal edema, and intraretinal edema comprising administering the compound of claim 1 to a subject with the condition or disorder.

13: A medicament comprising a compound of claim 1 in combination with an inert nontoxic pharmaceutically suitable excipient and/or in combination with a further active ingredient selected from the group consisting of ACE inhibitors, angiotensin receptor antagonists, beta-2 receptor agonists, phosphodiesterase (PDE) inhibitors, glucocorticoid receptor agonists, diuretics, recombinant angiotensin converting enzyme-2, acetylsalicylic acid, natriuretic peptides and derivatives thereof, and neprilysin inhibitors.

14: The medicament as claimed in claim 13 formulated for the treatment and/or prevention of cardiovascular, edematous and/or inflammatory disorders.

15: A method for the treatment and/or prophylaxis of cardiovascular, edematous and/or inflammatory disorders in humans or animals using an effective amount of the medicament of claim 13.


SEQUENCE LISTING (17 sequences)

SEQ ID NO: 1 - protein, 39 aa, Artificial Sequence
  [G14, (K44, D48)lac] ADM(14-52)
YGQPDIKSKPAVNDKDKDTFQYIQHALKQVTCTGFRCGG

SEQ ID NO: 2 - protein, 39 aa, Artificial Sequence
  [G14, (D44,K48)lac] ADM(14-52)
YGQPKIKSDPAVNDKDKDTFQYIQHALKQVTCTGFRCGG

SEQ ID NO: 3 - protein, 39 aa, Artificial Sequence
  [G14, (K44,E48)lac] ADM(14-52)
YGQPEIKSKPAVNDKDKDTFQYIQHALKQVTCTGFRCGG

SEQ ID NO: 4 - protein, 39 aa, Artificial Sequence
  [G14, (E44,K48)lac] ADM(14-52)
YGQPKIKSEPAVNDKDKDTFQYIQHALKQVTCTGFRCGG

SEQ ID NO: 5 - protein, 39 aa, Artificial Sequence
  [K14(ODD)] ADM(14-52)
  MISC_FEATURE (39)..(39): Lys39 is linked to palmitic acid
YGQPSIKSRPAVNDKDKDTFQYIQHALKQVTCTGFRCGK

SEQ ID NO: 6 - protein, 40 aa, Artificial Sequence
  [G14, OEG(12)[35-38]]ADM(14-52)
  MISC_FEATURE (19)..(19): Xaa is {2-[2-(2-aminoethoxy) ethoxy]ethoxy}acetic acid
YGQPSIKSRPAVNDKDKDXTFQYIQHALKQVTCTGFRCGK

SEQ ID NO: 7 - protein, 36 aa, Artificial Sequence
  [G14, OEG(13)[35-38]]ADM(14-52)
  MISC_FEATURE (15)..(15): Xaa is 3-{2-[2-(2-aminoethoxy) ethoxy]ethoxy}propanoic acid
YGQPSIKSRPAVNDXTFQYIQHALKQVTCTGFRCGG

SEQ ID NO: 8 - protein, 35 aa, Artificial Sequence
  [G14, OEG(16)[35-39]]ADM(14-52)
  MISC_FEATURE (14)..(14): Xaa is 1-amino-3,6,9,12-tetraoxapentadecan-15- oic acid
YGQPSIKSRPAVNXTFQYIQHALKQVTCTGFRCGG

SEQ ID NO: 9 - protein, 36 aa, Artificial Sequence
  [G14, OEG(13)[36-39]]ADM(14-52)
  MISC_FEATURE (14)..(14): Xaa is 3-{2-[2-(2-aminoethoxy) ethoxy]ethoxy}propanoic acid
YGQPSIKSRPAVNXDTFQYIQHALKQVTCTGFRCGG

SEQ ID NO: 10 - protein, 35 aa, Artificial Sequence
  [G14, OEG(16)[36-40]]ADM(14-52)
  MISC_FEATURE (13)..(13): Xaa is 1-amino-3,6,9,12-tetraoxapentadecan-15- oic acid
YGQPSIKSRPAVXDTFQYIQHALKQVTCTGFRCGG

SEQ ID NO: 11 - protein, 34 aa, Artificial Sequence
  [G14, OEG(19)[35-40]]ADM(14-52)
  MISC_FEATURE (13)..(13): Xaa is 1-amino-3,6,9,12,15-pentaoxaoctadecan- 18-oic acid
YGQPSIKSRPAVXTFQYIQHALKQVTCTGFRCGG

SEQ ID NO: 12 - protein, 34 aa, Artificial Sequence
  [G14, OEG(19)[36-41]]ADM(14-52)
  MISC_FEATURE (12)..(12): Xaa is 1-amino-3,6,9,12,15-pentaoxaoctadecan- 18-oic acid
YGQPSIKSRPAXDTFQYIQHALKQVTCTGFRCGG

SEQ ID NO: 13 - protein, 39 aa, Artificial Sequence
  [K14(ODD), (K44,D48)lac] ADM(14-52)
  MISC_FEATURE (39)..(39): Lys39 is linked to palmitic acid
YGQPDIKSKPAVNDKDKDTFQYIQHALKQVTCTGFRCGK

SEQ ID NO: 14 - protein, 36 aa, Artificial Sequence
  [K14(ODD), OEG(13)[35-38], (K44,D48)lac,]ADM(14-52)
  MISC_FEATURE (15)..(15): Xaa is  3-{2-[2-(2-aminoethoxy) ethoxy]ethoxy}propanoic acid
  MISC_FEATURE (36)..(36): Lys36 is linked to palmitic acid
YGQPDIKSKPAVNDXTFQYIQHALKQVTCTGFRCGK

SEQ ID NO: 15 - protein, 39 aa, Artificial Sequence
  [K14(ODD), (Dpr16,E21)lac]ADM(14-52)
  MISC_FEATURE (37)..(37): Xaa is Dpr
  MISC_FEATURE (39)..(39): Lys 39 is linked to palmitic acid
YGQPSIKSRPAVNDKDKDTFQYIQHALKQVTETGFRXGK

SEQ ID NO: 16 - protein, 39 aa, Artificial Sequence
  [K14(ODD), (Dpr16,E21)lac, (K44,D48)lac]ADM(14- 52)
  MISC_FEATURE (37)..(37): Xaa is Dpr
  MISC_FEATURE (39)..(39): Lys 39 is linked to palmitic acid
YGQPDIKSKPAVNDKDKDTFQYIQHALKQVTETGFRXGK

SEQ ID NO: 17 - protein, 36 aa, Artificial Sequence
  [K14(ODD), (Dpr16,E21)lac, OEG(13)[35-38], (K44,D48)lac]ADM(14-52)
  MISC_FEATURE (15)..(15): Xaa15 is 3-{2-[2-(2-aminoethoxy) ethoxy]ethoxy}propanoic acid
  MISC_FEATURE (34)..(34): Xaa34 is Dpr
  MISC_FEATURE (36)..(36): Lys36 is linked to palmitic acid
YGQPDIKSKPAVNDXTFQYIQHALKQVTETGFRXGK